(19) United States
(12) Patent Application Publication
(10) Pub. No.: US 2026/0356835 A1
(43) Pub. Date: Nov. 12, 2026

(54) BICYCLIC HETEROARYL COMPOUNDS

(71) Applicant: QuantX Biosciences US, Inc., PRINCETON, NJ (US)

(72) Inventors: Xianhai HUANG, Warren, NJ (US); Haifeng TANG, Metuchen, NJ (US); Adam SIMON, Northridge, CA (US); Dexi YANG, Livingston, NJ (US); Yaxiong SUN, Thousand Oaks, CA (US); Vladislav ROYTMAN, Berkeley, CA (US); Jingwu DUAN, Yardley, PA (US)

(21) Appl. No.: 19/486,711

(22) PCT Filed: May 22, 2024
(86) PCT No.: PCT/US2024/030564

§ 371 Date: Nov. 21, 2025

Related U.S. Application Data
(60) Provisional application No. 63/503,822, filed on May 23, 2023.

(51) Int. Cl. C07D 471/04; A61K 31/437; C07D 519/00

(52) U.S. Cl. CPC C07D 471/04; A61K 31/437; C07D 519/00

(57) ABSTRACT

Provided herein are novel compounds (e.g., Formula I), pharmaceutical compositions, and methods of use for treating a variety of diseases or disorders, such as cancer.

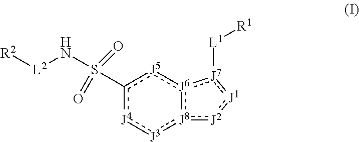

The present application claims the benefit of priority to U.S. Provisional Application No. 63/503,822, filed May 23, 2023, the entire contents of which are herein incorporated by reference.

In various embodiments, the present disclosure generally relates to novel bicyclic heteroaryl compounds, compositions comprising the same, methods of preparing and methods of using the same, e.g., for inhibiting PARG enzyme activity and/or for treating or preventing a proliferative disease described herein.

### BACKGROUND

Poly(ADP-ribose) glycohydrolase (PARG) is the main enzyme that catalyzes the degradation of poly(ADP-ribose). PARG is an endo- and exo-glycohydrolase that degrades PAR generated by the poly (ADP-ribose) polymerase (PARP) family of proteins such as PARP1, to coordinate DNA repair. Houl J. H. et al., Nature Communications, 10:5654 (2019). PARG expression was found to be upregulated in the majority of human cancers. Id. Houl et al. also showed that PARG inhibition sensitized cells to radiation-induced DNA damage, suppressed replication fork progression and impaired cancer cell survival, thus can be used in cancer treatment. Id.

Given the therapeutic potential of PARG inhibitors in cancer treatment, development of novel PARG inhibitors is needed.

### BRIEF SUMMARY

In various embodiments, the present disclosure is based in part on the discovery of certain novel bicyclic heteroaryl compounds that can be PARG inhibitors and can be used for inhibiting PARG enzyme activity in a cell (e.g., a cancer cell) and/or for treating or preventing a proliferative disease, such as cancer, as described herein.

In some embodiments, the present disclosure provides a compound of Formula I, or a pharmaceutically acceptable salt thereof:

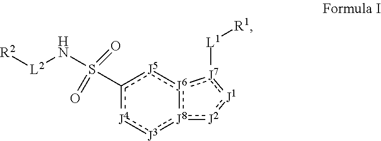

wherein the variables are defined herein. In some embodiments, the compound of Formula I can be characterized as having a structure according to a subformula selected from Formula I-1, I-2, I-3, I-1a, I-1b, I-1c, I-2a, I-2b, I-3a, I-3b, I-la-1, I-1a-2, I-2a-1, I-2a-2, I-3a-1, I-3a-2, I- 1b-1, I-1b-2, I-2b-1, I-2b-2, I-3b-1, I-3b-2, I-A, I-A1, I-A2, I-B, I-C, or I-D, as defined herein. In some embodiments, the present disclosure also provides a compound selected from Table 1 herein, or a pharmaceutically acceptable salt thereof.

Some embodiments of the present disclosure are directed to a pharmaceutical composition comprising one or more compounds of the present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, I-3b-2, I-A, I-A1, I-A2, I-B, I-C, or I-D), any of the compounds listed in Table 1 herein, or a pharmaceutically acceptable salt thereof) and optionally a pharmaceutically acceptable excipient. The pharmaceutical composition described herein can be formulated for different routes of administration, such as for oral administration or parenteral injection.

Certain embodiments of the present disclosure are directed to a method of treating a disease or disorder in which PARG activity is implicated, such as a proliferative disease herein. In some embodiments, the method comprises administering to a subject in need thereof a therapeutically effective amount of a compound of the present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-i, I-1b-2, I-2b-1, I-2b-2, I-3b-1, I-3b-2, I-A, I- A1, I-A2, I-B, I-C, or I-D), any of the compounds listed in Table 1 herein, or a pharmaceutically acceptable salt thereof) or a pharmaceutical composition herein. In some embodiments, the disease or disorder in which PARG activity is implicated is cancer.

The administering in the methods herein is not limited to any particular route of administration. For example, in some embodiments, the administering can be orally, nasally, transdermally, pulmonary, inhalationally, buccally, sublingually, intraperintoneally, subcutaneously, intramuscularly, intravenously, rectally, intrapleurally, intrathecally, or parenterally. In some embodiments, the administering is orally. In some embodiments, the administering is a parenteral injection, such as an intravenous injection.

Compounds of the present disclosure can be used as a monotherapy or in a combination therapy. In some embodiments according to the methods described herein, one or more compounds of the present disclosure can be administered as the only active ingredient(s). In some embodiments, the method herein further comprises administering to the subject an additional therapeutic agent, such as additional anticancer agents described herein.

It is to be understood that both the foregoing summary and the following detailed description are exemplary and explanatory only, and are not restrictive of the invention herein.

### DETAILED DESCRIPTION

In various embodiments, provided herein are novel compounds, pharmaceutical compositions, methods of preparation and methods of use. The compounds of the present disclosure are generally PARG inhibitors, which are also useful for treating various diseases or disorders, such as those described herein, e.g., cancer.

### Compounds

### Formula I

In some embodiments, the present disclosure provides a compound of Formula I, or a pharmaceutically acceptable salt thereof:

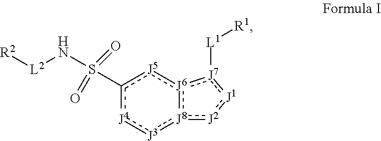

wherein:
J¹ is CR³, NR³, O, S, or N;
J² is CR⁴, NR⁴, O, S, or N;
J³ is CR⁵ or N;
J⁴ is CR⁶ or N;
J⁵ is CR⁷ or N;
J⁶ is C or N;
J⁷ is C or N;
J⁸ is C or N;
provided that the bicyclic ring containing J¹-J⁸ is a heteroaryl ring, preferably, the ring of J¹, J², J⁶, J⁷, and J⁸ has 1, 2, or 3 ring nitrogen atoms;
L¹ is null, an optionally substituted C₁₋₆ alkylene, an optionally substituted C₂₋₆ alkenylene, an optionally substituted C₂₋₆ alkynylene, an optionally substituted 3-8 membered ring, and R¹ is an optionally substituted 3-8 membered ring; or
L¹ is null or an optionally substituted 7-12 membered bicyclic ring structure; and R¹ is hydrogen, deuterium, halogen, CN, OH, NH₂, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring;
L² is an optionally substituted C₁₋₄ alkylene or an optionally substituted 3-5 membered carbocyclic or heterocyclic ring,
R² is hydrogen, deuterium, halogen, CN, OH, an optionally substituted Cia alkyl, an optionally substituted C₂₋₄ alkenyl, or an optionally substituted C₂₋₄ alkynyl, or an optionally substituted 3-5 membered carbocyclic or heterocyclic ring;
R³ is hydrogen, deuterium, halogen, CN, G¹, OG¹, NHG¹, NG¹G¹, C(O)G¹, C(O)NHG¹, or C(O)NG¹G¹;
R⁴ is hydrogen, deuterium, halogen, CN, an optionally substituted C₁₋₄ alkyl, an optionally substituted C₂₋₄ alkenyl, an optionally substituted C₂₋₄ alkynyl, an optionally substituted C₁₋₄ heteroalkyl, OG¹, NHG¹, NG¹G¹, or an optionally substituted 3-6 membered ring;
wherein G¹ at each occurrence is independently hydrogen, an optionally substituted C₃₋₄ alkyl, an optionally substituted C₂₋₄ alkenyl, an optionally substituted C₂₋₄ alkynyl, an optionally substituted C₁₋₄ heteroalkyl, or an optionally substituted 3-10 membered ring; or two G¹ together with the nitrogen atom they are both attached to are joined to form an optionally substituted 4-10 membered heterocyclic ring;
R⁵ is hydrogen, halogen, CN, OH, G², or L³-G², wherein L³ is O, NH, CO, C(O)NH, C(O)N(C₁₋₄ alkyl), SO₂, SO₂NH, SO₂N(C₁₋₄ alkyl), an optionally substituted C₁₋₄ alkylene, an optionally substituted C₁₋₄ heteroalkylene, an optionally substituted C₂₋₄ alkenylene, or an optionally substituted C₂₋₄ alkynylene, and G² is an optionally substituted 3-14 membered ring; and
R⁶ and R⁷ are each independently hydrogen, deuterium, halogen, CN, or an optionally substituted C₁₋₄ alkyl.

In some embodiments, the compound of Formula I (including any of the applicable sub-formulae as described herein) can comprise one or more asymmetric centers and/or axial chirality, and thus can exist in various stereoisomeric forms, e.g., enantiomers and/or diastereomers. In some embodiments, the compound of Formula I can exist in the form of an individual enantiomer and/or diastereomer, as applicable, or a mixture of stereoisomers, including racemic mixtures and mixtures enriched in one or more stereoisomers. In some embodiments, when applicable, the compound of Formula I (including any of the applicable sub-formulae as described herein) can exist as an individual enantiomer substantially free of the other enantiomer, such as having an enantiomeric excess (“ee”) of greater than 60%, preferably, greater than 80% ee, greater than 90% ee, greater than 95% ee, greater than 98% ee, or greater than 99% ee. In some embodiments, when applicable, the compound of Formula I (including any of the applicable sub-formulae as described herein) can also exist as a mixture of stereoisomers in any ratio, such as a racemic mixture.

In some embodiments, the compound of Formula I (including any of the applicable sub-formulae as described herein) can exist as an isotopically labeled compound, particularly, a deuterated analog, wherein one or more of the hydrogen atoms of the compound of Formula I is/are substituted with a deuterium atom with an abundance above its natural abundance, e.g., a CD₃ analog when the compound has a CH₃ group. Without wishing to be bound by theories, it is believed that in certain situations, substitution with deuterium can lead to a deuterated analog with a better pharmacokinetic profile. Deuterated analogs can be generally prepared by using commercially available deuterating reagents.

It should be apparent to those skilled in the art that in certain cases, the compound of Formula I may exist as a mixture of tautomers. The present disclosure is not limited to any specific tautomer. Rather, the present disclosure encompasses any and all of such tautomers whether or not explicitly drawn or referred to.

The combination of J¹-J⁸ in Formula I is not particularly limited, so long as the bicyclic ring containing J¹-J⁸ is a heteroaryl ring. Typically, the 5-membered ring of J¹, J², J⁶, J⁷, and J⁸ in Formula I has 1, 2, or 3 ring nitrogen atoms.

In some preferred embodiments, J⁶ is N and J⁸ is C.

In some preferred embodiments, J⁸ is N and J⁶ is C.

In some preferred embodiments, both J⁶ and J^{s} are C.

In some embodiments, J⁷ is N, and both J⁶ and J^{s} are C.

In some embodiments, J⁷ is C.

Typically, one of J¹ and J² is N, and the other of I and J² is defined herein.

In some embodiments, both J¹ and J² are N.

In some embodiments, J¹ is N and J² is CR⁴, wherein R⁴ is defined herein.

In some embodiments, J² is N and J¹ is CR³, wherein R³ is defined herein.

In some embodiments, J¹ is CR³ and J² is CR⁴, wherein R³ and R⁴ are as defined herein. For example, in some embodiments, both J¹ and J² are CH.

Typically, J³ is CR⁵, wherein R⁵ is defined herein.

In some embodiments, J³ can also be N.

Typically, neither of J⁴ and J⁵ is N.

In some embodiments, one of J⁴ and J⁵ is N.

In some embodiments, J⁴ is CR⁶, wherein R⁶ is defined herein. Typically, R⁶ is hydrogen or F.

In some embodiments, J⁵ is CR⁷, wherein R⁷ is defined herein. Typically, R⁷ is hydrogen.

In some embodiments, J⁴ and J⁵ are both CH.

In some embodiments, J⁴ is CF and J⁵ is CH.

In embodiments where J¹ is CR³, R³ is typically hydrogen, deuterium, or halogen. In some preferred embodiments, R³ is hydrogen. However, in some embodiments, R³ can be CN, G¹, OG¹, NHG¹, NG¹G¹, C(O)G¹, C(O)NHG¹, or C(O)NG¹G¹, wherein G¹ is defined herein. For example, in some embodiments, R³ can be CN, G¹, C(O)G¹, C(O)NHG¹, or C(O)NG¹G¹, wherein G¹ is defined herein. In some embodiments, R³ can be G. In some embodiments, G¹ at each occurrence can be independently selected from C₁₋₄ alkyl optionally substituted with F, C₁₋₄ heteroalkyl optionally substituted with F, or a 3-10 membered ring selected from C₃₋₁₀ carbocyclic ring, 4-10 membered heterocyclic ring, or 5-10 membered heteroaryl, or phenyl, wherein the 3-10 membered ring is optionally substituted with 1-3 substituents independently selected from halogen (e.g., F), CN, OH, C₁₋₄ alkyl optionally substituted with F, C₁₋₄ heteroalkyl optionally substituted with F, or a 3-4 membered ring such as cyclopropyl or cyclobutyl. In some embodiments, R³ can be G¹, and G¹ is C₁₋₄ alkyl optionally substituted with F or C₁₋₄ heteroalkyl optionally substituted with F. In some embodiments, R³ can be G¹, and G¹ is a 3-6 membered ring selected from C₃₋₆ carbocyclic ring, 4-6 membered heterocyclic ring, or a 5 or 6 membered heteroaryl, wherein the 3-6 membered ring is optionally substituted with 1-3 substituents independently selected from halogen (e.g., F), CN, OH, C₁₋₄ alkyl optionally substituted with F, C₁₋₄ heteroalkyl optionally substituted with F, or a 3-4 membered ring such as cyclopropyl or cyclobutyl.

In embodiments where J² is CR⁴, R⁴ is typically hydrogen, deuterium, or halogen. For example, in some embodiments, J² is CH. In some embodiments, J² is CF. In some embodiments, J² is C—Cl. However, in some embodiments, R⁴ can also be CN, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring. For example, in some embodiments, R⁴ can be CN, C₁₋₄ alkyl optionally substituted with F, C₁₋₄ heteroalkyl optionally substituted with F, or a 3-6 membered ring selected from C₃₋₆ carbocyclic ring, 4-6 membered heterocyclic ring, or a 5 or 6 membered heteroaryl, wherein the 3-6 membered ring is optionally substituted with 1-3 substituents independently selected from halogen (e.g., F), CN, OH, C₁₋₄ alkyl optionally substituted with F, C₁₋₄ heteroalkyl optionally substituted with F, or a 3-4 membered ring such as cyclopropyl or cyclobutyl. In some embodiments, R⁴ can be an optionally substituted C₂₋₄ alkynyl. In some embodiments, R⁴ can be OG¹, NHG¹, or NG¹G¹, wherein G¹ is defined herein.

In some preferred embodiments, the compound of Formula I has a structure according to Formula I-1:

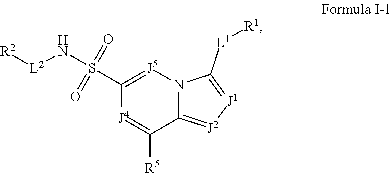

wherein L¹, L², R¹, R², R⁵, J¹, J², J⁴, and J⁵ include any of those described herein in any combination.

In some embodiments, J in Formula I-1 is N, and the compound can have a structure according to Formula I-1a:

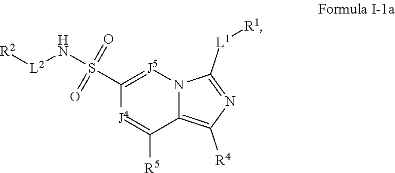

wherein L¹, L², R¹, R², R⁴, R⁵, J⁴, and J⁵ include any of those described herein in any combination. In some embodiments according to Formula I-1a, both J⁴ and J⁵ are CH. In some embodiments according to Formula I-1a, J⁴ is CF and J⁵ is CH. For example, in some embodiments, the compound of Formula I-1a can have a structure according to Formula I-1a-1 or I-1a-2:

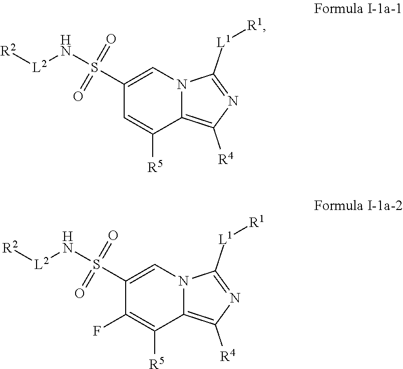

wherein L¹, L², R¹, R², R⁴, and R include any of those described herein in any combination. In some embodiments, R⁴ in Formula I-1a-I or I-1a-2 is hydrogen or halogen, such as hydrogen, F, or Cl.

In some embodiments, J² in Formula I-1 is N. For example, in some embodiments, the compound can have a structure according to Formula I-1b:

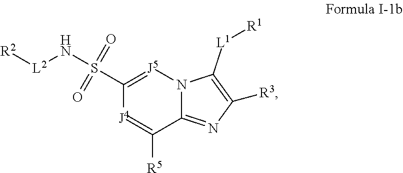

wherein L¹, L², R¹, R², R³, R⁵, J⁴, and J⁵ include any of those described herein in any combination. In some embodiments according to Formula I-1b, both J⁴ and J⁵ are CH. In some embodiments according to Formula I-1b, J⁴ is CF and J⁵ is CH. For example, in some embodiments, the compound of Formula I-1b can have a structure according to Formula I-1b-1 or I-1b-2:

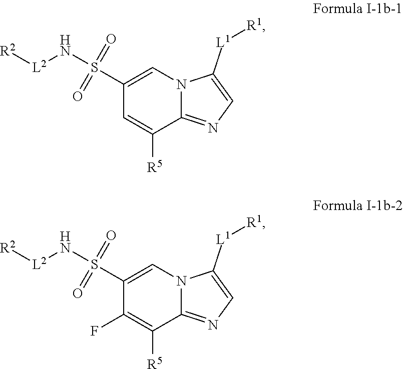

wherein L¹, L², R¹, R², and R⁵ include any of those described herein in any combination.

In some embodiments, both J¹ and J² in Formula I-1 are N, and the compound can have a structure according to Formula I-1c:

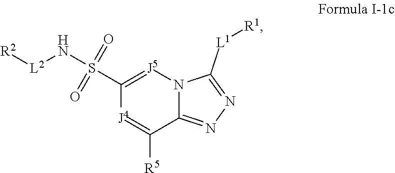

wherein L¹, L², R¹, R², R⁵, J⁴, and J⁵ include any of those described herein in any combination. In some preferred embodiments according to Formula I-1c, both J⁴ and J⁵ are CH. In some embodiments according to Formula I-1c, J⁴ is CF and J⁵ is CH.

In some preferred embodiments, the compound of Formula I has a structure according to Formula I-2:

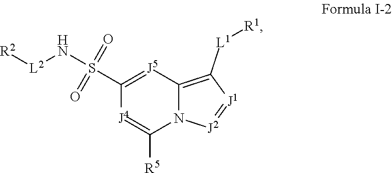

wherein L¹, L², R¹, R², R⁵, J¹, J², J⁴, and J⁵ include any of those described herein in any combination.

In some embodiments, J¹ in Formula I-2 is N, and the compound can have a structure according to Formula I-2a:

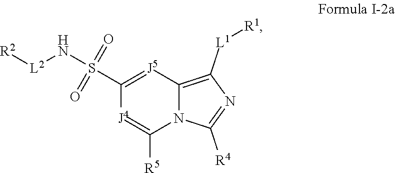

wherein L¹, L², R¹, R², R⁴, R⁵, J⁴, and J⁵ include any of those described herein in any combination. In some embodiments according to Formula I-2a, both J⁴ and J⁵ are CH. In some embodiments according to Formula I-2a, J⁴ is CF and J⁵ is CH. For example, in some embodiments, the compound of Formula I-2a can have a structure according to Formula I-2a-1 or I-2a-2:

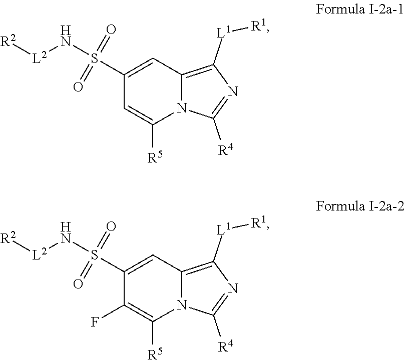

wherein L¹, L², R¹, R², and R⁵ include any of those described herein in any combination. In some embodiments, R⁴ in Formula I-2a-1 or I-2a-2 is hydrogen or halogen, preferably, R⁴ is hydrogen, F, or Cl.

In some embodiments, J² in Formula I-2 is N. For example, in some embodiments, the compound can have a structure according to Formula I-2b:

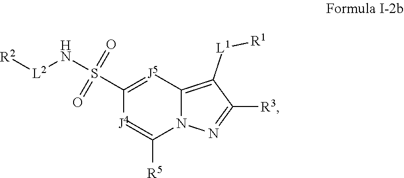

wherein L¹, L², R¹, R², R³, R⁵, J⁴, and J⁵ include any of those described herein in any combination. In some embodiments according to Formula I-2b, both J⁴ and J⁵ are CH. In some embodiments according to Formula I-2b, J⁴ is CF and J⁵ is CH. For example, in some embodiments, the compound of Formula I-2b can have a structure according to Formula I-2b-1 or I-2b-2:

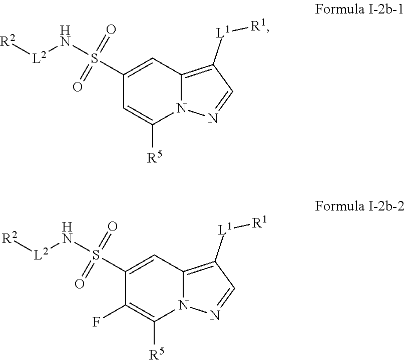

wherein L¹, L², R¹, R², and R⁵ include any of those described herein in any combination.

In some preferred embodiments, the compound of Formula I has a structure according to Formula 1-3:

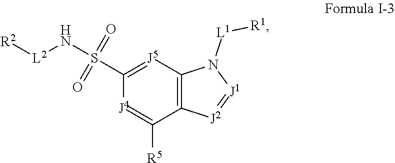

wherein L¹, L², R¹, R², R⁵, J¹, J², J⁴, and J⁵ include any of those described herein in any combination.

In some embodiments, J¹ in Formula I-3 is N, and the compound can have a structure according to Formula 1-3a:

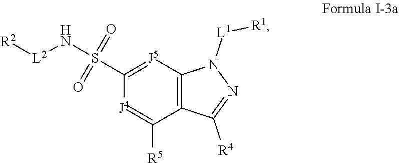

wherein L¹, L², R¹, R², R⁴, R⁵, J⁴, and J⁵ include any of those described herein in any combination. In some embodiments according to Formula I-3a, both J⁴ and J⁵ are CH. In some embodiments according to Formula I-3a, J⁴ is CF and J⁵ is CH. For example, in some embodiments, the compound of Formula I-3a can have a structure according to Formula I-3a-1 or I-3a-2:

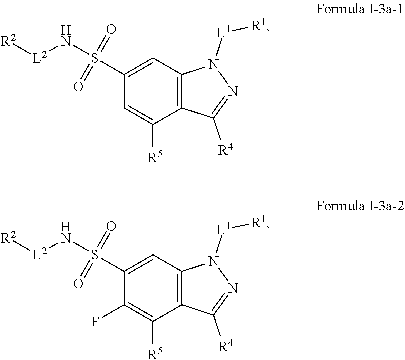

wherein L¹, L², R¹, R², and R⁵ include any of those described herein in any combination. In some embodiments, R⁴ in Formula I-3a-1 or I-3a-2 is hydrogen or halogen, preferably, R⁴ is hydrogen, F, or Cl.

In some embodiments, J² in Formula I-3 is N. For example, in some embodiments, the compound can have a structure according to Formula I-3b:

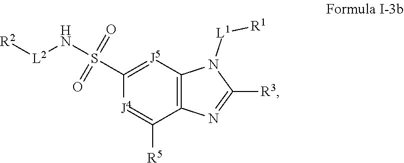

wherein L¹, L², R¹, R², R³, R⁵, J⁴, and J⁵ include any of those described herein in any combination. In some embodiments according to Formula I-3b, both J⁴ and J⁵ are CH. In some embodiments according to Formula I-3a, J⁴ is CF and J⁵ is CH. For example, in some embodiments, the compound of Formula I-3b can have a structure according to Formula I-3b-1 or I-3b-2:

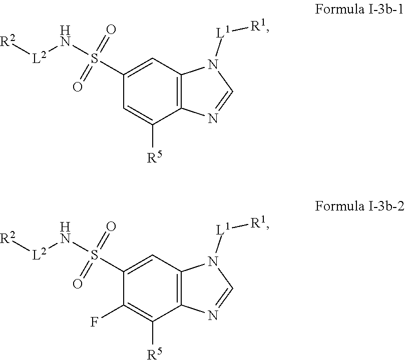

wherein L¹, L², R¹, R², and R⁵ include any of those described herein in any combination.

In some embodiments, in Formula I (e.g., any of the applicable subformulae), L¹ is null, an optionally substituted C₁₋₆ alkylene, an optionally substituted C₂₋₆ alkenylene, an optionally substituted C₂₋₆ alkynylene, an optionally substituted 3-8 membered ring, and R¹ is an optionally substituted 3-8 membered ring, such as an optionally substituted 3-6 membered ring. When substituted, the optionally substituted C₁₋₆ alkylene, C₂₋₆ alkenylene, C₂₋₆ alkynylene, 3-8 membered ring, or 3-6 membered ring can be preferably substituted with 1-3 substituents, each independently selected from (i) halogen (e.g., F), (ii) OH, (iii) CN, (iv) C₁₋₄ alkyl optionally substituted with F, (v) C₂₋₄ alkenyl optionally substituted with F, (vi) C₂₋₄ alkynyl optionally substituted with F, (vii) a 3-5 membered ring having 0-3 heteroatoms, such as cyclopropyl, optionally substituted with 1-3 substituents each independently oxo, CN, F, OH, C₁₋₂ alkyl optionally substituted with 1-3 F, or C₁₋₂ heteroalkyl optionally substituted with 1-3 F, and (viii) C₁₋₄ heteroalkyl having one or two heteroatoms independently N, S, or 0, wherein the S atom may be optionally oxidized, and the C₁₋₄ heteroalkyl is optionally substituted with F.

In some embodiments, in Formula I (e.g., any of the applicable subformulae), L¹ is an optionally substituted 3-8 membered ring, which contains 0-4 ring heteroatoms independently selected from N, O, and S, and R¹ is an optionally substituted 3-6 membered non-aromatic ring, such as a 3-4 membered cycloalkyl or a 4-5 membered heterocyclic ring. Typically, the 3-8 membered ring is a 5 or 6 membered heteroaryl ring having 1-3 ring heteroatoms.

For example, in some embodiments, in Formula I (e.g., any of the applicable subformulae), L¹ can be an optionally substituted 5-membered heteroaryl ring, such as an optionally substituted ring selected from thiazole, oxazole, imidazole, oxadiazole, or thiadiazole, and R¹ is an optionally substituted 3-6 membered non-aromatic ring, such as a 3-4 membered cycloalkyl or a 4-5 membered heterocyclic ring. In some embodiments, L¹ is

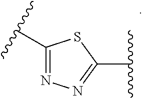 [SMILES: *c1nnc(*)s1]

In some embodiments, L¹ is

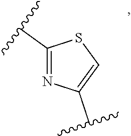 [SMILES: *c1csc(*)n1]

wherein the attaching point meta to the S atom is attached to R¹.

In some embodiments, in Formula I (e.g., any of the applicable subformulae), L¹ can be an optionally substituted 6-membered heteroaryl ring, such as an optionally substituted 6-membered heteroarylene having 1 or 2 ring nitrogens, e.g., an optionally substituted pyridylene or optionally substituted pyridazylene, and R¹ is an optionally substituted 3-6 membered non-aromatic ring, such as a 3-4 membered cycloalkyl or a 4-5 membered heterocyclic ring. For example, in some embodiments, L¹ is

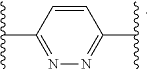 [SMILES: *c1ccc(*)nn1]

In some embodiments, L¹ is

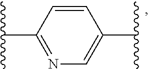 [SMILES: *c1ccc(*)nc1]

typically, the attaching point meta to the N atom is attached to R¹.

In some embodiments, in Formula I (e.g., any of the applicable subformulae), L¹ can also be an optionally substituted phenyl. In some embodiments, L¹ can be an optionally substituted 3-8 membered carbocyclic or heterocyclic ring.

In some embodiments, the inventors herein discovered that having a small 3-4 membered cyclic structure as R¹ can be beneficial for inhibiting PARG activity. In some embodiments, in Formula I (e.g., any of the applicable subformulae), L¹ can be any of those described herein, and R¹ can be a 3-4 membered ring, such as cyclopropyl or cyclobutyl, which is optionally substituted. When substituted, the 3-4 membered ring is typically substituted with 1-3 substituents each independently selected from deuterium, F, OH, CN, C₁₋₄ alkyl, C₂₋₄ alkenyl, C₂₋₄ alkynyl, and C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH. In some preferred embodiments, R¹ can be an optionally substituted cyclopropyl. Preferably, when substituted, the cyclopropyl is substituted with 1-3 substituents each independently selected from F, OH, CN, or C₁₋₂ alkyl optionally substituted with 1-3 F, for example, R¹ can be

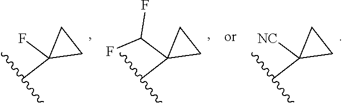

In some preferred embodiments, the compound of Formula I can be characterized as having a structure according to Formula 1-A:

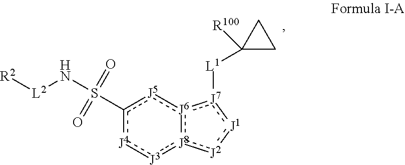

wherein:
R¹⁰⁰ is hydrogen, F, OH, CN, an optionally substituted alkyl, such as a C₁₋₄ alkyl optionally substituted with deuterium or F (e.g., CH₃, CD₃, CH₂F, CHF₂, etc.), or an optionally substituted heteroalkyl, such as a C₁₋₄ heteroalkyl optionally substituted with deuterium or F; preferably, R¹⁰⁰ is hydrogen, F, OH, CN, or methyl optionally substituted with deuterium or F (e.g., CH₃, CD₃, CH₂F, CHF₂, etc.), and
J¹, J², J³, J⁴, J⁵, J⁶, J⁷, J⁸, L¹, L², and R² include any of those described herein in any combination. Typically, in Formula I-A, L¹ is an optionally substituted 5-membered heteroarylene having 1-3 ring heteroatoms independently selected from S, N, and O, for example, an optionally substituted ring selected from thiazole, oxazole, imidazole, oxadiazole, or thiadiazole. In some embodiments, L¹ is

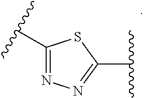 [SMILES: *c1nnc(*)s1]

In some embodiments, L¹ is

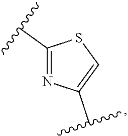 [SMILES: *c1csc(*)n1]

wherein the attaching point meta to the S atom is attached to the cyclopropyl ring in Formula I-A. In some embodiments, in Formula I-A, L¹ can be an optionally substituted 6-membered heteroarylene having 1 or 2 ring nitrogens, such as an optionally substituted pyridylene or optionally substituted pyridazylene, for example,

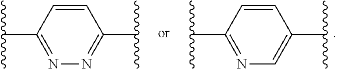 [SMILES: *c1ccc(*)nn1 ; *c1ccc(*)nc1]

Preferably, R¹⁰⁰ is F, CN, CH₃, CD₃, CH₂F, or CHF₂. In some embodiments according to Formula I-A, R¹⁰⁰ is F. In some embodiments according to Formula I-A, R¹⁰⁰ is CN. In some embodiments according to Formula I-A, R¹⁰⁰ is CHF₂. In some embodiments according to Formula I-A, the bicyclic heteroaryl moiety

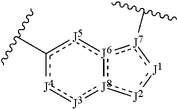

can be a bicyclic heteroaryl moiety as defined in Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, or I-3b-2. For clarity and to illustrate, in embodiments where

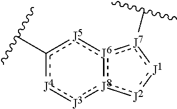

is said to be a bicyclic heteroaryl moiety as defined in Formula I-1a-1, then it should be understood that the moiety of

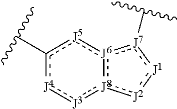

has a structure of

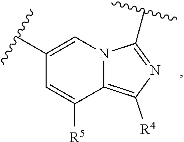

wherein R⁴ and R⁵ are defined herein, including those specifically defined for Formula I-1a-1. Similar expressions in connection with other formulae herein should be understood similarly.

In some preferred embodiments, the compound according to Formula I-A can have a structure of Formula I-A1 or I-A2:

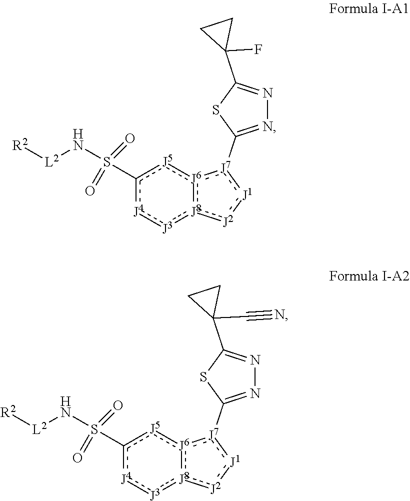

wherein J¹, J², J³, J⁴, J⁵, J⁶, J⁷, J⁸, L², and R² include any of those described herein in any combination. In some embodiments according to Formula I-A1 or I-A2, the bicyclic heteroaryl moiety

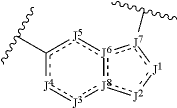

is a bicyclic heteroaryl moiety as defined in Formula I-1, I-2, I-3, I-1a, I-1b, I-1c, I-2a, I-2b, I-3a, I-3b, I-1a-1, I-1a-2, I-2a-1, I-2a-2, I-3a-1, I-3a- 2, I-1b-1, I-1b-2, I-2b-1, I-2b-2, I-3b-1, or I-3b-2.

In some embodiments, in Formula I (e.g, I-A), L¹ can also be an optionally substituted C₁₋₄ alkylene, an optionally substituted C₂₋₄ alkenylene, or an optionally substituted C₂₋₄ alkynylene, and R¹ is an optionally substituted 3-8 membered ring (e.g., as described herein).

In some embodiments, in Formula I (e.g., any of the applicable subformulae), L¹ can be null or an optionally substituted 7-12 membered bicyclic ring structure; and R¹ is hydrogen, deuterium, halogen, CN, OH, NH₂, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring. When substituted, the optionally substituted 7-12 membered bicyclic ring structure, C₁₋₄ alkyl, C₂₋₄ alkenyl, C₂₋₄ alkynyl, C₁₋₄ heteroalkyl, or 3-6 membered ring can be preferably substituted with 1-3 substituents, each independently selected from (i) halogen (e.g., F), (ii) OH, (iii) CN, (iv) C₁₋₄ alkyl optionally substituted with F, (v) C₂₋₄ alkenyl optionally substituted with F, (vi) C₂₋₄ alkynyl optionally substituted with F, (vii) a 3-5 membered ring having 0-3 heteroatoms, such as cyclopropyl, optionally substituted with 1-3 substituents each independently oxo, CN, F, OH, C₁₋₂ alkyl optionally substituted with 1-3 F, or C₁₋₂ heteroalkyl optionally substituted with 1-3 F, and (viii) C₁₋₄ heteroalkyl having one or two heteroatoms independently N, S, or O, wherein the S atom may be optionally oxidized, and the C₁₋₄ heteroalkyl is optionally substituted with F. Typically, in such embodiments, L¹ is an optionally substituted 8-10 membered fused bicyclic ring structure, such as a fused bicyclic heterocyclic or heteroaryl ring, and R¹ is hydrogen, deuterium, halogen (preferably, F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH. In some preferred embodiments, R¹ is hydrogen, F, OH, CN, or C₁₋₄ alkyl optionally substituted with 1-3 substituents independently selected from F and OH.

In some embodiments, L¹ is an optionally substituted 8-10 membered fused bicyclic ring structure, which has a first and second constituent ring, wherein the first constituent ring is a 5-membered heteroaryl ring having 1-3 ring heteroatoms independently selected from N, O, and S, and the second constituent ring is an aryl, heteroaryl, carbocyclic, or heterocyclic ring. In some embodiments, L¹ is an optionally substituted 8 or 9 membered fused bicyclic heteroaryl having a first and second constituent ring, wherein the first constituent ring is a 5-membered heteroaryl ring having 1-3 ring heteroatoms independently selected from N, O, and S, and the second constituent ring is phenyl, a 5-membered heteroaryl, or a 6-membered heteroaryl. In some embodiments, L¹ is an optionally substituted 8 or 9 membered fused bicyclic heterocyclyl ring having a first and second constituent ring, wherein the first constituent ring is a 5-membered heteroaryl ring having 1-3 ring heteroatoms independently selected from N, O, and S, and the second constituent ring is a 5-6 membered carbocyclic or heterocyclic ring. In some embodiments, the first constituent ring is a thiazole ring,

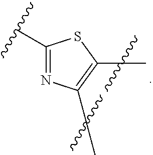 [SMILES: *c1nc(*)c(*)s1]

In some embodiments, the second constituent ring is a 5 or 6 membered heteroaryl, such as a pyridine ring, e.g.,

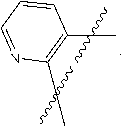 [SMILES: *c1cccnc1*]

In some embodiments, the second constituent ring is a 5 or 6 membered heterocyclyl, typically having 1 or 2 ring heteroatoms independently N, O, and S, wherein the S atom is optionally oxidized, for example, the second constituent ring can be

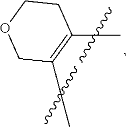 [SMILES: *C1=C(*)COCC1]

which is optionally substituted. In these embodiments, L¹ typically attaches to J⁷ through a ring atom of the first constituent ring and attaches to R¹ through a ring atom of the second constituent ring. For example, in some embodiments, L¹ can be a fused bicyclic heteroaryl having a structure of

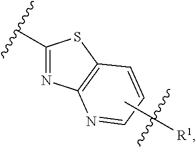

wherein the thiazole ring (first constituent ring) attaches to J⁷ through the carbon atom as shown, and R¹ is attached to a ring carbon atom of the pyridine ring (second constituent ring).

In embodiments where L¹ is an optionally substituted 8-10 membered fused bicyclic ring structure described herein, R¹ can be preferably hydrogen, deuterium, halogen (preferably, F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH. In some preferred embodiments, R¹ is hydrogen, F, OH, CN, or C₁₋₄ alkyl optionally substituted with 1-3 substituents independently selected from F and OH.

In some more specific embodiments, the compound of Formula I can be characterized as having a structure according to Formula I-B:

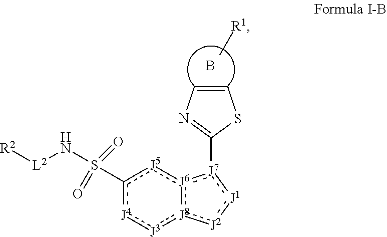

wherein:
Ring B is an optionally substituted 5 or 6 membered heterocyclyl or heteroaryl ring having 1-3 ring heteroatoms independently N, O, or S;
R¹ is hydrogen, deuterium, halogen, CN, OH, NH₂, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring; and
J¹, J², J³, J⁴, J⁵, J⁶, J⁷, J⁸, L², and R² include any of those described herein in any combination. When substituted, the 5 or 6 membered heterocyclyl or heteroaryl ring of Ring B is preferably substituted with 1-3 substituents independently selected from (i) halogen (e.g., F), (ii) OH, (iii) CN, (iv) C₁₋₄ alkyl optionally substituted with F, (v) C₂₋₄ alkenyl optionally substituted with F, (vi) C₂₋₄ alkynyl optionally substituted with F, (vii) oxo (as valency permits), (viii) C₁₋₄ heteroalkyl having one or two heteroatoms independently N, S, or O, wherein the S atom may be optionally oxidized, and the C₁₋₄ heteroalkyl is optionally substituted with F, and (ix) a 3-5 membered ring having 0-3 heteroatoms, such as cyclopropyl, optionally substituted with 1-3 substituents each independently oxo, CN, F, OH, C₁₋₂ alkyl optionally substituted with 1-3 F, or C₁₋₂ heteroalkyl optionally substituted with 1-3 F. In some embodiments, ring B is an optionally substituted 6-membered heteroaryl, such as an optionally substituted pyridine. To be clear, in Formula I-B, ring B is always connected to R¹, thus, when it is said that ring B is substituted, it should be understood that ring B has one or more substituents at the remaining positions. In some embodiments according to Formula I-B, R¹ is hydrogen, deuterium, halogen (preferably, F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH. In some embodiments according to Formula I-B, the bicyclic heteroaryl moiety

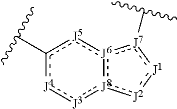

is a bicyclic heteroaryl moiety as defined in Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, or I-3b-2.

In some embodiments, in Formula I (e.g., any of the applicable subformulae), L¹ can also be null, i.e., R¹ is directly attached to J⁷ of the bicyclic heteroaryl. For example, in some embodiments, the compound of Formula I can be characterized as having a structure according to Formula I-C:

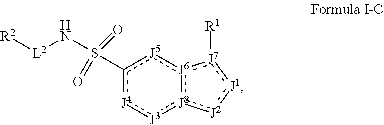

wherein J¹, J², J³, J⁴, J⁵, J⁶, J⁷, J⁸, R¹, L², and R² include any of those described herein in any combination. Typically, in Formula I-C, R¹ is an optionally substituted 5-membered heteroaryl having 1-3 ring heteroatoms independently selected from S, N, and O, for example, an optionally substituted ring selected from thiazole, oxazole, imidazole, oxadiazole, or thiadiazole. In some embodiments, in Formula I-C, R¹ can be an optionally substituted 6-membered heteroaryl having 1 or 2 ring nitrogens, such as an optionally substituted pyridyl or optionally substituted pyridazyl. When substituted, the optionally substituted 5-membered heteroaryl or optionally substituted 6-membered heteroaryl can be preferably substituted with 1-3 substituents, each independently selected from (i) halogen (e.g., F), (ii) OH, (iii) CN, (iv) C₁₋₄ alkyl optionally substituted with F, (v) C₂₋₄ alkenyl optionally substituted with F, (vi) C₂₋₄ alkynyl optionally substituted with F, (vii) a 3-5 membered ring having 0-3 heteroatoms, such as cyclopropyl, optionally substituted with 1-3 substituents each independently oxo, CN, F, OH, C₁₋₂ alkyl optionally substituted with 1-3 F, or C₁₋₂ heteroalkyl optionally substituted with 1-3 F, and (viii) C₁₋₄ heteroalkyl having one or two heteroatoms independently N, S, or O, wherein the S atom may be optionally oxidized, and the C₁₋₄ heteroalkyl is optionally substituted with F. For example, in some embodiments, in Formula I-C, R¹ can be a thiazole, oxazole, imidazole, oxadiazole, or thiadiazole, each of which is optionally substituted with a C₁₋₂ alkyl optionally substituted with F, such as CF₂H, CH₃, CF₃, etc. For example, in some embodiments, in Formula I-C, R¹ can be

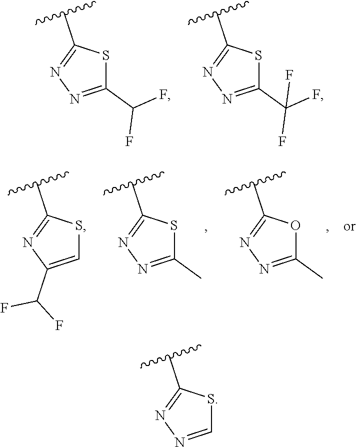

In some embodiments, in Formula I-C, R can be an optionally substituted C₁₋₄ alkyl, an optionally substituted C₂₋₄ alkenyl, or an optionally substituted C₂₋₄ alkynyl. In some embodiments according to Formula I-C, the bicyclic heteroaryl moiety

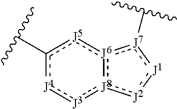

is a bicyclic heteroaryl moiety as defined in Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, or I-3b-2.

Exemplified L¹-R¹ suitable for Formula I (e.g., any of the applicable subformulae) include any of those respective groups as shown in the specific compounds listed in Table 1 herein.

In some specific embodiments, unless otherwise specified or contrary from context, L¹-R¹ in Formula I (e.g., any of the applicable subformulae) can be

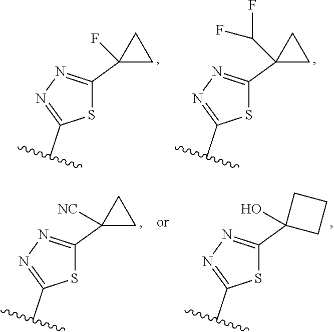 [SMILES: *c1nnc(C2(F)CC2)s1 ; *c1nnc(C2(C(F)F)CC2)s1 ; *c1nnc(C2(C#N)CC2)s1 ; *c1nnc(C2(O)CCC2)s1]

preferably,

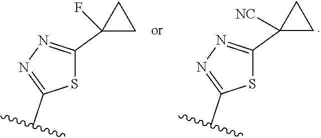 [SMILES: *c1nnc(C2(F)CC2)s1 ; *c1nnc(C2(C#N)CC2)s1]

In some specific embodiments, unless otherwise specified or contrary from context, L¹-R¹ in Formula I (e.g., any of the applicable subformulae) can be

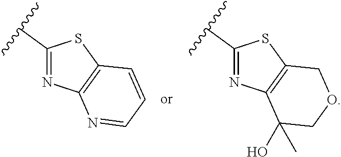 [SMILES: *c1nc2ncccc2s1 ; *c1nc2c(s1)COCC2(C)O]

In some specific embodiments, unless otherwise specified or contrary from context, L¹-R¹ in Formula I (e.g., any of the applicable subformulae) can be

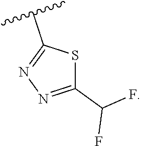 [SMILES: *c1nnc(C(F)F)s1]

In Formula I (e.g., any of the applicable subformulae), L² is typically an optionally substituted 3-5 membered carbocyclic or heterocyclic ring, such as an optionally substituted cyclopropyl ring or a fused or bridged C₄₋₅ carbocyclic ring. In some embodiments, L² can be an optionally substituted cyclopropyl ring. In some embodiments, L² can be

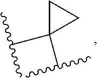 [SMILES: *C1(*)CC1]

optionally substituted with F, such as

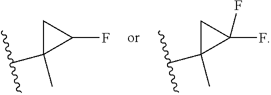

In some embodiments, L² can be

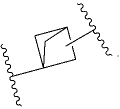

In some embodiments, L² can also be an optionally substituted C₁₋₄ alkylene, which can be straight chained or branched C₁₋₄ alkylene, such as C(CH₃)₂.

Various groups are suitable for R². Typically, R² can be hydrogen, F, C₁₋₂ alkyl optionally substituted with F (e.g., CH₃, CHF₂, or CF₃), CN, cyclopropyl, or C₂₋₃ alkynyl optionally substituted with F, such as

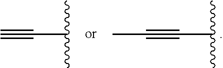

Exemplified L²-R² suitable for Formula I (e.g., any of the applicable subformulae) include any of those respective groups as shown in the specific compounds listed in Table 1 herein.

For example, in some embodiments, unless otherwise specified or contrary from context, in Formula I (e.g., any of the applicable subformulae), L²-R² can be

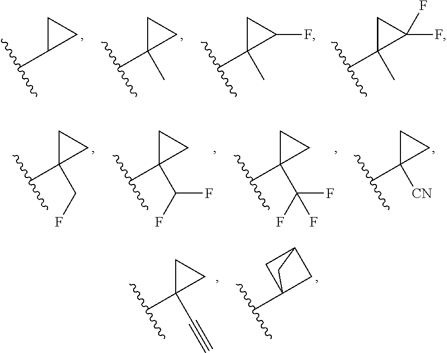

etc.

In some preferred embodiments, L² is a substituted cyclopropylene, and the compound of Formula I can be characterized as having a structure according to Formula I-D,

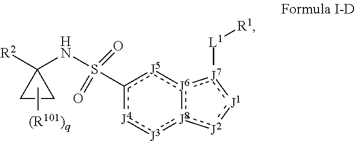

wherein:
q is 1 or 2;
R¹⁰¹ is F or methyl optionally substituted with F; and
J¹, J², J³, J⁴, J⁵, J⁶, J⁷, J⁸, L¹, R¹, and R² are defined herein. Typically, in such embodiments, R¹⁰¹ is F. In some embodiments, q is 1. In some embodiments, the moiety of

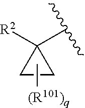

can have a stereochemistry selected from

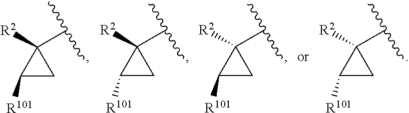

In some embodiments, q is 2. In some embodiments, the moiety of

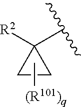

can have a structure of

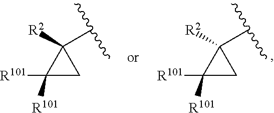

typically, the two R¹⁰ groups are the same. Typically, in Formula I-D, R² is hydrogen, F, C₁₋₂ alkyl optionally substituted with F (e.g., CH₃, CH₂F, CHF₂, or CF₃), CN, cyclopropyl, or C₂₋₃ alkynyl optionally substituted with F, such as

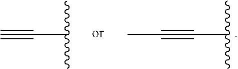

For example, in some embodiments, the moiety

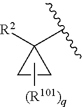

can be

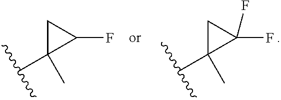

In some embodiments according to Formula I-D, the bicyclic heteroaryl moiety

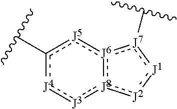

is a bicyclic heteroaryl moiety as defined in Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, or I-3b-2.

In Formula I and its subformulae, J³ is typically CR⁵, wherein R⁵ is defined herein.

In some embodiments, R⁵ can be hydrogen, halogen, or OH.

In some embodiments, R⁵ can be G², and G² is an optionally substituted 3-14 membered ring, preferably, a heteroaryl or heterocyclic ring.

In some embodiments, R⁵ can be an optionally substituted 4-12 membered heterocyclic ring having 1-4 ring heteroatoms independently selected from N, O, and S, wherein the S atom is optionally oxidized. The 4-12 membered heterocyclic ring is typically a monocyclic ring (typically has 4-7 ring members with 1 or 2 ring heteroatoms) or a bicyclic ring (typically has 5-12 ring members with 1-3 ring heteroatoms), which can be a fused, spiro, or bridged bicyclic ring.

In some embodiments, R⁵ can be an optionally substituted 4-7 membered heterocyclic ring having 1-3 ring heteroatoms independently selected from N, O, and S, wherein the S atom is optionally oxidized (e.g., S(O), SO₂, S(═NH)(═O)) for example, a monocyclic 5 or 6 membered heterocyclic ring having 1 or 2 ring heteroatoms, such as a pyrrolidine, piperidine, or piperazine ring. When substituted, the 4-7 membered heterocyclic ring can be typically substituted with 1-3 substituents each independently oxo, halogen (e.g., F), OH, CN, G^{A}, OG^{A}, C(O)G^{A}, C(O)NH₂, SO₂G^{A}, P(O)G^{A}G^{A}, C(O)NHG^{A}, C(O)NG^{A}G^{A}, SO₂NHG^{A}, S(═NH)(═O)G^{A}, S(═N-G^{A})(═O)G^{A}, SO₂NG^{A}G^{A}, NHC(O)G^{A}, or N(G^{A})C(O)G^{A}, wherein G^{A} at each occurrence is an optionally substituted group independently selected from (i) C₁₋₄ alkyl; (ii) C₁₋₄ heteroalkyl; and (iii) a 3-10 membered ring, or two G^{A} together with the intervening atom(s) are joined to form an optionally substituted 4-10 membered heterocyclic ring;
wherein when substituted, the C₁₋₄ alkyl or C₁₋₄ heteroalkyl is preferably substituted with 1-3 substituents each independently (1) halogen (preferably F), CN, OH, or NH₂, (2) C₁₋₄ heteroalkyl optionally substituted with F; or (3) an optionally substituted 3-10 membered ring; and
when substituted, the 3-10 membered ring or the 4-10 membered heterocyclic ring is preferably substituted with 1-3 substituents independently (1) oxo (as applicable), halogen (e.g., F, Cl), CN, OH, or NH₂; (2) C₁₋₄ alkyl optionally substituted with F; (3) C₁₋₄ heteroalkyl optionally substituted with F; or (4) a 3-6 membered ring optionally substituted 1-3 substituents independently selected from oxo, F, Cl, CN, OH, C₁₋₄ alkyl optionally substituted with F, and C₁₋₄ heteroalkyl optionally substituted with F.

In some more specific embodiments, R⁵ can have a structure according to F-1, F-2, or F-3 below:

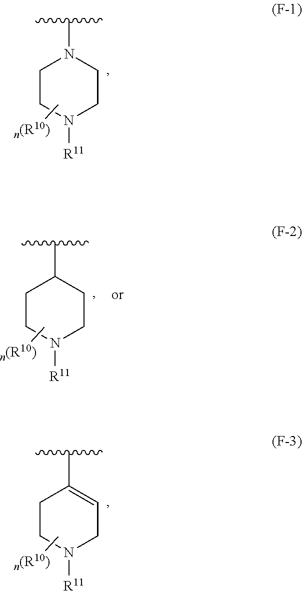

wherein:
n is an integer of 0-4,
(a) R¹⁰ at each occurrence is independently oxo, halogen (e.g., F), OH, CN, G^{A}, OG^{A}, C(O)G^{A}, SO₂G^{A}, P(O)G^{A}G^{A}, C(O)NHG^{A}, C(O)NG^{A}G^{A}, SO₂NHG^{A}, S(═NH)(═O)G^{A}, S(═N-G^{A})(═O)G^{A}, SO₂NG^{A}G^{A}, NHC(O)G^{A}, or N(G^{A})C(O)G^{A}, or
(b) two R¹⁰ are joined to form an optionally substituted 3-6 membered ring (e.g., cyclopropyl, cyclobutyl, or oxetane ring), and any remaining R¹⁰ are as defined in (a); or
(c) one R¹⁰ and R¹¹ are joined to form an optionally substituted 3-6 membered ring, and any remaining R¹⁰ are as defined in (a);
R¹¹ is hydrogen, G^{A}, C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, or defined in (c) above; wherein G^{A} at each occurrence is an optionally substituted group independently selected from (i) C₁₋₄ alkyl; (ii) C₁₋₄ heteroalkyl; and (iii) a 3-10 membered ring, or two G^{A} together with the intervening atom(s) are joined to form an optionally substituted 4-10 membered heterocyclic ring;
   wherein when substituted, the C₁₋₄ alkyl or C₁₋₄ heteroalkyl is preferably substituted with 1-3 substituents each independently (1) halogen (preferably F), CN, OH, or NH₂, (2) C₁₋₄ heteroalkyl optionally substituted with F; or (3) an optionally substituted 3-10 membered ring; and
   when substituted, the 3-10 membered ring or the 4-10 membered heterocyclic ring is preferably substituted with 1-3 substituents independently (1) oxo (as applicable), halogen (e.g., F, Cl), CN, OH, or NH₂; (2) C₁₋₄ alkyl optionally substituted with F; (3) C₁₋₄ heteroalkyl optionally substituted with F; or (4) a 3-6 membered ring optionally substituted 1-3 substituents independently selected from oxo, F, Cl, CN, OH, C₁₋₄ alkyl optionally substituted with F, and C₁₋₄ heteroalkyl optionally substituted with F.

To be clear, when it is said that two R¹⁰ are joined to form a ring structure, the two R¹⁰ can be attached to the same carbon, two adjacent carbon atoms, or two non-adjacent carbon atoms, thus, the ring formed can be a spiro ring, a fused ring, or a bridged ring. Similarly, when it is said that one R¹⁰ and R¹¹ are joined to form a ring structure, the R¹⁰ can be attached to a carbon atom adjacent to the nitrogen atom of (NR¹) or a non-adjacent carbon atom, thus forming a fused ring or a bridged ring.

In some embodiments, in F-1, F-2, or F-3, n is 0, 1, or 2; R¹⁰ at each occurrence is independently CN or G^{A}, and R¹¹ is hydrogen, G^{A}, C(O)G^{A}, SO₂G¹, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, wherein G^{A} is defined herein. In some embodiments, G^{A} at each occurrence is independently:
(1) a C₁₋₄ alkyl optionally substituted with 1-3 substituents each independently F, OH, C₁₋₄ alkoxy optionally substituted with 1-3 F, NH(C₁₋₄ alkyl), or N(C₁₋₃ alkyl)(C₁₋₃ alkyl); or
(2) a 3-10 membered ring, (C₁₋₄ alkylene)-(3-10 membered ring), or (C₁₋₄ heteroalkylene)-(3-10 membered ring), preferably, the 3-10 membered ring is a 3-6 membered ring selected from C₃₋₆ cycloalkyl, 4-6 membered heterocyclyl, 5 or 6-membered heteroaryl, or phenyl, wherein the 3-10 membered ring is optionally substituted with 1-3 substituents independently selected from oxo, F, Cl, OH, CN, C₁₋₄ alkyl optionally substituted with F, C₁₋₄ alkoxy optionally substituted with F, and optionally substituted 3-5 membered ring (e.g., cyclopropyl, cyclobutyl, azetidinyl, oxetanyl, etc.).

In some embodiments, in F-1, F-2, or F-3, n is 0, 1, or 2; R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃), and R¹¹ is hydrogen, G^{A}, C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, wherein G^{A} is defined herein.

In some embodiments, in F-1, F-2, or F-3, n is 0.

In some embodiments, in F-1, F-2, or F-3, n is 1.

In some embodiments, in F-1, F-2, or F-3, n is 2.

In some embodiments, in F-1, F-2, or F-3, two R¹⁰ are joined to form an optionally substituted 3-6 membered ring (e.g., cyclopropyl, cyclobutyl, or oxetane ring), and any remaining R¹⁰ are independently C₁₋₄ alkyl optionally substituted with F or C₁₋₄ heteroalkyl optionally substituted with F. For example, in some embodiments, R⁵ can have a structure according to

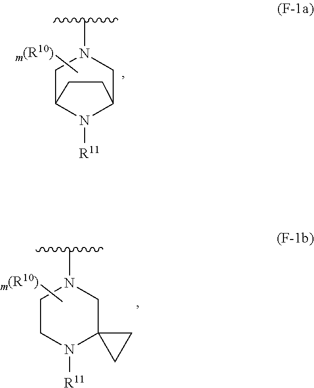

wherein m is 0, 1, or 2, R¹⁰ at each occurrence is independently C₁₋₄ alkyl optionally substituted with F or C₁₋₄ heteroalkyl optionally substituted with F, R¹¹ is defined herein.

In some embodiments, in F-1, F-2, or F-3, one R¹⁰ and R¹¹ are joined to form an optionally substituted 3-6 membered ring, and any remaining R¹⁰ are independently C₁₋₄ alkyl optionally substituted with F or C₁₋₄ heteroalkyl optionally substituted with F. For example, in some embodiments, R⁵ can have a structure according to

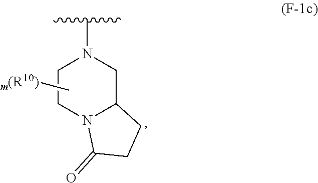

wherein m is 0, 1, or 2, R¹⁰ at each occurrence is independently C₁₋₄ alkyl optionally substituted with F or C₁₋₄ heteroalkyl optionally substituted with F.

In some embodiments, in F-1, F-2, or F-3, R¹¹ is C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, wherein G^{A} is defined herein. For example, in some embodiments, R¹¹ is C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, and G^{A} at each occurrence is independently (1) a C₁₋₄ alkyl optionally substituted with 1-3 substituents each independently F, OH, C₁₋₄ alkoxy optionally substituted with 1-3 F, NH(C₁₋₄ alkyl), or N(C₁₋₃ alkyl)(C₁₋₃ alkyl) or (2) a 3-6 membered ring selected from C₃₋₆ cycloalkyl, 4-6 membered heterocyclyl, 5 or 6-membered heteroaryl, or phenyl, wherein the 3-6 membered ring is optionally substituted with 1-3 substituents independently selected from oxo, F, Cl, OH, CN, C₁₋₄ alkyl optionally substituted with F, C₁₋₄ alkoxy optionally substituted with F, and optionally substituted 3-5 membered ring (e.g., cyclopropyl, cyclobutyl, azetidinyl, oxetanyl, etc.).

In some embodiments, in F-1, F-2, or F-3, R¹¹ can be C(O)G^{A1}, C(O)OG^{A1}SO₂G^{A1}, C(O)NHG^{A1}, or C(O)NG^{A1}G^{A1}, wherein G^{A1} at each occurrence is independently C₁₋₄ alkyl optionally substituted with F, such as methyl, ethyl, n-propyl, or isopropyl. In some embodiments, in F-1, F-2, or F-3, R¹¹ can be C(O)G^{A2}, SO₂G^{A2}, C(O)NHG^{A2} or SO₂NHG^{A2}, wherein G^{A2} is a 3-6 membered ring selected from C₃₋₆ cycloalkyl, 4-6 membered heterocyclyl, 5 or 6-membered heteroaryl, or phenyl, wherein the 3-6 membered ring is optionally substituted with 1-3 substituents independently selected from oxo, F, Cl, OH, CN, C₁₋₄ alkyl optionally substituted with F, C₁₋₄ alkoxy optionally substituted with F, and optionally substituted 3-5 membered ring (e.g., cyclopropyl, cyclobutyl, azetidinyl, oxetanyl, etc.). In some embodiments, G^{A2} can be a C₃₋₆ cycloalkyl, such as cyclopropyl or cyclobutyl, which is optionally substituted with 1 or 2 substituents each independently F, OH, CN, or CH₃. In some embodiments, G^{A2} can be a 4-6 membered heterocyclyl having 1 or 2 ring heteroatoms, such as pyrrolidine, piperidine, piperazine, etc., which is optionally substituted with 1 or 2 substituents each independently F, OH, CN, or CH₃.

In some specific embodiments, in F-1, F-2, or F-3, R¹¹ can be selected from:

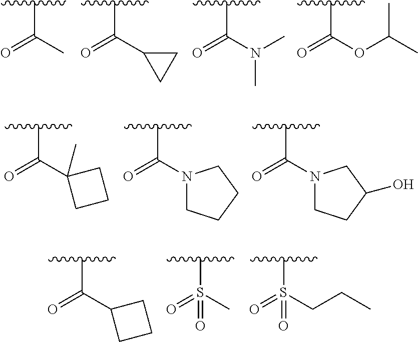

Typically, in such embodiments, n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

In some embodiments, in F-1, F-2, or F-3, R¹¹ can be G^{A}, wherein G^{A} is defined herein. For example, in some embodiments, R¹¹ can be an optionally substituted 5 or 6-membered heteroaryl, which is optionally substituted, preferably, when substituted, the 5 or 6-membered heteroaryl is substituted with 1-3 substituents independently selected from F, Cl, OH, CN, C₁₋₄ alkyl optionally substituted with F, C₁₋₄ alkoxy optionally substituted with F, and optionally substituted 3-5 membered ring (e.g., cyclopropyl, cyclobutyl, azetidinyl, oxetanyl, etc.). Typically, in such embodiments, n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃). In some embodiments, R¹¹ can be optionally substituted 5 or 6-membered heteroaryl having 2 or 3 heteroatoms each independently N, O, or S, such as optionally substituted imidazole or thiadiazole. For example, in some embodiments, R¹¹ can be

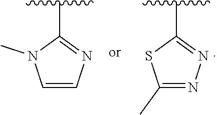

In some embodiments, R⁵ can be an optionally substituted 5-12 membered, such as 7-12 membered, heterocyclic ring having 1-3 ring heteroatoms independently selected from N, O, and S, wherein the S atom is optionally oxidized, for example, a 7-12 membered bicyclic heterocyclic ring which can be a spiro, fused, or bridged bicyclic heterocyclic ring.

In some embodiments, R⁵ can be a 7-12 membered spiro bicyclic heterocyclic ring having 1-3 ring heteroatoms each independently N, O, and S, wherein the S atom is optionally oxidized. For example, in some embodiments, R⁵ can be a spiro bicyclic heterocyclic ring having one ring being a 4, 5, or 6 membered ring and the other being a 3, 4 or 5 membered ring, such as the following:

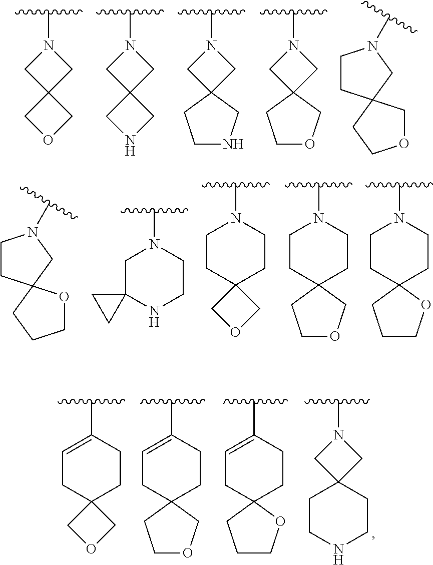

which is optionally substituted, e.g., with one or more R¹⁰ groups as defined herein. In some embodiments, when substituted, the substituents are each independently halogen (e.g., F), CN, cyclopropyl, C₁₋₄ alkyl optionally substituted with F (e.g., CH₃) or C₁₋₄ heteroalkyl optionally substituted with F (e.g., CH₂OCH₃).

In some preferred embodiments, R⁵ can have a spiro oxetane ring, for example, in some embodiments, R⁵ can be represented by the structure of

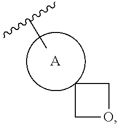

wherein ring A is a 4-8 membered carbocyclic or heterocyclic ring, which shares a single ring carbon atom with the oxetane ring. In some specific embodiments, R⁵ can be

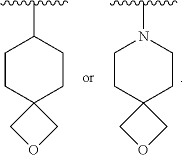 [SMILES: *C1CCC2(CC1)COC2 ; *N1CCC2(CC1)COC2]

In some embodiments, R⁵ can include a spiro oxidized thietane ring, for example, in some embodiments, R⁵ can be represented by the structure of

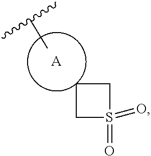

wherein ring A is a 4-8 membered carbocyclic or heterocyclic ring, which shares a single ring carbon atom with the oxidized thietane ring.

In some embodiments, R⁵ can be a 7-10 membered fused bicyclic heterocyclic ring having 1-3 ring heteroatoms each independently N, O, and S. For example, in some embodiments, R⁵ can be a 4,5-fused, 4,6-fused, 4,7-fused, 5,5-fused, 5,6-fused, 5,7-fused, or 6,6-fused bicyclic heterocyclic ring, such as the following:

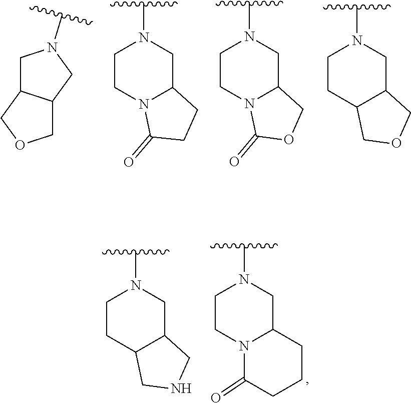

which is optionally substituted, e.g., with one or more R groups as defined herein. In some embodiments, when substituted, the substituents are each independently halogen (e.g., F), CN, cyclopropyl, C₁₋₄ alkyl optionally substituted with F (e.g., CH₃) or C₁₋₄ heteroalkyl optionally substituted with F (e.g., CH₂OCH₃).

In some embodiments, R⁵ can be a 7-10 membered bridged bicyclic heterocyclic ring having 1-3 ring heteroatoms each independently N, O, and S, such as a 2,1,1-bridged bicyclic, 2,2,1-bridged bicyclic, 2,2,2-bridged bicyclic, 2,3,1-bridged bicyclic, 2,4,1-bridged bicyclic, for example, R⁵ can be selected from the following:

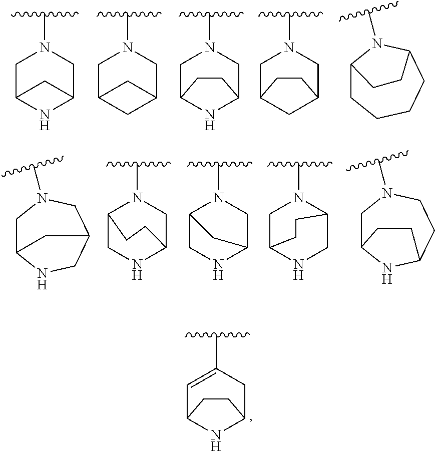

which is optionally substituted, e.g., with one or more R¹⁰ groups as defined herein. In some embodiments, when substituted, the substituents are each independently halogen (e.g., F), CN, cyclopropyl, C₁₋₄ alkyl optionally substituted with F (e.g., CH₃) or C₁₋₄ heteroalkyl optionally substituted with F (e.g., CH₂OCH₃).

In some embodiments, R⁵ can also be a 5 or 6 membered heteroaryl having 1-3 ring heteroatoms independently selected from N, O, and S, such as imidazole, pyrazole, thiadiazole, etc., which can be optionally substituted. When substituted, the 5 or 6 membered heteroaryl is preferably substituted with 1-3 substituents each independently halogen (e.g., F), OH, NH₂, CN, G^{B}, OG^{B}, C(O)G^{B}, SO₂G^{B}, P(O)G^{B}G^{B}, C(O)NHG^{B}, C(O)NG^{B}G^{B}, SO₂NHG^{B}, S(═NH)(═O)G^{B}, S(═N-G^{B})(═O)G^{B}, SO₂NG^{B}G^{B}, NHC(O)G^{B}, or N(G^{B})C(O)G^{B}, wherein G^{B} at each occurrence is independently selected from (i) C₁₋₄ alkyl optionally substituted with 1-3 F; (ii) C₁₋₄ heteroalkyl optionally substituted with 1-3 F; and (iii) a 3-7 membered ring, which is optionally substituted with 1-3 substituents independently selected from oxo, halogen (e.g., F), OH, CN, C₁₋₂ alkyl optionally substituted with F, and C₁₋₄ heteroalkyl having 1 or 2 heteroatoms independently O, N, or S, wherein the S is optionally oxidized, wherein the C₁₋₄ heteroalkyl is optionally substituted with 1-3 F, or two G^{B} together with the intervening atom(s) are joined to form an optionally substituted 4-7 membered heterocyclic ring. For example, in some embodiments, R⁵ can be an optionally substituted 5 or 6 membered heteroaryl, e.g., pyrazole, when substituted, the 5 or 6 membered heteroaryl is preferably substituted with 1-3 substituents independently (1) halogen (e.g., F, Cl), CN, OH, or NH₂; (2) C₁₋₄ alkyl optionally substituted with F; (3) C₁₋₄ heteroalkyl optionally substituted with F; or (4) a 3-6 membered ring optionally substituted 1-3 substituents independently selected from oxo, F, Cl, CN, OH, C₁₋₄ alkyl optionally substituted with F, and C₁₋₄ heteroalkyl optionally substituted with F. In some embodiments, the C₁₋₄ heteroalkyl has 1 or 2 heteroatoms independently O, N, or S, wherein the S is optionally oxidized.

In some embodiments, R⁵ can also be L³-G², wherein L³ is O, NH, CO, C(O)NH, C(O)N(C₁₋₄ alkyl), SO₂, SO₂NH, SO₂N(C₁₋₄ alkyl), an optionally substituted C₁₋₄ alkylene, an optionally substituted C₂₋₄ alkenylene, an optionally substituted C₂₋₄ alkynylene, or an optionally substituted C₁₋₄ heteroalkylene, and G² is an optionally substituted 3-14 membered ring, for example, any of the monocyclic or bicyclic heterocyclic ring as described herein.

In some specific embodiments, R⁵ can have a structure according to any of those corresponding R⁵ groups described in Table 1.

In some embodiments, the present disclosure also provides the following enumerated embodiments A1-29:

Embodiment A1. A compound of Formula I-A, or a pharmaceutically acceptable salt thereof:

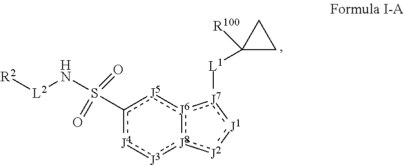

wherein:
R¹⁰⁰ is hydrogen, F, OH, CN, an optionally substituted alkyl, such as a C₁₋₄ alkyl optionally substituted with deuterium or F (e.g., CH₃, CD₃, CH₂F, CHF₂, etc.), or an optionally substituted heteroalkyl, such as a C₁₋₄ heteroalkyl optionally substituted with deuterium or F; preferably, R¹⁰⁰ is hydrogen, F, OH, CN, or methyl optionally substituted with deuterium or F (e.g., CH₃. CD₃, CH₂F, CHF₂, etc.); and
J¹, J², J³, J⁴, J⁵, J⁶, J⁷, J⁸, L¹, L², and R² are defined herein.

Embodiment A2. The compound of embodiment A1, or a pharmaceutically acceptable salt thereof, wherein R¹⁰⁰ is F or CN.

Embodiment A3. The compound of embodiment A1 or 2, or a pharmaceutically acceptable salt thereof, wherein L² is

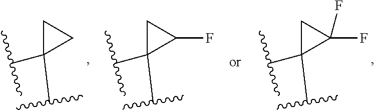 [SMILES: *C1(*)CC1 ; *C1(*)CC1F ; *C1(*)CC1(F)F]

and R² is hydrogen, F, C₁₋₂ alkyl optionally substituted with F (e.g., CH₃, CH₂F, CHF₂, or CF₃), CN, cyclopropyl, or C₂₋₃ alkynyl optionally substituted with F, such as

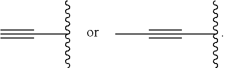

Embodiment A4. The compound of embodiment A2, or a pharmaceutically acceptable salt thereof, wherein L²-R² is

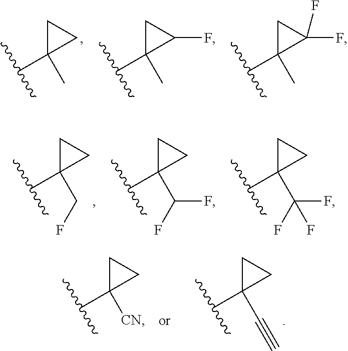

Embodiment A5. The compound of any of embodiments A1-4, or a pharmaceutically acceptable salt thereof, wherein J³ is CR⁵, wherein R⁵ is defined herein.

Embodiment A6. The compound of any of embodiments A1-4, or a pharmaceutically acceptable salt thereof, wherein

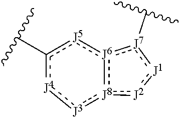

is

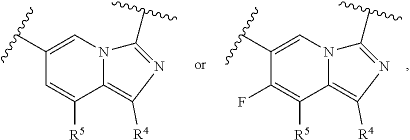

wherein R⁴ is hydrogen or halogen, preferably, R⁴ is hydrogen, F or Cl.

Embodiment A7. The compound of any of embodiments A1-4, or a pharmaceutically acceptable salt thereof, wherein

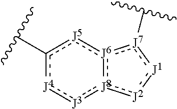

is

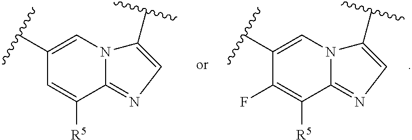

Embodiment A8. The compound of any of embodiments A1-4, or a pharmaceutically acceptable salt thereof, wherein

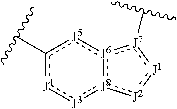

is

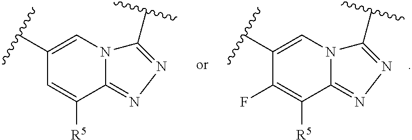

Embodiment A9. The compound of any of embodiments A1-4, or a pharmaceutically acceptable salt thereof, wherein

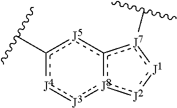

is

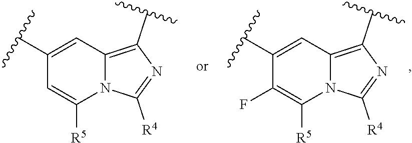

wherein R⁴ is hydrogen or halogen, preferably, R⁴ is hydrogen, F or Cl.

Embodiment A10. The compound of any of embodiments A1-4, or a pharmaceutically acceptable salt thereof, wherein

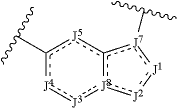

is

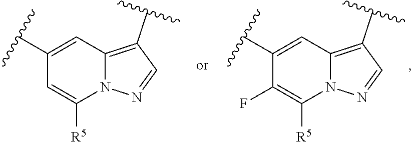

Embodiment A11. The compound of any of embodiments A1-4, or a pharmaceutically acceptable salt thereof, wherein

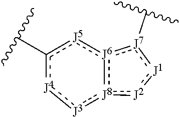

is

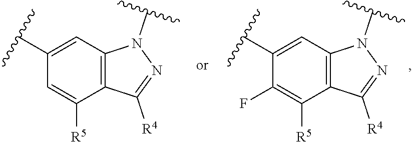

wherein R⁴ is hydrogen or halogen, preferably, R⁴ is hydrogen, F or Cl.

Embodiment A12. The compound of any of embodiments A1-4, or a pharmaceutically acceptable salt thereof, wherein

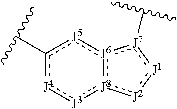

is

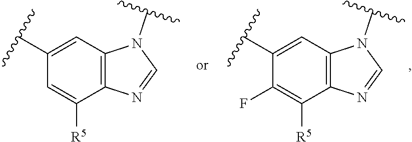

Embodiment A13. The compound of any of embodiments A1-12, or a pharmaceutically acceptable salt thereof, wherein L¹ is an optionally substituted C₁₋₆ alkylene, an optionally substituted C₂₋₆ alkenylene, an optionally substituted C₂₋₆ alkynylene, or an optionally substituted 3-8 membered ring.

Embodiment A14. The compound of any of embodiments A1-12, or a pharmaceutically acceptable salt thereof, wherein L³ is an optionally substituted 5-membered heteroaryl ring, such as an optionally substituted ring selected from thiazole, oxazole, imidazole, oxadiazole, or thiadiazole.

Embodiment A15. The compound of any of embodiments A1-12, or a pharmaceutically acceptable salt thereof, wherein L¹ is

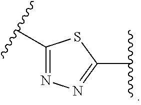 [SMILES: *c1nnc(*)s1]

Embodiment A16. The compound of any of embodiments A1-12, or a pharmaceutically acceptable salt thereof, wherein L¹ is

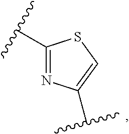 [SMILES: *c1csc(*)n1]

wherein the attaching point meta to the S atom is attached to the cyclopropyl ring in Formula I-A.

Embodiment A17. The compound of any of embodiments A1-12, or a pharmaceutically acceptable salt thereof, wherein L¹ is an optionally substituted 6-membered heteroarylene having 1 or 2 ring nitrogens, such as an optionally substituted pyridylene or optionally substituted pyridazylene.

Embodiment A18. The compound of any of embodiments A1-12, or a pharmaceutically acceptable salt thereof, wherein L¹ is

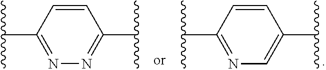 [SMILES: *c1ccc(*)nn1 ; *c1ccc(*)nc1]

Embodiment A19. The compound of any of embodiments A5-18, or a pharmaceutically acceptable salt thereof, wherein R⁵ is an optionally substituted 4-12 membered heterocyclic ring having 1-4 ring heteroatoms independently selected from N, O, and S, wherein the S atom is optionally oxidized.

Embodiment A20. The compound of any of embodiments A5-18, or a pharmaceutically acceptable salt thereof, wherein R⁵ is an optionally substituted monocyclic 5 or 6 membered heterocyclic ring having 1 or 2 ring heteroatoms.

Embodiment A21. The compound of any of embodiments A5-18, or a pharmaceutically acceptable salt thereof, wherein R⁵ has a structure according to F-1, F-2, or F-3 as defined herein.

Embodiment A22. The compound of any of embodiments A5-18, or a pharmaceutically acceptable salt thereof, wherein R⁵ has a structure according to F-1, F-2, or F-3, wherein R¹¹ is selected from:

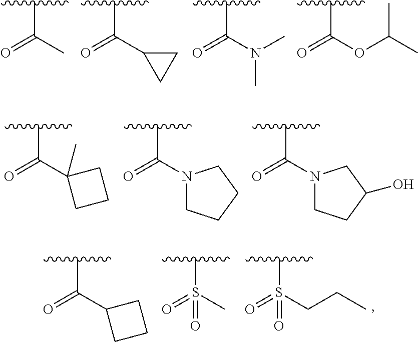

and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment A23. The compound of any of embodiments A5-18, or a pharmaceutically acceptable salt thereof, wherein R⁵ has a structure according to F-1, F-2, or F-3, wherein R¹¹ is an optionally substituted 5 or 6-membered heteroaryl having 2 or 3 heteroatoms each independently N, O, or S, such as optionally substituted imidazole or thiadiazole, and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment A24. The compound of any of embodiments A5-18, or a pharmaceutically acceptable salt thereof, wherein R⁵ is an optionally substituted 7-12 membered spiro bicyclic heterocyclic ring having 1-3 ring heteroatoms each independently N, O, and S, wherein the S atom is optionally oxidized, such as selected from:

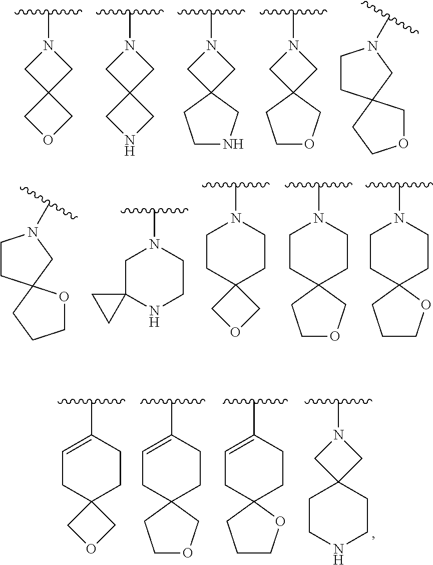

which is optionally substituted.

Embodiment A25. The compound of any of embodiments A5-18, or a pharmaceutically acceptable salt thereof, wherein R⁵ is an optionally substituted 7-10 membered fused bicyclic heterocyclic ring having 1-3 ring heteroatoms each independently N, O, and S, such as selected from:

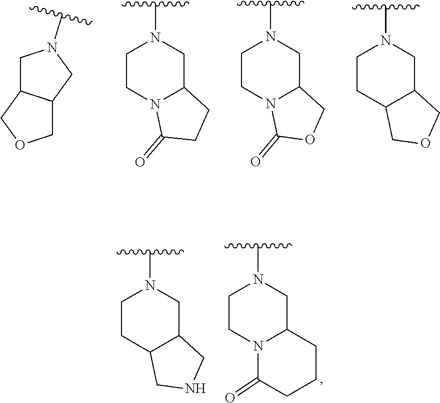

which is optionally substituted.

Embodiment A26. The compound of any of embodiments A5-18, or a pharmaceutically acceptable salt thereof, wherein R⁵ is an optionally substituted 7-10 membered bridged bicyclic heterocyclic ring having 1-3 ring heteroatoms each independently N, O, and S, such as selected from:

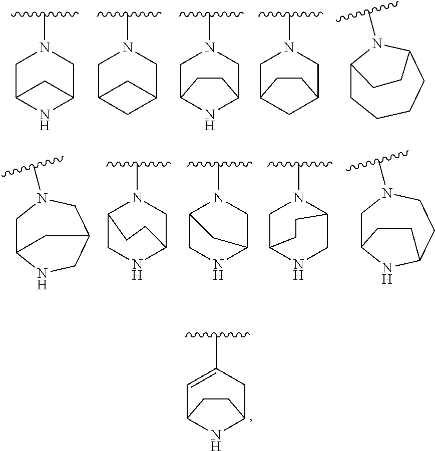

which is optionally substituted.

Embodiment A27. The compound of any of embodiments A5-18, or a pharmaceutically acceptable salt thereof, wherein R⁵ is a 5 or 6 membered heteroaryl having 1-3 ring heteroatoms independently selected from N, O, and S, such as imidazole, pyrazole, thiadiazole, etc., which is optionally substituted.

Embodiment A28. The compound of any of embodiments A5-18, or a pharmaceutically acceptable salt thereof, wherein R^{S} is

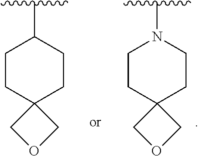 [SMILES: *C1CCC2(CC1)COC2 ; *N1CCC2(CC1)COC2]

Embodiment A29. The compound of any of embodiments A5-18, or a pharmaceutically acceptable salt thereof, wherein R⁵ is any of the corresponding R⁵ groups in Table 1.

In some embodiments, the present disclosure also provides the following enumerated embodiments B1-43:

Embodiment B1. A compound of Formula I-D, or a pharmaceutically acceptable salt thereof:

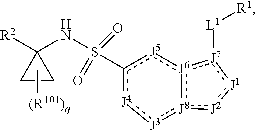

wherein:
q is 1 or 2;
R¹⁰¹ is F or methyl optionally substituted with F; and
J¹, J², J³, J⁴, J⁵, J⁶, J⁷, J⁸, L¹, R¹, and R² are defined herein.

Embodiment B2. The compound of embodiment B1, or a pharmaceutically acceptable salt thereof, wherein R¹⁰¹ is F.

Embodiment B3. The compound of embodiment B1 or 2, or a pharmaceutically acceptable salt thereof, wherein q is 1.

Embodiment B4. The compound of embodiment B1 or 2, or a pharmaceutically acceptable salt thereof, wherein q is 2.

Embodiment B5. The compound of any of embodiments B1-4, or a pharmaceutically acceptable salt thereof, wherein R² is hydrogen, F, C₁₋₂ alkyl optionally substituted with F (e.g., CH₃, CH₂F, CHF₂, or CF₃), CN, cyclopropyl, or C₂₋₃ alkynyl optionally substituted with F, such as

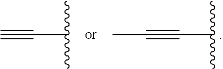

Embodiment B6. The compound of embodiment B1, or a pharmaceutically acceptable salt thereof, wherein the moiety of

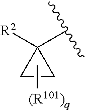

is

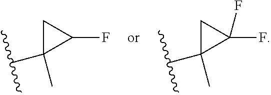

Embodiment B7. The compound of any of embodiments B1-6, or a pharmaceutically acceptable salt thereof, wherein J³ is CR⁵, wherein R⁵ is defined herein.

Embodiment B8. The compound of any of embodiments B1-6, or a pharmaceutically acceptable salt thereof, wherein

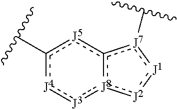

is

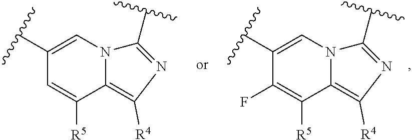

wherein R⁴ is hydrogen or halogen preferably, R⁴ is hydrogen, F or Cl.

Embodiment B9. The compound of any of embodiments B1-6, or a pharmaceutically acceptable salt thereof, wherein

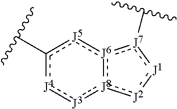

is

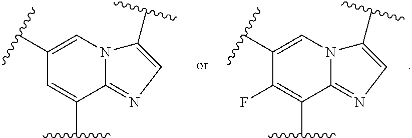 [SMILES: *c1cc(*)c2ncc(*)n2c1 ; *c1cn2c(*)cnc2c(*)c1F]

Embodiment B10. The compound of any of embodiments B1-6, or a pharmaceutically acceptable salt thereof, wherein

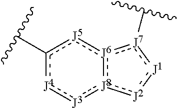

is

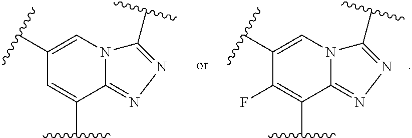 [SMILES: *c1cc(*)c2nnc(*)n2c1 ; *c1cn2c(*)nnc2c(*)c1F]

Embodiment B11. The compound of any of embodiments B1-6, or a pharmaceutically acceptable salt thereof, wherein

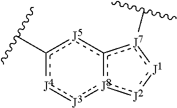

is

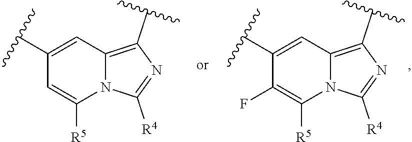

wherein R⁴ is hydrogen or halogen, preferably, R⁴ is hydrogen, F or Cl.

Embodiment B12. The compound of any of embodiments B1-6, or a pharmaceutically acceptable salt thereof, wherein

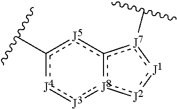

is

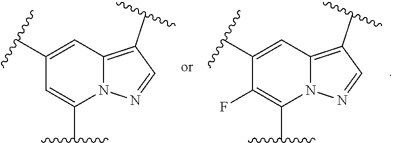 [SMILES: *c1cc(*)n2ncc(*)c2c1 ; *c1cc2c(*)cnn2c(*)c1F]

Embodiment B13. The compound of any of embodiments B1-6, or a pharmaceutically acceptable salt thereof, wherein

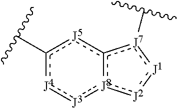

is

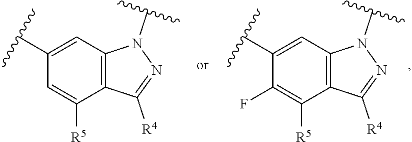

wherein R⁴ is hydrogen or halogen, preferably, R⁴ is hydrogen, F or Cl.

Embodiment B14. The compound of any of embodiments B1-6, or a pharmaceutically acceptable salt thereof, wherein

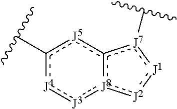

is

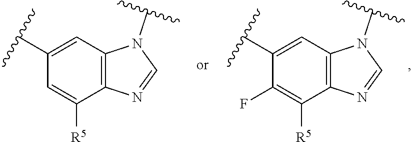

Embodiment B15. The compound of any of embodiments B1-14, or a pharmaceutically acceptable salt thereof, wherein L¹ is an optionally substituted C₁₋₆ alkylene, an optionally substituted C₂₋₆ alkenylene, an optionally substituted C₂₋₆ alkynylene, or an optionally substituted 3-8 membered ring, and R¹ is an optionally substituted 3-8 membered ring.

Embodiment B16. The compound of embodiment B15, or a pharmaceutically acceptable salt thereof, wherein L¹ is an optionally substituted 5-membered heteroaryl ring, such as an optionally substituted ring selected from thiazole, oxazole, imidazole, oxadiazole, or thiadiazole.

Embodiment B17. The compound of embodiment B15, or a pharmaceutically acceptable salt thereof, wherein L¹ is

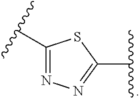 [SMILES: *c1nnc(*)s1]

Embodiment B18. The compound of embodiment B15, or a pharmaceutically acceptable salt thereof, wherein L¹ is

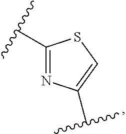 [SMILES: *c1csc(*)n1]

wherein the attaching point meta to the S atom is attached to R¹.

Embodiment B19. The compound of any of embodiments B1-14, or a pharmaceutically acceptable salt thereof, wherein L¹ is an optionally substituted 6-membered heteroarylene having 1 or 2 ring nitrogens, such as an optionally substituted pyridylene or optionally substituted pyridazylene.

Embodiment B20. The compound of embodiment B19, or a pharmaceutically acceptable salt thereof, wherein L¹ is

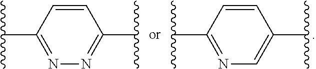

Embodiment B21. The compound of any of embodiments B15-20, or a pharmaceutically acceptable salt thereof, wherein R¹ is 3-4 membered ring, such as cyclopropyl or cyclobutyl, optionally substituted with 1-3 substituents each independently selected from deuterium, F, OH, CN, C₁₋₄ alkyl, C₂₋₄ alkenyl, C₂₋₄ alkynyl, and C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH.

Embodiment B22. The compound of any of embodiments B15-20, or a pharmaceutically acceptable salt thereof, wherein R¹ is an optionally substituted cyclopropyl, preferably, when substituted, the cyclopropyl is substituted with 1-3 substituents each independently selected from F, OH, CN, or C₁₋₂ alkyl optionally substituted with 1-3 F, for example, R¹ is

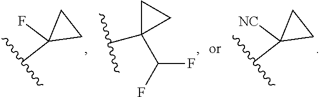

Embodiment B23. The compound of any of embodiments B1-14, or a pharmaceutically acceptable salt thereof, wherein L¹ is an optionally substituted 7-12 membered bicyclic ring structure; and R¹ is hydrogen, deuterium, halogen, CN, OH, NH₂, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring.

Embodiment B24. The compound of embodiment B23, or a pharmaceutically acceptable salt thereof, wherein L¹ is an optionally substituted 8-10 membered fused bicyclic ring structure, which has a first and second constituent ring, wherein the first constituent ring is a 5-membered heteroaryl ring having 1-3 ring heteroatoms independently selected from N, O, and S, and the second constituent ring is an aryl, heteroaryl, carbocyclic, or heterocyclic ring.

Embodiment B25. The compound of embodiment B23, or a pharmaceutically acceptable salt thereof, wherein L¹ is an optionally substituted 8 or 9 membered fused bicyclic heteroaryl having a first and second constituent ring, wherein the first constituent ring is a 5-membered heteroaryl ring having 1-3 ring heteroatoms independently selected from N, O, and S, and the second constituent ring is phenyl, a 5-membered heteroaryl, or a 6-membered heteroaryl.

Embodiment B26. The compound of embodiment B24 or 25, or a pharmaceutically acceptable salt thereof, wherein the first constituent ring is a thiazole ring,

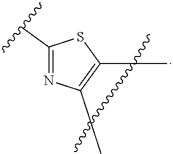

Embodiment B27. The compound of embodiment B23, or a pharmaceutically acceptable salt thereof, wherein L¹ is a fused bicyclic heteroaryl having a structure of

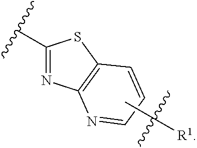

Embodiment B28. The compound of any of embodiments B23-27, or a pharmaceutically acceptable salt thereof, wherein R¹ is hydrogen, deuterium, halogen (preferably F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH.

Embodiment B29. The compound of any of embodiments B1-14, or a pharmaceutically acceptable salt thereof, wherein L¹ is null and R¹ is an optionally substituted 5-membered heteroaryl having 1-3 ring heteroatoms independently selected from S, N, and O, for example, an optionally substituted ring selected from thiazole, oxazole, imidazole, oxadiazole, or thiadiazole, or R¹ is an optionally substituted 6-membered heteroarylene having 1 or 2 ring nitrogens, such as an optionally substituted pyridylene or optionally substituted pyridazylene.

Embodiment B30. The compound of embodiment B29, or a pharmaceutically acceptable salt thereof, wherein R¹ is thiazole, oxazole, imidazole, oxadiazole, or thiadiazole, each of which is optionally substituted with a C₁₋₂ alkyl optionally substituted with F, such as CF₂H, CH₃, CF₃, etc.

Embodiment B31. The compound of embodiment B30, or a pharmaceutically acceptable salt thereof, wherein R¹ is

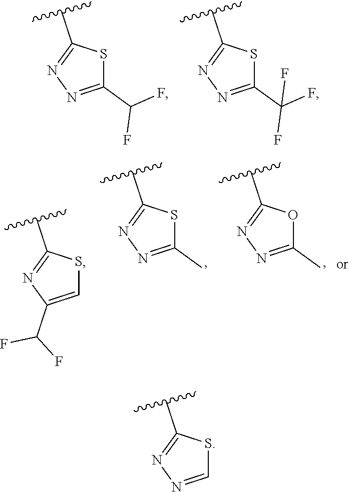

Embodiment B32. The compound of any of embodiments B1-14, or a pharmaceutically acceptable salt thereof, wherein L¹-R¹ is any of the corresponding L¹-R¹ groups in Table 1.

Embodiment B33. The compound of any of embodiments B7-32, or a pharmaceutically acceptable salt thereof, wherein R^{S} is an optionally substituted 4-12 membered heterocyclic ring having 1-4 ring heteroatoms independently selected from N, O, and S, wherein the S atom is optionally oxidized.

Embodiment B34. The compound of any of embodiments B7-32, or a pharmaceutically acceptable salt thereof, wherein R⁵ is an optionally substituted monocyclic 5 or 6 membered heterocyclic ring having 1 or 2 ring heteroatoms.

Embodiment B35. The compound of any of embodiments B7-32, or a pharmaceutically acceptable salt thereof, wherein R⁵ has a structure according to F-1, F-2, or F-3 as defined herein.

Embodiment B36. The compound of any of embodiments B7-32, or a pharmaceutically acceptable salt thereof, wherein R⁵ has a structure according to F-1, F-2, or F-3, wherein R¹¹ is selected from:

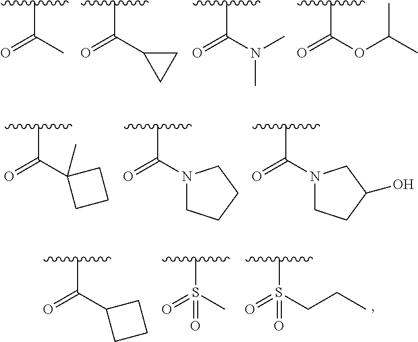

and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment B37. The compound of any of embodiments B7-32, or a pharmaceutically acceptable salt thereof, wherein R⁵ has a structure according to F-1, F-2, or F-3, wherein R¹¹ is an optionally substituted 5 or 6-membered heteroaryl having 2 or 3 heteroatoms each independently N, O, or S, such as optionally substituted imidazole or thiadiazole, and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment B38. The compound of any of embodiments B7-32, or a pharmaceutically acceptable salt thereof, wherein R⁵ is an optionally substituted 7-12 membered spiro bicyclic heterocyclic ring having 1-3 ring heteroatoms each independently N, O, and S, wherein the S atom is optionally oxidized.

Embodiment B39. The compound of any of embodiments B7-32, or a pharmaceutically acceptable salt thereof, wherein R⁵ is an optionally substituted 7-10 membered fused bicyclic heterocyclic ring having 1-3 ring heteroatoms each independently N, O, and S.

Embodiment B40. The compound of any of embodiments B7-32, or a pharmaceutically acceptable salt thereof, wherein R⁵ is an optionally substituted 7-10 membered bridged bicyclic heterocyclic ring having 1-3 ring heteroatoms each independently N, O, and S.

Embodiment B41. The compound of any of embodiments B7-32, or a pharmaceutically acceptable salt thereof, wherein R⁵ is a 5 or 6 membered heteroaryl having 1-3 ring heteroatoms independently selected from N, O, and S, such as imidazole, pyrazole, thiadiazole, etc., which is optionally substituted.

Embodiment B42. The compound of any of embodiments B7-32, or a pharmaceutically acceptable salt thereof, wherein R⁵ is

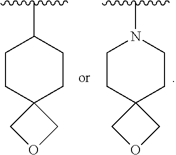

Embodiment B43. The compound of any of embodiments B7-32, or a pharmaceutically acceptable salt thereof, wherein R⁵ is any of the corresponding R⁵ groups in Table 1.

In some embodiments, the present disclosure also provides the following enumerated embodiments C1-26:

Embodiment C1. A compound of Formula I-B, or a pharmaceutically acceptable salt thereof:

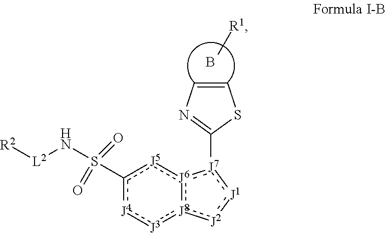

wherein:
Ring B is an optionally substituted 5 or 6 membered heterocyclyl or heteroaryl ring having 1-3 ring heteroatoms independently N, O, or S;
R¹ is hydrogen, deuterium, halogen, CN, OH, NH₂, an optionally substituted C₁₋₄ alkyl, an optionally substituted C₁₋₄ heteroalkyl, or an optionally substituted 3-6 membered ring; and
J¹, J², J³, J⁴, J⁵, J⁶, J⁷, J⁸, L¹, L², and R² are defined herein.

Embodiment C2. The compound of embodiment C1, or a pharmaceutically acceptable salt thereof, wherein ring B is an optionally substituted 6-membered heteroaryl, such as an optionally substituted pyridine, for example,

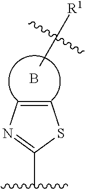

can have a structure of

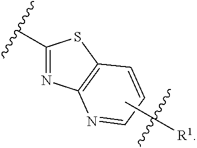

Embodiment C3. The compound of embodiment C1, or a pharmaceutically acceptable salt thereof, wherein ring B is an optionally substituted 6-membered heterocyclic ring, for example,

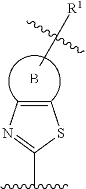

can have a structure of

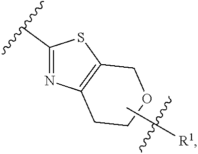

which is optionally substituted.

Embodiment C4. The compound of any of embodiments C₁₋₃, or a pharmaceutically acceptable salt thereof, wherein R¹ is hydrogen, deuterium, halogen (preferably F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH.

Embodiment C5. The compound of embodiment C1, or a pharmaceutically acceptable salt thereof, wherein

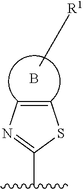

is

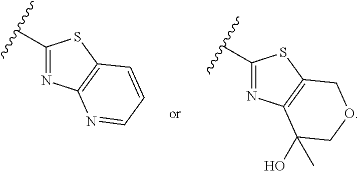 [SMILES: *c1nc2ncccc2s1 ; *c1nc2c(s1)COCC2(C)O]

Embodiment C6. The compound of any of embodiments C1-5, or a pharmaceutically acceptable salt thereof, wherein L² is is

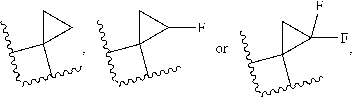

and R² is hydrogen, F, C₁₋₂ alkyl optionally substituted with F (e.g., CH₃, CH₂F, CHF₂, or CF₃), CN, cyclopropyl, or C₂₋₃ alkynyl optionally substituted with F, such as

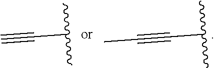

Embodiment C7. The compound of embodiment C6, or a pharmaceutically acceptable salt thereof, wherein L²-R² is

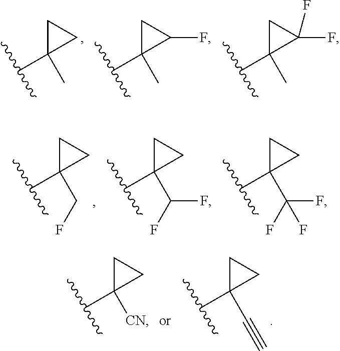

Embodiment C8. The compound of any of embodiments C1-7, or a pharmaceutically acceptable salt thereof, wherein J³ is CR⁵, wherein R⁵ is defined herein.

Embodiment C9. The compound of any of embodiments C1-7, or a pharmaceutically acceptable salt thereof, wherein

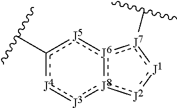

is

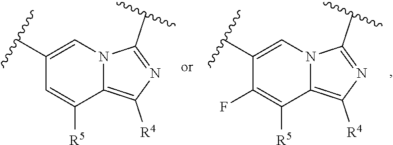

wherein R⁴ is hydrogen or halogen, preferably, R⁴ is hydrogen, F or Cl.

Embodiment C10. The compound of any of embodiments C1-7, or a pharmaceutically acceptable salt thereof, wherein

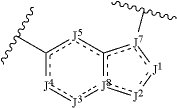

is

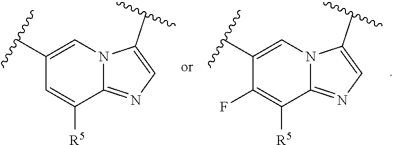

Embodiment C11. The compound of any of embodiments C1-7, or a pharmaceutically acceptable salt thereof, wherein

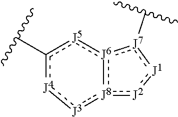

is

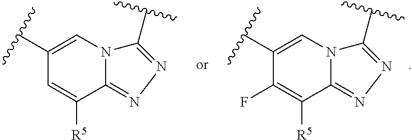

Embodiment C12. The compound of an embodiments C1-7, or a pharmaceutically acceptable salt thereof, wherein

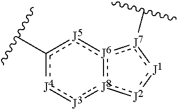

is

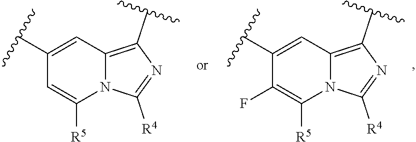

wherein R⁴ is hydrogen or halogen, preferably, R⁴ is hydrogen, F or Cl.

Embodiment C13. The compound of any of embodiments C1-7, or a pharmaceutically acceptable salt thereof, wherein

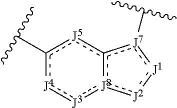

is

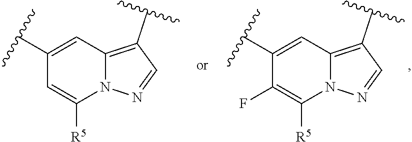

Embodiment C14. The compound of any of embodiments C1-7, or a pharmaceutically acceptable salt thereof, wherein

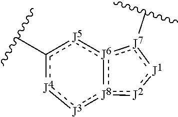

is

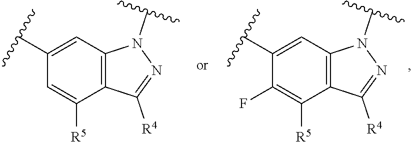

wherein R⁴ is hydrogen or halogen, preferably, R⁴ is hydrogen, F or Cl.

Embodiment C15. The compound of any of embodiments C1-7, or a pharmaceutically acceptable salt thereof, wherein

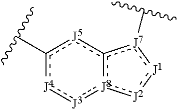

is

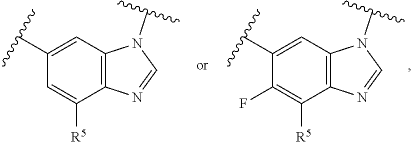

Embodiment C16. The compound of any of embodiments C8-15, or a pharmaceutically acceptable salt thereof, wherein R⁵ is an optionally substituted 4-12 membered heterocyclic ring having 1-4 ring heteroatoms independently selected from N, O, and S, wherein the S atom is optionally oxidized.

Embodiment C17. The compound of any of embodiments C8-15, or a pharmaceutically acceptable salt thereof, wherein R⁵ is an optionally substituted monocyclic 5 or 6 membered heterocyclic ring having 1 or 2 ring heteroatoms.

Embodiment C18. The compound of any of embodiments C8-15, or a pharmaceutically acceptable salt thereof, wherein R⁵ has a structure according to F-1, F-2, or F-3 as defined herein.

Embodiment C19. The compound of any of embodiments C8-15, or a pharmaceutically acceptable salt thereof, wherein R⁵ has a structure according to F-1, F-2, or F-3, wherein R¹¹ is selected from:

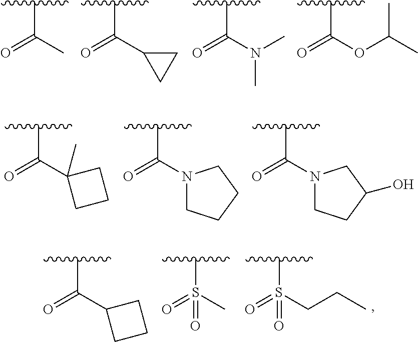

and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment C20. The compound of any of embodiments C8-15, or a pharmaceutically acceptable salt thereof, wherein R⁵ has a structure according to F-1, F-2, or F-3, wherein R¹¹ is an optionally substituted 5 or 6-membered heteroaryl having 2 or 3 heteroatoms each independently N, O, or S, such as optionally substituted imidazole or thiadiazole, and wherein n is 0, 1, or 2, and R¹⁰ at each occurrence is independently CN, cyclopropyl, C₁₋₄ alkyl optionally substituted with F (e.g., CH₃) or C₁₋₄ heteroalkyl optionally substituted with F (e.g., CH₂OCH₃).

Embodiment C21. The compound of any of embodiments C8-15, or a pharmaceutically acceptable salt thereof, wherein R⁵ is an optionally substituted 7-12 membered spiro bicyclic heterocyclic ring having 1-3 ring heteroatoms each independently N, O, and S, wherein the S atom is optionally oxidized, such as selected from:

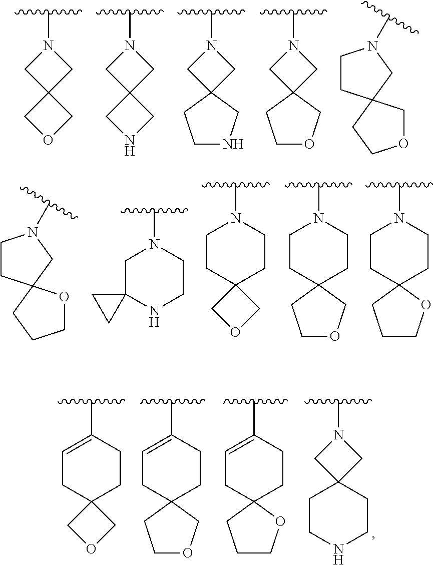

which is optionally substituted.

Embodiment C22. The compound of any of embodiments C8-15, or a pharmaceutically acceptable salt thereof, wherein R⁵ is an optionally substituted 7-10 membered fused bicyclic heterocyclic ring having 1-3 ring heteroatoms each independently N, O, and S, such as selected from:

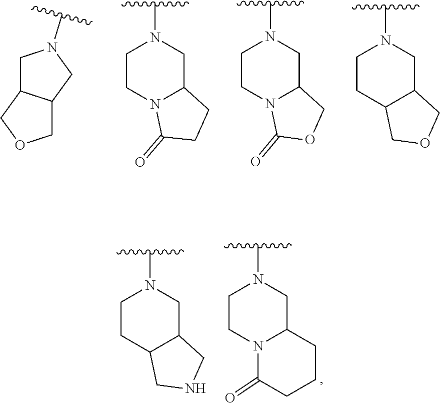

which is optionally substituted.

Embodiment C23. The compound of any of embodiments C8-15, or a pharmaceutically acceptable salt thereof, wherein R⁵ is an optionally substituted 7-10 membered bridged bicyclic heterocyclic ring having 1-3 ring heteroatoms each independently N, O, and S, such as selected from:

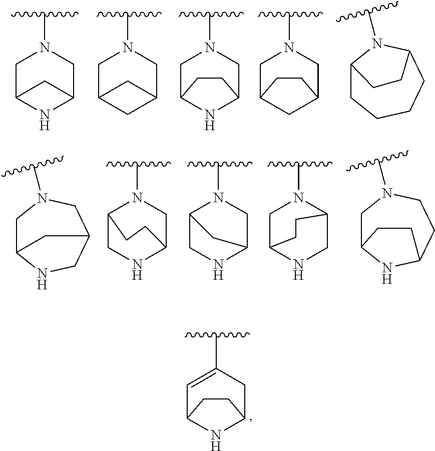

which is optionally substituted.

Embodiment C24. The compound of any of embodiments C8-15, or a pharmaceutically acceptable salt thereof, wherein R⁵ is a 5 or 6 membered heteroaryl having 1-3 ring heteroatoms independently selected from N, O, and S, such as imidazole, pyrazole, thiadiazole, etc., which is optionally substituted.

Embodiment C25. The compound of any of embodiments C8-15, or a pharmaceutically acceptable salt thereof, wherein R⁵ is

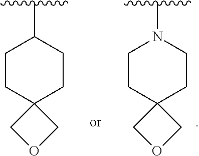 [SMILES: *C1CCC2(CC1)COC2 ; *N1CCC2(CC1)COC2]

Embodiment C26. The compound of any of embodiments C8-15, or a pharmaceutically acceptable salt thereof, wherein R⁵ is any of the corresponding R⁵ groups in Table 1.

In some embodiments, the present disclosure also provides a compound selected from Table 1 below, a deuterated analog thereof, a stereoisomer thereof, or a pharmaceutically acceptable salt thereof:

TABLE 1
List of Compounds

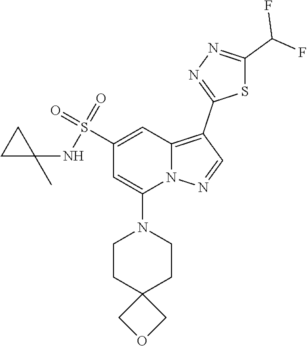 [SMILES: CC1(NS(=O)(=O)c2cc(N3CCC4(CC3)COC4)n3ncc(-c4nnc(C(F)F)s4)c3c2)CC1]

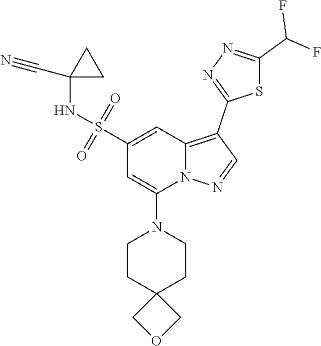 [SMILES: N#CC1(NS(=O)(=O)c2cc(N3CCC4(CC3)COC4)n3ncc(-c4nnc(C(F)F)s4)c3c2)CC1]

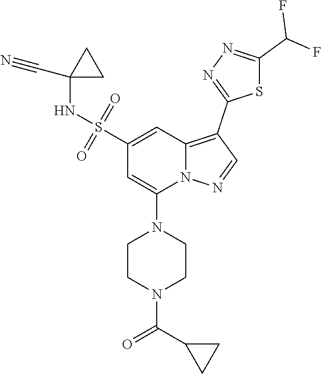 [SMILES: N#CC1(NS(=O)(=O)c2cc(N3CCN(C(=O)C4CC4)CC3)n3ncc(-c4nnc(C(F)F)s4)c3c2)CC1]

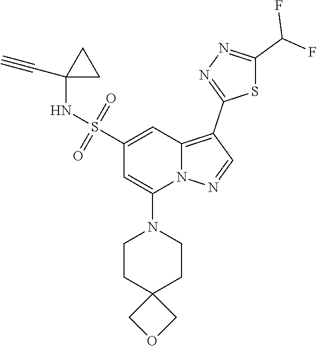 [SMILES: C#CC1(NS(=O)(=O)c2cc(N3CCC4(CC3)COC4)n3ncc(-c4nnc(C(F)F)s4)c3c2)CC1]

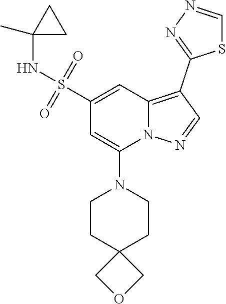 [SMILES: CC1(NS(=O)(=O)c2cc(N3CCC4(CC3)COC4)n3ncc(-c4nncs4)c3c2)CC1]

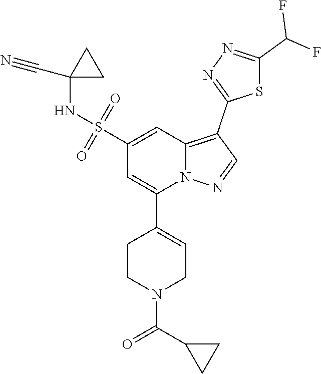 [SMILES: N#CC1(NS(=O)(=O)c2cc(C3=CCN(C(=O)C4CC4)CC3)n3ncc(-c4nnc(C(F)F)s4)c3c2)CC1]

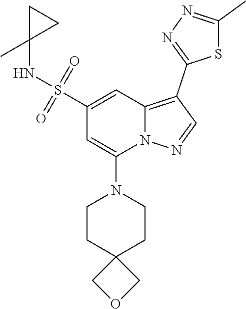

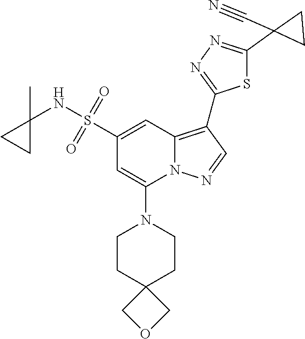 [SMILES: CC1(NS(=O)(=O)c2cc(N3CCC4(CC3)COC4)n3ncc(-c4nnc(C5(C#N)CC5)s4)c3c2)CC1]

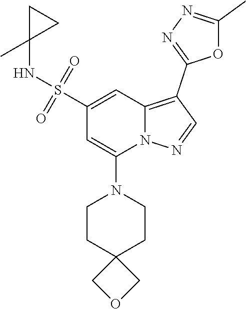

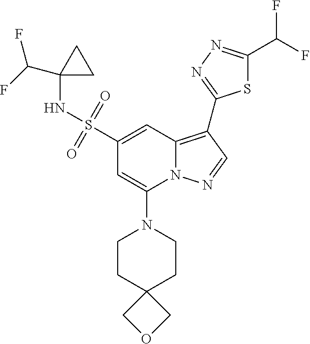 [SMILES: O=S(=O)(NC1(C(F)F)CC1)c1cc(N2CCC3(CC2)COC3)n2ncc(-c3nnc(C(F)F)s3)c2c1]

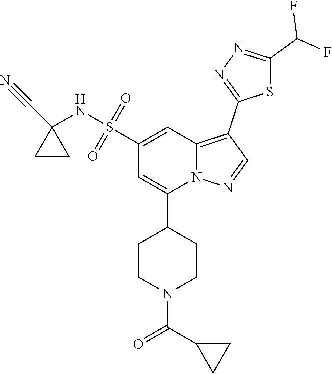 [SMILES: N#CC1(NS(=O)(=O)c2cc(C3CCN(C(=O)C4CC4)CC3)n3ncc(-c4nnc(C(F)F)s4)c3c2)CC1]

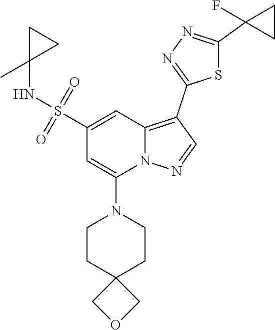 [SMILES: CC1(NS(=O)(=O)c2cc(N3CCC4(CC3)COC4)n3ncc(-c4nnc(C5(F)CC5)s4)c3c2)CC1]

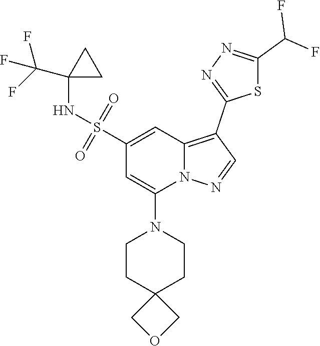 [SMILES: O=S(=O)(NC1(C(F)(F)F)CC1)c1cc(N2CCC3(CC2)COC3)n2ncc(-c3nnc(C(F)F)s3)c2c1]

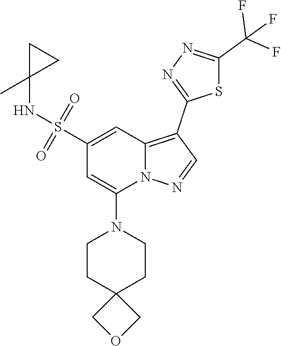 [SMILES: CC1(NS(=O)(=O)c2cc(N3CCC4(CC3)COC4)n3ncc(-c4nnc(C(F)(F)F)s4)c3c2)CC1]

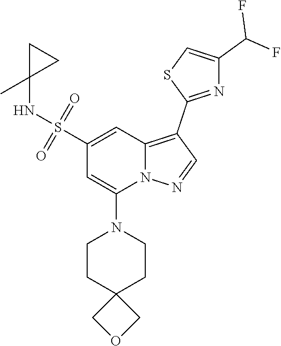 [SMILES: CC1(NS(=O)(=O)c2cc(N3CCC4(CC3)COC4)n3ncc(-c4nc(C(F)F)cs4)c3c2)CC1]

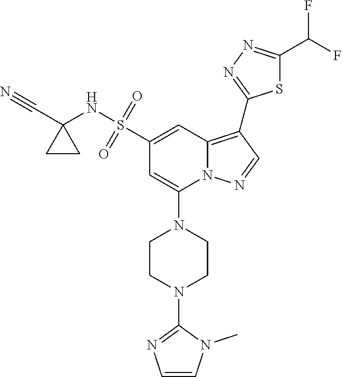 [SMILES: Cn1ccnc1N1CCN(c2cc(S(=O)(=O)NC3(C#N)CC3)cc3c(-c4nnc(C(F)F)s4)cnn23)CC1]

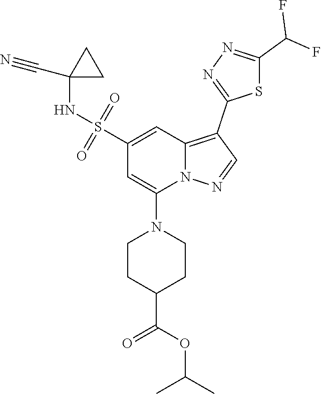 [SMILES: CC(C)OC(=O)C1CCN(c2cc(S(=O)(=O)NC3(C#N)CC3)cc3c(-c4nnc(C(F)F)s4)cnn23)CC1]

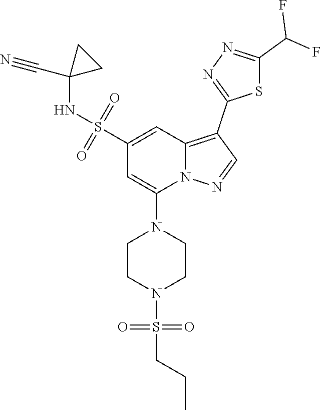 [SMILES: CCCS(=O)(=O)N1CCN(c2cc(S(=O)(=O)NC3(C#N)CC3)cc3c(-c4nnc(C(F)F)s4)cnn23)CC1]

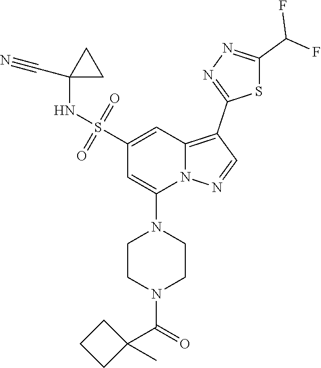 [SMILES: CC1(C(=O)N2CCN(c3cc(S(=O)(=O)NC4(C#N)CC4)cc4c(-c5nnc(C(F)F)s5)cnn34)CC2)CCC1]

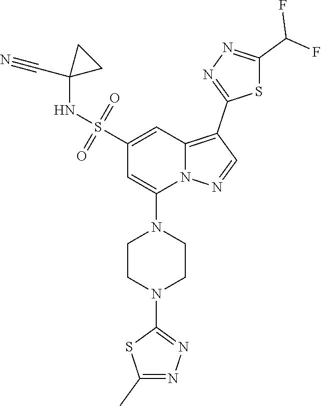 [SMILES: Cc1nnc(N2CCN(c3cc(S(=O)(=O)NC4(C#N)CC4)cc4c(-c5nnc(C(F)F)s5)cnn34)CC2)s1]

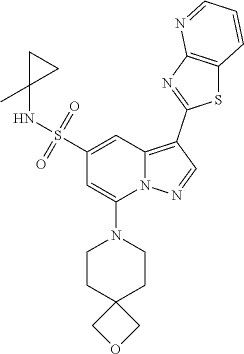 [SMILES: CC1(NS(=O)(=O)c2cc(N3CCC4(CC3)COC4)n3ncc(-c4nc5ncccc5s4)c3c2)CC1]

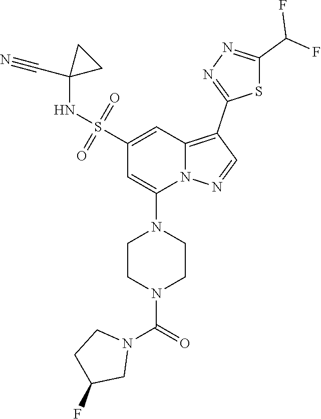 [SMILES: N#CC1(NS(=O)(=O)c2cc(N3CCN(C(=O)N4CC[C@H](F)C4)CC3)n3ncc(-c4nnc(C(F)F)s4)c3c2)CC1]

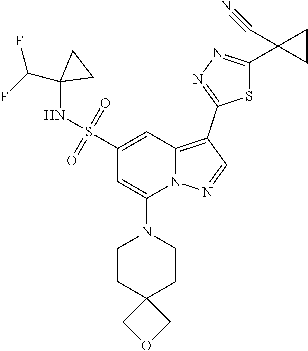 [SMILES: N#CC1(c2nnc(-c3cnn4c(N5CCC6(CC5)COC6)cc(S(=O)(=O)NC5(C(F)F)CC5)cc34)s2)CC1]

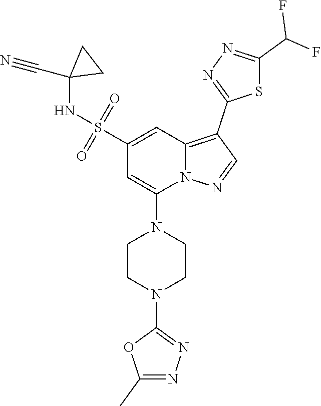 [SMILES: Cc1nnc(N2CCN(c3cc(S(=O)(=O)NC4(C#N)CC4)cc4c(-c5nnc(C(F)F)s5)cnn34)CC2)o1]

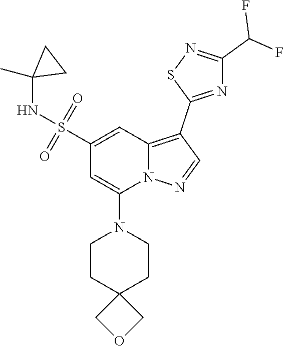 [SMILES: CC1(NS(=O)(=O)c2cc(N3CCC4(CC3)COC4)n3ncc(-c4nc(C(F)F)ns4)c3c2)CC1]

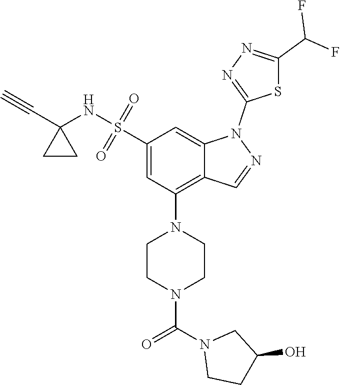 [SMILES: C#CC1(NS(=O)(=O)c2cc(N3CCN(C(=O)N4CC[C@H](O)C4)CC3)c3cnn(-c4nnc(C(F)F)s4)c3c2)CC1]

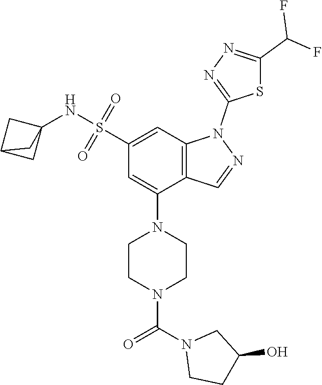 [SMILES: O=C(N1CCN(c2cc(S(=O)(=O)NC34CC(C3)C4)cc3c2cnn3-c2nnc(C(F)F)s2)CC1)N1CC[C@H](O)C1]

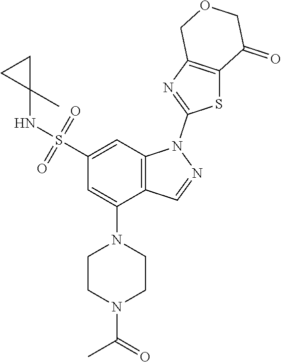 [SMILES: CC(=O)N1CCN(c2cc(S(=O)(=O)NC3(C)CC3)cc3c2cnn3-c2nc3c(s2)C(=O)COC3)CC1]

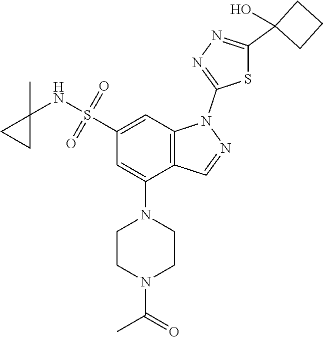 [SMILES: CC(=O)N1CCN(c2cc(S(=O)(=O)NC3(C)CC3)cc3c2cnn3-c2nnc(C3(O)CCC3)s2)CC1]

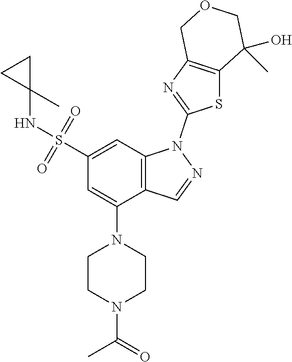 [SMILES: CC(=O)N1CCN(c2cc(S(=O)(=O)NC3(C)CC3)cc3c2cnn3-c2nc3c(s2)C(C)(O)COC3)CC1]

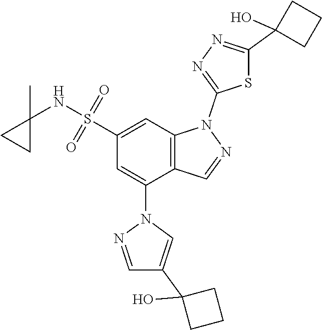 [SMILES: CC1(NS(=O)(=O)c2cc(-n3cc(C4(O)CCC4)cn3)c3cnn(-c4nnc(C5(O)CCC5)s4)c3c2)CC1]

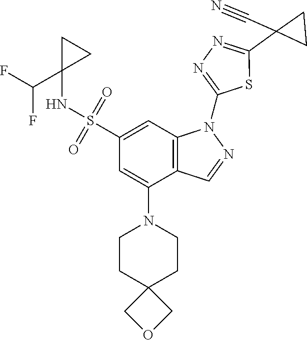 [SMILES: N#CC1(c2nnc(-n3ncc4c(N5CCC6(CC5)COC6)cc(S(=O)(=O)NC5(C(F)F)CC5)cc43)s2)CC1]

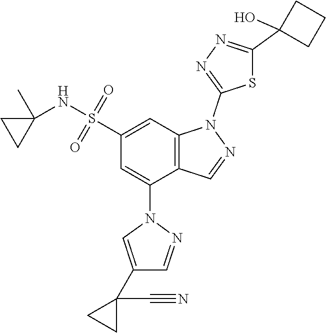 [SMILES: CC1(NS(=O)(=O)c2cc(-n3cc(C4(C#N)CC4)cn3)c3cnn(-c4nnc(C5(O)CCC5)s4)c3c2)CC1]

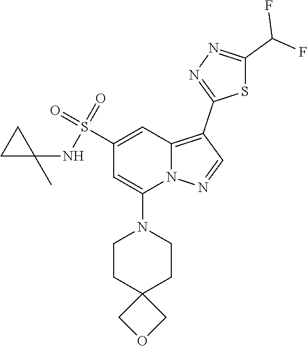 [SMILES: CC1(NS(=O)(=O)c2cc(N3CCC4(CC3)COC4)n3ncc(-c4nnc(C(F)F)s4)c3c2)CC1]

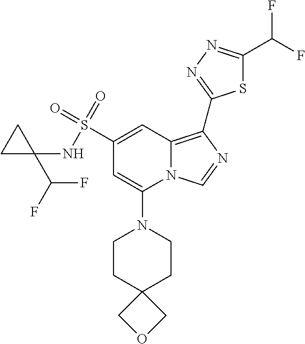 [SMILES: O=S(=O)(NC1(C(F)F)CC1)c1cc(N2CCC3(CC2)COC3)n2cnc(-c3nnc(C(F)F)s3)c2c1]

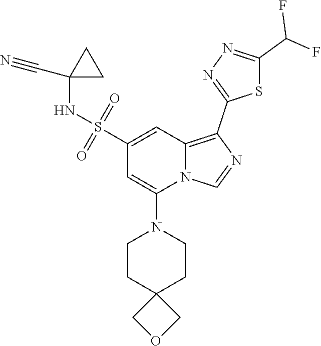 [SMILES: N#CC1(NS(=O)(=O)c2cc(N3CCC4(CC3)COC4)n3cnc(-c4nnc(C(F)F)s4)c3c2)CC1]

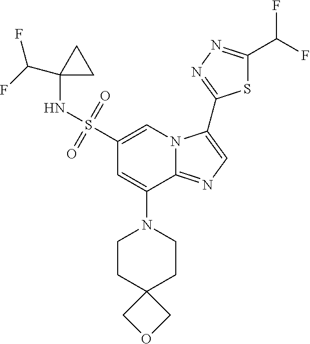 [SMILES: O=S(=O)(NC1(C(F)F)CC1)c1cc(N2CCC3(CC2)COC3)c2ncc(-c3nnc(C(F)F)s3)n2c1]

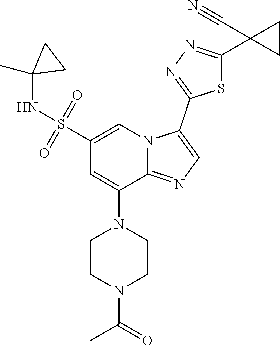 [SMILES: CC(=O)N1CCN(c2cc(S(=O)(=O)NC3(C)CC3)cn3c(-c4nnc(C5(C#N)CC5)s4)cnc23)CC1]

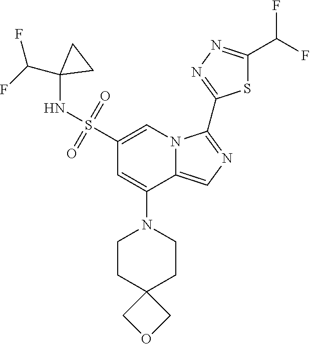 [SMILES: O=S(=O)(NC1(C(F)F)CC1)c1cc(N2CCC3(CC2)COC3)c2cnc(-c3nnc(C(F)F)s3)n2c1]

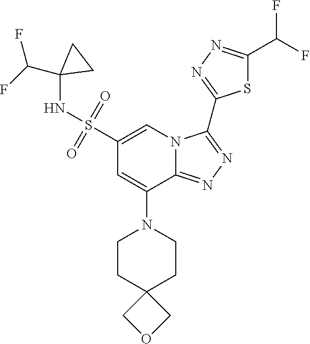 [SMILES: O=S(=O)(NC1(C(F)F)CC1)c1cc(N2CCC3(CC2)COC3)c2nnc(-c3nnc(C(F)F)s3)n2c1]

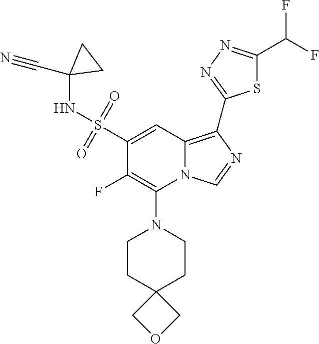 [SMILES: N#CC1(NS(=O)(=O)c2cc3c(-c4nnc(C(F)F)s4)ncn3c(N3CCC4(CC3)COC4)c2F)CC1]

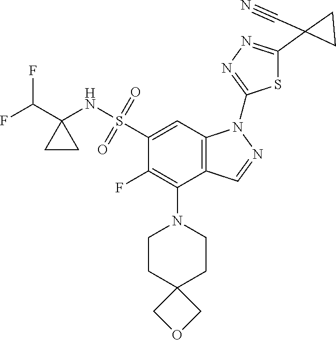 [SMILES: N#CC1(c2nnc(-n3ncc4c(N5CCC6(CC5)COC6)c(F)c(S(=O)(=O)NC5(C(F)F)CC5)cc43)s2)CC1]

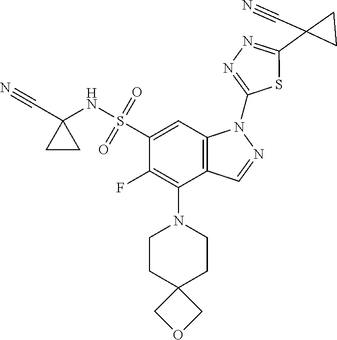 [SMILES: N#CC1(NS(=O)(=O)c2cc3c(cnn3-c3nnc(C4(C#N)CC4)s3)c(N3CCC4(CC3)COC4)c2F)CC1]

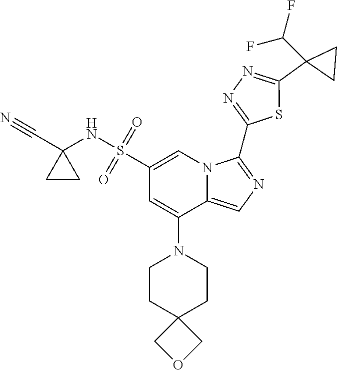 [SMILES: N#CC1(NS(=O)(=O)c2cc(N3CCC4(CC3)COC4)c3cnc(-c4nnc(C5(C(F)F)CC5)s4)n3c2)CC1]

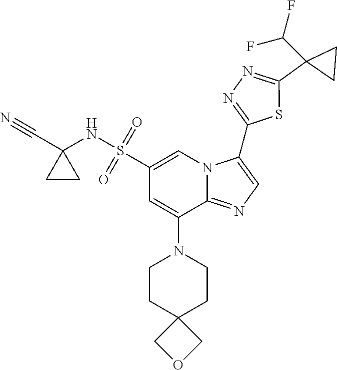 [SMILES: N#CC1(NS(=O)(=O)c2cc(N3CCC4(CC3)COC4)c3ncc(-c4nnc(C5(C(F)F)CC5)s4)n3c2)CC1]

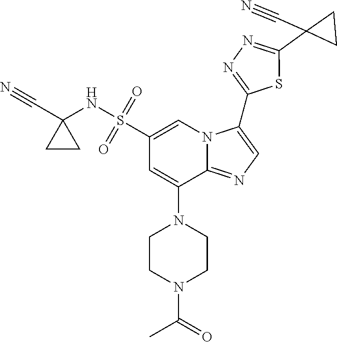 [SMILES: CC(=O)N1CCN(c2cc(S(=O)(=O)NC3(C#N)CC3)cn3c(-c4nnc(C5(C#N)CC5)s4)cnc23)CC1]

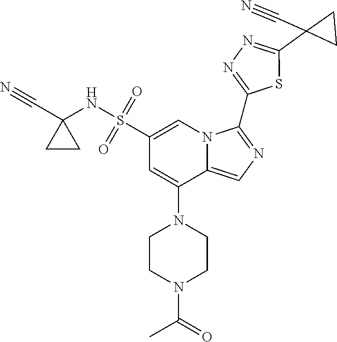 [SMILES: CC(=O)N1CCN(c2cc(S(=O)(=O)NC3(C#N)CC3)cn3c(-c4nnc(C5(C#N)CC5)s4)ncc23)CC1]

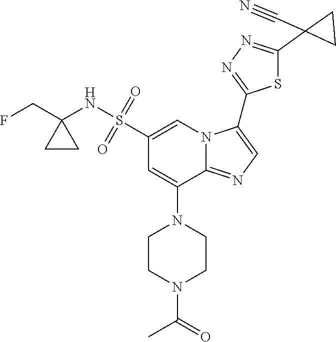 [SMILES: CC(=O)N1CCN(c2cc(S(=O)(=O)NC3(CF)CC3)cn3c(-c4nnc(C5(C#N)CC5)s4)cnc23)CC1]

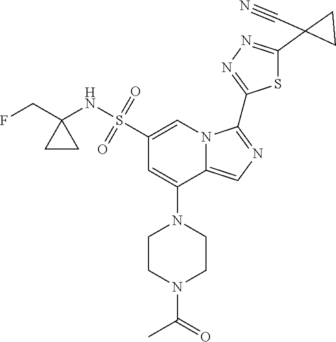 [SMILES: CC(=O)N1CCN(c2cc(S(=O)(=O)NC3(CF)CC3)cn3c(-c4nnc(C5(C#N)CC5)s4)ncc23)CC1]

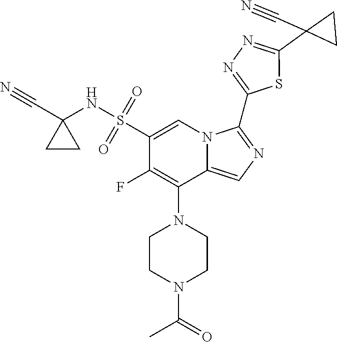 [SMILES: CC(=O)N1CCN(c2c(F)c(S(=O)(=O)NC3(C#N)CC3)cn3c(-c4nnc(C5(C#N)CC5)s4)ncc23)CC1]

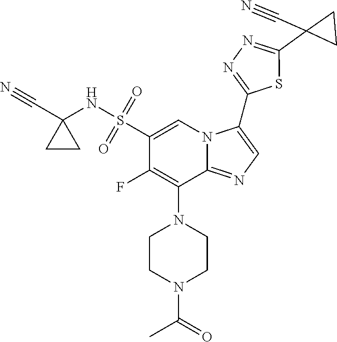 [SMILES: CC(=O)N1CCN(c2c(F)c(S(=O)(=O)NC3(C#N)CC3)cn3c(-c4nnc(C5(C#N)CC5)s4)cnc23)CC1]

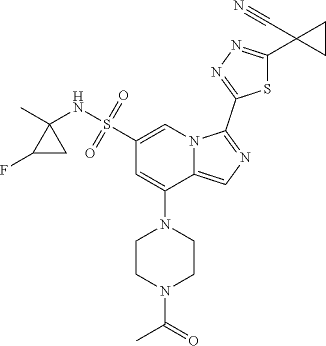 [SMILES: CC(=O)N1CCN(c2cc(S(=O)(=O)NC3(C)CC3F)cn3c(-c4nnc(C5(C#N)CC5)s4)ncc23)CC1]

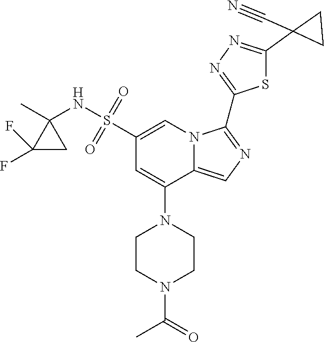 [SMILES: CC(=O)N1CCN(c2cc(S(=O)(=O)NC3(C)CC3(F)F)cn3c(-c4nnc(C5(C#N)CC5)s4)ncc23)CC1]

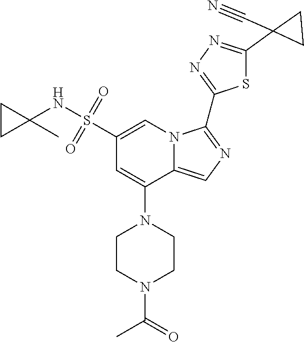 [SMILES: CC(=O)N1CCN(c2cc(S(=O)(=O)NC3(C)CC3)cn3c(-c4nnc(C5(C#N)CC5)s4)ncc23)CC1]

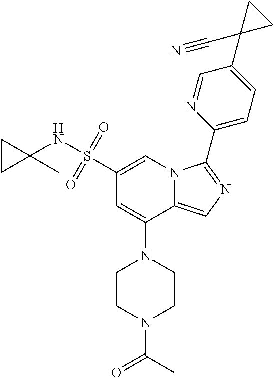 [SMILES: CC(=O)N1CCN(c2cc(S(=O)(=O)NC3(C)CC3)cn3c(-c4ccc(C5(C#N)CC5)cn4)ncc23)CC1]

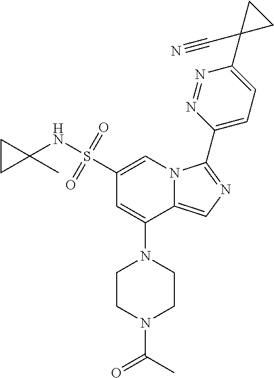 [SMILES: CC(=O)N1CCN(c2cc(S(=O)(=O)NC3(C)CC3)cn3c(-c4ccc(C5(C#N)CC5)nn4)ncc23)CC1]

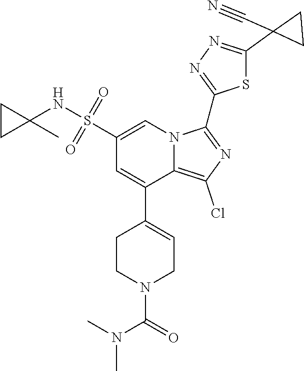 [SMILES: CN(C)C(=O)N1CC=C(c2cc(S(=O)(=O)NC3(C)CC3)cn3c(-c4nnc(C5(C#N)CC5)s4)nc(Cl)c23)CC1]

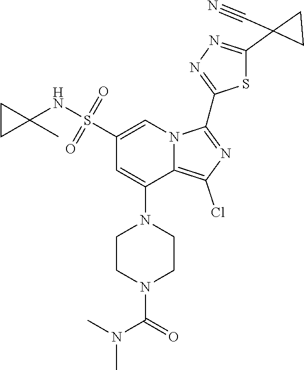 [SMILES: CN(C)C(=O)N1CCN(c2cc(S(=O)(=O)NC3(C)CC3)cn3c(-c4nnc(C5(C#N)CC5)s4)nc(Cl)c23)CC1]

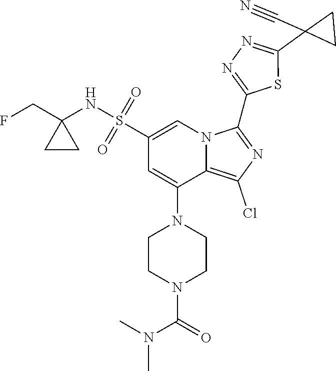 [SMILES: CN(C)C(=O)N1CCN(c2cc(S(=O)(=O)NC3(CF)CC3)cn3c(-c4nnc(C5(C#N)CC5)s4)nc(Cl)c23)CC1]

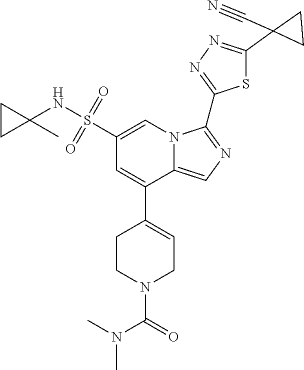 [SMILES: CN(C)C(=O)N1CC=C(c2cc(S(=O)(=O)NC3(C)CC3)cn3c(-c4nnc(C5(C#N)CC5)s4)ncc23)CC1]

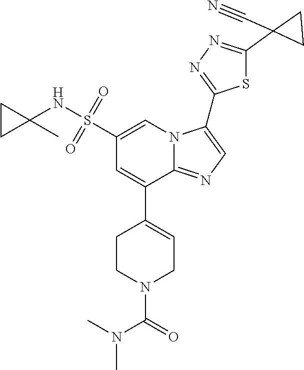 [SMILES: CN(C)C(=O)N1CC=C(c2cc(S(=O)(=O)NC3(C)CC3)cn3c(-c4nnc(C5(C#N)CC5)s4)cnc23)CC1]

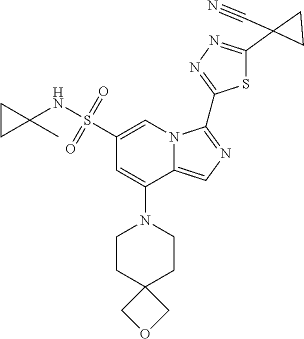 [SMILES: CC1(NS(=O)(=O)c2cc(N3CCC4(CC3)COC4)c3cnc(-c4nnc(C5(C#N)CC5)s4)n3c2)CC1]

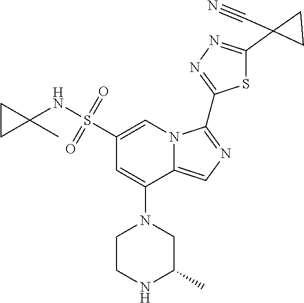 [SMILES: C[C@H]1CN(c2cc(S(=O)(=O)NC3(C)CC3)cn3c(-c4nnc(C5(C#N)CC5)s4)ncc23)CCN1]

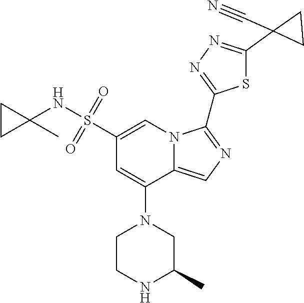 [SMILES: C[C@@H]1CN(c2cc(S(=O)(=O)NC3(C)CC3)cn3c(-c4nnc(C5(C#N)CC5)s4)ncc23)CCN1]

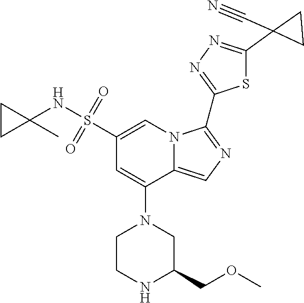 [SMILES: COC[C@@H]1CN(c2cc(S(=O)(=O)NC3(C)CC3)cn3c(-c4nnc(C5(C#N)CC5)s4)ncc23)CCN1]

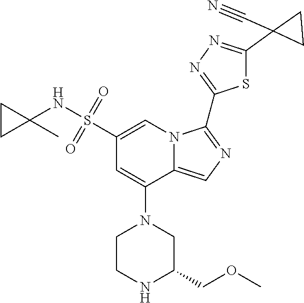 [SMILES: COC[C@H]1CN(c2cc(S(=O)(=O)NC3(C)CC3)cn3c(-c4nnc(C5(C#N)CC5)s4)ncc23)CCN1]

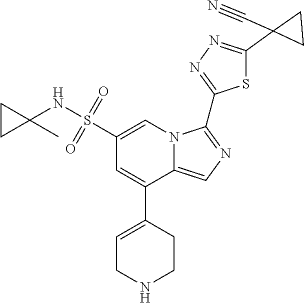 [SMILES: CC1(NS(=O)(=O)c2cc(C3=CCNCC3)c3cnc(-c4nnc(C5(C#N)CC5)s4)n3c2)CC1]

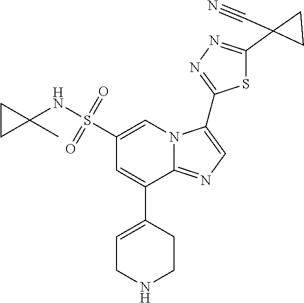 [SMILES: CC1(NS(=O)(=O)c2cc(C3=CCNCC3)c3ncc(-c4nnc(C5(C#N)CC5)s4)n3c2)CC1]

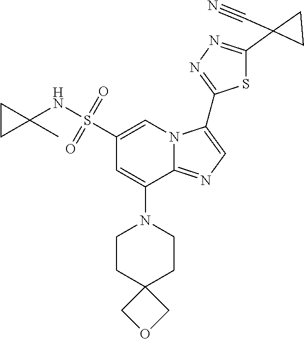 [SMILES: CC1(NS(=O)(=O)c2cc(N3CCC4(CC3)COC4)c3ncc(-c4nnc(C5(C#N)CC5)s4)n3c2)CC1]

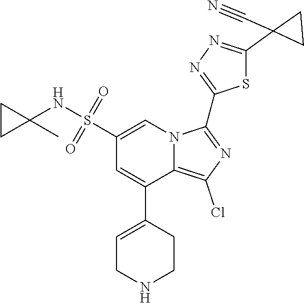 [SMILES: CC1(NS(=O)(=O)c2cc(C3=CCNCC3)c3c(Cl)nc(-c4nnc(C5(C#N)CC5)s4)n3c2)CC1]

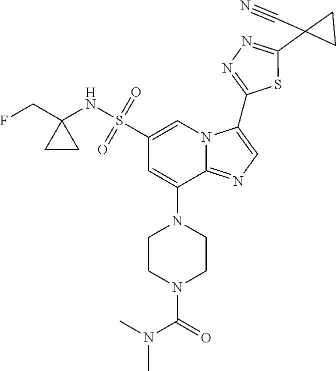 [SMILES: CN(C)C(=O)N1CCN(c2cc(S(=O)(=O)NC3(CF)CC3)cn3c(-c4nnc(C5(C#N)CC5)s4)cnc23)CC1]

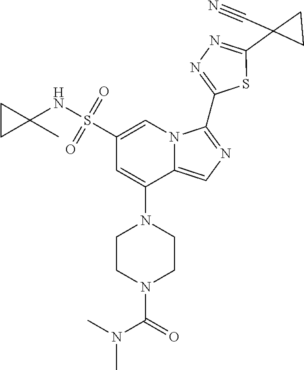 [SMILES: CN(C)C(=O)N1CCN(c2cc(S(=O)(=O)NC3(C)CC3)cn3c(-c4nnc(C5(C#N)CC5)s4)ncc23)CC1]

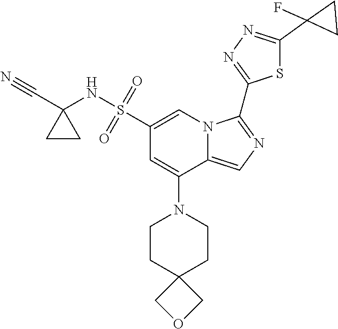 [SMILES: N#CC1(NS(=O)(=O)c2cc(N3CCC4(CC3)COC4)c3cnc(-c4nnc(C5(F)CC5)s4)n3c2)CC1]

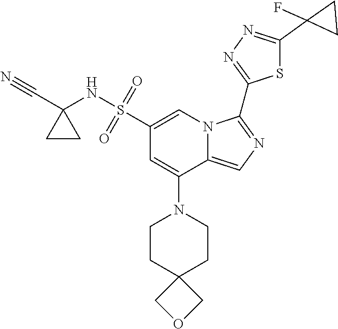 [SMILES: N#CC1(NS(=O)(=O)c2cc(N3CCC4(CC3)COC4)c3cnc(-c4nnc(C5(F)CC5)s4)n3c2)CC1]

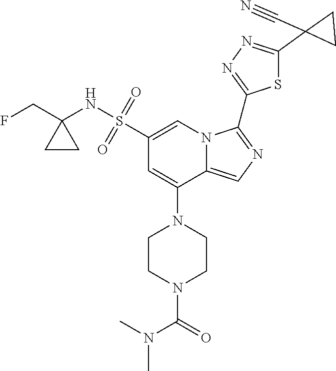 [SMILES: CN(C)C(=O)N1CCN(c2cc(S(=O)(=O)NC3(CF)CC3)cn3c(-c4nnc(C5(C#N)CC5)s4)ncc23)CC1]

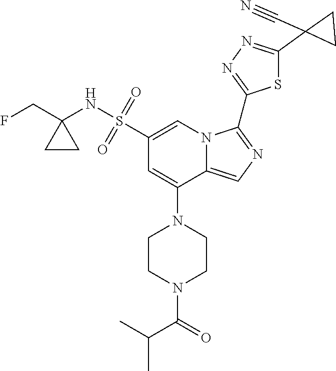 [SMILES: CC(C)C(=O)N1CCN(c2cc(S(=O)(=O)NC3(CF)CC3)cn3c(-c4nnc(C5(C#N)CC5)s4)ncc23)CC1]

In some embodiments, to the extent applicable, the genus of compounds described herein also excludes any specifically known single compounds prior to this disclosure. In some embodiments, to the extent applicable, any sub-genus or species of compounds prior to this disclosure that are entirely within a genus of compounds described herein can also be excluded from such genus herein.

### Method of Synthesis

The compounds of the present disclosure can be readily synthesized by those skilled in the art in view of the present disclosure. Exemplified synthesis are also shown in the Examples section.

As will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in “Protective Groups in Organic Synthesis”, 4^{th} ed. P. G. M. Wuts; T. W. Greene, John Wiley, 2007, and references cited therein. The reagents for the reactions described herein are generally known compounds or can be prepared by known procedures or obvious modifications thereof. For example, many of the reagents are available from commercial suppliers such as Aldrich Chemical Co. (Milwaukee, Wisconsin, USA), Sigma (St. Louis, Missouri, USA). Others may be prepared by procedures, or obvious modifications thereof, described in standard reference texts such as Fieser and Fieser's Reagents for Organic Synthesis, Volumes 1-15 (John Wiley and Sons, 1991), Rodd's Chemistry of Carbon Compounds, Volumes 1-5 and Supplemental (Elsevier Science Publishers, 1989), Organic Reactions, Volumes 1-40 (John Wiley and Sons, 1991), March's Advanced Organic Chemistry, (Wiley, 7^{th} Edition), and Larock's Comprehensive Organic Transformations (Wiley-VCH, 1999), and any of available updates as of this filing.

### Pharmaceutical Compositions

Certain embodiments are directed to a pharmaceutical composition comprising one or more compounds of the present disclosure.

The pharmaceutical composition can optionally contain a pharmaceutically acceptable excipient. In some embodiments, the pharmaceutical composition comprises a compound of the present disclosure (e.g., a compound of Formula I (e.g., Formula I-1, I-2, I-3, I-1a, I-1b, I-1c, I-2a, I-2b, J-3a, I-3b, I-1a-1, I-1a-2, I-2a-1, J-2a-2, I-3a-1, I-3a-2, I-1b-1, I-1b-2, I-2b-1, I-2b-2, I-3b-1, I-3b-2, I-A, I-A1, I-A2, I-B, I-C, or I-D), any of the compounds listed in Table 1 herein, or a pharmaceutically acceptable salt thereof) and a pharmaceutically acceptable excipient. Pharmaceutically acceptable excipients are known in the art. Non-limiting suitable excipients include, for example, encapsulating materials or additives such as antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. See also Remington's The Science and Practice of Pharmacy, 21st Edition, A. R. Gennaro (Lippincott, Williams & Wilkins, Baltimore, Md., 2005; incorporated herein by reference), which discloses various excipients used in formulating pharmaceutical compositions and known techniques for the preparation thereof.

The pharmaceutical composition can include any one or more of the compounds of the present disclosure. For example, in some embodiments, the pharmaceutical composition comprises a compound of Formula I or a pharmaceutically acceptable salt thereof, e.g., in a therapeutically effective amount. In any of the embodiments described herein, the pharmaceutical composition can comprise a therapeutically effective amount (e.g., for treating a cancer herein) of a compound selected from any of Examples 1-41, or any of the specific compounds disclosed in Table 1 herein, or a pharmaceutically acceptable salt thereof. In some preferred embodiments, the pharmaceutical composition can comprise a compound selected from the compounds according to Examples 1-41 that have an IC₅₀ value less than 100 nM, more preferably, less than 50 nM, as measured according to Biological Example 1.

The pharmaceutical composition herein can be formulated for delivery via any of the known routes of delivery, which include but not limited to administering orally, nasally, transdermally, pulmonary, inhalationally, buccally, sublingually, intraperintoneally, subcutaneously, intramuscularly, intravenously, rectally, intrapleurally, intrathecally or parenterally.

In some embodiments, the pharmaceutical composition can be formulated for oral administration. The oral formulations can be presented in discrete units, such as capsules, pills, cachets, lozenges, or tablets, each containing a predetermined amount of the active compound; as a powder or granules; as a solution or a suspension in an aqueous or non-aqueous liquid; or as an oil-in-water or water-in-oil emulsion. Excipients for the preparation of compositions for oral administration are known in the art. Non-limiting suitable excipients include, for example, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl cellulose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, and mixtures thereof.

In some embodiments, the pharmaceutical composition is formulated for parenteral administration (such as intravenous injection or infusion, subcutaneous or intramuscular injection). The parenteral formulations can be, for example, an aqueous solution, a suspension, or an emulsion. Excipients for the preparation of parenteral formulations are known in the art. Non-limiting suitable excipients include, for example, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water and mixtures thereof.

Compounds of the present disclosure can be used alone, in combination with each other, or in combination with one or more additional therapeutic agents, e.g., in combination with an additional anticancer therapeutic agent, such as a chemotherapeutic agent described herein, or those chemotherapeutic agents described in WO2023/057389, WO2021/055744, and WO2023/057394.

When used in combination with one or more additional therapeutic agents, compounds of the present disclosure or pharmaceutical compositions herein can be administered to the subject either concurrently or sequentially in any order with such additional therapeutic agents. In some embodiments, the pharmaceutical composition can comprise one or more compounds of the present disclosure and the one or more additional therapeutic agents in a single composition. In some embodiments, the pharmaceutical composition comprising one or more compounds of the present disclosure can be included in a kit which also comprises a separate pharmaceutical composition comprising the one or more additional therapeutic agents.

The pharmaceutical composition can include various amounts of the compounds of the present disclosure, depending on various factors such as the intended use and potency and selectivity of the compounds. In some embodiments, the pharmaceutical composition comprises a therapeutically effective amount of a compound of the present disclosure. In some embodiments, the pharmaceutical composition comprises a therapeutically effective amount of the compound of the present disclosure and a pharmaceutically acceptable excipient. As used herein, a therapeutically effective amount of a compound of the present disclosure is an amount effective to treat a disease or disorder as described herein, such as a cancer herein, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### Method of Treatment/Use

Compounds of the present disclosure have various utilities. For example, compounds of the present disclosure can be used as therapeutic active substances for the treatment and/or prophylaxis of a disease or disorder in which PARG activity is implicated. Accordingly, some embodiments of the present disclosure are also directed to methods of using one or more compounds of the present disclosure or pharmaceutical compositions herein for treating or preventing a disease or disorder in which PARG activity is implicated in a subject in need thereof, such as for treating cancer in a subject in need thereof.

In some embodiments, the present disclosure provides a method of inhibiting PARG enzyme activity in a cell, the method comprising contacting the cell with an effective amount of the compound of present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-l, I-2a-2, I-3a-1, I-3a-2, I-1b-1, I-1b-2, I-2b-1, I-2b-2, I-3b-1, I-3b-2, I-A, I-A1, I-A2, I-B, I-C, or I-D), any of the compounds listed in Table I herein, or a pharmaceutically acceptable salt thereof). In some embodiment, contacting the cell occurs in vitro. In some embodiment, contacting the cell occurs in vivo. In some embodiments, the method selectively inhibits PARG enzyme activity over PARP1 or ARH3 enzyme activity.

In some embodiments, the present disclosure provides a method of inhibiting cell proliferation, in vitro or in vivo, the method comprising contacting the cell with an effective amount of the compound of present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-1, I-2a-2, I-3a-1, I-3a- 2, I-1b-1, I-1b-2, I-2b-1, I-2b-2, I-3b-1, I-3b-2, I-A, I-A1, I-A2, I-B, I-C, or I-D), any of the compounds listed in Table 1 herein, or a pharmaceutically acceptable salt thereof).

In some embodiments, the present disclosure provides a method of treating a disease or disorder in which PARG activity is implicated in a subject in need thereof, the method comprising administering to the subject a therapeutically effective amount of the compound of present disclosure (e.g., a compound of Formula I (e.g., Formula I-1, I-2, I-3, I-1a, I-1b, I-1c, 1-2a, 1-2b, I-3a, I-3b, I-1a-1, 1-1a-2, T-2a-1, I-2a-2, T-3a-1, 1-3a-2, 1-1b-1, 1-1b-2, I-2b-1, I-2b-2, I-3b-1, I-3b-2, I-A, I-A1, I-A2, I-B, I-C, or I-D), any of the compounds listed in Table 1 herein, or a pharmaceutically acceptable salt thereof), or a pharmaceutical composition as defined herein. In some embodiments, the disease or disorder in which PARG activity is implicated is a proliferative disorder described herein. In some embodiments, the disease or disorder in which PARG activity is implicated is a cancer described herein. In some embodiments, the cancer can be selected from ovarian cancer, breast cancer, prostate cancer, pancreatic cancer, lung cancer (such as small cell lung carcinoma (SCLC)_, colorectal cancer, melanoma, sarcoma, and gastric cancer.

In some embodiments, the present disclosure provides a method of treating a proliferative disorder in a subject in need thereof, the method comprising administering to the subject a therapeutically effective amount of the compound of present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, I-3b-2, I- A, I-A1, I-A2, I-B, I-C, or I-D), any of the compounds listed in Table 1 herein, or a pharmaceutically acceptable salt thereof), or a pharmaceutical composition as defined herein. Examples of proliferative conditions include, but are not limited to, pre-malignant and malignant cellular proliferation, including but not limited to, malignant neoplasms and tumours, cancers, leukemias, psoriasis, bone diseases, fibroproliferative disorders (e.g., of connective tissues), and atherosclerosis. Any type of cell may be treated, including but not limited to, lung, colon, breast, ovarian, prostate, liver, pancreas, brain, and skin.

Preferably, the proliferative disorder is cancer. Thus, in some embodiments, the present disclosure provides a method of treating cancer in a subject in need thereof, the method comprising administering to the subject a therapeutically effective amount of the compound of present disclosure (e.g., a compound of Formula I (e.g., Formula I-1, I-2, I-3, I-1a, I-1b, I-1c, I-2a, I-2b, I-3a, I-3b, I-1a-1, I-1a-2, I-2a-l, I-2a-2, I-3a-1, I-3a-2, I-1b-i, I-1b-2, I-2b-l, I-2b-2, I-3b-1, I-3b-2, I-A, I-A1, I-A2, I-B, I-C, or I-D), any of the compounds listed in Table 1 herein, or a pharmaceutically acceptable salt thereof), or a pharmaceutical composition as defined herein. In some embodiments, the cancer can be selected from ovarian cancer, breast cancer, prostate cancer, pancreatic cancer, lung cancer (such as small cell lung carcinoma (SCLC)), colorectal cancer, melanoma, sarcoma, and gastric cancer. In a particular embodiment, the cancer is human cancer.

Compounds of the present disclosure can be used in the methods herein as a monotherapy or in a combination therapy. For example, in some embodiments, the compound of the present disclosure may be used in combination with conventional surgery or radiotherapy or chemotherapy. In some embodiments, such chemotherapy may include one or more of the following anti-tumour agents, (i) antiproliferative/antineoplastic drugs and combinations thereof, such as alkylating agents (for example cis-platin, oxaliplatin, carboplatin, cyclophosphamide, nitrogen mustard, melphalan, chlorambucil, busulphan, temozolamide and nitrosoureas); antimetabolites (for example gemcitabine and antifolates such as fluoropyrimidines like 5-fluorouracil and tegafur, raltitrexed, methotrexate, cytosine arabinoside, and hydroxyurea); antitumour antibiotics (for example anthracyclines like adriamycin, bleomycin, doxorubicin, daunomycin, epirubicin, idarubicin, mitomycin-C, dactinomycin and mithramycin); antimitotic agents (for example vinca alkaloids like vincristine, vinblastine, vindesine and vinorelbine and taxoids like taxol and taxotere and polokinase inhibitors); and topoisomerase inhibitors (for example epipodophyllotoxins like etoposide and teniposide, amsacrine, topotecan and camptothecin); (ii) cytostatic agents such as antioestrogens (for example tamoxifen, fulvestrant, toremifene, raloxifene, droloxifene and iodoxyfene), antiandrogens (for example bicalutamide, flutamide, nilutamide and cyproterone acetate), LHRH antagonists or LHRH agonists (for example goserelin, leuprorelin and buserelin), progestogens (for example megestrol acetate), aromatase inhibitors (for example as anastrozole, letrozole, vorazole and exemestane) and inhibitors of 5oc-reductase such as finasteride; (iii) anti-invasion agents [for example c-Src kinase family inhibitors like 4-(6-chloro-2,3-methylenedioxyanilino)-7-[2-(4-methylpiperazin-1-yl)ethoxy]-5-tetrahydropyran-4-yloxyquinazoline (AZD0530; International Patent Application WO 01/94341), N-(2-chloro-6-methylphenyl)-2-{6-[4-(2-hydroxyethyl)piperazin-1-yl]-2-methylpyrimidin-4-ylamino}thiazole-5-carboxamide (dasatinib, BMS-354825; J. Med. Chem., 2004, 47, 6658-6661) and bosutinib (SKI-606), and metalloproteinase inhibitors like marimastat, inhibitors of urokinase plasminogen activator receptor function or antibodies to Heparanase]; (iv) inhibitors of growth factor function: for example such inhibitors include growth factor antibodies and growth factor receptor antibodies (for example the anti-erbB2 antibody trastuzumab [Herceptin™], the anti-EGFR antibody panitumumab, the anti-erbB 1 antibody cetuximab [Erbitux, C225] and any growth factor or growth factor receptor antibodies disclosed by Stern et al. (Critical reviews in oncology/haematology, 2005, Vol. 54, pp 11-29); such inhibitors also include tyrosine kinase inhibitors, for example inhibitors of the epidermal growth factor family (for example EGFR family tyrosine kinase inhibitors such as N-(3-chloro-4-fluorophenyl)-7-methoxy-6-(3-morpholinopropoxy)quinazolin-4-amine (gefitinib, ZD1839), N-(3-ethynylphenyl)-6,7-bis(2-methoxyethoxy)quinazolin-4-amine (erlotinib, OSI-774) and 6-acrylamido-N-(3-chloro-4-fluorophenyl)-7-(3-morpholinopropoxy)-quinazolin-4-amine (Cl 1033), erbB2 tyrosine kinase inhibitors such as lapatinib); inhibitors of the hepatocyte growth factor family; inhibitors of the insulin growth factor family; inhibitors of the platelet-derived growth factor family such as imatinib and/or nilotinib (AMN107); inhibitors of serine/threonine kinases (for example Ras/Raf signalling inhibitors such as farnesyl transferase inhibitors, for example sorafenib (BAY 43-9006), tipifarnib (RI 15777) and lonafarnib (SCH66336)), inhibitors of cell signalling through MEK and/or AKT kinases, c-kit inhibitors, abl kinase inhibitors, PI3 kinase inhibitors, Plt3 kinase inhibitors, CSF-1 R kinase inhibitors, IGF receptor (insulin-like growth factor) kinase inhibitors; aurora kinase inhibitors (for example AZD1 152, PH739358, VX-680, MLN8054, R763, MP235, MP529, VX-528 AND AX39459) and cyclin dependent kinase inhibitors such as CDK2 and/or CDK4 inhibitors; (v) antiangiogenic agents such as those which inhibit the effects of vascular endothelial growth factor, [for example the anti-vascular endothelial cell growth factor antibody bevacizumab (Avastin™) and for example, a VEGF receptor tyrosine kinase inhibitor such as vandetanib (ZD6474), vatalanib (PTK787), sunitinib (SU1 1248), axitinib (AG-013736), pazopanib (GW 786034) and 4-(4-fluoro-2-methylindol-5-yloxy)-6-methoxy-7-(3-pyrrolidin-1-ylpropoxy)quinazoline (AZD2171; Example 240 within WO 00/47212), compounds such as those disclosed in International Patent Applications WO97/22596, WO 97/30035, WO 97/32856 and WO 98/13354 and compounds that work by other mechanisms (for example linomide, inhibitors of integrin αvβ3 function and angiostatin)]; (vi) vascular damaging agents such as Combretastatin A4 and compounds disclosed in International Patent Applications WO 99/02166, WO 00/40529, WO 00/41669, WO 01/92224, WO 02/04434 and WO 02/08213; (vii) an endothelin receptor antagonist, for example zibotentan (ZD4054) or atrasentan; (viii) antisense therapies, for example those which are directed to the targets listed above, such as ISIS 2503, an anti-ras antisense; (ix) gene therapy approaches, including for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; and (x) immunotherapy approaches, including for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies. Agents that may be combined with a PARG inhibitor also include those described in WO2023/057389, WO2021/055744, and WO2023/057394.

As understood herein, the term “combination” refers to simultaneous, separate or sequential administration. In one aspect of the invention “combination” refers to simultaneous administration. In another aspect of the invention “combination” refers to separate administration. In a further aspect of the invention “combination” refers to sequential administration. Where the administration is sequential or separate, the delay in administering the second component should not be such as to lose the beneficial effect of the combination.

Dosing regimen including doses for the methods described herein can vary and be adjusted, which can depend on the recipient of the treatment, the disorder, condition or disease being treated and the severity thereof, the composition containing the compound, the time of administration, the route of administration, the duration of treatment, the compound potency, its rate of clearance and whether or not another drug is co-administered.

### DEFINITIONS

It is meant to be understood that proper valences are maintained for all moieties and combinations thereof.

It is also meant to be understood that a specific embodiment of a variable moiety herein can be the same or different as another specific embodiment having the same identifier.

Suitable groups for the variables in compounds of Formula I, or a subformula thereof, as applicable, are independently selected. Non-limiting useful groups for the variables in compounds of Formula I, or a subformula thereof, as applicable, include any of the respective groups, individually or in any combination, as shown in the Examples or in the specific compounds described in Table 1 herein. In addition, it is to be understood that the definition of a variable in Formula 1 can have the same definition for the variable defined in a subformula of Formula L. Similarly, unless otherwise specified or contrary from context, the definition of a subformula of Formula I can have the same definition for the variable defined in connection with Formula I or another subformula of Formula I.

The described embodiments of the present disclosure can be combined. Such combination is contemplated and within the scope of the present disclosure. For example, it is contemplated that the definition(s) of any one or more of J¹, J², J³, J⁴, J⁵, J⁶, J⁷, J⁸, L¹, L², R¹, and R² of Formula I can be combined with the definition of any one or more of the other(s) of J¹, J², J³, J⁴, J⁵, J⁶, J⁷, J⁸, L¹, L², R¹, and R², as applicable, and the resulted compounds from the combination are within the scope of the present disclosure.

The symbol, [symbol] when displayed perpendicular to (or otherwise crossing) a bond, indicates the point at which the displayed moiety is attached to the remainder of the molecule. It should be noted that for a divalent structure (or multivalent structure), the immediately connected group or groups or appropriate variable(s) shown in a formula may be shown in the divalent structure (or multivalent structure) beyond the symbol, [symbol] to indicate direction of attachment. When the immediately connected group(s) or variable is not shown for either of the two attaching points of a divalent structure, it should mean that either direction of attachment to the remainder of the molecule is allowed, unless otherwise specified or obviously contrary from context.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high performance liquid chromatography (HPLC), chiral supercritical fluid chromatograph (SFC), and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E. L. Eliel, Ed., Univ. of Notre Dame Press, Notre Dame, IN 1972). The disclosure additionally encompasses compounds described herein as individual isomers substantially free of other isomers, and alternatively, as mixtures of various isomers including racemic mixtures. When a stereochemistry is specifically drawn, unless otherwise contradictory from context, it should be understood that with respect to that particular chiral center or axial chirality, the compound can exist predominantly as the as-drawn stereoisomer, such as with less than 20%, less than 10%, less than 5%, less than 1%, by weight, by HPLC or SFC area, or both, or with a non-detectable amount of the other stereoisomer(s), for example, the compound can have an enantiomeric excess of greater than 60%, greater than 80%, greater than 90%, greater than 95%, greater than 98%, or greater than 99%. The presence and/or amounts of stereoisomers can be determined by those skilled in the art in view of the present disclosure, including through the use of a chiral HPLC or chiral SFC. As understood by those skilled in the art, when a “*” is shown in the chemical structures herein, unless otherwise contradictory from context, it is to designate that the corresponding chiral center is enantiomerically pure or enriched in either of the configurations or is enantiomerically pure or enriched in the as-dawn configuration, such as with less than 20%, less than 10%, less than 5%, less than 1%, by weight, by HPLC or SFC area, or both, or with a non-detectable amount of the other stereoisomer(s). Also, when no stereochemistry is specifically drawn, and no “*” is used in the chemical structures, unless otherwise contradictory from context, it should be understood that such structures include the corresponding compound in any stereoisomeric forms, including individual isomers substantially free of other isomers and mixtures of various isomers including racemic mixtures.

When a range of values is listed, it is intended to encompass each value and sub-range within the range. For example “C₁₋₆” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

As used herein, the term “compound(s) of the present disclosure” refers to any of the compounds described herein according to Formula I (e.g., I-1, I-2, I-3, I-1a, I-1b, I-1c, I-2a, I-2b, I-3a, I-3b, I-1a-1, I-1a-2, I-2a-1, I-2a-2, I-3a-1, I-3a-2, I-1b-1, I-1b-2, I-2b-1, I-2b-2, I-3b-1, I-3b-2, I-A, I-A1, I-A2, I-B, I-C, or I-D), any of Examples 1-41, or any of the specific compounds disclosed in Table 1 herein, isotopically labeled compound(s) thereof (such as a deuterated analog wherein one or more of the hydrogen atoms is/are substituted with a deuterium atom with an abundance above its natural abundance, e.g., a CD₃ analog when the compound has a CH₃ group), possible regioisomers, possible geometric isomers, possible stereoisomers thereof (including diastereoisomers, enantiomers, and racemic mixtures), tautomers thereof, conformational isomers thereof, pharmaceutically acceptable esters thereof, and/or possible pharmaceutically acceptable salts thereof (e.g., acid addition salt such as HCl salt or base addition salt such as Na salt). In some embodiments, the compounds of the present disclosure can be selected from any of the enumerated embodiments A1-29. In some embodiments, the compounds of the present disclosure can be selected from any of the enumerated embodiments B1-43. In some embodiments, the compounds of the present disclosure can be selected from any of the enumerated embodiments C1-26. Hydrates and solvates of the compounds of the present disclosure are considered compositions of the present disclosure, wherein the compound(s) is in association with water or solvent, respectively.

Compounds of the present disclosure can exist in isotope-labeled or -enriched form containing one or more atoms having an atomic mass or mass number different from the atomic mass or mass number most abundantly found in nature. Isotopes can be radioactive or non-radioactive isotopes. Isotopes of atoms such as hydrogen, carbon, phosphorous, sulfur, fluorine, chlorine, and iodine include, but are not limited to ²H, ³H, ¹³C, ¹⁴C, ¹⁵N, ¹⁸O, ³²P, ³⁵S, ¹⁸F, ³⁶Cl, and ¹²⁵I. Compounds that contain other isotopes of these and/or other atoms are within the scope of this invention.

As used herein, the phrase “administration” of a compound, “administering” a compound, or other variants thereof means providing the compound or a prodrug of the compound to the individual in need of treatment.

As used herein, the term “alkyl” as used by itself or as part of another group refers to a straight- or branched-chain aliphatic saturated hydrocarbon. In some embodiments, the alkyl can include one to twelve carbon atoms (i.e., C₁₋₁₂ alkyl) or the number of carbon atoms designated. In one embodiment, the alkyl group is a straight chain C₁₋₁₀ alkyl group. In another embodiment, the alkyl group is a branched chain C₃₋₁₀ alkyl group. In another embodiment, the alkyl group is a straight chain C₁₋₆ alkyl group. In another embodiment, the alkyl group is a branched chain C₃₋₆ alkyl group. In another embodiment, the alkyl group is a straight chain C₁₋₄ alkyl group. For example, a C₁₋₄ alkyl group includes methyl, ethyl, propyl (n-propyl), isopropyl, butyl (n-butyl), sec-butyl, tert-butyl, and iso-butyl. As used herein, the term “alkylene” as used by itself or as part of another group refers to a divalent radical derived from an alkyl group. For example, non-limiting straight chain alkylene groups include —CH₂—CH₂—CH₂—CH₂—, —CH₂—CH₂—CH₂—, —CH₂—CH₂—, and the like.

As used herein, the term “alkenyl” as used by itself or as part of another group refers to a straight- or branched-chain aliphatic hydrocarbon containing one or more, for example, one, two or three carbon-to-carbon double bonds. In one embodiment, the alkenyl group is a C₂₋₆ alkenyl group. In another embodiment, the alkenyl group is a C₂₋₄ alkenyl group. Non-limiting exemplary alkenyl groups include ethenyl, propenyl, isopropenyl, butenyl, sec-butenyl, pentenyl, and hexenyl.

As used herein, the term “alkynyl” as used by itself or as part of another group refers to a straight- or branched-chain aliphatic hydrocarbon containing one or more, for example, one to three carbon-to-carbon triple bonds. In one embodiment, the alkynyl has one carbon-carbon triple bond. In one embodiment, the alkynyl group is a C₂₋₆ alkynyl group. In another embodiment, the alkynyl group is a C₂₋₄ alkynyl group. Non-limiting exemplary alkynyl groups include ethynyl, propynyl, butynyl, 2-butynyl, pentynyl, and hexynyl groups.

As used herein, the term “alkoxy” as used by itself or as part of another group refers to a radical of the formula OR^{a1}, wherein R^{a1} is an alkyl.

As used herein, the term “cycloalkoxy” as used by itself or as part of another group refers to a radical of the formula OR^{a1}, wherein R^{a1} is a cycloalkyl.

As used herein, the term “haloalkyl” as used by itself or as part of another group refers to an alkyl substituted with one or more fluorine, chlorine, bromine and/or iodine atoms. In preferred embodiments, the haloalkyl is an alkyl group substituted with one, two, or three fluorine atoms. In one embodiment, the haloalkyl group is a C₁₋₁₀ haloalkyl group. In one embodiment, the haloalkyl group is a C₁₋₆ haloalkyl group. In one embodiment, the haloalkyl group is a C₁₋₄ haloalkyl group.

As used herein, the term “heteroalkyl,” by itself or in combination with another term, means, unless otherwise stated, a stable straight or branched-chain alkyl group, e.g., having from 2 to 14 carbons, such as 2 to 10 carbons in the chain, in which one or more of the carbons has been replaced by a heteroatom selected from S, O, P and N, and wherein the nitrogen, phosphine, and sulfur atoms can optionally be oxidized and the nitrogen heteroatom can optionally be quaternized. The heteroatom(s) S, O. P and N may be placed at any interior position of the heteroalkyl group or at the position at which the alkyl group is attached to the remainder of the molecule. When the heteroalkyl is said to be substituted, the substituent(s) can replace one or more hydrogen atoms attached to the carbon atom(s) and/or the heteroatom(s) of the heteroalkyl. In some embodiments, the heteroalkyl is a C₁₋₄ heteroalkyl, which refers to the heteroalkyl defined herein having 1-4 carbon atoms. Examples of C₁₋₄ heteroalkyl include, but are not limited to, C₄ heteroalkyl such as —CH₂—CH₂—N(CH₃)—CH₃, C₃ heteroalkyl such as —CH₂—CH₂—O—CH₃, —CH₂—CH₂—NH—CH₃, —CH₂—S—CH₂—CH₃, —CH₂—CH₂—S(O)—CH₃, —CH₂—CH₂—S(O)₂—CH₃, C₂ heteroalkyl such as —CH₂—CH₂—OH, —CH₂—CH₂—NH₂, —CH₂—NH(CH₃), —O—CH₂—CH₃ and C₁ heteroalkyl such as, —CH₂—OH, —CH₂—NH₂, —O—CH₃. Preferably, the C₁₋₄ heteroalkyl (or C₁₋₄ heteroalkylene) herein contains 1 or 2 heteroatoms, such as one oxygen, one nitrogen, two oxygens, two nitrogens, or one oxygen and one nitrogen. Similarly, the term “heteroalkylene” by itself or as part of another substituent means a divalent radical derived from heteroalkyl, as exemplified, but not limited by, —CH₂—CH₂—O—CH₂—CH₂— and —O—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

“Carbocyclyl” or “carbocyclic” as used by itself or as part of another group refers to a radical of a non-aromatic cyclic hydrocarbon group having at least 3 carbon atoms, e.g., from 3 to 10 ring carbon atoms (“C₃₋₁₀ carbocyclyl”), and zero heteroatoms in the non-aromatic ring system. The carbocyclyl group can be either monocyclic (“monocyclic carbocyclyl”) or contain a fused, bridged or spiro ring system such as a bicyclic system (“bicyclic carbocyclyl”) and can be saturated or can be partially unsaturated. Non-limiting exemplary carbocyclyl groups include cyclopropyl, cyclobutyl, cyclopentyl, cyclohexyl, cycloheptyl, cyclooctyl, norbornyl, decalin, adamantyl, cyclopentenyl, and cyclohexenyl. As used herein, the term “carbocyclylene” as used by itself or as part of another group refers to a divalent radical derived from the carbocyclyl group defined herein.

In some embodiments, “carbocyclyl” is fully saturated, which is also referred to as cycloalkyl. In some embodiments, the cycloalkyl can have from 3 to 10 ring carbon atoms (“C₃₋₁₀ cycloalkyl”). In preferred embodiments, the cycloalkyl is a monocyclic ring. As used herein, the term “cycloalkylene” as used by itself or as part of another group refers to a divalent radical derived from a cycloalkyl group, for example,

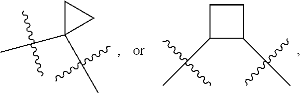 [SMILES: *C1(*)CC1 ; "," ; "or" ; *C1CCC1* ; ","]

etc.

Unless otherwise defined or contrary from context, a heteroatom herein refers to an atom selected from nitrogen, oxygen, sulfur, boron, phosphorus, and silicon.

“Heterocyclyl” or “heterocyclic” as used by itself or as part of another group refers to a radical of a 3-membered or larger, such as 3-to 14-membered, non-aromatic ring system having ring carbon atoms and at least one ring heteroatom, such as 1 to 4 ring heteroatoms, wherein each heteroatom is independently selected from nitrogen, oxygen, sulfur, boron, phosphorus, and silicon. In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged, or spiro ring system, such as a bicyclic system (“bicyclic heterocyclyl”), and can be saturated or can be partially unsaturated. Heterocyclyl bicyclic ring systems can include one or more heteroatoms in one or both rings, and the point of attachment can be on any ring. As used herein, the term “heterocyclylene” as used by itself or as part of another group refers to a divalent radical derived from the heterocyclyl group defined herein. The heterocyclyl or heterocylylene can be optionally linked to the rest of the molecule through a carbon or nitrogen atom.

Exemplary 3-membered heterocyclyl groups containing one heteroatom include, without limitation, azirdinyl, oxiranyl, thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” as used by itself or as part of another group refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₋₄ aryl”; e.g., anthracyl). As used herein, the term “arylene” as used by itself or as part of another group refers to a divalent radical derived from the aryl group defined herein.

“Aralkyl” as used by itself or as part of another group refers to an alkyl substituted with one or more aryl groups, preferably, substituted with one aryl group. Examples of aralkyl include benzyl, phenethyl, etc. When an aralkyl is said to be optionally substituted, either the alkyl portion or the aryl portion of the aralkyl can be optionally substituted.

“Heteroaryl” as used by itself or as part of another group refers to a radical of a 5-14 membered monocyclic, bicyclic, or tricyclic 4n+2 aromatic ring system (e.g., having 6 or 10 pi electrons shared in a cyclic array) having ring carbon atoms and at least one, preferably, 1-4, ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. In bicyclic heteroaryl groups wherein one ring does not contain a heteroatom (e.g., indolyl, quinolinyl, and the like), the point of attachment can be on either ring, i.e., either the ring bearing a heteroatom (e.g., 2-indolyl) or the ring that does not contain a heteroatom (e.g., 5-indolyl). As used herein, the term “heteroarylene” as used by itself or as part of another group refers to a divalent radical derived from the heteroaryl group defined herein.

Exemplary 5-membered heteroaryl groups containing one heteroatom include, without limitation, pyrrolyl, furanyl, and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” as used by itself or as part of another group refers to an alkyl substituted with one or more heteroaryl groups, preferably, substituted with one heteroaryl group. When a heteroaralkyl is said to be optionally substituted, either the alkyl portion or the heteroaryl portion of the heteroaralkyl can be optionally substituted.

As used herein, unless specified or otherwise contrary, a “ring structure”, “cyclic structure”, or simply “ring”, with a designated number of ring members, such as a “3-10 membered ring structure”, a “3-12 membered ring structure”, or a “5- or 6-membered ring”, should be understood as encompassing any ring structure (e.g., carbocyclic, heterocyclic, aryl, heteroaryl, etc.) having the designated number of ring members, which can be (1) monocyclic or polycyclic (as chemically feasible), such as a monocyclic ring or a bicyclic ring (including fused, spiro, and bridged bicyclic ring, and those ring systems where two monocyclic rings are connected through a single or double bond); (2) aromatic, partially unsaturated, or fully saturated; and in the case of a polycyclic structure, each ring can be independently aromatic, partially unsaturated, or fully saturated; and (3) containing no heteroatom (i.e., all ring members are carbon atoms) or 1-4 heteroatoms; or in the case of a polycyclic structure, each ring can independently have no ring heteroatom or 1-4 ring heteroatoms (e.g., O, N, S, etc.). When a ring is said to contain a ring sulfur or nitrogen atom, the sulfur or nitrogen atom can be optionally oxidized. One or more ring carbon atoms in a ring structure can be present as C(═O). A fully saturated ring refers to a ring in which none of the ring carbon atom(s) and any present ring heteroatom(s) (e.g., nitrogen) forms a double bond or triple bond with any other atom. The ring structure can be optionally substituted with one or more substituents described herein. The substituents of a ring structure herein can also have a cyclic structure, and in some cases, two substituents of a ring structure may be said to be joined to form a cyclic structure.

As commonly understood in the art, for clarity, when a structure can be characterized in multiple ways, as long as one such characterization falls within the scope of the definition of a variable herein, it can be said that the structure is a suitable definition for the variable. For example, when a monovalent variable is defined as an optionally substituted 6-membered ring, the variable encompasses, among other structures, (a) the structure of

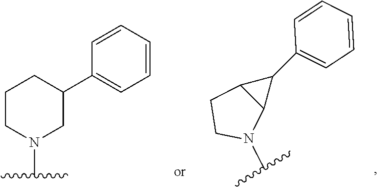 [SMILES: *N1CCCC(c2ccccc2)C1 ; *N1CCC2C(c3ccccc3)C21]

which can be viewed as a 6-membered monocyclic or bicyclic ring substituted with a phenyl group; and (b) the structure of

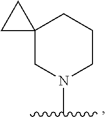 [SMILES: *N1CCCC2(CC2)C1]

which can be viewed as a 6-membered ring, wherein two substituents are joined to form a cyclopropyl ring; but the variable would not encompass

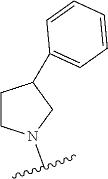 [SMILES: *N1CCC(c2ccccc2)C1]

because the attaching ring is not a 6-membered ring under any characterization of the structure. To further explain, when the variable is instead defined as an optionally substituted monocyclic 6-membered ring, then the variable does not encompass

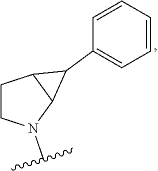 [SMILES: *N1CCC2C(c3ccccc3)C21]

but encompasses the structure of

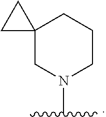 [SMILES: *N1CCCC2(CC2)C1]

And if the variable is defined as a 6-membered ring optionally substituted with halogen, then the variable can encompass structures such as,

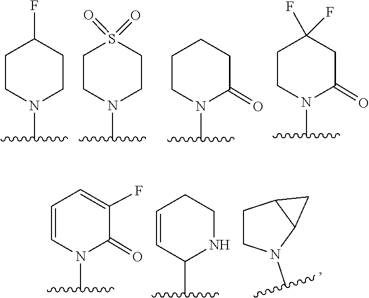

as each of which can be viewed as a 6-membered ring that is unsubstituted or substituted with 1 or two fluorine atoms.

As commonly understood in the art, alkylene, alkenylene, alkynylene, heteroalkylene, carbocyclylene, heterocyclylene, arylene, and heteroarylene refer to the corresponding divalent radicals of alkyl, alkenyl, alkynyl, heteroalkyl, carbocyclyl, heterocyclyl, aryl, and heteroaryl groups, respectively.

An “optionally substituted” group, such as an optionally substituted alkyl, optionally substituted alkenyl, optionally substituted alkynyl, optionally substituted carbocyclyl, optionally substituted heterocyclyl, optionally substituted aryl, and optionally substituted heteroaryl groups, refers to the respective group that is unsubstituted or substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent can be the same or different at each position. Typically, when substituted, the optionally substituted groups herein can be substituted with 1-5 substituents. Substituents can be a carbon atom substituent, a nitrogen atom substituent, an oxygen atom substituent or a sulfur atom substituent, as applicable, each of which can be optionally isotopically labeled, such as deuterated. Two of the optional substituents can join to form a ring structure, such as an optionally substituted cycloalkyl, heterocylyl, aryl, or heteroaryl ring. Substitution can occur on any available carbon, oxygen, or nitrogen atom, and can form a spirocycle. Typically, substitution herein does not result in an O—O, O—N, S—S, S—N(except SO₂—N bond), heteroatom-halogen, or —C(O)—S bond or three or more consecutive heteroatoms, with the exception of O—SO₂—O, O—SO₂—N, and N—SO₂—N, except that some of such bonds or connections may be allowed if in a stable aromatic system.

In a broad aspect, the permissible substituents herein include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms. Substituents can include any substituents described herein, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxy, a cycloalkoxy, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, an aryl, or a heteroaryl, each of which can be substituted, if appropriate.

Exemplary substituents include, but not limited to, alkyl, alkenyl, alkynyl, aryl, heteroaryl, -alkylene-aryl, -arylene-alkyl, -alkylene-heteroaryl, -alkenylene-heteroaryl, -alkynylene-heteroaryl, —OH, hydroxyalkyl, haloalkyl, —O-alkyl, —O-haloalkyl, -alkylene-O-alkyl, —O-aryl, —O-alkylene-aryl, —O-heteroaryl, —O-alkylene-heteroaryl, —O— cycloalkyl, —O-heterocycloalkyl, acyl, —C(O)-alkyl, —C(O)-haloalkyl, —C(O)-aryl, —C(O)-alkylene-aryl, —C(O)-heteroaryl, —C(O)-alkylene-heteroaryl, —C(O)-cycloalkyl, —C(O)-heterocycloalkyl, halo, —NO₂, —CN, —SF₅, —C(O)OH, —C(O)O-alkyl, —C(O)O-aryl, —C(O)O-alkylene-aryl, —S(O)-alkyl, —S(O)₂-alkyl, —S(O)-haloalkyl, —S(O)₂-haloalkyl, —S(O)-aryl, —S(O)₂-aryl, —S(O)-heteroaryl, —S(O)₂-heteroaryl, —S-alkyl, —S-aryl, —S-heteroaryl, —S-alkylene-aryl, —S-alkylene-heteroaryl, —S(O)₂-alkylene-aryl, —S(O)₂-alkylene-heteroaryl, —S-cycloalkyl, —S-heterocycloalkyl, —S(O)-cycloalkyl, —S(O)-heterocycloalkyl, —S(O)₂-cycloalkyl, —S(O)₂-heterocycloalkyl, —S(O)(═NH)-alkyl, —S(O)(═NH)-haloalkyl, —S(O)(═NH)-aryl, —S(O)(═NH)-alkylene-aryl, —S(O)(═NH)-heteroaryl, —S(O)(═NH)-alkylene-heteroaryl, —S(O)(═NH)-cycloalkyl, —S(O)(═NH)-heterocycloalkyl, —S(O)(=Nalkyl)-alkyl, —S(O)(=Nalkyl)-haloalkyl, —S(O)(=Nalkyl)-aryl, —S(O)(=Nalkyl)-alkylene-aryl, —S(O)(=Nalkyl)-heteroaryl, —S(O)(=Nalkyl)-alkylene-heteroaryl, —S(O)(=Nalkyl)-cycloalkyl, —S(O)(=Nalkyl)-heterocycloalkyl, cycloalkyl, heterocycloalkyl, —O—C(O)-alkyl, —O—C(O)-aryl, —O—C(O)-cycloalkyl, —C(═N—CN)—NH₂, —C(═NH)—NH₂, —C(═NH)—NH(alkyl), —N(Y₁)(Y₂), -alkylene-N(Y₁)(Y₂), —C(O)N(Y₁)(Y₂) and —S(O)₂N(Y₁)(Y₂), wherein Y₁ and Y₂ can be the same or different and are independently selected from the group consisting of hydrogen, alkyl, haloalkyl, aryl, -alkylene-aryl, heteroaryl, -alkylene-heteroaryl, cycloalkyl, heterocycloalkyl, and Y₁ and Y₂ with the nitrogen they linked can form a heterocyclic ring.

Some examples of suitable substituents include, but not limited to, (C₁-C₈)alkyl groups, (C₂-C₈)alkenyl groups, (C₂-C₈)alkynyl groups, (C₃-C₁₀)cycloalkyl groups, halogen (F, Cl, Br or I), halogenated (C₁-C₅)alkyl groups (for example but not limited to —CF₃), —O—(C₁-C₈)alkyl groups, —OH, —S—(C₁-C₈)alkyl groups, —SH, —NH(C₁-C₈)alkyl groups, —N((C₁-C₈)alkyl)₂ groups, —NH₂, —C(O)NH₂, —C(O)NH(C₁-C₈)alkyl groups, —C(O)N((C₁-C₈)alkyl)₂, —NHC(O)H, —NHC(O) (C₁-C₈)alkyl groups, —NHC(O) (C₃—Cs)cycloalkyl groups, —N((C₁-C₈)alkyl)C(O)H, —N((C₁-C₈)alkyl)C(O)(C₁-C₈)alkyl groups, —NHC(O)NH₂, —NHC(O)NH(C₁-C₈)alkyl groups, —N((C₁-C₈)alkyl)C(O)NH₂ groups, —NHC(O)N((C₁-C₈)alkyl)₂ groups, —N((C₁-C₈)alkyl)C(O)N((C₁-C₈)alkyl)₂ groups, —N((C₁-C₈)alkyl)C(O)NH((C₁-C₈)alkyl), —C(O)H, —C(O)(C₁-C₈)alkyl groups, —CN, —NO₂, —S(O)(C₁-C₈)alkyl groups, —S(O)₂(C₁-C₈)alkyl groups, —S(O)₂N((C₁-C₈)alkyl)₂ groups, —S(O)₂NH(C₁-C₈)alkyl groups, —S(O)₂NH(C₃-C₈)cycloalkyl groups, —S(O)₂NH₂ groups, —NHS(O)₂(C₁-C₈)alkyl groups, —N((C₁-C₈)alkyl)S(O)₂(C₁-C₈)alkyl groups, —(C₁-C₈)alkyl-O—(C₁-C₈)alkyl groups, —O—(C₁-C₈)alkyl-O—(C₁-C₈)alkyl groups, —C(O)OH, —C(O)O(C₁-C₈)alkyl groups, NHOH, NHO(C₁-C₈)alkyl groups, —O-halogenated (C₁-C₈)alkyl groups (for example but not limited to —OCF₃), —S(O)₂-halogenated (C₁-C₈)alkyl groups (for example but not limited to —S(O)₂CF₃), —S-halogenated (C₁-C₈)alkyl groups (for example but not limited to —SCF₃), —(C₁-C₆) heterocycle (for example but not limited to pyrrolidine, tetrahydrofuran, pyran or morpholine), —(C₁-C₆) heteroaryl (for example but not limited to tetrazole, imidazole, furan, pyrazine or pyrazole), -phenyl, —NHC(O)O—(C₁-C₆)alkyl groups, —N((C₁-C₆)alkyl)C(O)O—(C₁-C₆)alkyl groups, —C(═NH)—(C₁-C₈)alkyl groups, —C(═NOH)—(C₁-C₈)alkyl groups, or —C(N—O—(C₁-C₈)alkyl)-(C₁-C₆)alkyl groups.

Exemplary carbon atom substituents include, but are not limited to, deuterium, halogen, —CN, —NO₂, —N₃, hydroxyl, alkoxy, cycloalkoxy, aryloxy, amino, monoalkyl amino, dialkyl amino, amide, sulfonamide, thiol, acyl, carboxylic acid, ester, sulfone, sulfoxide, alkyl, haloalkyl, alkenyl, alkynyl, C₃₋₁₀ carbocyclyl, C₆₋₁₀ aryl, 3-10 membered heterocyclyl, 5-10 membered heteroaryl, etc. For example, exemplary carbon atom substituents can include F, Cl, —CN, —SO₂H, —SO₃H, —OH, —OC₁₋₆ alkyl, —NH₂, —N(C₁₋₆ alkyl)₂, —NH(C₁₋₆ alkyl), —SH, —SC₁₋₆ alkyl, —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂₋₀C₁₋₆ alkyl, —OSO₂C₁₋₆ alkyl, —SOC₁₋₆ alkyl, C₁₋₆ alkyl, C₁₋₆ haloalkyl, C₂₋₆ alkenyl, C₂₋₆ alkynyl, C₃₋₁₀ carbocyclyl, C₆₋₁₀ aryl, 3-10 membered heterocyclyl, 5-10 membered heteroaryl; or two geminal substituents can be joined to form ═O.

Nitrogen atoms can be substituted or unsubstituted as valency permits, and include primary, secondary, tertiary, and quaternary nitrogen atoms. Exemplary nitrogen atom substituents include, but are not limited to, hydrogen, acyl groups, esters, sulfone, sulfoxide, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two substituent groups attached to a nitrogen atom are joined to form a 3-14 membered heterocyclyl or 5-14 membered heteroaryl ring, wherein each alkyl, alkenyl, alkynyl, carbocyclyl, heterocyclyl, aryl, and heteroaryl can be further substituted as defined herein. In certain embodiments, the substituent present on a nitrogen atom is a nitrogen protecting group (also referred to as an amino protecting group). Nitrogen protecting groups are well known in the art and include those described in detail in Protective Groups in Organic Synthesis, T. W. Greene and P. G. M. Wuts, 3^{rd} edition, John Wiley & Sons, 1999, incorporated by reference herein. Exemplary nitrogen protecting groups include, but not limited to, those forming carbamates, such as Carbobenzyloxy (Cbz) group, p-Methoxybenzyl carbonyl (Moz or MeOZ) group, tert-Butyloxycarbonyl (BOC) group, Troc, 9-Fluorenylmethyloxycarbonyl (Fmoc) group, etc., those forming an amide, such as acetyl, benzoyl, etc., those forming a benzylic amine, such as benzyl, p-methoxybenzyl, 3,4-dimethoxybenzyl, etc., those forming a sulfonamide, such as tosyl, Nosyl, etc., and others such as p-methoxyphenyl.

Exemplary oxygen atom substituents include, but are not limited to, acyl groups, esters, sulfonates, C₁₋₁₀ alkyl, C₁₋₁₀ haloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, carbocyclyl, heterocyclyl, aryl, and heteroaryl can be further substituted as defined herein. In certain embodiments, the oxygen atom substituent present on an oxygen atom is an oxygen protecting group (also referred to as a hydroxyl protecting group). Oxygen protecting groups are well known in the art and include those described in detail in Protective Groups in Organic Synthesis, T. W. Greene and P. G. M. Wuts, 3d edition, John Wiley & Sons, 1999, incorporated herein by reference. Exemplary oxygen protecting groups include, but are not limited to, those forming alkyl ethers or substituted alkyl ethers, such as methyl, allyl, benzyl, substituted benzyls such as 4-methoxybenzyl, methoxylmethyl (MOM), benzyloxymethyl (BOM), 2-methoxyethoxymethyl (MEM), etc., those forming silyl ethers, such as trimethylsilyl (TMS), triethylsilyl (TES), triisopropylsilyl (TIPS), t-butyldimethylsilyl (TBDMS), etc., those forming acetals or ketals, such as tetrahydropyranyl (THP), those forming esters such as formate, acetate, chloroacetate, dichloroacetate, trichloroacetate, trifluoroacetate, methoxyacetate, etc., those forming carbonates or sulfonates such as methanesulfonate (mesylate), benzylsulfonate, and tosylate (Ts), etc.

Unless expressly stated to the contrary, combinations of substituents and/or variables are allowable only if such combinations are chemically allowed and result in a stable compound. A “stable” compound is a compound that can be prepared and isolated and whose structure and properties remain or can be caused to remain essentially unchanged for a period of time sufficient to allow use of the compound for the purposes described herein (e.g., therapeutic administration to a subject).

In some embodiments, the “optionally substituted” alkyl, alkylene, heteroalkyl, heteroalkylene, alkenyl, alkynyl, carbocyclic, carbocyclylene, cycloalkyl, cycloalkylene, alkoxy, cycloalkoxy, heterocyclyl, or heterocyclylene herein can each be independently unsubstituted or substituted with 1, 2, 3, or 4 substituents independently selected from deuterium, F, Cl, —OH, protected hydroxyl, oxo (as applicable), NH₂, protected amino, NH(C₁₋₄ alkyl) or a protected derivative thereof, N(C₁₋₄ alkyl((C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2, or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy phenyl, heteroaryl, and heterocyclyl, is optionally substituted with 1, 2, or 3 substituents independently selected from deuterium, F, —OH, oxo (as applicable), C₁₋₄ alkyl, fluoro-substituted C₁₋₄ alkyl (e.g., CF₃), C₁₋₄ alkoxy and fluoro-substituted C₁₋₄ alkoxy. In some embodiments, the “optionally substituted” aryl, arylene, heteroaryl or heteroarylene group herein can each be independently unsubstituted or substituted with 1, 2, 3, or 4 substituents independently selected from deuterium, F, Cl, —OH, —CN, NH₂, protected amino, NH(C₁₋₄ alkyl) or a protected derivative thereof, N(C₁₋₄ alkyl((C₁₋₄ alkyl), —S(═O)(C₁₋₄ alkyl), —SO₂(C₁₋₄ alkyl), C₁₋₄ alkyl, C₂₋₄ alkenyl, C₂₋₄ alkynyl, C₁₋₄ alkoxy, C₃₋₆ cycloalkyl, C₃₋₆ cycloalkoxy, phenyl, 5 or 6 membered heteroaryl containing 1, 2 or 3 ring heteroatoms independently selected from O, S, and N, 3-7 membered heterocyclyl containing 1 or 2 ring heteroatoms independently selected from O, S, and N, wherein each of the alkyl, alkenyl, alkynyl, alkoxy, cycloalkyl, cycloalkoxy, phenyl, heteroaryl, and heterocyclyl, is optionally substituted with 1, 2, or 3 substituents independently selected from deuterium, F, —OH, oxo (as applicable), C₁₋₄alkyl, fluoro-substituted C₁₋₄ alkyl, C₁₋₄ alkoxy and fluoro-substituted C₁₋₄ alkoxy.

“Halo” or “halogen” refers to fluorine (fluoro, —F), chlorine (chloro, —Cl), bromine (bromo, —Br), or iodine (iodo, —I).

The term “pharmaceutically acceptable salt” refers to those salts which are, within the scope of sound medical judgment, suitable for use in contact with the tissues of humans and lower animals without undue toxicity, irritation, allergic response, and the like, and are commensurate with a reasonable benefit/risk ratio. Pharmaceutically acceptable salts are well known in the art.

The term “tautomers” or “tautomeric” refers to two or more interconvertible compounds resulting from tautomerization. The exact ratio of the tautomers depends on several factors, including for example temperature, solvent, and pH. Tautomerizations are known to those skilled in the art. Exemplary tautomerizations include keto-to-enol, amide-to-imide, lactam-to-lactim, enamine-to-imine, and enamine-to-(a different enamine) tautomerizations.

The term “subject” (alternatively referred to herein as “patient”) as used herein, refers to an animal, preferably a mammal, most preferably a human, who has been the object of treatment, observation or experiment.

As used herein, the terms “treat,” “treating,” “treatment,” and the like refer to eliminating, reducing, or ameliorating a disease or condition, and/or symptoms associated therewith. Although not precluded, treating a disease or condition does not require that the disease, condition, or symptoms associated therewith be completely eliminated. As used herein, the terms “treat,” “treating,” “treatment,” and the like may include “prophylactic treatment,” which refers to reducing the probability of redeveloping a disease or condition, or of a recurrence of a previously-controlled disease or condition, in a subject who does not have, but is at risk of or is susceptible to, redeveloping a disease or condition or a recurrence of the disease or condition. The term “treat” and synonyms contemplate administering a therapeutically effective amount of a compound described herein to a subject in need of such treatment.

The term “effective amount” refers to that amount of a compound or combination of compounds as described herein that is sufficient to effect the intended application including, but not limited to, prophylaxis or treatment of diseases. A therapeutically effective amount may vary depending upon the intended application (in vitro or in vivo), or the subject and disease condition being treated (e.g., the weight, age and gender of the subject), the severity of the disease condition, the manner of administration, etc. which can readily be determined by one of ordinary skill in the art. The term also applies to a dose that will induce a particular response in target cells and/or tissues. The specific dose will vary depending on the particular compounds chosen, the dosing regimen to be followed, whether the compound is administered in combination with other compounds, timing of administration, the tissue to which it is administered, and the physical delivery system in which the compound is carried.

As used herein, the singular form “a”, “an”, and “the”, includes plural references unless it is expressly stated or is unambiguously clear from the context that such is not intended.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

Headings and subheadings are used for convenience and/or formal compliance only, do not limit the subject technology, and are not referred to in connection with the interpretation of the description of the subject technology. Features described under one heading or one subheading of the subject disclosure may be combined, in various embodiments, with features described under other headings or subheadings. Further it is not necessarily the case that all features under a single heading or a single subheading are used together in embodiments.

### EXAMPLES

The various starting materials, intermediates, and compounds of embodiments herein can be isolated and purified where appropriate using conventional techniques such as precipitation, filtration, crystallization, evaporation, distillation, and chromatography. Characterization of these compounds can be performed using conventional methods such as by melting point, mass spectrum, nuclear magnetic resonance, and various other spectroscopic analyses. The abbreviations used in the Examples section should be understood as having their ordinary meanings in the art unless specifically indicated otherwise or obviously contrary from context. The examples are illustrative only and do not limit the claimed invention in any way.

Exemplary embodiments of steps for performing the synthesis of products described herein are described in greater detail infra.

### Intermediate A

### 2-(5-(benzylthio)-7-chloropyrazolo[1,5-a]pyridin-3-yl)-5-(difluoromethyl)-1,3,4-thiadiazole (A)

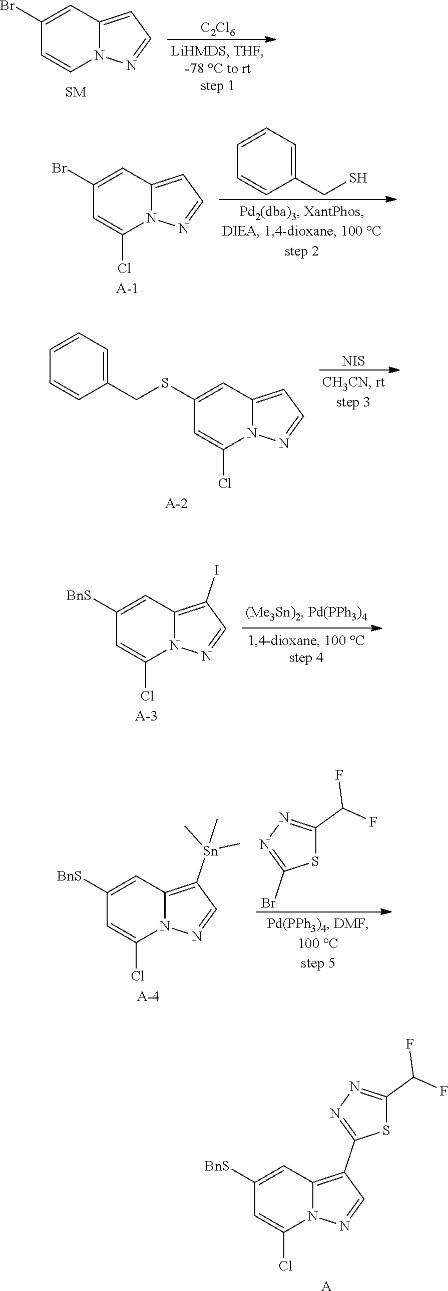

### Step 1: 5-bromo-7-chloropyrazolo[1,5-a]pyridine (A-1)

To a solution of 5-bromopyrazolo[1,5-a]pyridine (3 g, 15.2 mmol) in dry THF (70 mL) was added LiHMDS (1 M in THE solution, 18.3 mL, 18.3 mmol) dropwise at −78 °C under N₂. After stirring for 2 hrs, a solution of C₂Cl₆ (3.24 g, 13.7 mmol) in dry THF (5 mL) was added dropwise at −78 °C under N₂ atmosphere. Then the reaction mixture was allowed to warm to room temperature and stirred at room temperature for 6 hrs. LCMS showed the reaction completed. The reaction mixture was worked up with water and extracted with ethyl acetate. The combined organic layers were washed with saturated brine, dried over anhydrous Na₂SO₄, filtered, and concentrated under reduced pressure to give a crude product which was purified by flash column chromatography on silica gel (eluting with 8% EtOAc in PE) to afford 5-bromo-7-chloro-pyrazolo[1,5-a]pyridine (2.7 g). LCMS (ESI, m/z): [M+H]⁺=230.9.

### Step 2: 5-(benzylthio)-7-chloropyrazolo[1,5-a]pyridine (A-2)

To a solution of 5-bromo-7-chloro-pyrazolo[1,5-a]pyridine (2.9 g, 12.5 mmol) and phenylmethanethiol (1.87 g, 15.0 mmol) in 1,4-dioxane (100 mL) was added DIEA (1.62 g, 12.5 mmol, 2.18 mL), Xantphos (1.45 g, 2.51 mmol) and Pd₂(dba)₃ (1.15 g, 1.25 mmol) under N₂. The reaction mixture was degassed, then stirred at 100 °C for 3 hrs under N₂ atmosphere. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 13% EtOAc in PE) to afford 5-benzylsulfanyl-7-chloro-pyrazolo[1,5-a]pyridine (700 mg). LCMS (ESI, m/z): [M+H]⁺=274.9.

### Step 3: 5-(benzylthio)-7-chloro-3-iodopyrazolo[1,5-a]pyridine (A-3)

To a solution of 5-benzylsulfanyl-7-chloro-pyrazolo[1,5-a]pyridine (587 mg, 2.14 mmol) in acetonitrile (15 mL) was added NIS (514.3 mg, 2.29 mmol) at room temperature under N₂. The reaction mixture was stirred at room temperature for 1 hr. LCMS showed the reaction completed. The reaction mixture was worked up with water and extracted by EtOAc. The combined organic layers were washed with saturated brine, dried over anhydrous Na₂SO₄, filtered, and concentrated under reduced pressure. The residue was purified by flash column chromatography on silica gel (eluting with 15% EtOAc in PE) to afford 5-benzylsulfanyl-7-chloro-3-iodo-pyrazolo[1,5-a]pyridine (788 mg). LCMS (ESI, m/z): [M+H]⁺=400.8.

### Step 4: 5-(benzylthio)-7-chloro-3-(trimethylstannyl)pyrazolo[1,5-a]pyridine (A-4)

To a solution of 5-benzylsulfanyl-7-chloro-3-iodo-pyrazolo[1,5-a]pyridine (600 mg, 1.5 mmol) in 1,4-dioxane (10 mL) was added trimethyl(trimethylstannyl)stannane (588 mg, 1.8 mmol) and Pd(PPh₃)₄ (86 mg, 0.075 mmol) under N₂. The reaction mixture was degassed with N₂, then stirred at 100 °C for 1.5 hrs. LCMS showed the reaction completed. The reaction mixture was concentrated under reduced pressure to afford the crude product which was used directly for the next step without further purification. LCMS (EST, m/z): [M+H]⁺=437.0.

### Step 5: 2-(5-(benzylthio)-7-chloropyrazolo[1,5-a]pyridin-3-yl)-5-(difluoromethyl)-1,3,4-thiadiazole (A)

To a solution of (5-benzylsulfanyl-7-chloro-pyrazolo[1,5-a]pyridin-3-yl)-trimethyl-stannane (300 mg, 0.69 mmol) in DMF (10 mL) was added 2-bromo-5-(difluoromethyl)-1,3,4-thiadiazole (162 mg, 0.75 mmol) and Pd(PPh₃)₄ (80 mg, 0.068 mmol) under N₂. The reaction mixture was degassed, then stirred at 100 °C for 5 hrs under N₂ atmosphere. LCMS showed the reaction completed. Then the reaction mixture was worked up with water and extracted with EtOAc. The organic layers were washed with saturated brine, dried over anhydrous Na₂SO₄, filtered, and concentrated under reduced pressure. The residue was purified by flash column chromatography on silica gel (eluting with 5%-10% EtOAc in PE) to afford 2-(5-(benzylthio)-7-chloropyrazolo[1,5-a]pyridin-3-yl)-5-(difluoromethyl)-1,3,4-thiadiazole (230 mg). LCMS (ESI, m/z): [M+H]⁺=409.1.

### Intermediate B

### 7-chloro-N-(1-cyanocyclopropyl)-3-(5-(difluoromethyl)-1,3,4-thiadiazol-2-yl)pyrazolo[1,5-a]pyridine-5-sulfonamide (B)

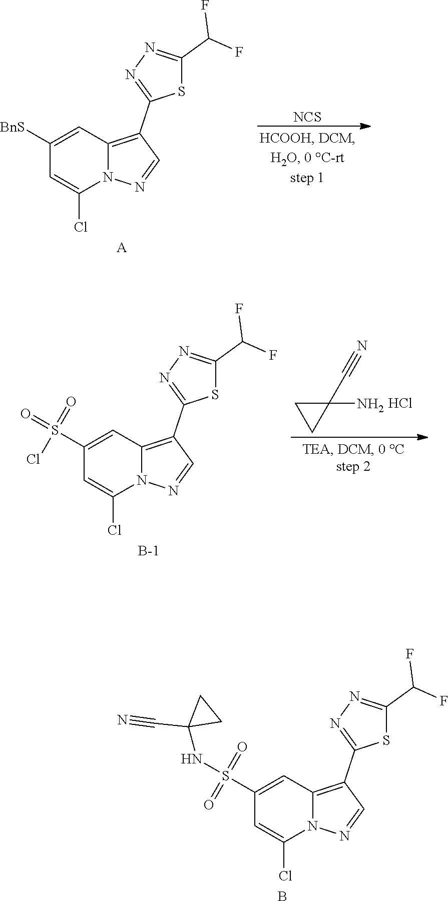

### Step 1: 7-chloro-3-(5-(difluoromethyl)-1,3,4-thiadiazol-2-yl)pyrazolo[1,5-a]pyridine-5-sulfonyl chloride (B-1)

To a solution of 2-(5-benzylsulfanyl-7-chloro-pyrazolo[1,5-a]pyridin-3-yl)-5-(difluoromethyl)-1,3,4-thiadiazole (500 mg, 1.22 mmol) in H₂O (0.9 mL), HCOOH (1.9 mL) and DCM (5 mL) was added NCS (653 mg, 4.89 mmol) at 0 °C under N₂. The mixture was stirred at room temperature for 1.5 hrs. LCMS showed the reaction completed. The reaction mixture was worked up with water and extracted with ethyl acetate. The combined organic layers were washed with saturated brine, dried over anhydrous Na₂SO₄, filtered, and concentrated to afford the crude product (420 mg) which was used directly in the next step without further purification. LCMS (ESI, m/z): [M+H]⁺=385.1.

### Step 2: 7-chloro-N-(1-cyanocyclopropyl)-3-(5-(difluoromethyl)-1,3,4-thiadiazol-2-yl)pyrazolo[1,5-a]pyridine-5-sulfonamide (B)

To a solution of 1-aminocyclopropanecarbonitrile hydrochloride (27.7 mg, 0.234 mmol) in DCM (1 mL) was added TEA (79 mg, 0.779 mmol, 0.109 mL). After stirring for 5 mins, 7-chloro-3-[5-(difluoromethyl)-1,3,4-thiadiazol-2-yl]pyrazolo[1,5-a]pyridine-5-sulfonyl chloride (100 mg, 0.260 mmol) in DCM (1 mL) was added at 0 °C under N₂. The reaction mixture was stirred at 0 °C for 2 hrs. LCMS showed the reaction completed. The reaction mixture was worked up with water and extracted with ethyl acetate. The combined organic layers were washed with saturated brine, dried over anhydrous Na₂SO₄, filtered, and concentrated under reduced pressure. The residue was purified by flash column chromatography on silica gel (eluting with 5% MeOH in DCM) to afford 7-chloro-N-(1-cyanocyclopropyl)-3-[5-(difluoromethyl)-1,3,4-thiadiazol-2-yl]pyrazolo[1,5-a]pyridine-5-sulfonamide (12 mg). LCMS (ESI, m/z): [M+H]⁺=431.0.

### Intermediate C

### 2-bromo-5-(1-fluorocyclopropyl)-1,3,4-thiadiazole (C)

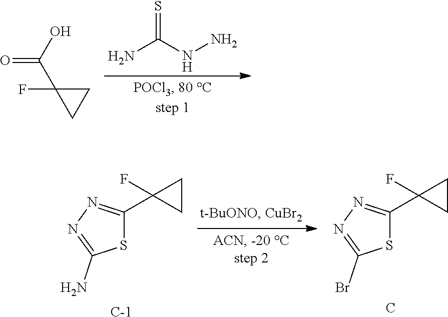

### Step 1: 5-(1-fluorocyclopropyl)-1,3,4-thiadiazol-2-amine (C-1)

To a solution of 1-fluorocyclopropanecarboxylic acid (2 g, 19.2 mmol) in POCl₃ (20 mL) was added aminothiourea (1.75 g, 19.2 mmol) under N₂. The mixture was stirred at 80 °C for 1 hr. LCMS showed the reaction completed. The reaction mixture was cooled to room temperature, poured into water carefully, then neutralized with saturated aqueous NaHCO₃ to pH=7-8. The resulting mixture was extracted with EtOAc. The combined organic layers were dried over anhydrous Na₂SO₄, filtered, and concentrated under reduced pressure to afford 5-(1-fluorocyclopropyl)-1,3,4-thiadiazol-2-amine (2.17 g). LCMS (ESI, m/z): [M+H]⁺=160.0.

### Step 2: 2-bromo-5-(1-fluorocyclopropyl)-1,3,4-thiadiazole (C)

To a solution of 5-(1-fluorocyclopropyl)-1,3,4-thiadiazol-2-amine (2.17 g, 13.6 mmol) in ACN (50 mL) was added CuBr₂ (4.57 g, 20.5 mmol) and tert-butyl nitrite (1.72 g, 16.6 mmol, 1.98 mL) at −20 °C under N₂. The mixture was stirred at −20 °C for 5 hrs. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 13% EtOAc in PE) to afford 2-bromo-5-(1-fluorocyclopropyl)-1,3,4-thiadiazole (1.4 g). LCMS (ESI, m/z): [M+H]⁺=222.8.

### Intermediate D

### 2-bromo-4-(difluoromethyl)thiazole (D)

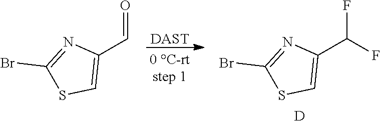

To a solution of 2-bromothiazole-4-carbaldehyde (2.0 g, 10.4 mmol) in DCM (20 mL) was added DAST (6.72 g, 41.7 mmol) at 0 °C under N₂. The reaction mixture was stirred at room temperature for 16 hrs. The reaction mixture was quenched with saturated aqueous NaHCO₃, then extracted with DCM. The combined organic layers were washed with saturated brine, dried over anhydrous Na₂SO₄, filtered, and concentrated under reduced pressure. The residue was purified by flash column chromatography on silica gel (eluting with 5% EtOAc in PE) to afford 2-bromo-4-(difluoro methyl) thiazole (1.5 g). ¹H NMR (400 MHz, DMSO-d₆) δ 8.18 (s 1H), 7.07 (t, J=54.0 Hz, 1H).

### Intermediate E

### isopropyl piperazine-1-carboxylate hydrochloride (E)

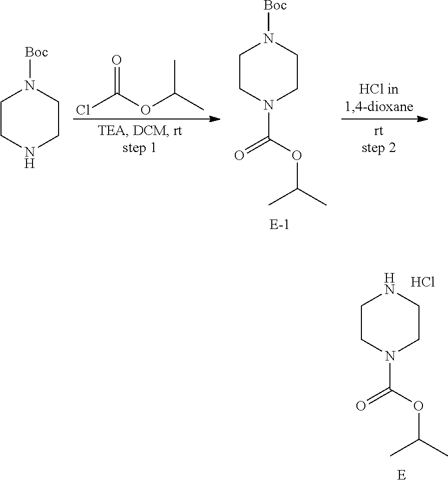

### Step 1: 1-(tert-butyl) 4-isopropyl piperazine-1,4-dicarboxylate (E-1)

To a mixture of tert-butyl piperazine-1-carboxylate (1 g, 5.37 mmol) and TEA (1.63 g, 16.1 mmol, 2.25 mL) in DCM (10 mL) was added isopropyl carbonochloridate (790 mg, 6.44 mmol) at room temperature under N₂. The mixture was stirred at room temperature for 3 hrs. After completion, the reaction mixture was worked up with water and extracted with DCM. The combined organic layers were washed with aqueous HCl (1 M) and then with saturated brine, dried over anhydrous Na₂SO₄, filtered, and concentrated to afford 1-(tert-butyl) 4-isopropyl piperazine-1,4-dicarboxylate (1.3 g).

### Step 2: isopropyl piperazine-1-carboxylate hydrochloride (E)

1-(tert-butyl) 4-isopropyl piperazine-1,4-dicarboxylate (1 g, 3.67 mmol) was added to a solution of HCl in 1,4-dioxane solution (4.0 M, 11 mL). The reaction mixture was stirred for 1 hr at room temperature. LCMS showed the reaction completed. The reaction mixture was concentrated under reduced pressure to give isopropyl piperazine-1-carboxylate hydrochloride (750 mg) which was used directly in the next step without further purification.

### Intermediate F

### (1-methylcyclobutyl)(piperazin-1-yl)methanone hydrochloride (F)

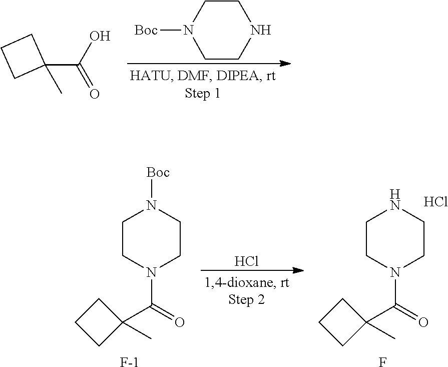

### Step 1: tert-butyl 4-(1-methylcyclobutanecarbonyl)piperazine-1-carboxylate (F-1)

To a solution of 1-methylcyclobutanecarboxylic acid (505 mg, 4.42 mmol), tert-butyl piperazine-1-carboxylate (750 mg, 4.03 mmol) and DIPEA (1.56 g, 12.1 mmol, 2.10 mL) in DMF (5 mL) was added HATU (1.68 g, 4.42 mmol) under N₂. The reaction mixture was stirred at room temperature for 3 hrs. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 0-30% EtOAc in PE) to give tert-butyl 4-(1-methylcyclobutanecarbonyl)piperazine-1-carboxylate (0.95 g).

### Step 2: (1-methylcyclobutyl)-piperazin-1-yl-methanone hydrochloride salt (F)

tert-butyl 4-(1-methylcyclobutanecarbonyl)piperazine-1-carboxylate (300 mg, 1.06 mmol) was added to HCl in 1,4-dioxane (4 M, 6 mL) at 0 °C. The mixture was warmed to room temperature and stirred for 3 hrs under N₂ atmosphere. TLC showed the reaction completed. The reaction mixture was concentrated to afford (1-methylcyclobutyl)(piperazin-1-yl)methanone hydrochloride (200 mg). LCMS (ESI, m/z): [M+H]⁺=183.1.

### Intermediate G

### 2-methyl-5-(piperazin-1-yl)-1,3,4-thiadiazole hydrochloride (G)

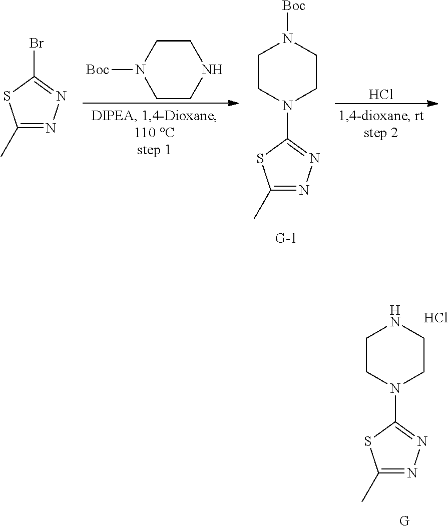

### Step 1: tert-butyl 4-(5-methyl-1,3,4-thiadiazol-2-yl)piperazine-1-carboxylate (G-1)

To a solution of 2-bromo-5-methyl-1,3,4-thiadiazole (500 mg, 2.79 mmol) and DIPEA (256 mg, 5.58 mmol) in 1,4-dioxane (7 mL) was added tert-butyl piperazine-1-carboxylate (520 mg, 2.79 mmol) at room temperature under N₂. The reaction mixture was stirred at 110 °C for 5 hrs. LCMS showed the reaction completed. The reaction mixture was cooled to room temperature and worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 30% EtOAc in PE) to afford tert-butyl 4-(5-methyl-1,3,4-thiadiazol-2-yl)piperazine-1-carboxylate (680 mg). LCMS (ESI, m/z): [M+H]⁺=285.4.

### Step 2: 2-methyl-5-piperazin-1-yl-1,3,4-thiadiazole hydrochloride (G)

tert-butyl 4-(5-methyl-1,3,4-thiadiazol-2-yl)piperazine-1-carboxylate (680 mg, 2.39 mmol) was added to a solution of HCl in 1,4 dioxane (4 M, 4 mL). The mixture was stirred at room temperature for 2 hrs. LCMS showed the reaction completed. The reaction mixture was concentrated under reduced pressure to afford 2-methyl-5-piperazin-1-yl-1,3,4-thiadiazole hydrochloride (520 mg). LCMS (ESI, m/z): [M+H]⁺=184.26.

### Intermediate H

### (S)-(3-fluoropyrrolidin-1-yl)(piperazin-1-yl)methanone hydrochloride (H)

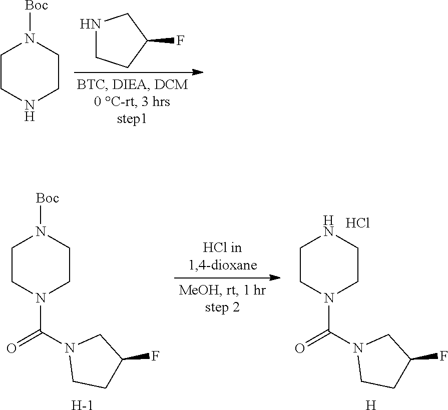

### Step 1: tert-butyl (S)-4-(3-fluoropyrrolidine-I-carbonyl) piperazine-1-carboxylate (H-1)

To a solution of tert-butyl piperazine-1-carboxylate (1.92 g, 10.3 mmol) and bis(trichloromethyl) carbonate (1.53 g, 5.17 mmol) in DCM (40 mL) was added DIEA (4.01 g, 30.9 mmol, 5.40 mL) dropwise at 0 °C under N₂. The mixture was stirred at room temperature for 3 hrs under N₂ atmosphere. Then (S)-3-fluoropyrrolidine (920 mg, 10.3 mmol) was added and stirred for another 30 min. After completion, the reaction mixture was worked up with water, then extracted with EtOAc. The combined organic layers were washed with saturated brine, dried over anhydrous Na₂SO₄, filtered, and concentrated under reduced pressure. The residue was purified via prep-HPLC (0.05% NH₄HCO₃) to afford tert-butyl (S)-4-(3-fluoropyrrolidine-1-carbonyl) piperazine-1-carboxylate (800 mg). LCMS (ESI, m/z): [M+H-tBu]⁺=246.2.

### Step 2: (S)-(3-fluoropyrrolidin-1-yl) (piperazin-1-yl) methanone (H)

To a solution of tert-butyl (S)-4-(3-fluoropyrrolidine-1-carbonyl) piperazine-1-carboxylate (500 mg, 1.66 mmol) in MeOH (5 mL) was added HCl (4.0 M in 1,4-dioxane, 2.5 mL). The mixture was stirred at room temperature for 3 hrs. After completion, the mixture was concentrated under reduced pressure to afford (S)-(3-fluoropyrrolidin-1-yl)(piperazin-1-yl)methanone hydrochloride (330 mg). LCMS (ESI, m/z): [M+H]⁺=202.3.

### Intermediate I

### 2-methyl-5-(piperazin-1-yl)-1,3,4-oxadiazole hydrochloride (I)

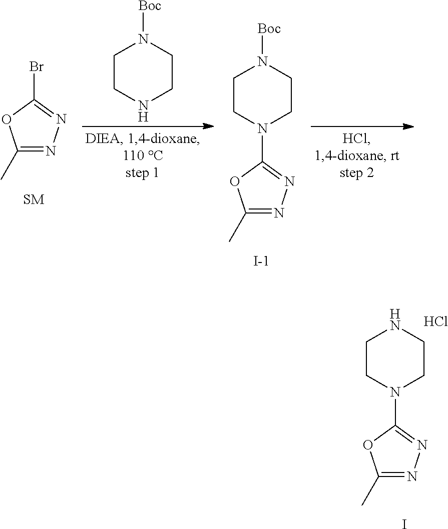

### Step 1: tert-butyl 4-(5-methyl-1,3,4-oxadiazol-2-yl)piperazine-1-carboxylate (I-1)

To a solution of 2-bromo-5-methyl-1,3,4-oxadiazole (500 mg, 3.07 mmol) in 1,4-dioxane (7 mL) were added tert-butyl piperazine-1-carboxylate (571 mg, 3.07 mmol) and DIEA (786 mg, 6.14 mmol) at room temperature. The mixture was stirred at 110 °C for 5 hrs under N₂ atmosphere. LCMS showed the reaction completed. The reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 30% EtOAc in PE) to afford tert-butyl 4-(5-methyl-1,3,4-oxadiazol-2-yl)piperazine-1-carboxylate (650 mg). LCMS (ESI, m/z): [M+H]⁺=269.2.

### Step 2: 2-methyl-5-piperazin-1-yl-1,3,4-oxadiazole (I)

A mixture of tert-butyl 4-(5-methyl-1,3,4-oxadiazol-2-yl)piperazine-1-carboxylate (300 mg, 1.12 mmol) in a solution of HCl (4.0 M in 1,4-dioxane, 4 mL) was stirred at room temperature for 2 hrs. LCMS showed the reaction completed. The reaction mixture was concentrated under reduced pressure to afford 2-methyl-5-(piperazin-1-yl)-1,3,4-oxadiazole hydrochloride (230 mg) which was used directly for the next step without further purification. LCMS (ESI, m/z): [M+H]⁺=169.1.

### Intermediate J

### 5-bromo-3-(difluoromethyl)-1,2,4-thiadiazole (J)

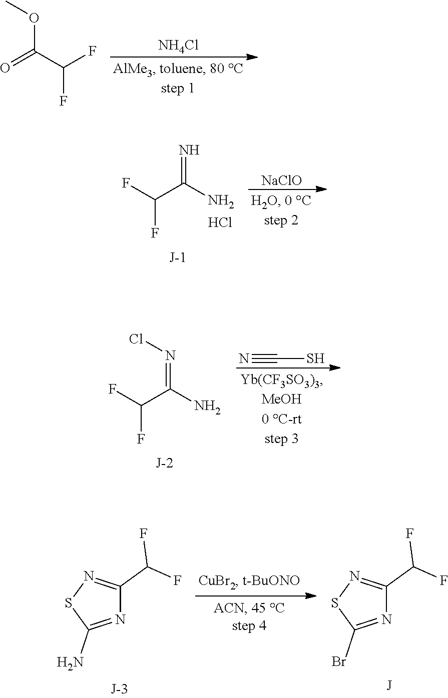

### Step 1: 2,2-difluoroacetimidamide hydrochloride (J-1)

To a stirred suspension of NH₄Cl (8.2 g, 153 mmol) in dry toluene (6 mL) at 0 °C was added trimethylaluminum (10.4 g, 143.8 mmol) under N₂ and stirred until effervescence ceased. Then Methyl 2,2-difluoroacetate (4.8 g, 43.6 mmol) was added. The resulting mixture was stirred overnight at 80 °C under N₂ atmosphere. After completion, the reaction mixture was cooled to 0 °C and quenched with methanol dropwise. The resulting mixture was stirred for 90 minutes at 0 °C until the solid formed. The mixture was filtered through celite, then the filtrate was concentrated under reduced pressure to afford 2,2-difluoroacetimidamide hydrochloride (2.2 g).

### Step 2: (E)-N′-chloro-2,2-difluoroacetimidamide (J-2)

To a solution of 2,2-difluoroacetimidamide hydrochloride (2.2 g, 16.9 mmol) in water (30 mL) was added aqueous NaClO (w/w %=7.5%, 12 mL). The mixture was stirred at 0 °C for 30 mins. LCMS showed the reaction completed. The reaction mixture was worked up with water, then extracted with EtOAc. The combined organic layers were washed with saturated brine, dried over anhydrous Na₂SO₄, filtered, and concentrated to give N′-chloro-2,2-difluoro-acetamidine (500 mg). LCMS (ESI, m/z): [M+H]⁺=128.9.

### Step 3: 3-(difluoromethyl)-1,2,4-thiadiazol-5-amine (J-3)

A solution of N′-chloro-2,2-difluoro-acetamidine (500 mg, 3.9 mmol) in methanol (9 mL) was treated with thiocyanic acid (230 mg, 3.9 mmol) for 5 min at 0 °C, followed by the addition of ytterbium(III) trifluoromethanesulfonate hydrate (242 mg, 0.39 mmol) in portions at room temperature for 5 hrs under N₂. LCMS showed the reaction completed. The reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 0-5% MeOH in DCM) to afford 3-(difluoromethyl)-1,2,4-thiadiazol-5-amine (300 mg). LCMS (ESI, m/z): [M+H]⁺=151.9. ¹H NMR (400 MHz, DMSO-d₆) δ 8.26 (s, 2H), 6.78 (t, J=52 Hz, 1H).

### Step 4: 5-bromo-3-(difluoromethyl)-1,2,4-thiadiazole (J)

To a solution of 3-(difluoromethyl)-1,2,4-thiadiazol-5-amine (300 mg, 1.98 mmol) in CH₃CN (3 mL) was added CuBr₂ (532 mg, 2.38 mmol), followed by tert-Butyl nitrite (266 mg, 2.58 mmol) at room temperature. The reaction mixture was stirred at 45 °C for 4 hrs. LCMS showed the reaction completed. The reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 0-10% EtOAc in PE) to give 5-bromo-3-(difluoromethyl)-1,2,4-thiadiazole (100 mg). LCMS (ESI, m/z): [M+H]⁺=214.8.

### Intermediate K

### [(3S)-3-hydroxypyrrolidin-1-yl]-piperazin-1-yl-methanone (K)

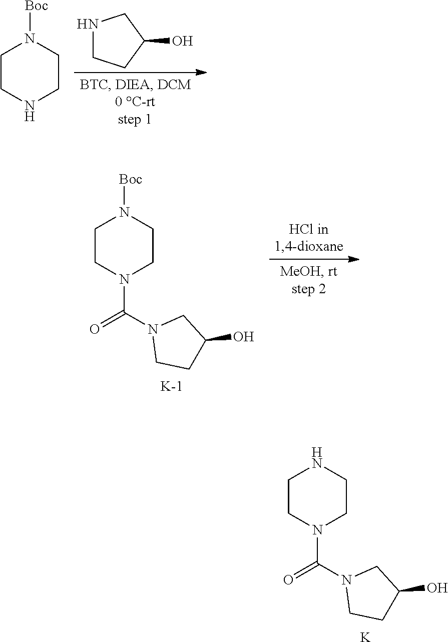

### Step 1: tert-butyl 4-[(3S)-3-hydroxypyrrolidine-I-carbonyl]piperazine-1-carboxylate (K-1)

To a solution of tert-butyl piperazine-1-carboxylate (1.92 g, 10.3 mmol) and bis(trichloromethyl) carbonate (1.53 g, 5.17 mmol) in DCM (40 mL) was added DIEA (4.01 g, 31.0 mmol) dropwise at 0 °C under N₂. The reaction mixture was allowed to warm to room temperature and stirred for 3 hrs. TLC indicated the total consumption of the starting material. (3S)-pyrrolidin-3-ol (900 mg, 10.3 mmol) was added to the mixture at 0 °C under N₂. The reaction mixture was stirred at room temperature for 30 mins. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified prep-HPLC (0.05% NH₄HCO₃) to afford tert-butyl 4-[(3S)-3-hydroxypyrrolidine-1-carbonyl]piperazine-1-carboxylate (800 mg). LCMS (ESI, m/z): [M+H]⁺=300.3.

### Step 2: [(3S)-3-hydroxypyrrolidin-1-yl]-piperazin-1-yl-methanone (K)

To a solution of tert-butyl 4-[(3S)-3-hydroxypyrrolidine-1-carbonyl]piperazine-1-carboxylate (500 mg, 1.67 mmol) in MeOH (5 mL) was added HCl (4.0 M in 1,4-dioxane, 1.25 ml, 5.0 mmol). The reaction mixture was stirred at room temperature for 3 hrs. LCMS indicated the total consumption of the starting material. The reaction mixture was concentrated under reduced pressure to afford (S)-(3-hydroxypyrrolidin-1-yl)(piperazin-I-yl)methanone hydrochloride (400 mg). LCMS (ESI, m/z): [M+H]⁺=200.3.

### Intermediate L

### 2-bromo-4H-pyrano[3,4-d]thiazol-7(6H)-one (L)

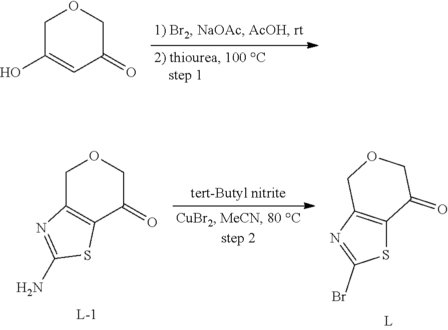 [SMILES: O=C1C=C(O)COC1 ; Nc1nc2c(s1)C(=O)COC2 ; O=C1COCc2nc(Br)sc21]

### Step 1: 2-amino-4H-pyrano[3,4-d]thiazol-7(6H)-one (L-1)

3-hydroxy-2H-pyran-5-one (5 g, 43.8 mmol) and anhydrous Sodium acetate (5.4 g, 65.8 mmol) were suspended in acetic acid (50 mL) with stirring. Br₂ (7 g, 43.8 mmol) was added dropwise at room temperature under N₂. The reaction is stirred at room temperature for 2 hrs, then Thiourea (3.34 g, 43.8 mmol) was added under N₂. After heating at 100 °C for 16 hrs, the reaction mixture was cooled and concentrated under reduced pressure. The residue was slurried with water, then with hot EtOAc. After drying in vacuum, 2-amino-4H-pyrano[3,4-d]thiazol-7-one (5.5 g) was obtained. LCMS (ESI, m/z): [M+H]⁺=171.1.

### Step 2: 2-bromo-4H-pyrano[3,4-d]thiazol-7(6H)-one (L)

To a solution of 2-amino-4H-pyrano[3,4-d]thiazol-7-one (5 g, 29.3 mmol) in ACN (50 mL) was added tert-Butyl nitrite (6.06 g, 58.8 mmol) dropwise at 0 °C followed by addition of CuBr₂ (13.1 g, 58.7 mmol). The mixture was stirred at 80 °C for 16 hrs under N₂. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 25% EtOAc in PE) to afford 2-bromo-4H-pyrano[3,4-d]thiazol-7-one (2.3 g). LCMS (ESI, m/z): [M+H]⁺=233.9.

### Intermediate M

### 1-(5-bromo-1,3,4-thiadiazol-2-yl)cyclobutan-1-ol (M)

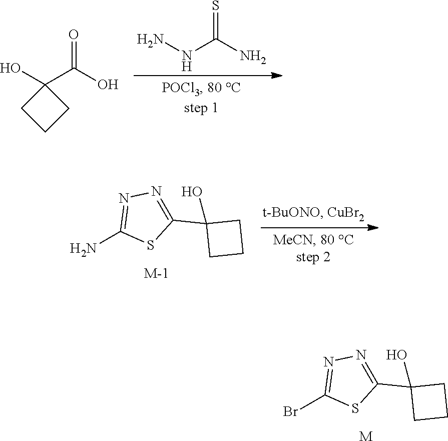

### Step 1: 1-(5-amino-1,3,4-thiadiazol-2-yl)cyclobutanol (M-1)

To a solution of 1-hydroxycyclobutanecarboxylic acid (1.00 g, 8.61 mmol) in phosphoryl trichloride (5.30 mL) was added aminothiourea (785 mg, 8.61 mmol) under N₂. The mixture was stirred at 80 °C for 1 hr under N₂ atmosphere. After completion, the reaction mixture was quenched by adding into water carefully, then neutralized with saturated aqueous NaHCO₃ to pH=8-9. The resulting mixture was extracted with EtOAc. The combined organic layers were washed with saturated brine, dried over Na₂SO₄, filtered, and concentrated under reduced pressure to afford 1-(5-amino-1,3,4-thiadiazol-2-yl)cyclobutanol (796 mg). LCMS (ESI, m/z): [M+H]⁺=172.1.

### Step 2: 1-(5-bromo-1,3,4-thiadiazol-2-yl)cyclobutanol (M)

To a mixture of tert-butyl nitrite (2.53 g, 24.5 mmol) and CuBr₂ (4.11 g, 18.4 mmol) in MeCN (84 mL) was added 1-(5-amino-1,3,4-thiadiazol-2-yl)cyclobutanol (2.10 g, 12.3 mmol). The mixture was stirred at 80 °C for 1 hr. After completion, the reaction mixture was worked up with aqueous saturated NH₄Cl, then extracted with DCM. The combined organic layers were washed with saturated brine, dried over anhydrous Na₂SO₄, filtered, and concentrated under reduced pressure. The residue was purified by flash column chromatography on silica gel (eluting with 20% EtOAc in PE) to afford 1-(5-bromo-1,3,4-thiadiazol-2-yl)cyclobutanol (183 mg). LCMS (ESI, m/z): [M+H]⁺=235.0.

### Intermediate N

### 1-(5-bromo-1,3,4-thiadiazol-2-yl)cyclopropane-1-carbonitrile (N)

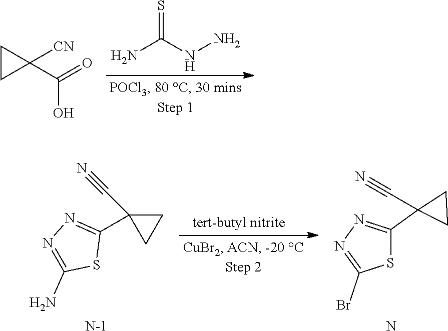

### Step 1: 1-(5-amino-1,3,4-thiadiazol-2-yl) cyclopropane-1-carbonitrile (N-1)

To a solution of 1-cyanocyclopropanecarboxylic acid (10 g, 90.0 mmol) in POCl₃ (100 mL) was added amino thiourea (8.20 g, 90.0 mmol) under N₂. The mixture was stirred at 80 °C for 30 mins under N₂ atmosphere. LCMS showed the reaction completed. The reaction mixture was concentrated under reduced pressure. The residue was diluted with DCM, then neutralized by saturated aqueous NaHCO₃ till pH 8-9. Then the resulting mixture was extracted with DCM. The combined organic layers were washed with saturated brine, dried over anhydrous Na₂SO₄, filtered, and concentrated under reduced pressure to afford 1-(5-amino-1,3,4-thiadiazol-2-yl) cyclopropane carbonitrile (3.4 g). LCMS (ESI, m/z): [M+H]⁺=167.1.

### Step 2: 1-(5-bromo-1,3,4-thiadiazol-2-yl) cyclopropane-1-carbonitrile (N)

To a solution of 1-(5-amino-1,3,4-thiadiazol-2-yl) cyclopropane carbonitrile (3.84 g, 23.1 mmol) in ACN (200 mL) was added tert-butyl nitrite (2.91 g, 28.2 mmol) and CuBr₂ (7.74 g, 34.7 mmol) under N₂. The mixture was stirred at −20 °C for 5 hrs under N₂ atmosphere. LCMS showed the reaction completed. The reaction mixture was worked up with water and extracted with EtOAc. The combined organic layers were washed with saturated brine, dried over anhydrous Na₂SO₄, and concentrated under reduced pressure. The residue was purified by flash column chromatography on silica gel (eluting with 9% EtOAc in PE) to afford 1-(5-bromo-1,3,4-thiadiazol-2-yl) cyclopropane carbonitrile (1.2 g). LCMS (ESI, m/z): [M+H]⁺=300.0.

### Intermediate O

### 1-(1H-pyrazol-4-yl)cyclopropane-1-carbonitrile (O)

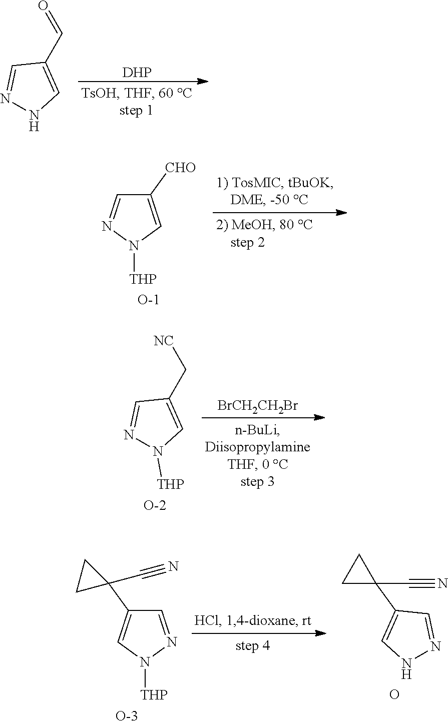

### Step 1: 1-tetrahydropyran-2-ylpyrazole-4-carbaldehyde (O-1)

To a mixture of 1H-pyrazole-4-carbaldehyde (2 g, 20.8 mmol) in THF (60 mL) was added 3,4-dihydro-2H-pyran (3.50 g, 41.6 mmol) and 4-methylbenzenesulfonic acid (358 mg, 2.08 mmol). The mixture was stirred at 60 °C for 2 hrs. After completion, the reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 50% EtOAc in PE) to give 1-tetrahydropyran-2-ylpyrazole-4-carbaldehyde (3.27 g). LCMS (ESI, m/z): [M+H]⁺=181.2.

### Step 2: 2-(1-tetrahydropyran-2-ylpyrazol-3-yl)acetonitrile (0-2)

To a solution of TosMIC (3.64 g, 18.6 mmol) in 1,2-Dimethoxyethane (22.6 mL) was added a solution of KOtBu (1 M in THF, 35.5 mL, 35.5 mmol) dropwise at −50 °C under N₂. The mixture was stirred at −50 °C for 20 mins. Then a solution of 1-tetrahydropyran-2-ylpyrazole-3-carbaldehyde (3.2 g, 17.8 mmol) in 1,2-Dimethoxyethane (22.6 mL) was added dropwise. The reaction mixture was stirred at −50 °C for 30 mins under N₂ atmosphere. After completion, MeOH (22.6 mL) was added to the reaction mixture, then stirred at 80 °C for 1 hr. The reaction mixture was cooled to room temperature and concentrated under reduced pressure. The residue was worked up with water, then acidified to pH=5-6 with AcOH. The resulting mixture was extracted with DCM. The combined organic layers were washed with saturated brine, dried over anhydrous Na₂SO₄, filtered, and concentrated under reduced pressure. The residue was purified by flash column chromatography on silica gel (eluting with 33% EtOAc in PE) to give 2-(1-tetrahydropyran-2-ylpyrazol-3-yl)acetonitrile (2.1 g). LCMS (ESI, m/z): [M+H]⁺=108.2.

### Step 3: 1-(1-tetrahydropyran-2-ylpyrazol-4-yl)cyclopropanecarbonitrile (0-3)

To a solution of Diisopropylamine (2.97 g, 29.3 mmol) in THE (34 mL) was added n-BuLi (1.6 M in hexane, 16.7 mL, 26.7 mmol) dropwise at −30 °C under N₂. The mixture was stirred at room temperature for 1 hr. Then 2-(1-tetrahydropyran-2-ylpyrazol-4-yl)acetonitrile (1.70 g, 8.89 mmol) in THE (8.5 mL) was added to the mixture at 0 °C under N₂. The mixture was stirred at room temperature for 1 hr. Then BrCH₂CH₂Br (5.12 g, 26.7 mmol) was added at 0 °C. The reaction mixture was stirred for an additional 1 hr at 0 °C under N₂ atmosphere. After completion, the reaction mixture was quenched with saturated aqueous NH₄Cl, then extracted with DCM. The combined organic layers were washed with saturated brine, dried over anhydrous Na₂SO₄, filtered, and concentrated under reduced pressure. The residue was purified by flash column chromatography on silica gel (eluting with 33% EtOAc in PE) to give 1-(1-tetrahydropyran-2-ylpyrazol-4-yl)cyclopropanecarbonitrile (1.0 g). LCMS (ESI, m/z): [M+H-THP]⁺=134.2.

### Step 4: 1-(1H-pyrazol-4-yl)cyclopropanecarbonitrile (O)

To a solution of 1-(1-tetrahydropyran-2-ylpyrazol-4-yl)cyclopropanecarbonitrile (900 mg, 4.14 mmol) in 1,4-dioxane (10 mL) was added HCl (4 M in 1,4-dioxane, 3.1 mL, 12.4 mmol) dropwise. The mixture was stirred at room temperature for 1 hr. After completion, the reaction mixture was filtered to get the filter cake which was further washed with a mixture of PE and EtOAc (10:1). The solid was dispensed in saturated aqueous sodium bicarbonate, then extracted with DCM. The combined organic layers were dried over anhydrous Na₂SO₄, filtered, and concentrated to give 1-(1H-pyrazol-4-yl)cyclopropanecarbonitrile (506 mg). LCMS (ESI, m/z): [M+H]⁺=134.1.

### Intermediate P

### 5-chloro-1-iodoimidazo[1,5-a]pyridine-7-sulfonyl chloride (P)

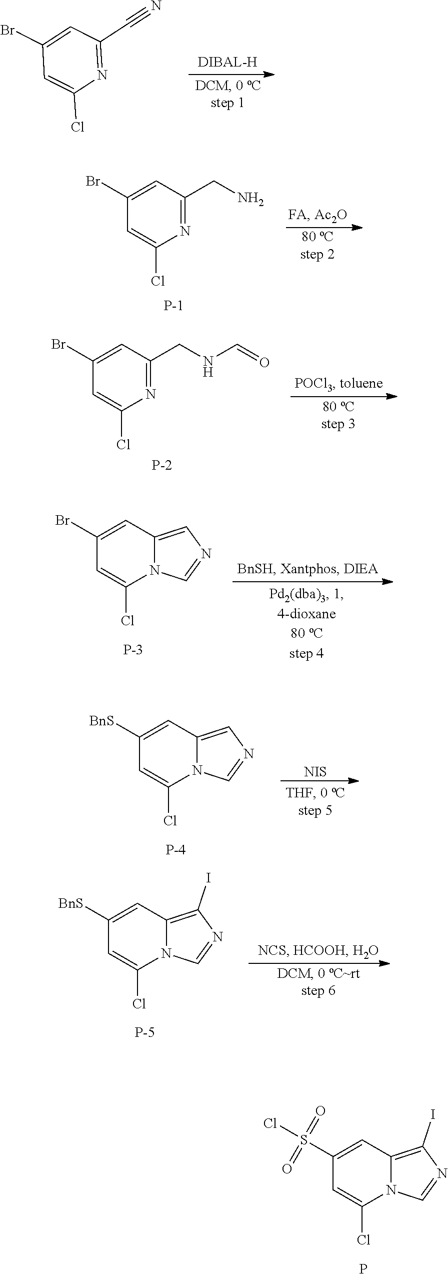

### Step 1: (4-bromo-6-chloropyridin-2-yl)methanamine (P-1)

To a solution of 4-bromo-6-chloro-pyridine-2-carbonitrile (6 g, 27.6 mmol) in DCM (100 mL) was added DIBAL-H (1.0 mol/L in n-hexane, 82.8 mL, 82.8 mmol) dropwise at 0 °C under N₂. The reaction solution was stirred at 0 °C for 2 hrs. LCMS showed the reaction completed. The mixture was worked up with satd. aq. ammonium chloride solution, then was extracted with EtOAc. The combined organic layers were washed with saturated brine, dried over anhydrous Na₂SO₄, filtered, and concentrated to afford the crude product which was used directly for the next step without further purification. LCMS (ESI, m/z): [M+H]⁺=221.0.

### Step 2: N-((4-bromo-6-chloropyridin-2-yl)methyl)formamide (P-2)

To a solution of (4-bromo-6-chloro-2-pyridyl)methanamine (6 g, 27.1 mmol) in formic acid (80 mL) was added acetic anhydride (16 mL) under N₂. The reaction mixture was stirred at 80 °C for 16 hrs. LCMS showed the reaction completed. The mixture was concentrated to give the crude product which was used directly for the next step without further purification. LCMS (ESI, m/z): [M+H]⁺=249.0.

### Step 3: 7-bromo-5-chloro-imidazo[1,5-a]pyridine (P-3)

To a solution of N-[(4-bromo-6-chloro-2-pyridyl)methyl]formamide (7.1 g, 28.5 mmol) in toluene (60 mL) was added POCl₃ (6 mL) under N₂. The reaction solution was stirred at 80 °C for 3 hrs. LCMS showed the reaction completed. The mixture was concentrated and worked up with water. The solution was neutralized to pH=8 by aqueous saturated NaHCO₃ and extracted with EtOAc. The combined organic layers were washed with saturated brine, dried over anhydrous Na₂SO₄, filtered, and concentrated to give a residue. The residue was purified by flash column chromatography on silica gel (eluting with 10% EtOAc in PE) to give 7-bromo-5-chloro-imidazo[1,5-a]pyridine (4.35 g). LCMS (ESI, m/z): [M+H]⁺=233.1.

### Step 4: 7-benzylsulfanyl-5-chloro-imidazo[1,5-a]pyridine (P-4)

To a solution of 7-bromo-5-chloro-imidazo[1,5-a]pyridine (4.35 g, 18.8 mmol) in 1,4-dioxane (60 mL) was added phenylmethanethiol (1.87 g, 15.0 mmol), DIEA (2.43 g, 18.8 mmol), Xantphos (2.17 g, 3.76 mmol) and Pd₂(dba)₃ (593 mg, 0.648 mmol) under N₂. The reaction solution was degassed, then stirred at 80 °C for 3 hrs under N₂ atmosphere. LCMS showed the reaction completed. The reaction mixture was worked up with water, then extracted with EtOAc. The combined organic layers were washed with saturated brine, dried over anhydrous Na₂SO₄, filtered, and concentrated to give a residue. The residue was purified by flash column chromatography on silica gel (eluting with 25% EtOAc in PE) to give 7-benzylsulfanyl-5-chloro-imidazo[1,5-a]pyridine (3.7 g). LCMS (ESI, m/z): [M+H]⁺=275.1.

### Step 5: 7-(benzylthio)-5-chloro-1-iodoimidazo[1,5-a]pyridine (P-5)

To a solution of 7-benzylsulfanyl-5-chloro-imidazo[1,5-a]pyridine (1.5 g, 5.46 mmol) in THE (20 mL) was added NIS (1.84 g, 8.19 mmol) at 0 °C under N₂. The reaction mixture was stirred at 0 °C for 2 hrs. LCMS showed the reaction completed. The reaction was worked up with water and extracted with EtOAc. The combined organic layers were washed with saturated brine, dried over anhydrous Na₂SO₄, filtered, and concentrated under reduced pressure to give the crude. The crude was purified by flash column chromatography on silica gel (eluting with 25% EtOAc in PE) to give 7-benzylsulfanyl-5-chloro-1-iodo-imidazo[1,5-a]pyridine (1.66 g). LCMS (ESI, m/z): [M+H]⁺=401.0.

### Step 6: 5-chloro-1-iodoimidazo[1,5-a]pyridine-7-sulfonyl chloride (P)

To a solution of 7-benzylsulfanyl-5-chloro-1-iodo-imidazo[1,5-a]pyridine (1.66 g, 4.14 mmol) in dichloromethane was added acetic acid (9.5 mL), water (3.0 mL) and NCS (1.66 g, 12.4 mmol) portion-wise at 0 °C. The mixture was stirred at 25 °C for 2 hrs. LCMS showed the reaction completed. The reaction was quenched with water and extracted with DCM. The combined organic layers were washed with saturated brine, dried over anhydrous Na₂SO₄, filtered, and concentrated under reduced pressure to give the crude product. The crude product was purified by flash column chromatography on silica gel (eluting with 10% EtOAc in PE) to give 5-chloro-1-iodo-imidazo[1,5-a]pyridine-7-sulfonyl chloride (950 mg). LCMS (ESI, m/z): [M+H]⁺=376.9.

### Intermediate Q

### 2-(6-(benzylthio)-4-chloro-1H-indazol-1-yl)-5-(difluoromethyl)-1,3,4-thiadiazole (Q)

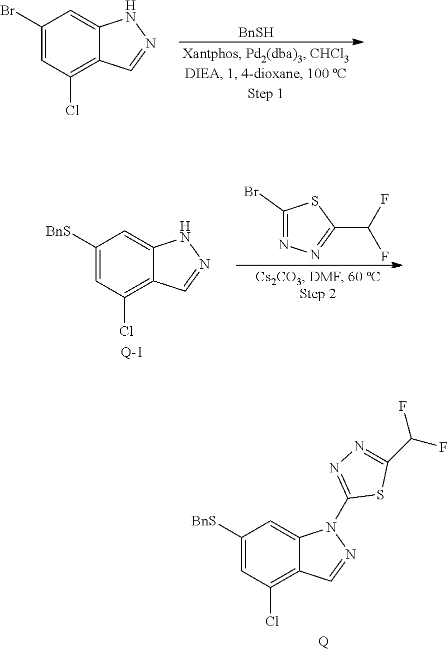

### Step 1: 6-(benzylthio)-4-chloro-1H-indazole (Q-1)

To a mixture of 6-bromo-4-chloro-1H-indazole (5.0 g, 21.6 mmol), phenyl methanethiol (8.05 g, 64.8 mmol) and DIEA (8.38 g, 64.8 mmol) in 1,4-dioxane (50 ml) was added Xantphos (1.25 g, 2.16 mmol) and Pd₂(dba)₃-CHCl₃ (1.12 g, 1.08 mmol) under N₂. The mixture was degassed, stirred at 100 °C for 3 hrs under N₂ atmosphere. TLC indicated the total consumption of the starting material. The reaction mixture was worked up with water, then extracted with EtOAc. The combined organic layers were washed with saturated brine, dried over anhydrous Na₂SO₄, and concentrated under reduced pressure. The residue was purified by flash column chromatography on silica gel (eluting with 10% EtOAc in PE) to afford the product 6-benzylsulfanyl-4-chloro-1H-indazole (5.5 g). LCMS (ESI, m/z): [M+H]⁺=275.1.

### Step 2: 2-(6-(benzylthio)-4-chloro-1H-indazol-1-yl)-5-(difluoromethyl)-1,3,4-thiadiazole (Q)

To a mixture of 6-benzylsulfanyl-4-chloro-1H-indazole (3.3 g, 12.0 mmol) and 2-bromo-5-(difluoromethyl)-1,3,4-thiadiazole (3.87 g, 18.0 mmol) in DMF (50 ml) was added Cs₂CO₃ (5.87 g, 18.0 mmol). The mixture was stirred at 60 °C for 2 hrs under N₂ atmosphere. After completion, the mixture was worked up with water, then extracted with EtOAc. The combined organic layers were washed with saturated brine, dried over anhydrous Na₂SO₄, and concentrated under reduced pressure. The residue was purified by flash column chromatography on silica gel (eluting with 1% EtOAc in PE) to afford 2-(6-benzylsulfanyl-4-chloro-indazol-1-yl)-5-(difluoromethyl)-1,3,4-thiadiazole (4.0 g). LCMS (ESI, m/z): [M+H]⁺=409.2.

### Example 1

### 3-(5-(difluoromethyl)-1,3,4-thiadiazol-2-yl)-N-(1-methylcyclopropyl)-7-(2-oxa-7-azaspiro[3.5]nonan-7-yl)pyrazolo[1,5-a]pyridine-5-sulfonamide (1)

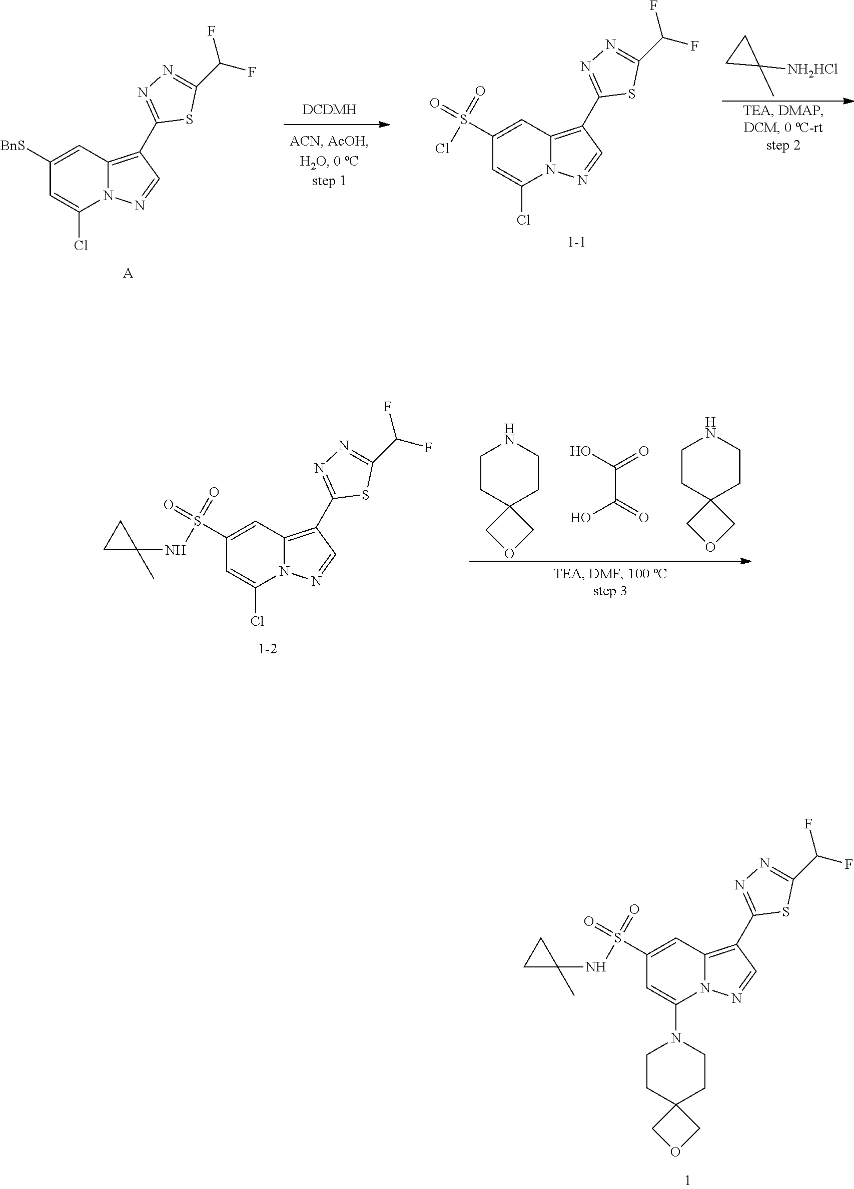

### Step 1: 7-chloro-3-(5-(difluoromethyl)-1,3,4-thiadiazol-2-yl)pyrazolo[1,5-a]pyridine-5-sulfonyl chloride (1-1)

To a suspension of 2-(5-benzylsulfanyl-7-chloro-pyrazolo[1,5-a]pyridin-3-yl)-5-(difluoromethyl)-1,3,4-thiadiazole (375 mg, 0.917 mmol), acetic acid (0.34 mL) and water (0.23 mL) in acetonitrile (9.2 mL) was added 1,3-dichloro-5,5-dimethyl-imidazolidine-2,4-dione (271 mg, 1.38 mmol) portion wise at 0 °C under N₂. The resulting mixture was stirred at 0 °C for 2 hrs. LCMS showed the reaction completed. The reaction mixture was concentrated to give the crude product which was used directly for the next step without further purification. LCMS (ESI, m/z): [M+H]⁺=384.9.

### Step 2: 7-chloro-3-(5-(difluoromethyl)-1,3,4-thiadiazol-2-yl)-N-(1-methyl cyclopropyl)pyrazolo[1,5-a]pyridine-5-sulfonamide (1-2)

To a suspension of 1-methylcyclopropan-1-amine hydrochloride (195.8 mg, 1.82 mmol), TEA (462 mg, 4.57 mmol, 0.64 mL), DMAP (11.1 mg, 0.091 mmol) in DCM (5 mL) was added 7-chloro-3-[5-(difluoromethyl)-1,3,4-thiadiazol-2-yl]pyrazolo[1,5-a]pyridine-5-sulfonyl chloride (352 mg, 0.914 mmol) at 0 °C under N₂. The resulting mixture was stirred at room temperature overnight. LCMS showed the reaction completed. The reaction mixture was concentrated under reduced pressure. The residue was purified by flash column chromatography on silica gel (eluting with 30% EtOAc in PE) to afford 7-chloro-3-(5-(difluoromethyl)-1,3,4-thiadiazol-2-yl)-N-(1-methylcyclopropyl)pyrazolo[1,5-a]pyridine-5-sulfonamide (90 mg). LCMS (ESI, m/z): [M+H]⁺=420.2.

### Step 3: 3-(5-(difluoromethyl)-1,3,4-thiadiazol-2-yl)-N-(1-methylcyclopropyl)-7-(2-oxa-7-azaspiro[3.5]nonan-7-yl)pyrazolo[1,5-a]pyridine-5-sulfonamide (1)

To a mixture of 2-oxa-7-azaspiro[3.5]nonane hemi-oxalate (54.5 mg, 0.317 mmol) and 7-chloro-3-[5-(difluoromethyl)-1,3,4-thiadiazol-2-yl]-N-(1-methylcyclopropyl)-pyrazolo[1,5-a]pyridine-5-sulfonamide (90 mg, 0.215 mmol) in DMF (8 mL) was added TEA (173 mg, 1.71 mmol, 0.24 mL) under N₂. The reaction mixture was stirred at 100 °C for 16 hrs. LCMS showed the reaction completed. The reaction mixture was worked up with water and extracted with EtOAc. The organic layers were washed with saturated brine, dried over anhydrous Na₂SO₄, filtered, and concentrated under reduced pressure. The residue was purified by flash column chromatography on silica gel (eluting with 5% MeOH in DCM) to afford the product which was further purified by prep-HPLC (0.05% NH₄HCO₃) to afford 3-(5-(difluoromethyl)-1,3,4-thiadiazol-2-yl)-N-(1-methylcyclopropyl)-7-(2-oxa-7-azaspiro[3.5]nonan-7-yl)pyrazolo[1,5-a]pyridine-5-sulfonamide. LCMS (ESI, m/z): [M+H]⁺=511.2. ¹H NMR (400 MHz, DMSO-d₆) δ 8.95 (s, 1H), 8.45 (s, 1H), 8.39 (s, 1H), 7.66 (t, J=52.0 Hz, 1H), 6.82 (s, 1H), 4.43 (s, 4H), 3.47-3.40 (m, 4H), 2.09-2.02 (m, 4H), 1.13 (s, 3H), 0.74-0.67 (m, 2H), 0.49-0.42 (m, 2H).

Examples 2-24 were prepared according to the general procedures herein for Example 1, and in an analogous manner to that used to synthesize the example compounds with the appropriate intermediates. The starting materials were either prepared as described in the Intermediates section, commercially available, or prepared from commercially available reagents using conventional reactions well known in the art.

| | | | LC-MS |
|---|---|---|---|
| | | | (ESI, |
| Example | | | m/z): |
| Number | Structure | NMR | [M + H]⁺ |
| 2 | 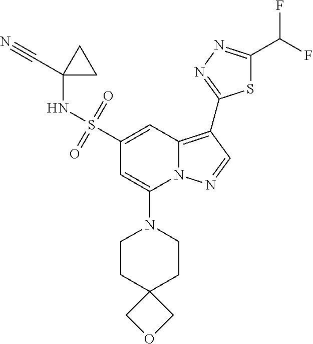 [SMILES: N#CC1(NS(=O)(=O)c2cc(N3CCC4(CC3)COC4)n3ncc(-c4nnc(C(F)F)s4)c3c2)CC1] | ¹H NMR (400 MHz, DMSO-d₆) δ 8.97 (s, 1H), 8.43 (s, 1H), 7.66 (t, J = 52.0 Hz, 1H), 6.82 (s, 1H), 4.42 (s, 4H), 3.48-3.42 (m, 4H), 2.08-2.02 (m, 4H), 1.48-1.42 (m, 2H), 1.39-1.33 (m, 2H). | 522.2 |
| | N-(1-cyanocyclopropyl)-3-(5- | | |
| | (difluoromethyl)-1,3,4-thiadiazol-2-yl)-7-(2- | | |
| | oxa-7-azaspiro[3.5]nonan-7-yl)pyrazolo[1,5- | | |
| | a]pyridine-5-sulfonamide | | |
| 3 | 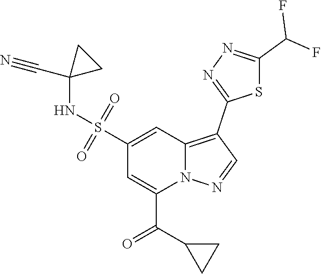 [SMILES: N#CC1(NS(=O)(=O)c2cc(C(=O)C3CC3)n3ncc(-c4nnc(C(F)F)s4)c3c2)CC1] | ¹H NMR (400 MHz, DMSO-d₆) δ 8.99 (s, 1H), 8.47 (s, 1H), 7.66 (t, J = 52 Hz, 1H), 6.85 (s, 1H), 3.98-3.51 (m, 8H), 2.12-2.03 (m, 1H), 1.48-1.42 (m, 2H), 1.38-1.32 (m, 2H), 0.83-0.74 (m, 4H). | 549.2 |
| | N-(1-cyanocyclopropyl)-7-[4- | | |
| | (cyclopropanecarbonyl)piperazin-1-yl]-3-[5- | | |
| | (difluoromethyl)-1,3,4-thiadiazol-2- | | |
| | yl]pyrazolo[1,5-a]pyridine-5-sulfonamide | | |
| 4 | 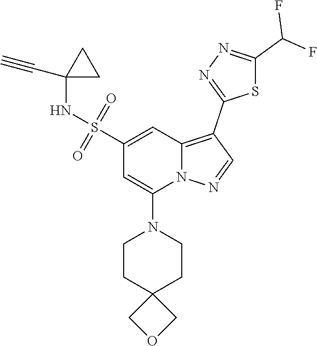 [SMILES: C#CC1(NS(=O)(=O)c2cc(N3CCC4(CC3)COC4)n3ncc(-c4nnc(C(F)F)s4)c3c2)CC1] | ¹H NMR (400 MHz, DMSO-d₆) δ 8.94 (s, 1H), 8.42 (d, J = 4.0 Hz, 1H), 7.66 (t, J = 52 Hz, 1H), 6.86 (d, J = 4 Hz, 1H), 4.42 (s, 4H), 3.46-3.40 (m, 4H), 2.71 (s, 1H), 2.07-2.04 (m, 4H), 1.24-1.21 (m, 2H), 1.06-1.03 (m, 2H). | 429.9 |
| | 3-(5-(difluoromethyl)-1,3,4-thiadiazol-2-yl)- | | |
| | N-(1-ethynylcyclopropyl)-7-(2-oxa-7- | | |
| | azaspiro[3.5]nonan-7-yl)pyrazolo[1,5- | | |
| | a]pyridine-5-sulfonamide | | |
| 5 | 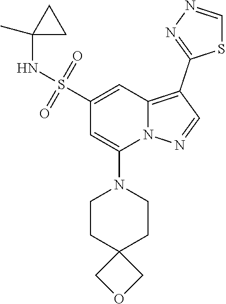 [SMILES: CC1(NS(=O)(=O)c2cc(N3CCC4(CC3)COC4)n3ncc(-c4nncs4)c3c2)CC1] | ¹H NMR (400 MHz, DMSO-d₆) δ 9.57 (s, 1H), 8.84 (s, 1H), 8.43(s, 1H), 8.41(br s, 1H), 6.77 (s, 1H), 4.42 (s, 4H), 3.44-3.39 (m, 4H), 2.08-2.02 (t, J = 8.0 Hz, 4H), 1.12 (s, 3H), 0.73-0.67 (m, 2H), 0.47-0.41 (m, 2H). | 461.1 |
| | N-(1-cyanocyclopropyl)-3-(5- | | |
| | (difluoromethyl)-1,3,4-thiadiazol-2-yl)-7-(4- | | |
| | (propylsulfonyl)piperazin-1-yl)pyrazolo[1,5- | | |
| | a]pyridine-5-sulfonamide | | |
| 6 | 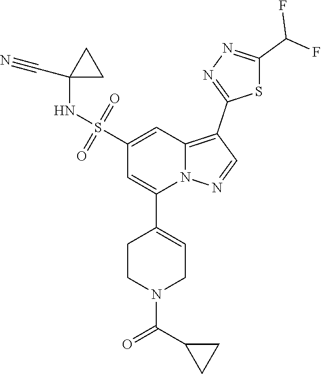 [SMILES: N#CC1(NS(=O)(=O)c2cc(C3=CCN(C(=O)C4CC4)CC3)n3ncc(-c4nnc(C(F)F)s4)c3c2)CC1] | ¹H NMR (400 MHz, DMSO-d₆) δ 9.02 (s, 1H), 8.81 (s, 1H), 7.54 (t, J = 45.8 Hz, 1H), 7.40 (s, 1H), 6.78-6.70 (m, 1H), 4.55 (br s, 1H), 4.27 (br s, 1H), 3.97 (br s, 1H), 3.77 (br s, 1H), 2.82 (br s, 1H), 2.69 (br s, 1H), 1.99-2.11 (m, 1H) 1.50-1.43 (m, 2H), 1.39-1.32 (m, 2H), 0.94-0.67 (m, 4H). | 545.9 |
| | N-(1-cyanocyclopropyl)-7-(1- | | |
| | (cyclopropanecarbonyl)-1,2,3,6- | | |
| | tetrahydropyridin-4-yl)-3-(5-(difluoromethyl)- | | |
| | 1,3,4-thiadiazol-2-yl)pyrazolo[1,5-a]pyridine- | | |
| | 5-sulfonamide | | |
| 7 | 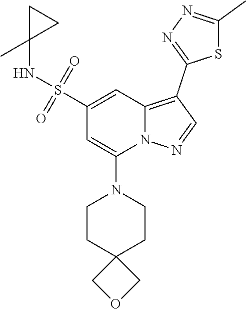 | ¹H NMR (400 MHz, DMSO-d₆) δ 8.77 (s, 1H), 8.36 (s, 1H), 8.33 (s, 1H), 6.77 (s, 1H), 4.43 (s, 4H), 3.42-3.32 (m, 4H), 2.80 (s, 3H), 2.09-2.02 (m, 4H), 1.12 (s, 3H), 0.74-0.67 (m, 2H), 0.48-0.41 (m, 2H). | 475.3 |
| | 3-(5-methyl-1,3,4-thiadiazol-2-yl)-N-(1- | | |
| | methylcyclopropyl)-7-(2-oxa-7- | | |
| | azaspiro[3.5]nonan-7-yl)pyrazolo[1,5- | | |
| | a]pyridine-5-sulfonamide | | |
| 8 | 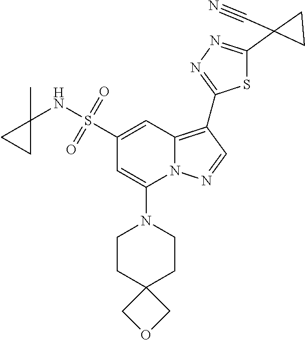 [SMILES: CC1(NS(=O)(=O)c2cc(N3CCC4(CC3)COC4)n3ncc(-c4nnc(C5(C#N)CC5)s4)c3c2)CC1] | ¹H NMR (400 MHz, DMSO-d₆) δ 8.83 (s, 1H), 8.41 (s, 1H), 8.33 (s, 1H), 6.78 (s, 1H), 4.42 (s, 4H), 3.44-3.38 (m, 4H), 2.17-2.10 (m, 2H), 2.06-2.00 (m, 4H), 1.96- 1.89 (m, 2H), 1.11 (s, 3H), 0.72- 0.66 (m, 2H), 0.46- 0.40 (m, 2H). | 526.2 |
| | 3-(5-(1-cyanocyclopropyl)-1,3,4-thiadiazol-2- | | |
| | yl)-N-(1-methylcyclopropyl)-7-(2-oxa-7- | | |
| | azaspiro[3.5]nonan-7-yl)pyrazolo[1,5- | | |
| | a]pyridine-5-sulfonamide | | |
| 9 | 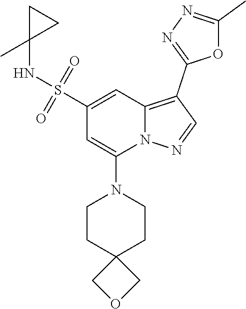 | ¹H NMR (400 MHz, DMSO-d₆) δ 8.75 (s, 1H), 8.44 (s, 1H), 8.21 (s, 1H), 6.78 (s, 1H), 4.42 (s, 4H), 3.44-3.28 (m, 4H), 2.61 (s, 3H), 2.08-2.01 (m, 4H), 1.12 (s, 3H), 0.72-0.67 (m, 2H), 0.48-0.42 (m, 2H). | 459.3 |
| | 3-(5-methyl-1,3,4-oxadiazol-2-yl)-N-(1- | | |
| | methylcyclopropyl)-7-(2-oxa-7- | | |
| | azaspiro[3.5]nonan-7-yl)pyrazolo[1,5- | | |
| | a]pyridine-5-sulfonamide | | |
| 10 | 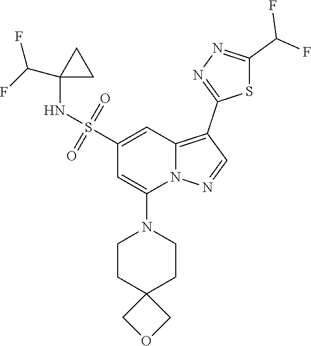 [SMILES: O=S(=O)(NC1(C(F)F)CC1)c1cc(N2CCC3(CC2)COC3)n2ncc(-c3nnc(C(F)F)s3)c2c1] | ¹H NMR (400 MHz, DMSO-d₆) δ 9.16 (br s, 1 H), 8.94 (s, 1 H), 8.33 (s, 1 H), 7.65 (t, J = 56 Hz, 1 H), 6.80 (s, 1 H), 5.75 (t, J = 56 Hz, 1 H), 4.42 (s, 4 H), 3.43-3.39 (m, 4 H), 2.07-2.03 (m, 4 H), 0.98-0.90 (m, 4 H). | 547.2 |
| | 3-(5-(difluoromethyl)-1,3,4-thiadiazol-2-yl)- | | |
| | N-(1-(difluoromethyl)cyclopropyl)-7-(2-oxa- | | |
| | 7-azaspiro[3.5]nonan-7-yl)pyrazolo[1,5- | | |
| | a]pyridine-5-sulfonamide | | |
| 11 | 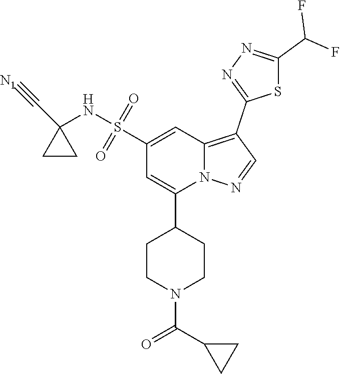 | ¹H NMR (400 MHz, DMSO-d₆) δ 9.06 (s, 1H), 8.76 (s, 1H), 7.68 (t, J = 52.0 Hz, 1H), 7.36 (s, 1H), 4.65-4.45 (m, 2H), 4.00-3.90 (m, 1H), 3.62-3.52 (m, 1H), 2.88-2.80 (m, 1H), 2.24-2.14 (m, 2H), 2.10-2.02 (m, 1H), 1.80-1.56 (m, 2H), 1.52-1.25 (m, 4H), 0.85-0.61 (m, 4H). | 548.2 |
| | N-(1-cyanocyclopropyl)-7-[1- | | |
| | (cyclopropanecarbonyl)-4-piperidyl]-3-[5- | | |
| | (difluoromethyl)-1,3,4-thiadiazol-2- | | |
| | yl]pyrazolo[1,5-a]pyridine-5-sulfonamide | | |
| 12 | 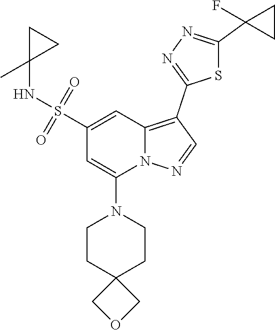 [SMILES: CC1(NS(=O)(=O)c2cc(N3CCC4(CC3)COC4)n3ncc(-c4nnc(C5(F)CC5)s4)c3c2)CC1] | ¹H NMR (400 MHz, DMSO-d₆) δ 8.85 (s, 1H), 8.40 (s, 1H), 8.36 (s, 1H), 6.77 (s, 1H), 4.42 (s, 4H), 3.44-3.35 (m, 4H), 2.08-2.01 (m, 4H), 1.86-1.73 (m, 2H), 1.57-1.50 (m, 2H), 1.11 (s, 3H), 0.72-0.66 (m, 2H), 0.47-0.40 (m, 2H). | 519.2 |
| | 3-(5-(1-fluorocyclopropyl)-1,3,4-thiadiazol-2- | | |
| | yl)-N-(1-methylcyclopropyl)-7-(2-oxa-7- | | |
| | azaspiro[3.5]nonan-7-yl)pyrazolo[1,5- | | |
| | a]pyridine-5-sulfonamide | | |
| 13 | 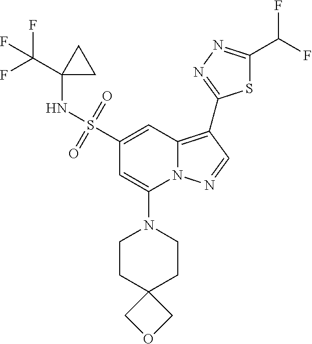 [SMILES: O=S(=O)(NC1(C(F)(F)F)CC1)c1cc(N2CCC3(CC2)COC3)n2ncc(-c3nnc(C(F)F)s3)c2c1] | ¹H NMR (400 MHz, DMSO-d₆) δ 9.55 (br s, 1H), 8.95 (s, 1H), 8.34 (s, 1H), 7.66 (t, J = 53.3 Hz, 1H), 6.79 (s, 1H), 4.42 (s, 4H), 3.46- 3.40 (m, 4H), 2.10-2.01 (m, 4H), 1.27-1.19 (m, 2H), 1.17-1.10 (m, 2H). | 565.2 |
| | 3-[5-(difluoromethyl)-1,3,4-thiadiazol-2-yl]-7- | | |
| | (2-oxa-7-azaspiro[3.5]nonan-7-yl)-N-[1- | | |
| | (trifluoromethyl)cyclopropyl]pyrazolo[1,5- | | |
| | a]pyridine-5-sulfonamide | | |
| 14 | 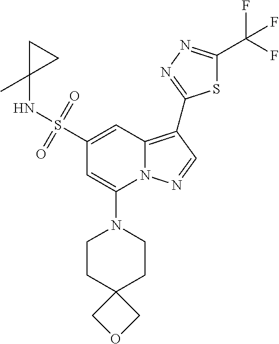 [SMILES: CC1(NS(=O)(=O)c2cc(N3CCC4(CC3)COC4)n3ncc(-c4nnc(C(F)(F)F)s4)c3c2)CC1] | ¹H NMR (400 MHz, DMSO-d6) δ 9.00 (s, 1H), 8.47 (s, 1H), 8.39 (s, 1H), 6.84 (s, 1H), 4.42 (s, 4H), 3.46-3.40 (m, 4H), 2.10-2.02 (m, 4H), 1.13 (s, 3H), 0.74- 0.67 (m, 2H), 0.49- 0.42 (m, 2H). | 529.2 |
| | N-(1-methylcyclopropyl)-7-(2-oxa-7- | | |
| | azaspiro[3.5]nonan-7-yl)-3-(5- | | |
| | (trifluoromethyl)-1,3,4-thiadiazol-2- | | |
| | yl)pyrazolo[1,5-a]pyridine-5-sulfonamide | | |
| 15 | 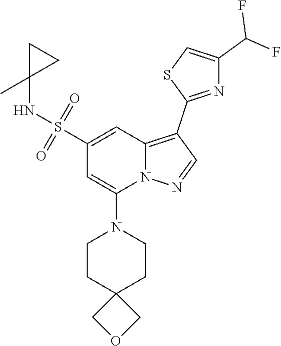 [SMILES: CC1(NS(=O)(=O)c2cc(N3CCC4(CC3)COC4)n3ncc(-c4nc(C(F)F)cs4)c3c2)CC1] | ¹H NMR (400 MHz, DMSO-d₆) δ 8.81 (s, 1H), 8.40 (s, 1H), 8.32 (s, 1 H), 8.08 (s, 1H), 7.18 (t, J = 54.6 Hz, 1H), 6.74 (s, 1H), 4.41 (s, 4H), 3.43-3.37 (m, 4H), 2.07-2.01 (m, 4H), 1.13 (s, 3H), 0.74- 0.67 (m, 2H), 0.48-0.41 (m, 2H). | 510.2 |
| | 3-(4-(difluoromethyl)thiazol-2-yl)-N-(1- | | |
| | methylcyclopropyl)-7-(2-oxa-7- | | |
| | azaspiro[3.5]nonan-7-yl)pyrazolo[1,5- | | |
| | a]pyridine-5-sulfonamide | | |
| 16 | 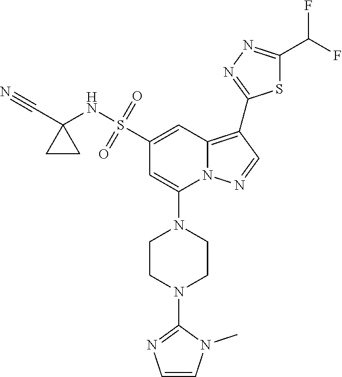 [SMILES: Cn1ccnc1N1CCN(c2cc(S(=O)(=O)NC3(C#N)CC3)cc3c(-c4nnc(C(F)F)s4)cnn23)CC1] | ¹HNMR (400 MHz, DMSO-d₆) δ 9.01 (s, 1H), 8.49 (s, 1H), 8.16 (s, 1H), 7.67 (t, J = 53.2 Hz, 1H), 6.93 (s, 1H), 6.91 (s, 1H), 6.64 (s, 1H), 3.74-3.66 (m, 4H), 3.52 (s, 3H), 3.27- 3.22 (m, 4H), 1.51- 1.43 (m, 2H), 1.43- 1.36 (m, 2H). | 561.3 |
| | N-(1-cyanocyclopropyl)-3-(5- | | |
| | (difluoromethyl)-1,3,4-thiadiazol-2-yl)-7-(4- | | |
| | (1-methyl-1H-imidazol-2-yl)piperazin-1- | | |
| | yl)pyrazolo[1,5-a]pyridine-5-sulfonamide | | |
| 17 | 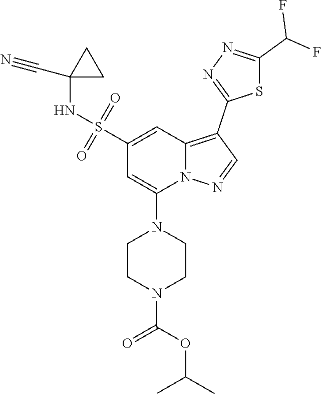 [SMILES: CC(C)OC(=O)N1CCN(c2cc(S(=O)(=O)NC3(C#N)CC3)cc3c(-c4nnc(C(F)F)s4)cnn23)CC1] | ¹H NMR (400 MHz, DMSO-d₆) δ 9.50 (br s, 1H), 9.01 (s, 1H), 8.49 (s, 1H), 7.67 (t, J = 53.3 Hz, 1H), 6.86 (s, 1H), 4.88-4.79 (m, 1H), 3.68-3.61 (m, 4H), 3.56-3.51 (m, 4H), 1.52-1.44 (m, 2H), 1.42-1.35 (m, 2H), 1.23 (d, J = 8 Hz, 6H). | 567.3 |
| | Isopropyl 4-(5-(N-(1- | | |
| | cyanocyclopropyl)sulfamoyl)-3-(5- | | |
| | (difluoromethyl)-1,3,4-thiadiazol-2- | | |
| | yl)pyrazolo[1,5-a]pyridin-7-yl)piperazine-1- | | |
| | carboxylate | | |
| 18 | 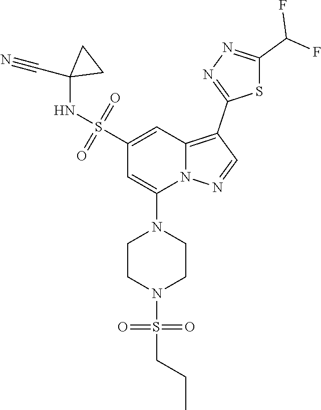 [SMILES: CCCS(=O)(=O)N1CCN(c2cc(S(=O)(=O)NC3(C#N)CC3)cc3c(-c4nnc(C(F)F)s4)cnn23)CC1] | ¹H NMR (400 MHz, DMSO-d₆) δ 8.99 (s, 1H), 8.48 (s, 1H), 7.67 (t, J = 54 Hz, 1H), 6.87 (s, 1H), 3.66-3.59 (m, 4H), 3.50-3.44 (m, 4H), 3.14 (t, J = 8 Hz, 2H), 1.80-1.72 (m, 2H), 1.48-1.41 (m, 2H), 1.38-1.31 (m, 2H), 1.02 (t, J = 8 Hz, 3H) | 587.1 |
| | N-(1-cyanocyclopropyl)-3-(5- | | |
| | (difluoromethyl)-1,3,4-thiadiazol-2-yl)-7-(4- | | |
| | (propylsulfonyl)piperazin-1-yl)pyrazolo[1,5- | | |
| | a]pyridine-5-sulfonamide | | |
| 19 | 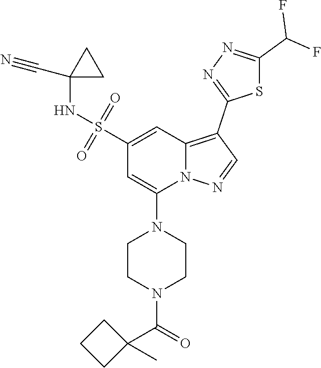 [SMILES: CC1(C(=O)N2CCN(c3cc(S(=O)(=O)NC4(C#N)CC4)cc4c(-c5nnc(C(F)F)s5)cnn34)CC2)CCC1] | ¹H NMR (400 MHz, DMSO-d₆) δ 8.99 (s, 1H), 8.47 (s, 1H), 7.67 (t, J = 54.0 Hz, 1H), 6.86 (s, 1H), 3.75-3.69 (m, 2H), 3.66-3.49 (m, 6H), 2.49-2.43 (m, 2H), 2.02-1.91 (m, 1H), 1.88-1.82 (m, 2H), 1.69-1.62 (m, 1H), 1.47-1.39 (m, 5H), 1.37-1.30 (m 2H). | 577.3 |
| | N-(1-cyanocyclopropyl)-3-[5- | | |
| | (difluoromethyl)-1,3,4-thiadiazol-2-yl]-7-[4- | | |
| | (1-methylcyclo butanecarbonyl)piperazin-1- | | |
| | yl]pyrazolo[1,5-a]pyridine-5-sulfonamide | | |
| 20 | 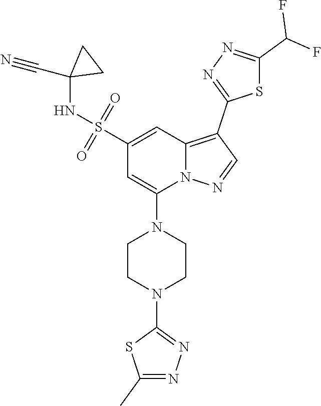 [SMILES: Cc1nnc(N2CCN(c3cc(S(=O)(=O)NC4(C#N)CC4)cc4c(-c5nnc(C(F)F)s5)cnn34)CC2)s1] | ¹H NMR (400 MHz, DMSO-d₆) δ 9.02 (s, 1H), 8.50 (s, 1H), 7.68 (t, J = 54.0 Hz, 1H), 6.90 (s, 1H), 3.74-3.68 (m, 8H), 2.56 (s, 3H), 1.49-1.43 (m, 2H), 1.40-1.33 (m, 2H). | 579.0 |
| | N-(1-cyanocyclopropyl)-3-[5- | | |
| | (difluoromethyl)-1,3,4-thiadiazol-2-yl]-7-[4- | | |
| | (5-methyl-1,3,4-thiadiazol-2-yl)piperazin-1- | | |
| | yl]pyrazolo[1,5-a]pyridine-5-sulfonamide | | |
| 21 | 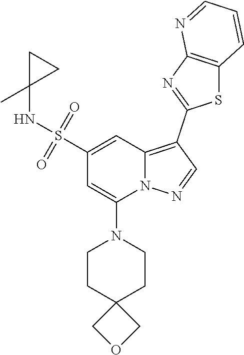 [SMILES: CC1(NS(=O)(=O)c2cc(N3CCC4(CC3)COC4)n3ncc(-c4nc5ncccc5s4)c3c2)CC1] | ¹H NMR (400 MHz, DMSO-d6) δ 8.99 (s, 1H), 8.70-8.60 (m, 2H), 8.58 (s, 1H), 8.47 (s, 1H), 7.44 (dd, J = 8.0 Hz, 4.7 Hz, 1H), 6.83 (s, 1H), 4.43 (s, 4H), 3.49-3.41 (m, 4H), 2.11-2.01 (m, 4H), 1.15 (s, 3H), 0.76- 0.69 (m, 2H), 0.50- 0.43 (m, 2H). | 511.3 |
| | N-(1-methylcyclopropyl)-7-(2-oxa-7- | | |
| | azaspiro[3.5]nonan-7-yl)-3-(thiazolo[4,5- | | |
| | b]pyridin-2-yl)pyrazolo[1,5-a]pyridine-5- | | |
| | sulfonamide | | |
| 22 | 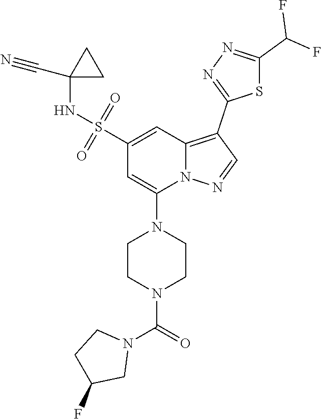 [SMILES: N#CC1(NS(=O)(=O)c2cc(N3CCN(C(=O)N4CC[C@H](F)C4)CC3)n3ncc(-c4nnc(C(F)F)s4)c3c2)CC1] | ¹HNMR (400 MHz, DMSO-d₆) δ 9.42 (br s, 1H), 8.99 (s, 1H), 8.48 (s, 1H), 7.67 (t, J = 53.2 Hz, 1H), 6.86 (s, 1H), 5.32 (d, J = 53.2 Hz, 1H), 3.76- 3.41 (m, 12H), 2.17- 1.91 (m, 2H), 1.49- 1.42 (m, 2H), 1.41- 1.34 (m, 2H). | 596.3 |
| | (S)-N-(1-cyanocyclopropyl)-3-(5- | | |
| | (difluoromethyl)-1,3,4-thiadiazol-2-yl)-7-(4- | | |
| | (3-fluoropyrrolidine-1-carbonyl)piperazin-1- | | |
| | yl)pyrazolo[1,5-a]pyridine-5-sulfonamide | | |
| 23 | 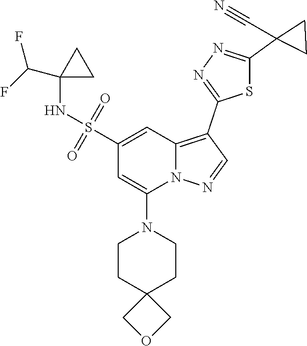 [SMILES: N#CC1(c2nnc(-c3cnn4c(N5CCC6(CC5)COC6)cc(S(=O)(=O)NC5(C(F)F)CC5)cc34)s2)CC1] | ¹H NMR (400 MHz, DMSO-d₆) δ 9.12 (br s, 1H), 8.83 (s, 1H), 8.29 (s, 1H), 6.77 (s, 1H), 5.76 (t, J = 56.0 Hz, 1H), 4.42 (s, 4H), 3.44- 3.38 (m, 4H), 2.18-2.10 (m, 2H), 2.08-2.01 (m, 4H), 1.97-1.90 (m, 2H), 0.98-0.88(m, 4H). | 562.3 |
| | 3-[5-(1-cyanocyclopropyl)-1,3,4-thiadiazol-2- | | |
| | yl]-N-[1-(difluoromethyl)cyclopropyl]-7-(2- | | |
| | oxa-7-azaspiro[3.5]nonan-7-yl)pyrazolo[1,5- | | |
| | a]pyridine-5-sulfonamide | | |
| 24 | 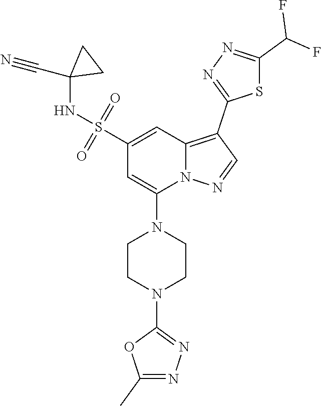 [SMILES: Cc1nnc(N2CCN(c3cc(S(=O)(=O)NC4(C#N)CC4)cc4c(-c5nnc(C(F)F)s5)cnn34)CC2)o1] | ¹H NMR (400 MHz, DMSO-d₆) δ 9.01 (s, 1H), 8.50 (s, 1H), 7.66 (t, J = 54 Hz, 1H), 6.89 (s, 1H), 3.67 (s, 8H), 2.38 (s, 3H), 1.48-1.33 (m, 4H). | 563.0 |
| | N-(1-cyanocyclopropyl)-3-[5- | | |
| | (difluoromethyl)-1,3,4-thiadiazol-2-yl]-7-[4- | | |
| | (5-methyl-1,3,4-oxadiazol-2-yl)piperazin-1- | | |
| | yl]pyrazolo[1,5-a]pyridine-5-sulfonamide | | |

### Example 25

### 3-(3-(difluoromethyl)-1,2,4-thiadiazol-5-yl)-N-(1-mnethylcyclopropyl)-7-(2-oxa-7-azaspiro[3.5]nonan-7-yl)pyrazolo[1,5-a]pyridine-5-sulfonamnide (25)

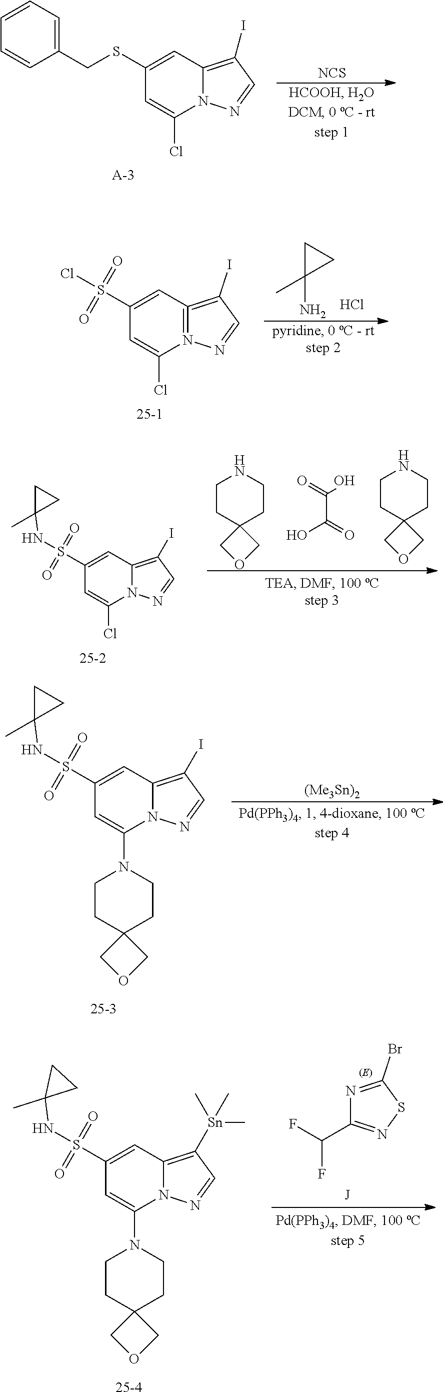

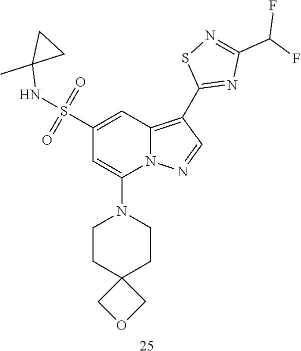

### Step 1: 7-chloro-3-iodopyrazolo[1,5-a]pyridine-5-sulfonyl chloride (25-1)

To a solution of 5-benzylsulfanyl-7-chloro-3-iodo-pyrazolo[1,5-a]pyridine (A-3, 403 mg, 1.01 mmol) in DCM (12 mL), formic acid (1.5 mL), and water (0.7 mL) was added NCS (537 mg, 4.02 mmol) at 0 °C. The reaction mixture was stirred at room temperature for 1 hr. LCMS showed the reaction completed. The reaction mixture was worked up with water, then extracted with DCM. The combined organic layers were washed with saturated brine, dried over anhydrous Na₂SO₄, filtered, and concentrated under reduced pressure. The residue was used directly in the next step without further purification.

### Step 2: 7-chloro-3-iodo-N-(1-methylcyclopropyl)pyrazolo[1,5-a]pyridine-5-sulfonamide (25-2)

To a solution of 1-methylcyclopropan-1-amine hydrochloride (102 mg, 0.95 mmol) in pyridine (6 mL) was added 7-chloro-3-iodo-pyrazolo[1,5-a]pyridine-5-sulfonyl chloride (360 mg, 0.95 mmol) at 0 °C under N₂. The mixture was stirred at room temperature for 30 mins. LCMS showed the reaction completed. The reaction mixture was worked up with water, then extracted with EtOAc. The combined organic layers were washed with water and saturated brine, dried over anhydrous Na₂SO₄, filtered, and concentrated under reduced pressure. The residue was purified by flash column chromatography on silica gel (eluting with 20% EtOAc in PE) to afford 7-chloro-3-iodo-N-(1-methylcyclopropyl)pyrazolo[1,5-a]pyridine-5-sulfonamide (290 mg). LCMS (ESI, m/z): [M+H]⁺=411.9.

### Step 3: 3-iodo-N-(1-methylcyclopropyl)-7-(2-oxa-7-azaspiro[3.5]nonan-7-yl)pyrazolo[1,5-a]pyridine-5-sulfonamide (25-3)

To a mixture of 7-chloro-3-iodo-N-(1-methylcyclopropyl)pyrazolo[1,5-a]pyridine-5-sulfonamide (482 mg, 0.70 mmol) and 2-oxa-7-azaspiro[3.5]nonane hemioxalate (269 mg, 1.72 mmol) in DMF (5.0 mL) was added TEA (712 mg, 7.0 mmol) under N₂. The mixture was stirred at 100 °C for 5 hrs. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 50% EtOAc in PE) to afford 3-iodo-N-(1-methylcyclopropyl)-7-(2-oxa-7-azaspiro[3.5]nonan-7-yl)pyrazolo[1,5-a]pyridine-5-sulfonamide (123 mg). LCMS (ESI, m/z): [M+H]⁺=503.1.

### Step 4: N-(1-methylcyclopropyl)-7-(2-oxa-7-azaspiro[3.5]nonan-7-yl)-3 (trimethyllstannyl)pyrazolo[1,5-a]pyridine-5-sulfonamide (25-4)

To a solution of 3-iodo-N-(1-methylcyclopropyl)-7-(2-oxa-7-azaspiro[3.5]nonan-7-yl)pyrazolo[1,5-a]pyridine-5-sulfonamide (100 mg, 0.20 mmol) in 1,4-dioxane (1 mL) were added trimethyl(trimethylstannyl)stannane (78 mg, 0.24 mmol) and Pd(PPh₃)₄ (23 mg, 0.02 mmol) under N₂. The reaction mixture was degassed, then stirred at 100 °C for 1.5 hrs under N₂ atmosphere. After completion, the reaction mixture was worked up with water, then extracted with EtOAc. The combined organic layers were washed with saturated brine, dried over anhydrous Na₂SO₄, filtered, and concentrated to afford the crude product which was used in the next step without further purification. LCMS (ESI, m/z): [M+H]⁺=539.1.

### Step 5: 3-(3-(difluoromethyl)-1,2,4-thiadiazol-5-yl)-N-(1-methylcyclopropyl)-7-(2-oxa-7-azaspiro[3.5]nonan-7-yl)pyrazolo[1,5-a]pyridine-5-sulfonamide (25)

To a solution of N-(1-methylcyclopropyl)-7-(2-oxa-7-azaspiro[3.5]nonan-7-yl)-3-(trimethylstannyl)pyrazolo[1,5-a]pyridine-5-sulfonamide (60 mg, 0.11 mmol) in 1,4-dioxane (40 mL) was added 5-bromo-3-(difluoromethyl)-1,2,4-thiadiazole (48 mg, 0.22 mmol) and Pd(PPh₃)₄ (13 mg, 0.01 mmol) under N₂. The mixture was degassed, then stirred at 100 °C for 5 hrs under N₂ atmosphere. LCMS showed the reaction completed. The reaction mixture was worked up with water, then extracted with EtOAc. The combined organic layers were washed with saturated brine, dried over anhydrous Na₂SO₄, filtered, and concentrated under reduced pressure. The residue was purified by prep-HPLC (0.1% FA) to afford 3-(3-(difluoromethyl)-1,2,4-thiadiazol-5-yl)-N-(1-methylcyclopropyl)-7-(2-oxa-7-azaspiro[3.5]nonan-7-yl)pyrazolo[1,5-a]pyridine-5-sulfonamide. LCMS (ESI, m/z): [M+H]⁺=509.2. ¹H NMR (400 MHz, DMSO-d₆) δ 9.06 (s, 1H), 8.48 (s, 1H), 8.29 (s, 1H), 7.30 (t, J=54 Hz, 1H), 6.84 (s, 1H), 4.42 (s, 4H) 3.45-3.39 (m, 4H), 2.08-2.01 (m, 4H), 1.13 (s, 3H), 0.75-0.68 (m, 2H), 0.49-0.42 (m, 2H).

### Example 26

### 1-[5-(difluoromethyl)-1,3,4-thiadiazol-2-yl]-N-(1-ethynylcyclopropyl)-4-[4-[(3S)-3-hydroxypyrrolidine-1-carbonyl]piperazin-1-yl]indazole-6-sulfonamide (26)

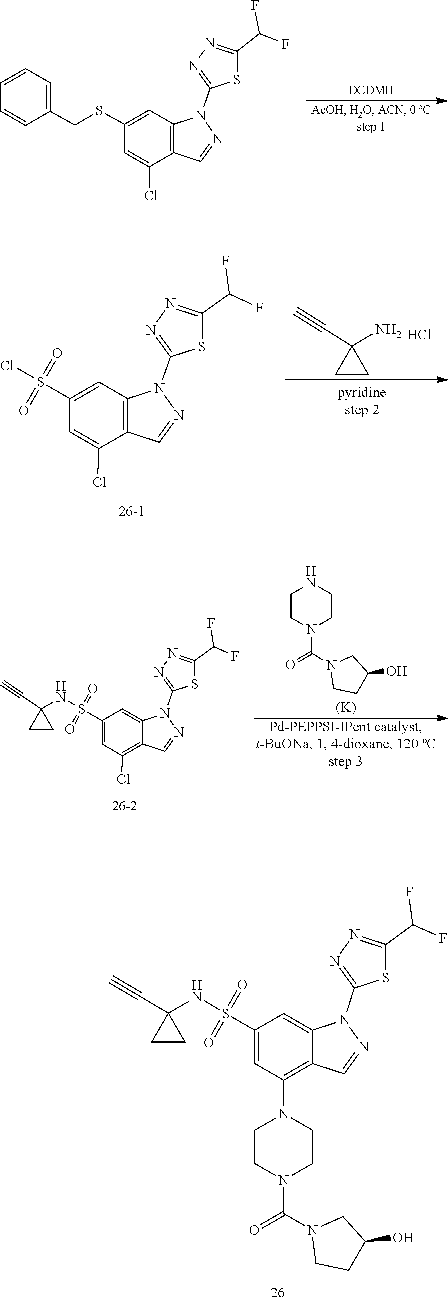

### Step 1: 4-chloro-1-(5-(difluoromethyl)-1,3,4-thiadiazol-2-yl)-1H-indazole-6-sulfonyl chloride (26-1)

To a solution of 2-(6-benzylsulfanyl-4-chloro-indazol-1-yl)-5-(difluoromethyl)-1,3,4-thiadiazole (2 g, 4.89 mmol) in MeCN (20 mL) was added AcOH (0.42 mL) and water (0.26 mL). 1,3-Dichloro-5,5-dimethylhydantoin (1.45 g, 7.36 mmol) was added portion wise at 0 °C under N₂. The reaction mixture was stirred at 0 °C for 2 hrs. After completion, the reaction mixture was concentrated to give the crude product which was used for the next step directly without further purification. LCMS (ESI, m/z): [M+H]⁺=385.3.

### Step 2: 4-chloro-1-(5-(difluoromethyl)-1,3,4-thiadiazol-2-yl)-N-(1-ethynylcyclopropyl)-1H-indazole-6-sulfonamide (26-2)

To a solution of 1-ethynylcyclopropan-1-amine hydrochloride (610 mg, 5.19 mmol) in pyridine (20 mL) was added 4-chloro-1-(5-(difluoromethyl)-1,3,4-thiadiazol-2-yl)-1H-indazole-6-sulfonyl chloride (2.0 g, 5.19 mmol) portion wise under N, at room temperature. The resulting mixture was stirred at room temperature for 12 hrs. After completion, the reaction mixture was concentrated under reduced pressure. The residue was purified by flash column chromatography on silica gel (eluting with 35% EtOAc in PE) to afford 4-chloro-1-(5-(difluoromethyl)-1,3,4-thiadiazol-2-yl)-N-(1-ethynylcyclopropyl)-1H-indazole-6-sulfonamide (1.1 g). LCMS (ESI, m/z): [M+H]⁺=430.3.

### Step 3: 1-[5-(difluoromethyl)-1,3,4-thiadiazol-2-yl]-N-(1-ethynylcyclopropyl)-4-[4-[(3S)-3-hydroxypyrrolidine-1-carbonyl]piperazin-1-yl]indazole-6-sulfonamide (26)

To a solution of 4-chloro-1-[5-(difluoromethyl)-1,3,4-thiadiazol-2-yl]-N-(1-ethynylcyclopropyl) indazole-6-sulfonamide (100 mg, 0.233 mmol), [(3S)-3-hydroxypyrrolidin-1-yl]-piperazin-1-yl-methanone (69.5 mg, 0.349 mmol) and t-BuONa (44.7 mg, 0.465 mmol) in 1,4-dioxane (5 mL) was added Pd-PEPPSI-IPent catalyst (36.8 mg, 0.047 mmol) under N₂. The mixture was degassed, then stirred at 100 °C for 2 hrs. The reaction mixture was worked up with water and extracted with ethyl acetate. The combined organic layers were washed with saturated brine, dried over anhydrous sodium sulfate, and concentrated under reduced pressure. The residue was purified by flash column chromatography on silica gel (eluting with 10% MeOH in DCM) to afford the product which was further purified on prep-HPLC (0.1% FA) to afford 1-[5-(difluoromethyl)-1,3,4-thiadiazol-2-yl]-N-(1-ethynylcyclopropyl)-4-[4-[(3S)-3-hydroxypyrrolidine-1-carbonyl]piperazin-1-yl]indazole-6-sulfonamide. LCMS (ESI, m/z): [M+H]⁺=593.3. ¹H NMR (400 MHz, DMSO-d₆) δ 8.87 (s, 1H), 8.71 (br s, 1H), 8.43 (s, 1H), 7.54 (t, J=52.0 Hz, 1H), 7.15 (s, 1H), 4.84 (br s, 1H), 4.18-4.12 (m, 1H), 3.50-3.29 (m, 8H), 3.28-3.24 (m, 3H), 3.10-3.04 (m, 1H), 2.60 (s, 1H), 1.82-1.66 (m, 2H), 1.13-1.08 (m, 2H), 0.95-0.90 (m, 2H).

Examples 27-33 were prepared according to the general procedures herein for Example 26, and in an analogous manner to that used to synthesize the example compounds with the appropriate intermediates. The starting materials were either prepared as described in the Intermediates section, commercially available, or prepared from commercially available reagents using conventional reactions well known in the art.

| | | | LC-MS |
|---|---|---|---|
| | | | (ESI, |
| Example | | | m/z): |
| Number | Structure | NMR | [M + H]⁺ |
| 27 | 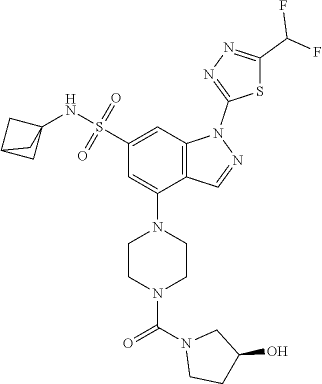 [SMILES: O=C(N1CCN(c2cc(S(=O)(=O)NC34CC(C3)C4)cc3c2cnn3-c2nnc(C(F)F)s2)CC1)N1CC[C@H](O)C1] | ¹H NMR (400 MHz, DMSO-d₆) δ 8.94 (s, 1H), 8.82 (br s, 1H), 8.47 (s, 1H), 7.60 (t, J = 52.0 Hz, 1H), 7.17 (s, 1H), 4.91 (br s, 1H), 4.28-4.21 (s, 1H), 3.55- 3.38 (m, 8H), 3.37- 3.30 (m, 3H), 3.18- 3.12 (m, 1H), 2.27 (s, 1H), 1.95-1.80 (m, 2H), 1.78-1.60 (m, 6H). | 595.3 |
| | N-(1-bicyclo [1.1.1] pentanyl)-1-[5- | | |
| | (difluoromethyl)-1,3,4-thiadiazol-2-yl]-4-[4- | | |
| | [(3S)-3-hydroxypyrrolidine-1-carbonyl] | | |
| | piperazin-1-yl] indazole-6-sulfonamide | | |
| 28 | 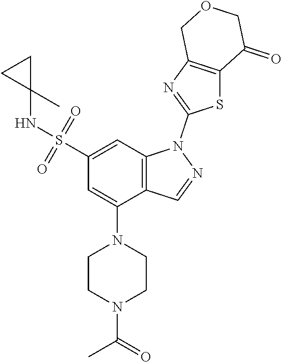 [SMILES: CC(=O)N1CCN(c2cc(S(=O)(=O)NC3(C)CC3)cc3c2cnn3-c2nc3c(s2)C(=O)COC3)CC1] | ¹H NMR (400 MHz, DMSO-d6) δ 8.84 (s, 1H), 8.37 (s, 1H), 8.25 (br s, 1H), 7.08 (s, 1H), 4.99 (s, 2H), 4.32 (s, 2H), 3.70-3.60 (m, 4H), 3.43-3.31 (m, 4H), 2.01 (s, 3H), 1.01 (s, 3H), 0.60-0.54 (m, 2H), 0.38-0.32 (m, 2H). | 531.2 |
| | 4-(4-acetylpiperazin-1-yl)-N-(1- | | |
| | methylcyclopropyl)-1-(7-oxo-6,7-dihydro-4H- | | |
| | pyrano[3,4-d] thiazol-2-yl)-1H-indazole-6- | | |
| | sulfonamide | | |
| 29 | 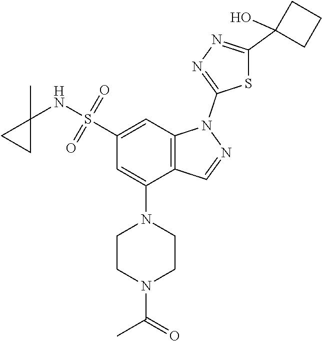 [SMILES: CC(=O)N1CCN(c2cc(S(=O)(=O)NC3(C)CC3)cc3c2cnn3-c2nnc(C3(O)CCC3)s2)CC1] | ¹H NMR (400 MHz, DMSO-d₆) δ 8.84 (s, 1H), 8.51 (s, 1H), 7.11 (s, 1H), 6.90 (br s, 1H), 3.82- 3.65 (m, 4H), 3.51-3.36 (m, 4H), 2.72-2.57 (m, 2H), 2.48-2.37 (m, 2H), 2.09 (s, 3H), 2.01-1.85 (m, 2H), 1.07 (s, 3H), 0.71- 0.60 (m, 2H), 0.45-0.35 (m, 2H). | 532.3 |
| | 4-(4-acetylpiperazin-1-yl)-1-[5-(1- | | |
| | hydroxycyclobutyl)-1,3,4-thiadiazol-2-yl]-N- | | |
| | (1-methylcyclopropyl)indazole-6-sulfonamide | | |
| 30 | 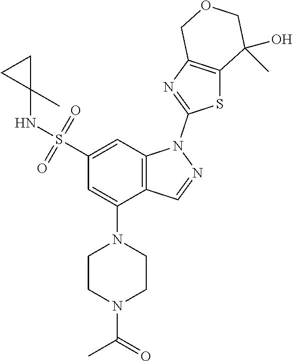 [SMILES: CC(=O)N1CCN(c2cc(S(=O)(=O)NC3(C)CC3)cc3c2cnn3-c2nc3c(s2)C(C)(O)COC3)CC1] | ¹H NMR (400 MHz, DMSO-d₆) δ 8.76 (s, 1H), 8.41 (s, 1H), 8.26 (br s, 1H), 7.07 (s, 1H), 5.75 (s, 1H), 4.78-4.68 (m, 2H), 3.76-3.68 (m, 6H), 3.45-3.36 (m, 4H), 2.08 (s, 3H), 1.48 (s, 3H), 1.07 (s, 3H), 0.68-0.62 (m, 2H), 0.43-0.36 (m, 2H). | 547.3 |
| | 4-(4-acetylpiperazin-1-yl)-1-(7-hydroxy-7- | | |
| | methyl-6,7-dihydro-4H-pyrano[3,4-d] thiazol- | | |
| | 2-yl)-N-(1-methylcyclopropyl)-1H-indazole- | | |
| | 6-sulfonamide | | |
| 31 | 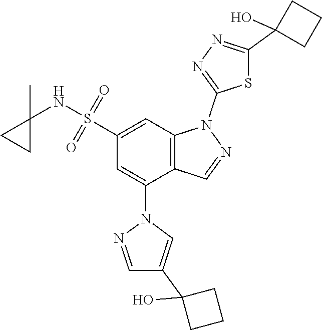 [SMILES: CC1(NS(=O)(=O)c2cc(-n3cc(C4(O)CCC4)cn3)c3cnn(-c4nnc(C5(O)CCC5)s4)c3c2)CC1] | ¹H NMR (400 MHz, DMSO-d₆) δ 9.10 (s, 1H), 8.91 (s, 1H), 8.65 (s, 1H), 8.14 (s, 1H), 8.03 (s, 1H), 6.95 (br s, 1H), 5.64 (s, 1H), 2.71- 2.59 (m, 2H), 2.48- 2.30 (m, 6H), 2.04- 1.68 (m, 4H), 1.10 (s, 3H), 0.74-0.66 (m, 2H), 0.49-0.41 (m, 2H). | 542.3 |
| | 4-[4-(1-hydroxycyclobutyl)pyrazol-1-yl]-1-[5- | | |
| | (1-hydroxycyclobutyl)-1,3,4-thiadiazol-2-yl]- | | |
| | N-(1-methylcyclopropyl)indazole-6- | | |
| | sulfonamide | | |
| 32 | 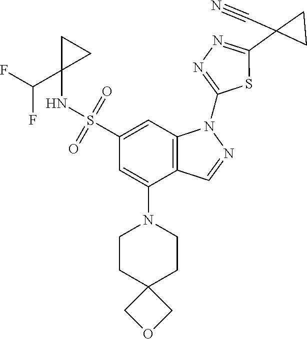 [SMILES: N#CC1(c2nnc(-n3ncc4c(N5CCC6(CC5)COC6)cc(S(=O)(=O)NC5(C(F)F)CC5)cc43)s2)CC1] | ¹H NMR (400 MHz, DMSO-d₆) δ 8.97 (br s, 1H), 8.81 (s, 1H), 8.34 (s, 1H), 7.11 (s, 1H), 5.79 (t, J = 56.0 Hz, 1H), 4.40 (s, 4H), 3.89- 3.79 (m, 4H), 2.16-2.09 (m, 2H), 2.05-1.98 (m, 4H), 1.95-1.89 (m, 2H), 0.96-0.88 (m, 2H), 0.82-0.76 (m, 2H). | 562.1 |
| | 1-(5-(1-cyanocyclopropyl)-1,3,4-thiadiazol-2- | | |
| | yl)-N-(1-(difluoromethyl)cyclopropyl)-4-(2- | | |
| | oxa-7-azaspiro[3.5]nonan-7-yl)-1H-indazole- | | |
| | 6-sulfonamide | | |
| 33312 | 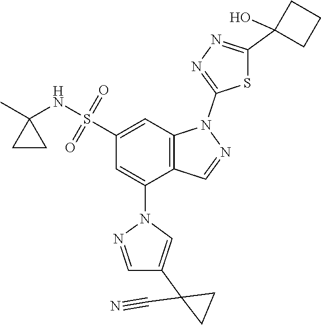 [SMILES: CC1(NS(=O)(=O)c2cc(-n3cc(C4(C#N)CC4)cn3)c3cnn(-c4nnc(C5(O)CCC5)s4)c3c2)CC1] | ¹HNMR (400 MHz, DMSO-d₆) δ 9.07 (s, 1H), 8.95 (s, 1H), 8.82 (s, 1H), 8.14 (s, 1H), 8.06 (s, 1H), 6.91 (br s, 1H), 2.68-2.61 (m, 2H), 2.53-2.41 (m, 2H), 1.96-1.85 (m, 2H), 1.80-1.73 (m, 2H), 1.62-1.55 (m, 2H), 1.11 (s, 3H), 0.72- 0.65 (m, 2H), 0.48- 0.41 (m, 2H). | 537.3 |
| | 4-(4-(1-cyanocyclopropyl)-1H-pyrazol-1-yl)- | | |
| | 1-(5-(1-hydroxycyclobutyl)-1,3,4-thiadiazol- | | |
| | 2-yl)-N-(1-methylcyclopropyl)-1H-indazole- | | |
| | 6-sulfonamide | | |

### Example 34

### 1-(5-(difluoromethyl)-1,3,4-thiadiazol-2-yl)-N-(1-methylcyclopropyl)-5-(2-oxa-7-azaspiro[3.5]nonan-7-yl)imidazo[1,5-a]pyridine-7-sulfonamide (34)

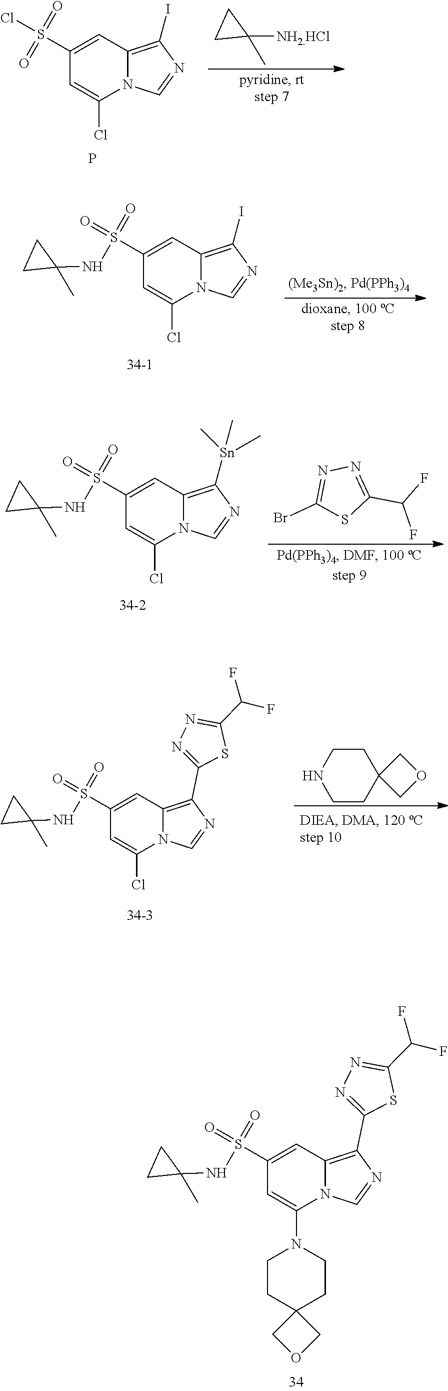

### Step 1: 5-chloro-1-iodo-N-(1-methylcyclopropyl)imidazo[1,5-a]pyridine-7-sulfonamide (34-1)

To a solution of 1-methylcyclopropan-1-amine hydrochloride (306 mg, 3.28 mmol) in pyridine (3 mL) was added 5-chloro-1-iodo-imidazo[1,5-a]pyridine-7-sulfonyl chloride (950 mg, 2.52 mmol) at room temperature under N₂. The mixture was stirred at temperature for 1 hr. LCMS showed the reaction completed. The reaction was worked up with water and extracted with DCM. The combined organic layers were washed with saturated brine, dried over anhydrous Na₂SO₄, filtered, and concentrated under reduced pressure to give the crude product. The crude product was purified by flash column chromatography on silica gel (eluting with 30% EtOAc in PE) to give 5-chloro-1-iodo-N-(1-methylcyclopropyl)imidazo[1,5-a]pyridine-7-sulfonamide (637 mg). LCMS (ESI, m/z): [M+H]⁺=412.0.

### Step 2: 5-chloro-N-(1-methylcyclopropyl)-1-(trimethylstannyl)imidazo[1,5-a]pyridine-7-sulfonamide (34-2)

To a solution of 5-chloro-1-iodo-N-(1-methylcyclopropyl)imidazo[1,5-a]pyridine-7-sulfonamide (637 mg, 1.55 mmol) in 1,4-dioxane (10 mL) was added trimethyl(trimethylstannyl)stannane (761 mg, 2.32 mmol) and Pd(PPh₃)₄ (179 mg, 0.155 mmol) under argon. The mixture was degassed and stirred at 100 °C for 2 hrs under argon atmosphere. LCMS showed the reaction completed. The resulting mixture was concentrated under reduced pressure to give the crude product which was directly used for the next step without purification. LCMS (ESI, m/z): [M+H]⁺=450.1.

### Step 3: 5-chloro-1-(5-(difluoromethyl)-1,3,4-thiadiazol-2-yl)-N-(1-methylcyclopropyl)imidazo[1,5-a]pyridine-7-sulfonamide (34-3)

To a solution of 5-chloro-N-(1-methylcyclopropyl)-1-trimethylstannyl-imidazo[1,5-a]pyridine-7-sulfonamide (800 mg, 1.78 mmol) in DMF (10 mL) was added 2-bromo-5-(difluoromethyl)-1,3,4-thiadiazole (374 mg, 1.74 mmol) and Pd(PPh₃)₄ (155 mg, 0.133 mmol) under argon. The mixture was degassed, then stirred at 90 °C for 2 hrs under argon atmosphere. LCMS showed the desired product was detected. Water was added into the mixture and the resulting mixture was extracted with EtOAc. The combined organic layers were washed with saturated brine, dried over anhydrous Na₂SO₄, filtered, and concentrated under reduced pressure to give the crude product. The crude product was purified by flash column chromatography on silica gel (eluting with 50% EtOAc in PE) to give 5-chloro-1-[5-(difluoromethyl)-1,3,4-thiadiazol-2-yl]-N-(1-methylcyclopropyl)imidazo[1,5-a]pyridine-7-sulfonamide (178 mg). LCMS (ESI, m/z): [M+H]⁺=420.1.

### Step 4: 1-(5-(difluoromethyl)-1,3,4-thiadiazol-2-yl)-N-(1-methylcyclopropyl)-5-(2-oxa-7-azaspiro[3.5]nonan-7-yl)imidazo[1,5-a]pyridine-7-sulfonamide (34)

To a solution of 5-chloro-1-[5-(difluoromethyl)-1,3,4-thiadiazol-2-yl]-N-(1-methylcyclopropyl)imidazo[1,5-a]pyridine-7-sulfonamide (170 mg, 0.405 mmol) in DMAc (4 mL) was added 2-oxa-7-azaspiro[3.5]nonane (103 mg, 0.81 mmol) and DIPEA (157 mg, 1.21 mmol) under argon. The reaction mixture was stirred at 120 °C under microwave for 2 hrs under argon atmosphere. LCMS showed the reaction completed. Water was added and the resulting mixture was extracted with EtOAc. The combined organic layers were washed with saturated brine, dried over anhydrous Na₂SO₄, filtered, and concentrated under reduced pressure to give the crude product. The crude product was purified by prep-HPLC (0.1% FA) to give 1-[5-(difluoromethyl)-1,3,4-thiadiazol-2-yl]-N-(1-methylcyclopropyl)-5-(2-oxa-7-azaspiro[3.5]nonan-7-yl)imidazo[1,5-a]pyridine-7-sulfonamide. LCMS (ESI, m/z): [M+H]⁺=511.2. ¹HNMR (400 MHz, DMSO-d₆) δ 8.56 (s, 1H), 8.33 (d, J=1.6 Hz, 1H), 8.30 (br s, 1H), 7.57 (t, J=53.6 Hz, 1H), 6.59 (d, J=1.6 Hz, 1H), 4.35 (s, 4H), 3.06-3.03 (m, 4H), 2.03-2.00 (m, 4H), 1.06 (s, 3H), 0.65-0.62 (m, 2H), 0.39-0.36 (m, 2H).

### Example 35

### 1-(5-(difluoromethyl)-1,3,4-thiadiazol-2-yl)-N-(1-(difluoromethyl)cyclopropyl)-5-(2-oxa-7-azaspiro[3.5]nonan-7-yl)imidazo[1,5-a]pyridine-7-sulfonamide (35)

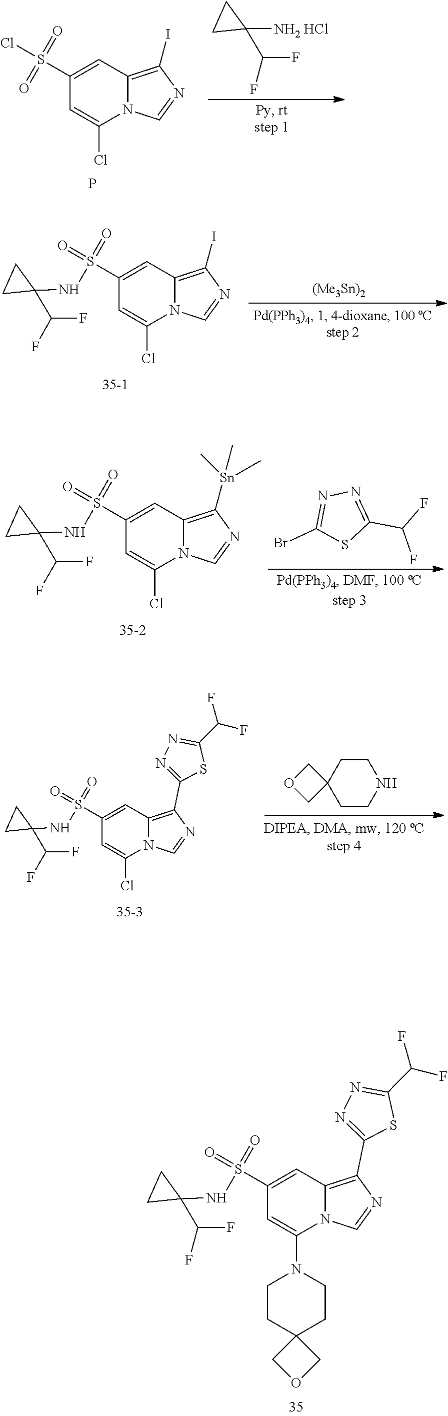

### Step 1: 5-chloro-N-(1-(difluoromethyl)cyclopropyl)-1-iodoimidazo[1,5-a]pyridine-7-sulfonamide (35-1)

To a solution of 1-(difluoromethyl)cyclopropan-1-amine hydrochloride (267 mg, 1.86 mmol) in pyridine (6 mL) was added 5-chloro-1-iodoimidazo[1,5-a]pyridine-7-sulfonyl chloride (550 mg, 1.46 mmol) at room temperature under N₂. The mixture was stirred at room temperature for 30 mins. LCMS showed the reaction completed. The reaction mixture was worked up with water and extracted with DCM. The combined organic layers were washed with saturated brine, dried over anhydrous Na₂SO₄, filtered, and concentrated under reduced pressure. The residue was purified by flash column chromatography on silica gel (eluting with 50% EtOAc in PE) to afford 5-chloro-N-[1-(difluoromethyl)cyclopropyl]-1-iodo-imidazo[1,5-a]pyridine-7-sulfonamide (400 mg). LCMS (ESI, m/z): [M+H]⁺=448.0.

### Step 2: 5-chloro-N-(1-(difluoromethyl)cyclopropyl)-1-(trimethylstannyl)imidazo[1,5-a]pyridine-7-sulfonamide (35-2)

To a solution of 5-chloro-N-[1-(difluoromethyl)cyclopropyl]-1-iodo-imidazo[1,5-a]pyridine-7-sulfonamide (400 mg, 0.894 mmol) in 1,4-dioxane (10 mL) was added trimethyl(trimethylstannyl)stannane (439 mg, 1.34 mmol) and Pd(PPh₃)₄ (103 mg, 0.089 mmol) under N₂. The mixture was degassed, then stirred at 100 °C for 2 hrs under N₂ atmosphere. LCMS showed the reaction completed. The reaction mixture was worked up with water and extracted with DCM. The combined organic layers were washed with saturated brine, dried over anhydrous Na₂SO₄, filtered, and concentrated under reduced pressure to afford the crude product which was directly used for the next step without purification. LCMS (ESI, m/z): [M+H]⁺=484.1.

### Step 3: 5-chloro-1-(5-(difluoromethyl)-1,3,4-thiadiazol-2-yl)-N-(1-(difluoromethyl)cyclopropyl)imidazo[1,5-a]pyridine-7-sulfonamide (35-3)

To a solution of 5-chloro-N-[1-(difluoromethyl)cyclopropyl]-1-trimethylstannyl-imidazo[1,5-a]pyridine-7-sulfonamide (600 mg, 1.24 mmol) in DMF (6 mL) was added 2-bromo-5-(difluoromethyl)-1,3,4-thiadiazole (231 mg, 1.07 mmol) and Pd(PPh₃)₄ (96 mg, 0.083 mmol) under N₂. The mixture was degassed, then stirred at 100 °C for 3 hrs under N₂ atmosphere. LCMS showed the reaction completed. The reaction was worked with water and extracted with EtOAc. The combined organic layers were washed with brine, dried over anhydrous Na₂SO₄, filtered, and concentrated under reduced pressure. The residue was purified by flash column chromatography on silica gel (eluting with 50% EtOAc in PE) to give 5-chloro-N-[1-(difluoromethyl)-cyclopropyl]-1-[5-(difluoromethyl)-1,3,4-thiadiazol-2-yl]imidazo[1,5-a]pyridine-7-sulfonamide (300 mg). LCMS (ESI, m/z): [M+H]⁺=456.1.

### Step 4: 1-(5-(difluoromethyl)-1,3,4-thiadiazol-2-yl)-N-(1-(difluoromethyl)cyclopropyl)-5-(2-oxa-7-azaspiro[3.5]nonan-7-yl)imidazo[1,5-a]pyridine-7-sulfonamide (35)

To a solution of 2-oxa-7-azaspiro[3.5]nonane (34 mg, 0.267 mmol) and DIEA (85 mg, 0.658 mmol) in DMAc (5 mL) was added 5-chloro-N-[1-(difluoromethyl)-cyclopropyl]-1-[5-(difluoromethyl)-1,3,4-thiadiazol-2-yl]imidazo[1,5-a]pyridine-7-sulfonamide (100 mg, 0.219 mmol) under N₂. The mixture was stirred at 120 °C under microwave for 1 hr. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by prep-HPLC (0.1% FA) to afford 1-(5-(difluoromethyl)-1,3,4-thiadiazol-2-yl)-N-(1-(difluoromethyl)cyclopropyl)-5-(2-oxa-7-azaspiro[3.5]nonan-7-yl)imidazo[1,5-a]pyridine-7-sulfonamide. LCMS (ESI, m/z): [M+H]⁺=547.2. ¹HNMR (400 MHz, DMSO-d₆) δ 9.10 (br s, 1H), 8.64 (s, 1H), 8.37 (s, 1H), 7.64 (t, J=53.2 Hz, 1H), 6.66 (s, 1H), 5.78 (t, J=56.0 Hz, 1H), 4.43 (s, 4H), 3.15-3.08 (m, 4H), 2.13-2.06 (m, 4H), 0.99-0.90 (m, 4H).

### Example 36

### N-(1-cyanocyclopropyl)-1-(5-(difluoromethyl)-1,3,4-thiadiazol-2-yl)-5-(2-oxa-7-azaspiro[3.5]nonan-7-yl)imidazo[1,5-a]pyridine-7-sulfonamide (36)

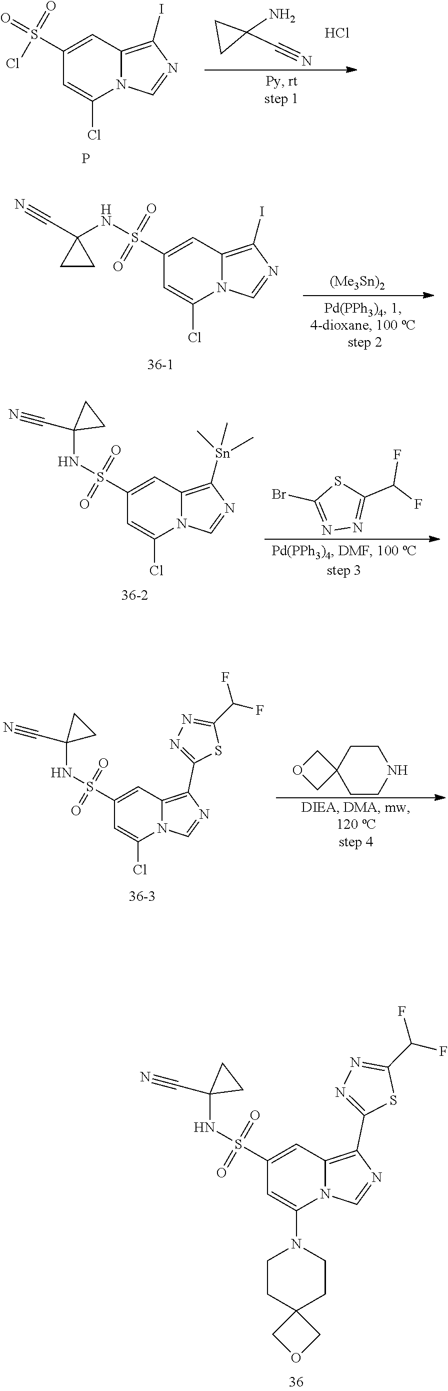

### Step 1: 5-chloro-N-(1-cyanocyclopropyl)-1-(trimethylstannyl)imidazo[1,5-a]pyridine-7-sulfonamide (36-1)

To a solution of 1-Amino-1-cyclopropanecarbonitrile hydrochloride (57.2 mg, 0.482 mmol) in pyridine (1 mL) was added 5-chloro-1-iodoimidazo[1,5-a]pyridine-7-sulfonyl chloride (140 mg, 0.371 mmol) at room temperature under N₂. The mixture was stirred at room temperature for 1 hr. LCMS showed the reaction completed. The reaction mixture was worked up with water and extracted with DCM. The combined organic layers were washed with saturated brine, dried over anhydrous Na₂SO₄, filtered, and concentrated under reduced pressure. The residue was purified by flash column chromatography on silica gel (eluting with 30% EtOAc in PE) to afford 5-chloro-N-(1-cyanocyclopropyl)-1-iodoimidazo[1,5-a]pyridine-7-sulfonamide (120 mg). LCMS (ESI, m/z): [M+H]⁺=423.0.

### Step 2: 5-chloro-N-(1-cyanocyclopropyl)-1-(trimethylstannyl)imidazo[1,5-a]pyridine-7-sulfonamide (36-2)

To a solution of 5-chloro-N-(1-cyanocyclopropyl)-1-iodoimidazo[1,5-a]pyridine-7-sulfonamide (100 mg, 0.237 mmol) in 1,4-dioxane (3 mL) was added trimethyl(trimethylstannyl)stannane (117 mg, 0.356 mmol) and Pd(PPh₃)₄ (28 mg, 0.024 mmol) under N₂. The reaction mixture was degassed, then stirred at 100 °C for 2 hrs under N₂ atmosphere. LCMS showed the reaction completed. The reaction mixture was worked up with water and extracted with DCM. The combined organic layers were washed with saturated brine, dried over anhydrous Na₂SO₄, filtered, and concentrated under reduced pressure to afford the crude product which was directly used for the next step without purification. LCMS (ESI, m/z): [M+H]⁺=459.1.

### Step 3: 5-chloro-N-(1-cyanocyclopropyl)-1-(5-(difluoromethyl)-1,3,4-thiadiazol-2-yl)imidazo[1,5-a]pyridine-7-sulfonamide (36-3)

To a solution of 5-chloro-N-(1-cyanocyclopropyl)-1-(trimethylstannyl)imidazo[1,5-a]pyridine-7-sulfonamide (200 mg, 0.435 mmol) in DMF (3 ml) was added 2-bromo-5-(difluoromethyl)-1,3,4-thiadiazole (70 mg, 0.326 mmol) and Pd(PPh₃)₄ (25 mg, 0.0217 mmol) under N₂. The reaction mixture was degassed, then stirred at 100 °C for 3 hrs under N₂ atmosphere. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by flash column chromatography on silica gel (eluting with 50% EtOAc in PE) to give 5-chloro-N-(1-cyanocyclopropyl)-1-(5-(difluoromethyl)-1,3,4-thiadiazol-2-yl)imidazo[1,5-a]pyridine-7-sulfonamide (60 mg). LCMS (ESI, m/z): [M+H]⁺=431.0.

### Step 4: N-(1-cyanocyclopropyl)-1-(5-(difluoromethyl)-1,3,4-thiadiazol-2-yl)-5-(2-oxa-7-azaspiro[3.5]nonan-7-yl)imidazo[1,5-a]pyridine-7-sulfonamide (36)

To a solution of 5-chloro-N-(1-cyanocyclopropyl)-1-(5-(difluoromethyl)-1,3,4-thiadiazol-2-yl)imidazo[1,5-a]pyridine-7-sulfonamide (60 mg, 0.139 mmol) in DMAc (2 mL) was added 2-oxa-7-azaspiro[3.5]nonane (23 mg, 0.263 mol) and DIPEA (54 mg, 0.417 mmol) under N₂. The reaction mixture was stirred at 120 °C under microwave for 1 hr. LCMS showed the reaction completed. The reaction mixture was worked up with water and extracted with EtOAc. The combined organic layers were washed with saturated brine, dried over anhydrous Na₂SO₄, filtered, and concentrated under reduced pressure. The residue was purified by prep-HPLC (0.1% FA) to afford N-(1-cyanocyclopropyl)-1-(5-(difluoromethyl)-1,3,4-thiadiazol-2-yl)-5-(2-oxa-7-azaspiro[3.5]nonan-7-yl)imidazo[1,5-a]pyridine-7-sulfonamide. LCMS (ESI, m/z): [M+H]⁺=522.3. ¹HNMR (400 MHz, DMSO-d₆) δ 8.68 (s, 1H), 8.48 (s, 1H), 7.65 (t, J=52.0 Hz, 1H), 6.67 (s, 1H), 4.43 (s, 4H), 3.17-3.11 (m, 4H), 2.13-2.06 (m, 4H), 1.51-1.44 (m, 2H), 1.41-1.33 (m, 2H).

### Example 37

### 3-(5-(difluoromethyl)-1,3,4-thiadiazol-2-yl)-N-(1-(difluoromethyl)cyclopropyl)-8-(2-oxa-7-azaspiro[3.5]nonan-7-yl)imidazo[1,2-a]pyridine-6-sulfonamide (37)

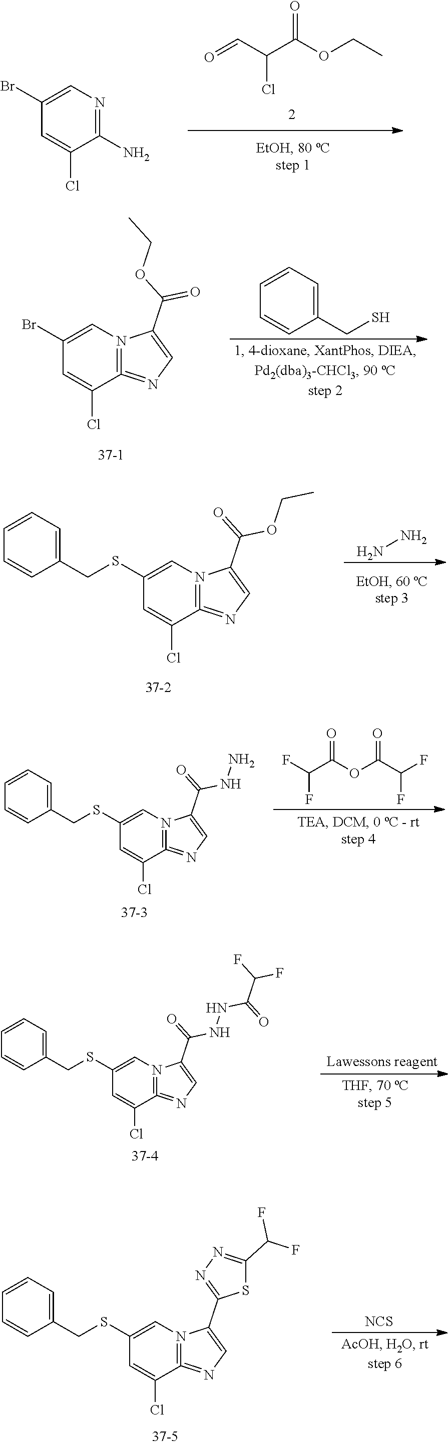

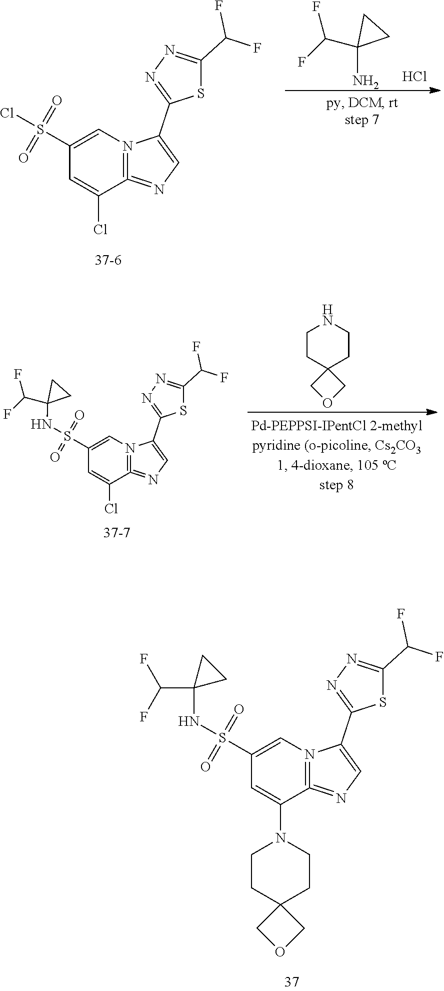

### Step 1: Ethyl 6-bromo-8-chloroimidazo[1,2-a]pyridine-3-carboxylate (37-1)

A solution of 5-bromo-3-chloropyridin-2-amine (20 g, 96 mmol), ethyl 2-chloro-3-oxopropanoate (16 g, 106 mmol) in EtOH (300 mL) was stirred for 3 hrs at 80 °C under N₂. The reaction mixture was allowed to cool down to room temperature and concentrated under reduced pressure. The residue was purified by flash column chromatography on silica gel (eluting with 0% to 10% MeOH in DCM) to afford the product. The product was slurried with ethyl acetate to afford ethyl 6-bromo-8-chloroimidazo[1,2-a]pyridine-3-carboxylate (12 g). LCMS (ESI, m/z): [M+H]⁺=302.9.

### Step 2: Ethyl 6-(benzylthio)-8-chloroimidazo[1,2-a]pyridine-3-carboxylate (37-2)

A solution of ethyl 6-bromo-8-chloroimidazo[1,2-a]pyridine-3-carboxylate (8.5 g, 26.0 mmol) in 1,4-dioxane (150 mL) was added phenylmethanethiol (3.3 g, 27 mmol), Xantphos (3.0 g, 5.2 mmol), DIEA (10 g, 78 mmol), Pd₂(dba)₃·CHCl₃ (2.7 g, 2.6 mmol) under N₂. The mixture was degassed with N₂, then stirred for 3 hrs at 90 °C. After completion, the reaction mixture was allowed to cool down to room temperature and concentrated under reduced pressure. The residue was purified by flash column chromatography on silica gel (eluting with 0% to 30% EtOAc in PE) to afford ethyl 6-(benzylthio)-8-chloroimidazo[1,2-a]pyridine-3-carboxylate (7.6 g). LCMS (ESI, m/z): [M+H]⁺=347.2.

### Step 3: 6-(Benzylthio)-8-chloroimidazo[1,2-a]pyridine-3-carbohydrazide (37-3)

A solution of ethyl 6-(benzylthio)-8-chloroimidazo[1,2-a]pyridine-3-carboxylate (7.6 g, 20 mmol), hydrazine hydrate (80% in water, 25.5 g, 408 mmol) in EtOH (100 mL) was stirred for 2 hrs at 60 °C under N₂. After completion, the reaction mixture was allowed to cool down to room temperature and concentrated under reduced pressure. The residue was slurried with diethyl ether and filtered to afford 6-(benzylthio)-8-chloroimidazo[1,2-a]pyridine-3-carbohydrazide (6.7 g). LCMS (ESI, m/z): [M+H]⁺=333.1.

### Step 4: 6-(Benzylthio)-8-chloro-N′-(2,2-difluoroacetyl)imidazo[1,2-a]pyridine-3-carbohydrazide (37-4)

To a solution of 6-(benzylthio)-8-chloroimidazo[1,2-a]pyridine-3-carbohydrazide (6.7 g, 19 mmol), TEA (2.5 g, 25 mmol) in DCM (100 mL) was added 2,2-difluoroacetic anhydride (4.0 g, 23 mmol) at 0 °C under N₂. The mixture was warmed to room temperature and stirred for 2 hrs. After completion, the reaction mixture was worked up with water, extracted with DCM and dried over anhydrous Na₂SO₄. After filtration, the filtrate was concentrated under reduced pressure. The residue was slurried with ethyl acetate and filtered to afford 6-(benzylthio)-8-chloro-N′-(2,2-difluoroacetyl)imidazo[1,2-a]pyridine-3-carbohydrazide (4.5 g). LCMS (ESI, m/z): [M+H]⁺=411.1.

### Step 5: 2-(6-(Benzylthio)-8-chloroimidazo[1,2-a]pyridin-3-yl)-5-(difluoromethyl)-1,3,4-thiadiazole (37-5)

A solution of 6-(benzylthio)-8-chloro-N′-(2,2-difluoroacetyl)imidazo[1,2-a]pyridine-3-carbohydrazide (5 g, 11 mmol), Lawesson's Reagent (5.5 g, 14 mmol) in THE (80 mL) was stirred for 2 hrs at 70 °C under N₂. After completion, the reaction mixture was allowed to cool down to room temperature and concentrated under reduced pressure. The residue was purified by flash column chromatography on silica gel (eluting with 0% to 50% EtOAc in PE) to afford 2-(6-(benzylthio)-8-chloroimidazo[1,2-a]pyridin-3-yl)-5-(difluoromethyl)-1,3,4-thiadiazole (4.3 g). LCMS (ESI, m/z): [M+H]⁺=409.0.

### Step 6: 8-Chloro-3-(5-(difluoromethyl)-1,3,4-thiadiazol-2-yl)imidazo[1,2-a]pyridine-6-sulfonyl chloride (37-6)

To a solution of 2-(6-(benzylthio)-8-chloroimidazo[1,2-a]pyridin-3-yl)-5-(difluoromethyl)-1,3,4-thiadiazole (500 mg, 1 mmol) in water (2 mL) and AcOH (6 mL) was added NCS (607 mg, 5 mmol) under N₂. The mixture was stirred for 2 hrs at room temperature. After completion, the reaction mixture was worked up with water and extracted with DCM. The combined organic layers were dried over anhydrous Na₂SO₄, filtered, and concentrated under reduced pressure. The crude product was used for the next step directly without further purification. LCMS (ESI, m/z): [M+H]⁺=384.9.

### Step: 7: 8-chloro-3-(5-(difluoromethyl)-1,3,4-thiadiazol-2-yl)-N-(1-(difluoromethyl)cyclopropyl)imidazo[1,2-a]pyridine-6-sulfonamide (37-7)

Into a round-bottom flask was added 1-(difluoromethyl)cyclopropan-I-amine hydrochloride (142 mg, 0.99 mmol), pyridine (524 mg, 7 mmol), 4A molecular sieve and DCM (6 mL) under N₂. A solution of 8-chloro-3-(5-(difluoromethyl)-1,3,4-thiadiazol-2-yl)imidazo[1,2-a]pyridine-6-sulfonyl chloride (300 mg, 0.79 mmol) in DCM (2 ml) was added dropwise at room temperature. The mixture was stirred for 16 hrs at room temperature. After completion, the reaction mixture was filtered and the filtrated was concentrated under reduced pressure. The residue was purified by flash column chromatography on silica gel (eluting with 0% to 50% ethyl acetate in petroleum ether) to afford 8-chloro-3-(5-(difluoromethyl)-1,3,4-thiadiazol-2-yl)-N-(1-(difluoromethyl)cyclopropyl)-imidazo[1,2-a]pyridine-6-sulfonamide (170 mg). LCMS (ESI, m/z): [M+H]⁺=456.0.

### Step 8: 3-(5-(difluoromethyl)-1,3,4-thiadiazol-2-yl)-N-(1-(difluoromethyl)cyclopropyl)-8-(2-oxa-7-azaspiro[3.5]nonan-7-yl)imidazo[1,2-a]pyridine-6-sulfonamide (37)

A solution of 8-chloro-3-(5-(difluoromethyl)-1,3,4-thiadiazol-2-yl)-N-(1-(difluoromethyl)cyclopropyl)imidazo[1,2-a]pyridine-6-sulfonamide (130 mg, 0.26 mmol) in 1,4-dioxane (4 mL) was added 2-oxa-7-azaspiro[3.5]nonane (67 mg, 0.53 mmol), Pd-PEPPSI-IPentCl catalyst (44 mg, 0.045 mmol), Cs₂CO₃ (259 mg, 0.79 mmol) under N₂. The mixture was degassed with N₂, then stirred for 3 hrs at 105 °C. After completion, the reaction mixture was allowed to cool down to room temperature and concentrated under reduced pressure. The residue was purified by flash column chromatography on silica gel (eluting with 0% to 15% MeOH in DCM) to afford the crude product which was further purified by prep-HPLC (0.1% NH₄HCO₃) to afford 3-(5-(difluoromethyl)-1,3,4-thiadiazol-2-yl)-N-(1-(difluoromethyl)cyclopropyl)-8-(2-oxa-7-azaspiro[3.5]nonan-7-yl)imidazo[1,2-a]pyridine-6-sulfonamide. LCMS (ESI, m/z): [M+H]⁺=547.1. ¹H NMR (400 MHz, DMSO-d₆) δ 9.59 (s, 1H), 9.10 (s, 1H), 8.60 (s, 1H), 7.70 (t, J=53.2 Hz, 1H), 6.99 (s, 1H), 5.78 (t, J=55.6H, 1H), 4.41 (s, 4H), 3.57-3.53 (m, 4H), 2.02-1.97 (m, 4H), 0.99-0.92 (m, 4H). ¹⁹F NMR (400 MHz, DMSO-d₆) δ −109.15, −120.30

### Example 38

### 8-(4-acetylpiperazin-1-yl)-3-(5-(1-cyanocyclopropyl)-1,3,4-thiadiazol-2-yl)-N-(1-methylcyclopropyl)imidazo[1,2-a]pyridine-6-sulfonamide (38)

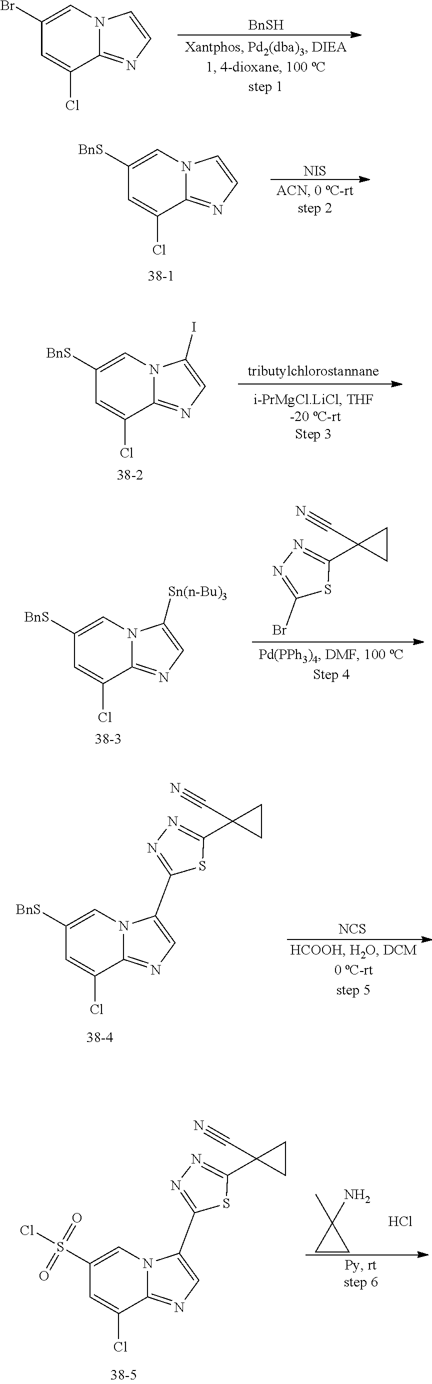

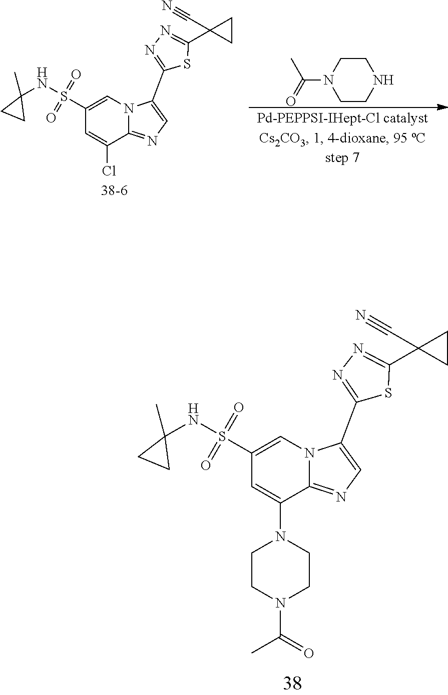

### Step 1: 6-(benzylthio)-8-chloroimidazo[1,2-a]pyridine (38-1)

To a solution of 6-bromo-8-chloroimidazo[1,2-a]pyridine (5.00 g, 21.6 mmol) in 1,4-dioxane (50 ml) was added phenyl methanethiol (2.95 g, 23.8 mmol), DIEA (8.38 g, 64.8 mmol), Xantphos (1.25 g, 2.16 mmol) and Pd₂(dba)₃-CHCl₃ (1.12 g, 1.08 mmol) under N₂. The mixture was degassed, then stirred at 100 °C for 3 hrs under N₂ atmosphere. The reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 100% dichloromethane) to afford 6-(benzylthio)-8-chloroimidazo[1,2-a]pyridine (5.9 g). LCMS (ESI, m/z): [M+H]⁺=275.1.

### Step 2: 6-(benzylthio)-8-chloro-3-iodoimidazo[1,2-a]pyridine (38-2)

To a solution of 6-benzylsulfanyl-8-chloro-imidazo[1,2-a]pyridine (5.0 g, 18.2 mmol) in ACN (50 ml) was added NIS (4.50 g, 20.0 mmol) in portions at 0 °C. The mixture was allowed to warm to room temperature and stirred for 16 hrs. After completion, the reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 15% EtOAc in PE) to afford 6-(benzylthio)-8-chloro-3-iodoimidazo[1,2-a]pyridine (7.26 g). LCMS (ESI, m/z): [M+H]⁺=401.0.

### Step 3: 6-(benzylthio)-8-chloro-3-(tributylstannyl)imidazo[1,2-a]pyridine (38-3)

To a solution of 6-benzylsulfanyl-8-chloro-3-iodo-imidazo[1,2-a]pyridine (2.0 g, 4.99 mmol) in anhydrous THF (40 mL) was added i-PrMgCl·LiCl (1.3 M in THF, 4.2 mL, 5.49 mmol) dropwise at −20 °C under Ar. The mixture was stirred at −20 °C for 15 mins. Then tributylchlorostannane (1.79 g, 5.49 mmol) was added dropwise at −20 °C. The resulting mixture was allowed to warm to room temperature and stirred for 1 hr under N₂ atmosphere. TLC showed the reaction completed. The reaction mixture was quenched with saturated ammonium chloride aqueous solution, then extracted with EtOAc. The combined organic layers were washed with saturated brine, dried over anhydrous Na₂SO₄, filtered, and concentrated to give the crude product which was directly used for the next step without further purification. LCMS (ESI, m/z): [M+H]⁺=563.1.

### Step 4: 1-(5-(6-(benzylthio)-8-chloroimidazo[1,2-a]pyridin-3-yl)-1,3,4-thiadiazol-2-yl)cyclopropane-1-carbonitrile (38-4)

To a solution of 6-(benzylthio)-8-chloro-3-(tributylstannyl)imidazo[1,2-a]pyridine (1.8 g, 3.19 mmol) in DMF (100 mL) was added 1-(5-bromo-1,3,4-thiadiazol-2-yl)cyclopropanecarbonitrile (881 mg, 3.83 mmol) and Pd(PPh₃)₄ (369 mg, 0.319 mmol) under N₂. The mixture was degassed, then stirred at 100 °C for 16 hrs under N₂. LCMS showed the reaction completed. The reaction mixture was worked up with water, then extracted with EtOAc. The combined organic layers were washed with saturated brine, dried over anhydrous sodium Na₂SO₄, filtered, and concentrated under reduced pressure to give the residue. The residue was purified by flash column chromatography on silica gel (eluting with 80% EtOAc in PE) to afford 1-(5-(6-benzylsulfanyl-8-chloro-imidazo[1,2-a]pyridin-3-yl)-1,3,4-thiadiazol-2-yl)cyclopropanecarbonitrile (1.2 g). LCMS (ESI, m/z): [M+H]⁺=424.2.

### Step 5: 8-chloro-3-(5-(1-cyanocyclopropyl)-1,3,4-thiadiazol-2-yl)imidazo[1,2-a]pyridine-6-sulfonyl chloride (38-5)

To a solution of 1-[5-(6-benzylsulfanyl-8-chloro-imidazo[1,2-a]pyridin-3-yl)-1,3,4-thiadiazol-2-yl]cyclopropanecarbonitrile (900 mg, 2.12 mmol) in formic acid (3.2 mL), water (1.5 mL) and DCM (5 mL) was added 1-chloropyrrolidine-2,5-dione (850 mg, 6.37 mmol) at 0 °C under N₂. The mixture was allowed to warm to room temperature and stirred for 1 hr under N₂ atmosphere. LCMS showed the reaction completed. The reaction mixture was worked up with water, then extracted with DCM. The combined organic layers were washed with saturated brine, dried over anhydrous Na₂SO₄, filtered, and concentrated under reduced pressure to give the residue. The residue was purified by flash column chromatography on silica gel (eluting with 50% EtOAc in PE) to afford 8-chloro-3-[5-(1-cyanocyclopropyl)-1,3,4-thiadiazol-2-yl]imidazo[1,2-a]pyridine-6-sulfonyl chloride (320 mg). LCMS (ESI, m/z): [M+H]⁺=400.0.

### Step 6: 8-chloro-3-(5-(1-cyanocyclopropyl)-1,3,4-thiadiazol-2-yl)-N-(1-methylcyclopropyl)imidazo[1,2-a]pyridine-6-sulfonamide (38-6)

To a solution of 1-methylcyclopropanamine hydrochloride (103 mg, 0.959 mmol) in pyridine (4 mL) was added 8-chloro-3-[5-(1-cyanocyclopropyl)-1,3,4-thiadiazol-2-yl]imidazo[1,2-a]pyridine-6-sulfonyl chloride (320 mg, 0.799 mmol) at room temperature under N₂. The mixture was stirred at room temperature for 1 hr. LCMS showed the reaction completed. The reaction mixture was concentrated under reduced pressure. The residue was purified by flash column chromatography on silica gel (eluting with 80% EtOAc in PE) to give 8-chloro-3-[5-(1-cyanocyclopropyl)-1,3,4-thiadiazol-2-yl]-N-(1-methylcyclopropyl)imidazo[1,2-a]pyridine-6-sulfonamide (200 mg). LCMS (ESI, m/z): [M+H]⁺=435.1.

### Step 7: 8-(4-acetylpiperazin-1-yl)-3-(5-(1-cyanocyclopropyl)-1,3,4-thiadiazol-2-yl)-N-(1-methylcyclopropyl)imidazo[1,2-a]pyridine-6-sulfonamide (38)

To a solution of 8-chloro-3-[5-(1-cyanocyclopropyl)-1,3,4-thiadiazol-2-yl]-N-(1-methylcyclopropyl)imidazo[1,2-a]pyridine-6-sulfonamide (150 mg, 0.345 mmol) in 1,4-dioxane (20 mL) was added 1-piperazin-1-ylethanone (133 mg, 1.03 mmol), Cs₂CO₃ (225 mg, 0.689 mmol) and Pd-PEPPST-IHept-C1 catalyst (67.0 mg, 0.069 mmol) under N₂. The reaction mixture was degassed, then stirred at 95 °C for 1.5 hrs under N₂. LCMS showed the reaction completed. The reaction mixture was worked up with water, then extracted with EtOAc. The combined organic layers were washed with saturated brine, dried over anhydrous Na₂SO₄, filtered, and concentrated under reduced pressure. The residue was purified by flash column chromatography on silica gel (eluting with 5% MeOH in DCM) which was further purified by prep-HPLC (0.05% NH₄HCO₃) to afford 8-(4-acetylpiperazin-1-yl)-3-[5-(1-cyanocyclopropyl)-1,3,4-thiadiazol-2-yl]-N-(1-methylcyclopropyl)imidazo[1,2-a]pyridine-6-sulfonamide. LCMS (ESI, m/z): [M+H]⁺=527.3. ¹HNMR (400 MHz, DMSO-d₆) δ 9.63 (s, 1H), 8.50 (s, 1H), 8.37 (s, 1H), 6.96 (s, 1H), 3.73-3.54 (m, 8H), 2.22-2.15 (m, 2H), 2.08 (s, 3H), 2.01-1.94 (m, 2H), 1.13 (s, 3H), 0.73-0.67 (m, 2H), 0.47-0.40 (m, 2H).

### Example 39

### 3-(5-(difluoromethyl)-1,3,4-thiadiazol-2-yl)-N-(1-(difluoromethyl)cyclopropyl)-8-(2-oxa-7-azaspiro[3.5]nonan-7-yl)imidazo[1,5-a]pyridine-6-sulfonamide (39)

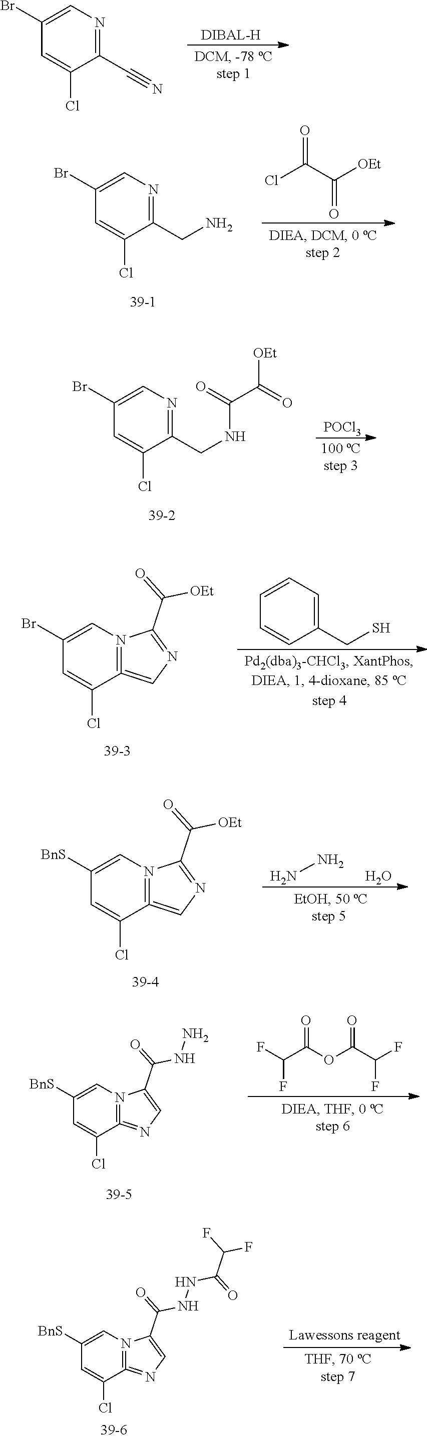

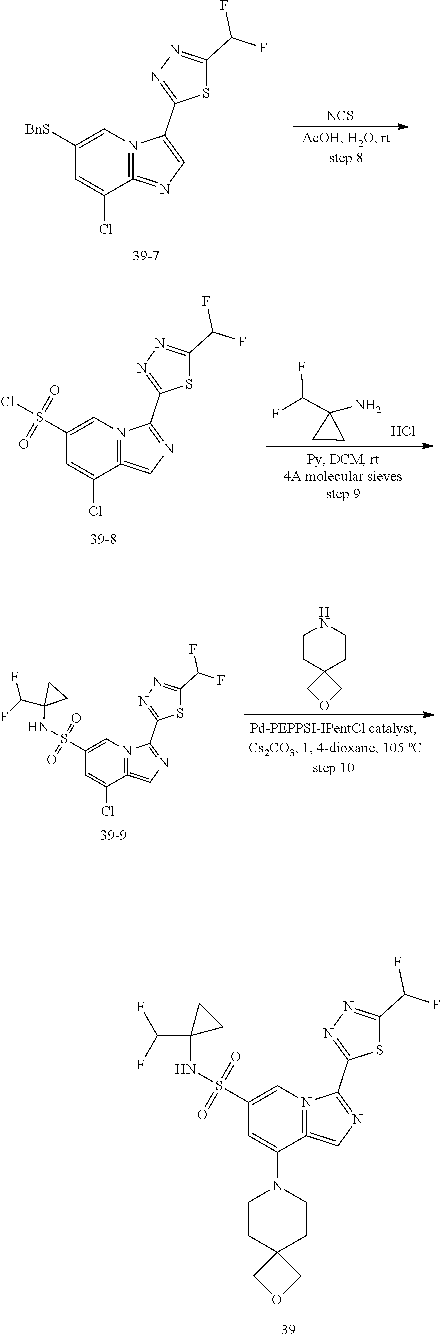

### Step 1: (5-bromo-3-chloropyridin-2-yl)methanamine (39-1)

To a stirred solution of 5-bromo-3-chloropyridine-2-carbonitrile (1 g, 4.6 mmol) in dry DCM (10 mL) was added DIBAL-H (1 mol/L in DCM, 14 mL, 14 mmol) dropwise at −78 °C under N₂. The resulting mixture was stirred at −78 °C for 1 hr. After completion, the reaction mixture was quenched by the addition of ice water and extracted with ethyl acetate. The combined organic layers were washed with saturated brine, dried over anhydrous Na₂SO₄. After filtration, the filtrate was concentrated under reduced pressure. The residue was purified by flash column chromatography on silica gel (eluting with 0 to 10% MeOH in DCM) to afford (5-bromo-3-chloropyridin-2-yl)methanamine (500 mg). LCMS (ESI, m/z): [M+H]⁺=220.9.

### Step 2: Ethyl 2-(((5-bromo-3-chloropyridin-2-yl)methyl)amino)-2-oxoacetate (39-2)

To a solution of (5-bromo-3-chloropyridin-2-yl)methanamine (100 mg, 0.45 mmol) and TEA (91.4 mg, 0.93 mmol) in DCM (2 mL) was added ethyl 2-chloro-2-oxoacetate (62 mg, 0.45 mmol) at 0 °C under N₂. The resulting mixture was stirred for 4 hrs at 0 °C. After completion, the reaction mixture was worked up with water and extracted with DCM. The combined organic layers were washed with saturated brine, dried over anhydrous Na₂SO₄. After filtration, the filtrate was concentrated under reduced pressure. The crude product was used for the next step directly without further purification. LCMS (ESI, m/z): [M+H]⁺=321.1.

### Step 3: Ethyl 6-bromo-8-chloroimidazo[1,5-a]pyridine-3-carboxylate (39-3)

Into a vial were added ethyl 2-(((5-bromo-3-chloropyridin-2-yl)methyl)amino)-2-oxoacetate (100 mg, 0.31 mmol) and phosphorus oxychloride (1 mL) under N₂. The resulting mixture was stirred for 8 hrs at 110 °C. After completion, the reaction mixture was quenched by the addition of water carefully, then extracted with ethyl acetate. The combined organic layers were dried over anhydrous Na₂SO₄, filtered, then concentrated under reduced pressure. The residue was purified by prep-TLC (PE:EtOAc=1:1) to afford ethyl 6-bromo-8-chloroimidazo[1,5-a]pyridine-3-carboxylate (80 mg). LCMS (ESI, m/z): [M+H]⁺=302.5.

### Step 4: Ethyl 6-(benzylthio)-8-chloroimidazo[1,5-a]pyridine-3-carboxylate (39-4)

To a solution of ethyl 6-bromo-8-chloroimidazo[1,5-a]pyridine-3-carboxylate (3.78 g, 12.5 mmol) in 1,4-dioxane (50 mL) was added XantPhos (1.44 g, 2.5 mmol), Pd₂(dba)₃·CHCl₃ (1.29 g, 1.2 mmol) and DIEA (3.22 g, 24.9 mmol) under N₂. The resulting mixture was degassed with N₂, then stirred for 2 hrs at 85 °C. After completion, the reaction mixture was concentrated under reduced pressure. The residue was purified by flash column chromatography on silica gel (eluting with 30% EtOAc in PE) to afford ethyl 6-(benzylsulfanyl)-8-chloroimidazo[1,5-a]pyridine-3-carboxylate (3 g). LCMS (ESI, m/z): [M+H]⁺=347.1.

### Step 5: 6-(Benzylthio)-8-chloroimidazo[1,5-a]pyridine-3-carbohydrazide (39-5)

Into a round-bottom flask was added ethyl 6-(benzylsulfanyl)-8-chloroimidazo[1,5-a]pyridine-3-carboxylate (3 g, 8.7 mmol), hydrazine hydrate (80% in H₂O, 8 mL, 132 mmol) and EtOH (30 mL) under N₂. The resulting mixture was stirred for 2 hrs at 50 °C. After completion, the precipitate was collected by filtration and washed with EtOH to afford 6-(benzylsulfanyl)-8-chloroimidazo[1,5-a]pyridine-3-carbohydrazide (2.5 g). LCMS (ESI, m/z): [M+H]⁺=333.1.

### Step 6: 6-(Benzylthio)-8-chloro-N′-(2,2-difluoroacetyl)imidazo[1,5-a]pyridine-3-carbohydrazide (39-6)

Into a round-bottom flask was added 6-(benzylsulfanyl)-8-chloroimidazo[1,5-a]pyridine-3-carbohydrazide (2 g, 6 mmol) and DIEA (1.6 g, 12 mmol) in THF (25 ml). The resulting mixture was cooled to 0 °C. Then 2,2-difluoroacetic anhydride (1.2 g, 6.9 mmol) was added dropwise at 0 °C under N₂. The resulting mixture was stirred for 4 hrs at 0 °C. After completion, the reaction mixture was worked up with water and extracted with ethyl acetate. The combined organic layers were washed with saturated brine, dried over anhydrous Na₂SO₄. After filtration, the filtrate was concentrated under reduced pressure. The residue was purified by trituration with ethyl ether to afford 6-(benzylsulfanyl)-8-chloro-N′-(2,2-difluoroacetyl)imidazo[1,5-a]pyridine-3-carbohydrazide (1 g). LCMS (ESI, m/z): [M+H]⁺=411.1.

### Step 7: 2-(6-(Benzylthio)-8-chloroimidazo[1,5-a]pyridin-3-yl)-5-(difluoromethyl)-1,3,4-thiadiazole (39-7)

Into a round-bottom flask was added 6-(benzylsulfanyl)-8-chloro-N′-(2,2-difluoroacetyl)imidazo[1,5-a]pyridine-3-carbohydrazide (1 g, 2.4 mmol), Lawesson's reagent (1.3 g, 3.2 mmol) and THF (10 mL) under N₂. The resulting mixture was stirred for 4 hrs at 70 °C. After completion, the resulting mixture was concentrated under reduced pressure. The residue was purified by flash column chromatography on silica gel (eluting with 10% EtOAc in PE) to afford 2-(6-(benzylthio)-8-chloroimidazo[1,5-a]pyridin-3-yl)-5-(difluoromethyl)-1,3,4-thiadiazole (560 mg). LCMS (ESI, m/z): [M+H]⁺=409.0.

### Step 8: 8-chloro-3-(5-(difluoromethyl)-1,3,4-thiadiazol-2-yl)imidazo[1,5-a]pyridine-6-sulfonyl chloride (39-8)

To a solution of 2-(6-(benzylthio)-8-chloroimidazo[1,5-a]pyridin-3-yl)-5-(difluoromethyl)-1,3,4-thiadiazole (500 mg, 1.2 mmol) in water (2 mL) and AcOH (6 mL) was added NCS (653 mg, 4.9 mmol) portion wise at room temperature. The resulting mixture was stirred for 3 hrs at room temperature. After completion, the reaction mixture was worked up with water and extracted with DCM. The combined organic layers were washed with saturated brine, dried over anhydrous MgSO₄. After filtration, the filtrate was concentrated under reduced pressure. The crude product was used for the next step directly without further purification. LCMS (ESI, m/z): [M+H]⁺=385.2.

### Step 9: 8-chloro-3-(5-(difluoromethyl)-1,3,4-thiadiazol-2-yl)-N-(1-(difluoromethyl)cyclopropyl)imidazo[1,5-a]pyridine-6-sulfonamide (39-9)

Into a round-bottom flask was added 1-(difluoromethyl)cyclopropan-I-amine hydrochloride (187 mg, 1.3 mmol), pyridine (308 mg, 3.9 mmol), 4A molecular sieve and DCM (10 mL). The mixture was stirred for 20 mins, then 8-chloro-3-(5-(difluoromethyl)-1,3,4-thiadiazol-2-yl)imidazo[1,5-a]pyridine-6-sulfonyl chloride (500 mg, 1.3 mmol) was added under N₂ at room temperature. The resulting mixture was stirred for 3 hrs at room temperature. The reaction mixture was filtered, and the filtrate was concentrated under reduced pressure. The residue was purified by flash column chromatography on silica gel (eluting with 10% EtOAc in PE) to afford 8-chloro-3-(5-(difluoromethyl)-1,3,4-thiadiazol-2-yl)-N-(1-(difluoromethyl)cyclopropyl)imidazo[1,5-a]pyridine-6-sulfonamide (200 mg). LCMS (ESI, m/z): [M+H]⁺=455.9.

### Step 10: 8-chloro-3-(5-(difluoromethyl)-1,3,4-thiadiazol-2-yl)-N-(1-(difluoromethyl)cyclopropyl)imidazo[1,5-a]pyridine-6-sulfonamide (39)

A solution of 8-chloro-3-(5-(difluoromethyl)-1,3,4-thiadiazol-2-yl)-N-(1-(difluoromethyl)cyclopropyl)imidazo[1,5-a]pyridine-6-sulfonamide (130 mg, 0.285 mmol), 2-oxa-7-azaspiro[3.5]nonane (181 mg, 1.42 mmol), Pd-PEPPSI-IPentCl catalyst (49 mg, 0.05 mmol) and Cs₂CO₃ (215 mg, 0.66 mmol) in 1,4-dioxane (4 mL) was degassed with N₂, then stirred for 16 hrs at 105 °C under N₂ atmosphere. After completion, the mixture was allowed to cool down to room temperature, worked up with water and extracted with EtOAc. The combined organic layers were washed with saturated brine, dried over anhydrous Na₂SO₄, filtered, and concentrated under reduced pressure. The residue was purified by prep-TLC (PE:EtOAc=1:2) to afford the product which was further purified by prep-HPLC (0.1% FA) to afford 3-(5-(difluoromethyl)-1,3,4-thiadiazol-2-yl)-N-(1-(difluoromethyl)cyclopropyl)-8-(2-oxa-7-azaspiro[3.5]nonan-7-yl)imidazo[1,5-a]pyridine-6-sulfonamide. LCMS (ESI, n/z): [M+H]⁺=547.3. ¹H NMR (400 MHz, DMSO-d₆) δ 9.54 (s, 1H), 9.13 (s, 1H), 7.96 (s, 1H), 7.68 (t, J=53 Hz, 1H), 6.68 (s, 1H), 5.79 (t, J=56 Hz, 1H), 4.41 (s, 4H), 3.25-3.21 (m, 4H), 2.08-2.04 (m, 4H), 0.98-0.94 (m, 4H). ¹⁹F NMR (400 MHz, DMSO-d₆) δ −109.31, −120.27.

### Example 40

### 3-(5-(Difluoromethyl)-1,3,4-thiadiazol-2-yl)-N-(1-(difluoromethyl)cyclopropyl)-8-(2-oxa-7-azaspiro[3.5]nonan-7-yl)-[1,2,4]triazolo[4,3-a]pyridine-6-sulfonamide (40)

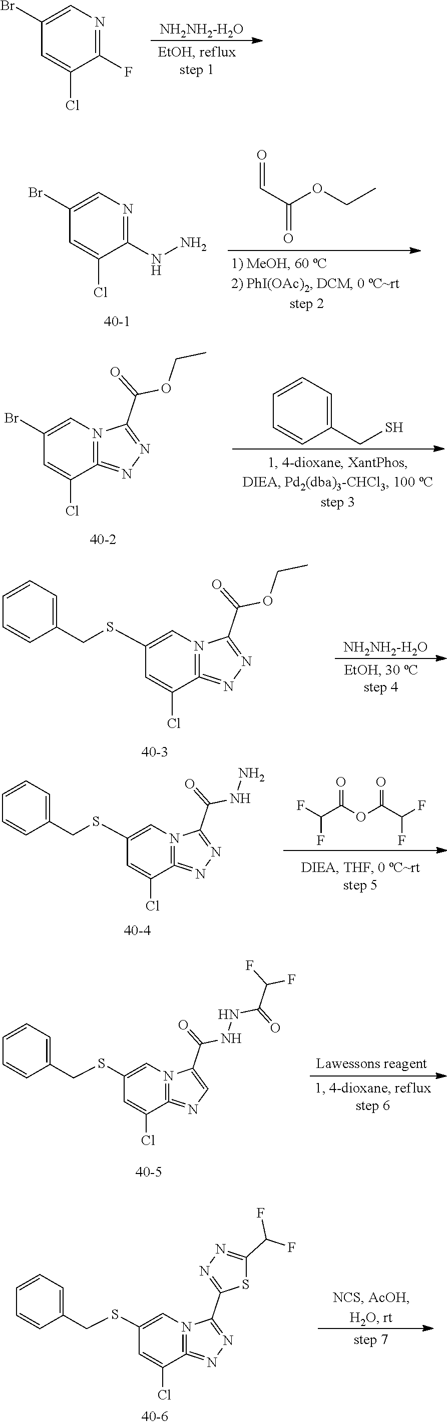

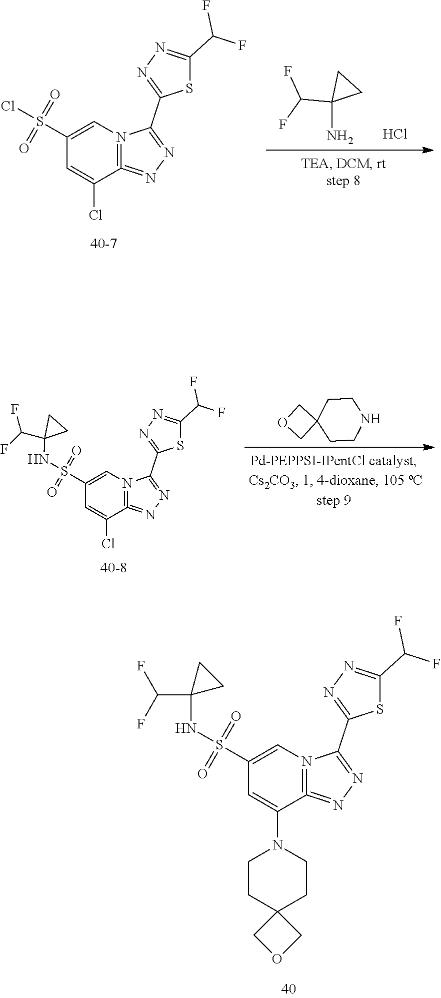

### Step 1: 5-Bromo-3-chloro-2-hydrazineylpyridine (40-1)

Into a round-bottomed flask fitted with a reflux condenser was added 5-bromo-3-chloro-2-fluoropyridine (19 g, 90 mmol), hydrazine hydrate (98%, 19 mL, 383 mmol) and EtOH (200 mL) under N₂. The resulting mixture was stirred at reflux for 3 hrs. The reaction mixture was allowed to cool down to room temperature and concentrated under reduced pressure to obtain the crude product. The crude product was slurried with water at 25 °C for 30 minutes, then filtered and dried under vacuum to afford 5-bromo-3-chloro-2-hydrazineylpyridine (19.3 g). LCMS (ESI, m/z): [M+H]⁺=221.9.

### Step 2: Ethyl 6-bromo-8-chloro-[1,2,4]triazolo[4,3-a]pyridine-3-carboxylate (40-2)

Into an oven-dried round-bottomed flask was added 5-bromo-3-chloro-2-hydrazineylpyridine (14 g, 62 mmol), ethyl 2-oxoacetate (7.6 g, 74 mmol) and MeOH (160 mL) under N₂. The resulting mixture was stirred at 60 °C for 2 hrs. Then the reaction mixture was cooled down to room temperature and concentrated under reduced pressure. The residue was dissolved in DCM (160 mL) and added phenyl-λ³-iodanediyl diacetate (26 g, 81 mmol) portion wise at 0 °C. After addition, the mixture was allowed to warm to room temperature and stirred for 16 hrs. Upon completion of the reaction, the resulting mixture was concentrated under reduced pressure. The residue was purified by reversed-phase flash column chromatography on C18 silica gel (eluting with 0% to 50% MeCN in water (0.05% NH₄HCO₃)) to afford ethyl 6-bromo-8-chloro-[1,2,4]triazolo[4,3-a]pyridine-3-carboxylate (16.2 g). LCMS (ESI, m/z): [M+H]⁺=304.0.

### Step 3: Ethyl 6-(benzylthio)-8-chloro-[1,2,4]triazolo[4,3-a]pyridine-3-carboxylate (40-3)

Into an oven-dried round-bottomed flask was added ethyl 6-bromo-8-chloro-[1,2,4]triazolo[4,3-a]pyridine-3-carboxylate (11 g, 37 mmol), phenylmethanethiol (3.6 g, 29 mmol), Pd₂(dba)₃·CHCl₃ (3.8 g, 3.7 mmol), XantPhos (4.3 g, 7.4 mmol), DIEA (14.3 g, 110 mmol) and 1,4-dioxane (400 mL) under N₂. The reaction mixture was degassed with N₂, and then stirred at 90 °C for 3 hrs. The reaction mixture was allowed to cool down to room temperature and concentrated under reduced pressure. The residue was worked up with water and extracted with ethyl acetate. The combined organic layers were washed with saturated brine, dried over anhydrous Na₂SO₄, filtered, and concentrated to dryness under reduced pressure. The residue was purified by reversed-phase flash column chromatography on C18 silica gel (eluting with 0% to 50% MeCN in water (0.05% NH₄HCO₃)) to afford ethyl 6-(benzylthio)-8-chloro-[1,2,4]triazolo[4,3-a]pyridine-3-carboxylate (11 g). LCMS (ESI, m/z): [M+H]⁺=348.0.

### Step 4: 6-(Benzylthio)-8-chloro-[1,2,4]triazolo[4,3-a]pyridine-3-carbohydrazide (40-4)

Into a round-bottomed flask was added ethyl 6-(benzylthio)-8-chloro-[1,2,4]triazolo[4,3-a]pyridine-3-carboxylate (8.0 g, 22 mmol), EtOH (80 mL) and hydrazine hydrate (98%, 4.6 g, 90 mmol) at room temperature under N₂. The resulting mixture was stirred for 1 hr at 30 °C. The mixture was allowed to cool down to room temperature. The precipitate was collected by filtration and washed with EtOH. The resulting solid was dried under vacuum to afford 6-(benzylthio)-8-chloro-[1,2,4]triazolo[4,3-a]pyridine-3-carbohydrazide (7.0 g). The crude product was used for the next step directly without further purification. LCMS (ESI, m/z): [M+H]⁺=334.1.

### Step 5: 6-(Benzylthio)-8-chloro-N′-(2,2-difluoroacetyl)-[1,2,4]triazolo[4,3-a]pyridine-3-carbohydrazide (40-5)

Into a round-bottomed flask was added 6-(benzylthio)-8-chloro-[1,2,4]triazolo[4,3-a]pyridine-3-carbohydrazide (6.5 g, 18 mmol), THF (70 mL), DIEA (4.6 g, 36 mmol) and 2,2-difluoroacetic anhydride (3.7 g, 21 mmol) at 0 °C under N₂. The resulting mixture was stirred for 3 hrs at room temperature. The reaction mixture was concentrated under reduced pressure and worked up with water. The resulting mixture was extracted with EtOAc. The combined organic layers were washed with saturated brine, dried over anhydrous Na₂SO₄. After filtration, the filtrate was concentrated under reduced pressure. The residue was purified by reversed-phase flash column chromatography on C18 silica gel (eluting with 30% to 50% MeCN in water (0.05% NH₄HCO₃)) to afford 6-(benzylthio)-8-chloro-N-(2,2-difluoroacetyl)-[1,2,4]triazolo[4,3-a]pyridine-3-carbohydrazide (7.0 g). LCMS (ESI, m/z): [M+H]⁺=412.1.

### Step 6: 2-(6-(Benzylthio)-8-chloro-[1,2,4]triazolo[4,3-a]pyridin-3-yl)-5-(difluoromethyl)-1,3,4-thiadiazole (40-6)

Into a round-bottomed flask was added 6-(benzylthio)-8-chloro-N-(2,2-difluoroacetyl)-[1,2,4]triazolo[4,3-a]pyridine-3-carbohydrazide (4.2 g, 10 mmol), 1,4-dioxane (80 mL) and 2,4-bis(4-methoxyphenyl)-1,3,2,4-dithiadiphosphetane 2,4-disulfide (6.1 g, 15 mmol) at room temperature under N₂. The resulting mixture was stirred for 16 hrs at reflux under N₂ atmosphere. The reaction mixture was allowed to cool down to room temperature, worked up with water and extracted with EtOAc. The combined organic layers were washed with saturated brine, dried over anhydrous Na₂SO₄. After filtration, the filtrate was concentrated under reduced pressure. The residue was purified by flash column chromatography on silica gel (eluting with 20% EA in PE) to afford 2-(6-(benzylthio)-8-chloro-[1,2,4]triazolo[4,3-a]pyridin-3-yl)-5-(difluoromethyl)-1,3,4-thiadiazole (4.0 g). LCMS (ESI, m/z): [M+H]⁺=410.1.

### Step 7: 8-Chloro-3-(5-(difluoromethyl)-1,3,4-thiadiazol-2-yl)-[1,2,4]triazolo[4,3-a]pyridine-6-sulfonyl chloride (40-7)

To a mixture of 2-(6-(benzylthio)-8-chloro-[1,2,4]triazolo[4,3-a]pyridin-3-yl)-5-(difluoromethyl)-1,3,4-thiadiazole (1.4 g, 3.4 mmol) in AcOH (7.4 mL) and H₂O (2.8 mL) was added NCS (1.8 g, 14 mmol) portion wise at room temperature. The resulting mixture was stirred for 1 hr at room temperature. The reaction mixture was worked up with water, then extracted with EtOAc. The combined organic layers were washed with saturated brine, dried over anhydrous Na₂SO₄. After filtration, the filtrate was concentrated under reduced pressure to afford 8-chloro-3-(5-(difluoromethyl)-1,3,4-thiadiazol-2-yl)-[1,2,4]triazolo[4,3-a]pyridine-6-sulfonyl chloride (1.3 g). The crude product was used for the next step directly without further purification. LCMS (ESI, m/z): [M+H]⁺=386.0.

### Step 8: 8-Chloro-3-(5-(difluoromethyl)-1,3,4-thiadiazol-2-yl)-N-(1-(difluoromethyl)cyclopropyl)-[1,2,4]triazolo[4,3-a]pyridine-6-sulfonamide (40-8)

To a stirred solution of 1-(difluoromethyl)cyclopropan-1-amine hydrochloride (775 mg, 5.18 mmol) and TEA (788 mg, 7.78 mmol) in DCM (20 mL) was added 8-chloro-3-(5-(difluoromethyl)-1,3,4-thiadiazol-2-yl)-[1,2,4]triazolo[4,3-a]pyridine-6-sulfonyl chloride (1.0 g, 2.6 mmol) portion wise at room temperature under N₂. The resulting mixture was stirred for 30 min at room temperature. The reaction mixture was worked up with water, and then extracted with EtOAc. The combined organic layers were washed with saturated brine, dried over anhydrous Na₂SO₄. After filtration, the filtrate was concentrated under reduced pressure. The residue was purified by reversed-phase flash column chromatography on C18 silica gel (eluting with 30% to 50% MeCN in water (0.05% NH₄HCO₃)) to afford 8-chloro-3-(5-(difluoromethyl)-1,3,4-thiadiazol-2-yl)-N-(1-(difluoromethyl)cyclopropyl)-[1,2,4]triazolo[4,3-a]pyridine-6-sulfonamide (790 mg). LCMS (ESI, m/z): [M+H]⁺=457.1.

### Step 9: 3-(5-(Difluoromethyl)-1,3,4-thiadiazol-2-yl)-N-(1-(difluoromethyl)cyclopropyl)-8-(2-oxa-7-azaspiro[3.5]nonan-7-yl)-[1,2,4]triazolo[4,3-a]pyridine-6-sulfonamide (40)

Into a vial was added 8-chloro-3-[5-(difluoromethyl)-1,3,4-thiadiazol-2-yl]-N-[1-(difluoromethyl)cyclopropyl]-[1,2,4]triazolo[4,3-a]pyridine-6-sulfonamide (150 mg, 0.328 mmol), 2-oxa-7-azaspiro[3.5]nonane (208 mg, 1.64 mmol), Cs₂CO₃ (321 mg, 0.985 mmol), 1,4-dioxane (1.5 mL) and Pd-PEPPSI-IPentCl catalyst (55 mg, 0.057 mmol) under N₂. The resulting mixture was degassed with N₂, then stirred for 1.5 h at 105 °C. The reaction mixture was allowed to cool down to room temperature and worked up with water at room temperature. The resulting mixture was extracted with EtOAc. The combined organic layers were washed with saturated brine, dried over anhydrous Na₂SO₄. After filtration, the filtrate was concentrated under reduced pressure. The crude product was purified by achiral-HPLC eluting with ACN:MeOH=80:20 (1%, 2 M aqueous NH₃ in MeOH) to afford 3-(5-(difluoromethyl)-1,3,4-thiadiazol-2-yl)-N-(1-(difluoromethyl)cyclopropyl)-8-(2-oxa-7-azaspiro[3.5]nonan-7-yl)-[1,2,4]triazolo[4,3-a]pyridine-6-sulfonamide. LCMS (ESI, m/z): [M+H]⁺=548.2. ¹H NMR (400 MHz, DMSO-d₆) δ 9.26 (s, 1H), 9.20 (s, 1H), 7.74 (t, J=53.2 Hz, 1H), 6.90 (s, 1H), 5.79 (t, J=56.0 Hz, 1H), 4.41 (s, 4H), 3.73-3.68 (m, 4H), 2.03-1.99 (m, 4H), 0.98-0.94 (m, 4H). ¹⁹F NMR (400 MHz, DMSO-d₆) δ −109.74, −120.58.

### Example 41

### N-(1-cyanocyclopropyl)-1-(5-(difluoromethyl)-1,3,4-thiadiazol-2-yl)-6-fluoro-5-(2-oxa-7-azaspiro[3.5]nonan-7-yl)imidazo[1,5-a]pyridine-7-sulfonamide (41)

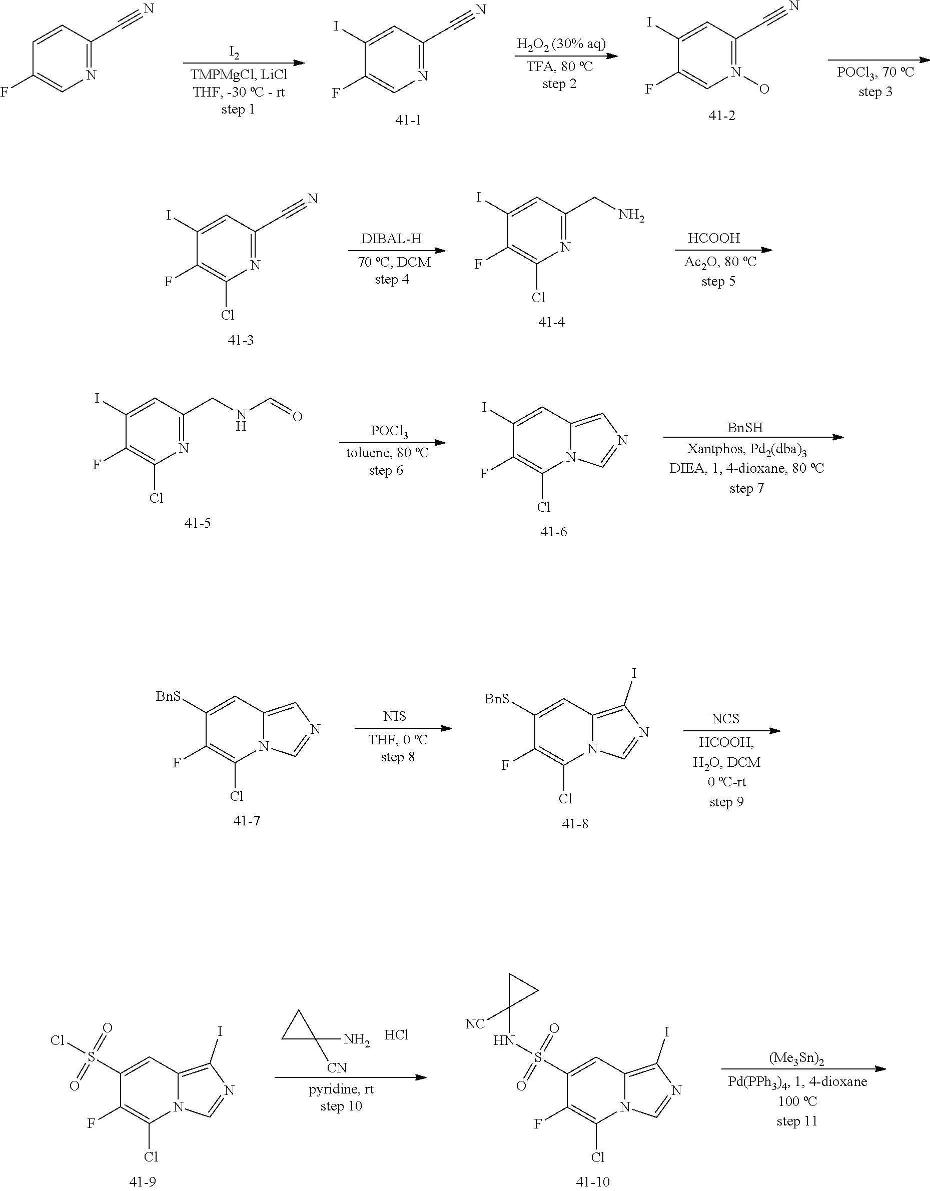

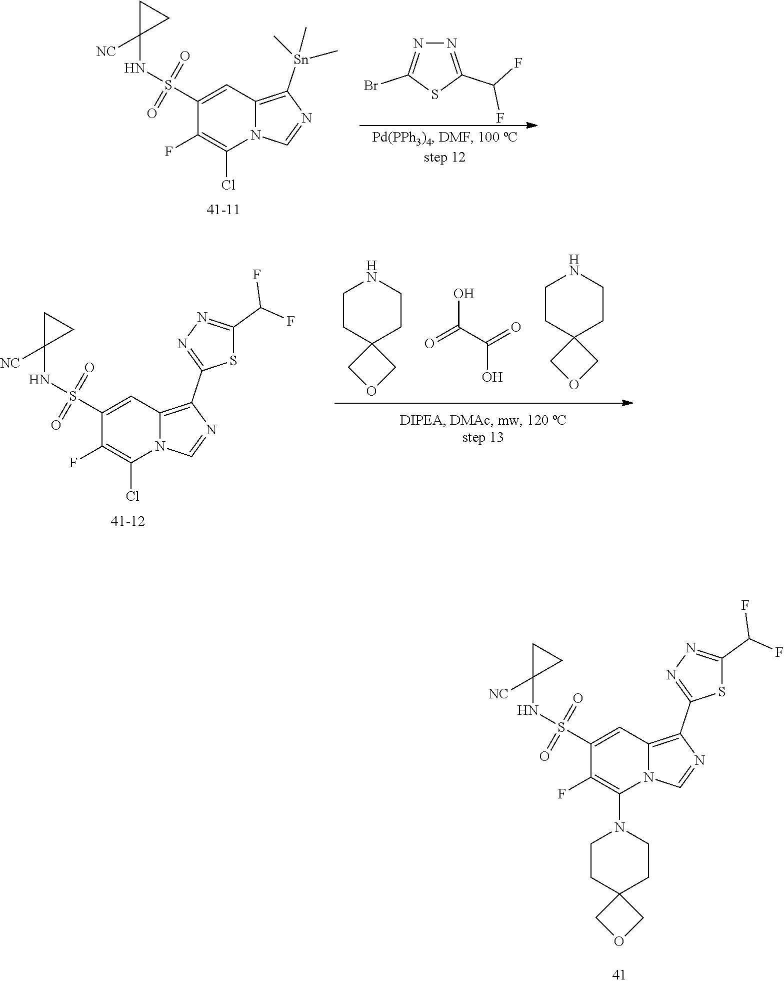

### Step 1: 5-fluoro-4-iodo-pyridine-2-carbonitrile (41-1)

To a solution of 5-fluoropyridine-2-carbonitrile (18.5 g, 152 mmol) in dry THF (200 mL) was added TMPMgCl·LiCl (1.0 M in THF, 228 mL, 228 mmol) dropwise at −30 °C under N₂. The mixture was stirred at −30 °C for 1 hr. Then a solution of iodine (46.2 g, 182 mmol) in dry THF (150 mL) was added at −30 °C under N₂. The mixture was allowed to warm up to room temperature and stirred at room temperature for 1 hr. LCMS showed the reaction completed. The reaction mixture was quenched with saturated aqueous NH₄Cl solution, then extracted with EtOAc. The combined organic layers were washed with saturated brine, dried over anhydrous Na₂SO₄, filtered, and concentrated under reduced pressure. The residue was purified by flash column chromatography on silica gel (eluting with 7% EtOAc in PE) to afford 5-fluoro-4-iodo-pyridine-2-carbonitrile (26 g). LCMS (ESI, m/z): [M+H]⁺=249.0.

### Step 2: 5-fluoro-4-iodo-1-oxido-pyridin-1-ium-2-carbonitrile (41-2)

To a solution of 5-fluoro-4-iodo-pyridine-2-carbonitrile (26 g, 104.8 mmol) in TFA (200 mL) was added H₂O₂ (30% in water, 160 mL) dropwise at 0 °C. The mixture was stirred at 80 °C for 4 hrs. After completion, the reaction mixture was quenched with saturated aqueous Na₂SO₃ solution, then extracted with EtOAc. The combined organic layers were washed with saturated brine, dried over anhydrous Na₂SO₄, filtered, and concentrated under reduced pressure. The residue was purified by flash column chromatography on silica gel (eluting with 17% EtOAc in PE) to give 5-fluoro-4-iodo-1-oxido-pyridin-1-ium-2-carbonitrile (10 g). LCMS (ESI, m/z): [M+H]⁺=265.0.

### Step 3: 6-chloro-5-fluoro-4-iodo-pyridine-2-carbonitrile (41-3)

A solution of 5-fluoro-4-iodo-1-oxido-pyridin-1-ium-2-carbonitrile (10 g, 37.9 mmol) in POCl₃ (50 mL) was stirred at 70 °C for 1 hr under N₂. LCMS showed the reaction completed. The reaction mixture was concentrated under reduced pressure to remove most POCl₃. Then the residue was worked up with water, then extracted with EtOAc. The combined organic layers were washed with saturated aqueous sodium bicarbonate and saturated brine, dried over anhydrous Na₂SO₄, filtered, and concentrated to give a residue. The residue was purified by flash column chromatography on silica gel (eluting with 5% EtOAc in PE) to afford 6-chloro-5-fluoro-4-iodo-pyridine-2-carbonitrile (4.5 g). LCMS (ESI, m/z): [M+H]⁺=282.9.

### Step 4: (6-chloro-5-fluoro-4-iodo-2-pyridyl)methanamine (41-4)

To a solution of 6-chloro-5-fluoro-4-iodo-pyridine-2-carbonitrile (4.2 g, 14.9 mmol) in DCM (100 mL) was added DIBAL-H (1.0 M in hexanes, 74.5 mL, 74.5 mmol) dropwise at 0 °C under N₂. The mixture was stirred at 0 °C for 1 hr under N₂ atmosphere. LCMS showed the reaction completed. The reaction mixture was quenched with saturated aqueous NH₄Cl solution, then extracted with EtOAc. The combined organic layers were washed with saturated brine, dried over anhydrous Na₂SO₄, filtered, and concentrated to give the crude product which was used directly in the next step without further purification. LCMS (ESI, m/z): [M+H]⁺=287.0.

### Step 5: N-[(6-chloro-5-fluoro-4-iodo-2-pyridyl)methyl]formamide (41-5)

To a solution of (6-chloro-5-fluoro-4-iodo-2-pyridyl)methanamine (3.5 g, 12.2 mmol) in formic acid (40 mL) was added acetic anhydride (8 mL) under N₂. The mixture was stirred at 80 °C for 4 hrs under N₂ atmosphere. LCMS showed the reaction completed. The reaction mixture was concentrated under reduced pressure to afford the crude product which was used directly in the next step without further purification. LCMS (ESI, m/z): [M+H]⁺=315.0.

### Step 6: 5-chloro-6-fluoro-7-iodo-imidazo[1,5-a]pyridine (41-6)

To a solution of N-[(6-chloro-5-fluoro-4-iodo-2-pyridyl)methyl]formamide (3.2 g, 10.2 mmol) in toluene (20 mL) was added POCl₃ (2 mL) under N₂. The reaction solution was stirred at 80 °C for 4 hrs under N₂ atmosphere. LCMS showed the reaction completed. The reaction mixture was concentrated under reduced pressure. The residue was adjusted to pH=8 by saturated aqueous sodium bicarbonate, then extracted with EtOAc. The combined organic layers were washed with saturated brine, dried over anhydrous Na₂SO₄, filtered, and concentrated under reduced pressure. The residue was purified by flash column chromatography on silica gel (eluting with 20% EtOAc in PE) to give 5-chloro-6-fluoro-7-iodo-imidazo[1,5-a]pyridine (2.47 g). LCMS (ESI, m/z): [M+H]⁺=297.0.

### Step 7: 7-benzylsulfanyl-5-chloro-6-fluoro-imidazo[1,5-a]pyridine (41-7)

To a solution of 5-chloro-6-fluoro-7-iodo-imidazo[1,5-a]pyridine (2.47 g, 8.3 mmol) in 1,4-dioxane (40 mL) was added phenylmethanethiol (1.09 g, 8.75 mmol), DIEA (1.08 g, 8.33 mmol), Xantphos (964 mg, 1.67 mmol) and Pd₂(dba)₃ (763 mg, 0.83 mmol) under N₂. The reaction mixture was degassed, then stirred at 80 °C for 3 hrs under N₂. LCMS showed the reaction completed. The reaction mixture was worked up with water, then extracted with EtOAc. The combined organic layers were washed with saturated brine, dried over anhydrous Na₂SO₄, filtered, and concentrated under reduced pressure to give a residue. The residue was purified by flash column chromatography on silica gel (eluting with 20% EtOAc in PE) to give 7-benzylsulfanyl-5-chloro-6-fluoro-imidazo[1,5-a]pyridine (1.8 g). LCMS (ESI, m/z): [M+H]⁺=293.1.

### Step 8: 7-benzylsulfanyl-5-chloro-6-fluoro-1-iodo-imidazo[1,5-a]pyridine (41-8)

To a solution of 7-benzylsulfanyl-5-chloro-6-fluoro-imidazo[1,5-a]pyridine (900 mg, 3.07 mmol) in THF (20 mL) was added 1-iodopyrrolidine-2,5-dione (830 mg, 3.69 mmol) at 0 °C. The mixture was stirred at 0 °C for 2 hrs. LCMS showed the reaction completed. The reaction mixture was worked up with water, then extracted with EtOAc. The combined organic layers were washed with saturated brine, dried over anhydrous Na₂SO₄, filtered, and concentrated to give a residue. The residue was purified by flash column chromatography on silica gel (eluting with 10% EtOAc in PE) to afford 7-benzylsulfanyl-5-chloro-6-fluoro-1-iodo-imidazo[1,5-a]pyridine (900 mg). LCMS (ESI, m/z): [M+H]⁺=419.0.

### Step 9: 5-chloro-6-fluoro-1-iodoimidazo[1,5-a]pyridine-7-sulfonyl chloride (41-9)

To a solution of 7-benzylsulfanyl-5-chloro-6-fluoro-1-iodo-imidazo[1,5-a]pyridine (800 mg, 1.91 mmol) in formic acid (3 mL), water (0.3 mL) and DCM (10 mL) was added 1-chloropyrrolidine-2,5-dione (765 mg, 5.73 mmol) at 0 °C under N₂. The mixture was allowed to warm to room temperature and stirred for 1 hr. LCMS showed the reaction completed. The reaction mixture was worked up with water, then extracted with DCM. The combined organic layers were washed with saturated brine, dried over anhydrous Na₂SO₄, filtered, and concentrated under reduced pressure to give the residue. The residue was purified by flash column chromatography on silica gel (eluting with 30% EtOAc in PE) to afford 5-chloro-6-fluoro-1-iodo-imidazo[1,5-a]pyridine-7-sulfonyl chloride (500 mg). LCMS (ESI, m/z): [M+H]⁺=395.0.

### Step 10: 5-chloro-N-(1-cyanocyclopropyl)-6-fluoro-I-iodoimidazo[1,5-a]pyridine-7-sulfonamide (41-10)

To a solution of 1-aminocyclopropanecarbonitrile hydrochloride (180 mg, 1.52 mmol) in pyridine (7 mL) was added 5-chloro-6-fluoro-1-iodo-imidazo[1,5-a]pyridine-7-sulfonyl chloride (500 mg, 1.27 mmol) at room temperature under N₂. The mixture was stirred at room temperature for 1 hr. LCMS showed the reaction completed. The reaction mixture was worked up with water, then extracted with EtOAc. The combined organic layers were washed with saturated brine, dried over anhydrous Na₂SO₄, filtered, and concentrated under reduced pressure to give the residue. The residue was purified by flash column chromatography on silica gel (eluting with 60% EtOAc in PE) to afford 5-chloro-N-(1-cyanocyclopropyl)-6-fluoro-1-iodo-imidazo[1,5-a]pyridine-7-sulfonamide (500 mg). LCMS (ESI, m/z): [M+H]⁺=441.0.

### Step 11: 5-chloro-N-(1-cyanocyclopropyl)-6-fluoro-1-(trimethylstannyl)imidazo[1,5-a]pyridine-7-sulfonamide (41-11)

To a solution of 5-chloro-N-(1-cyanocyclopropyl)-6-fluoro-1-iodo-imidazo[1,5-a]pyridine-7-sulfonamide (500 mg, 1.13 mmol) in 1,4-dioxane (5 ml) was added trimethyl(trimethylstannyl)stannane (744 mg, 2.27 mmol) and Pd(PPh₃)₄ (131.1 mg, 0.113 mmol) under N₂. The mixture was degassed, then stirred at 100 °C for 7 hrs under N₂ atmosphere. After completion, the reaction mixture was concentrated under reduced pressure to give the crude product which was directly used for the next step without further purification. LCMS (ESI, m/z): [M+H]⁺=479.1.

### Step 12: 5-chloro-N-(1-cyanocyclopropyl)-1-(5-(difluoromethyl)-1,3,4-thiadiazol-2-yl)-6-fluoroimidazo[1,5-a]pyridine-7-sulfonamide (41-12)

To a solution of 5-chloro-N-(1-cyanocyclopropyl)-6-fluoro-1-trimethylstannyl-imidazo[1,5-a]pyridine-7-sulfonamide (500 mg, 1.05 mmol) in DMF (7 mL) was added 2-bromo-5-(difluoromethyl)-1,3,4-thiadiazole (270 mg, 1.26 mmol) and Pd(PPh₃)₄ (121 mg, 0.105 mmol) under N₂. The reaction mixture was degassed, then stirred at 100 °C for 7 hrs under N₂ atmosphere. LCMS showed the reaction completed. The reaction mixture was worked up with water, then extracted with EtOAc. The combined organic layers were washed with saturated brine, dried over anhydrous Na₂SO₄, filtered, and concentrated under reduced pressure to give the residue. The residue was purified by flash column chromatography on silica gel (eluting with 50% EtOAc in PE) to give 5-chloro-N-(1-cyanocyclopropyl)-1-[5-(difluoromethyl)-1,3,4-thiadiazol-2-yl]-6-fluoro-imidazo[1,5-a]pyridine-7-sulfonamide (280 mg). LCMS (ESI, m/z): [M+H]⁺=449.1.

### Step 13: N-(1-cyanocyclopropyl)-1-(5-(difluoromethyl)-1,3,4-thiadiazol-2-yl)-6-fluoro-5-(2-oxa-7-azaspiro[3.5]nonan-7-yl)imidazo[1,5-a]pyridine-7-sulfonamide (41)

To a solution of 5-chloro-N-(1-cyanocyclopropyl)-1-[5-(difluoromethyl)-1,3,4-thiadiazol-2-yl]-6-fluoro-imidazo[1,5-a]pyridine-7-sulfonamide (100 mg, 0.223 mmol) in N,N-dimethylacetamide (3 mL) was added 2-Oxa-7-azaspiro[3.5]nonane hemioxalate (76.5 mg, 0.445 mmol) under N₂. The mixture was stirred at 120 °C under microwave for 5 hrs under N₂ atmosphere. LCMS showed the reaction completed. The reaction mixture was worked up with water, then extracted with EtOAc. The combined organic layers were washed with saturated brine, dried over anhydrous Na₂SO₄, filtered, and concentrated under reduced pressure to give the residue. The residue was purified by prep-HPLC (0.1% FA) to afford N-(1-cyanocyclopropyl)-1-[5-(difluoromethyl)-1,3,4-thiadiazol-2-yl]-6-fluoro-5-(2-oxa-7-azaspiro[3.5]nonan-7-yl)imidazo[1,5-a]pyridine-7-sulfonamide. LCMS (ESI, m/z): [M+H]⁺=540.2. ¹HNMR (400 MHz, DMSO-d₆) δ 9.77 (br s, 1H), 8.69 (s, 1H), 8.54 (d, J=6.0 Hz, 1H), 7.65 (t, J=53.2 Hz, 1H), 4.43 (s, 4H), 3.22 (m, 4H), 2.06 (s, 4H), 1.51-1.45 (m, 2H), 1.40-1.33 (m, 2H).

### Biological Example 1

### PARG Biochemical Enzymatic Assay

TEV-His8-PolyADP-Ribose Polymerase 1 (379-1014) (PARP1) and N-his8-TEV-hPARG (389-976) were used to transform BL21(DE3) competent cells followed by growth for 1 hour at 37 °C and plating on Kanamycin resistant agar plates. A single colony was then inoculated into 1 ml of Luria Broth and expansion into a 1 liter scale for growth at 37 °C for 3 hours. Expression was induced via 0.2 mM IPTG induction for 20 hours at 16 °C in BL21(DE3) E. coli. Purification was then performed using a Nickel column, size exclusion chromatography and HPLC analysis.

For substrate preparation, 2 mM nicked DNA and 2 μM PARP1 protein were mixed for 10 minutes at 25 °C 10 μM biotin-NAD+ and 100 μM NAD+ were then added and incubated for 60 minutes at 30 °C to yield PARylated PARP1 substrate.

For compound screening, compounds were serially diluted to a 3× final concentration in assay buffer (Tris pH7.5 50 mM, KCl 50 mM, Tween-20 0.01%, BSA 0.1 mg/ml, EDTA 3 mM, EGTA 0.4 mM, DTT 1 mM) containing 1% DMSO. A top concentration of 1.5 μM for a 10-point dose response curve using a 1:3 dilution curve was used. 2.5×PARG (156.25 pM working concentration, 62.5 pM final concentration) was prepared in assay buffer and 4 ul added to a 384 well assay plate containing 2 μl of serially diluted compound. The plate was then centrifuged for 10 seconds and incubated for 60 minutes at 23 °C. 4 μl of 2.5× Biotin-Parylated PARP1 (12.5 nM working concentration, 5 nM final concentration) was then added to the plate and incubated for 10 minutes at 23 °C. 4 μl of 100 μl of Detection reagent containing 0.44 μg/ml of Streptavidin-Europium cryptate and 4 μg/ml of anti-6His-XL665 monoclonal antibody was added to the plate and incubated for 60 minutes at 23 °C. The Homogeneous Time Resolved Fluorescent (HTRF) signal was then measured on an Envision plate reader using and excitation wavelength of 337 nm and emission wavelengths of 615 nm and 665 nm. Normalized percent inhibition was then calculated using the following equation: % Inhibition normalized=(compound signal-min signal)/(max signal-min signal)*100%, where Maximum signal was substrate only wells and Minimum signal was enzyme and substrate well. IC₅₀ was calculated by fitting % Inhibition values and log of compound concentrations to nonlinear regression (log(inhibitor) vs. response—Variable slope(four parameters)) with GraphPad 9.4.1

For biochemical activity, bracket is denoted as: A<100 nM; 100 nM<B<500 nM; C>500 nM

| | PARG inhibition HTRF assay |
|---|---|
| Example | IC₅₀ (nM) |
| 1 | A |
| 2 | A |
| 3 | A |
| 4 | A |
| 5 | A |
| 6 | A |
| 7 | A |
| 8 | A |
| 9 | B |
| 10 | A |
| 11 | A |
| 12 | A |
| 13 | B |
| 14 | A |
| 15 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | C |
| 28 | B |
| 29 | B |
| 30 | C |
| 31 | B |
| 32 | A |
| 33 | B |
| 34 | A |
| 35 | A |
| 36 | A |
| 37 | A |
| 38 | A |
| 39 | A |
| 40 | A |
| 41 | A |

### Biological Example 2

### Cellular Viability Assay

Kuramochi cells were seeded at a density of 1000 cells per well in a 384-well, white, sterile, clear bottom plate, using a Multidrop liquid dispenser and incubated for 24 hours at 37 °C in a 5% CO₂ humidified environment. 24 hours later, compounds were added starting at a top dose of 10 μM for a 9-point dose response curve at a 1:3 dilution using an iDOT HT liquid handler and incubated for 5 days at 37 °C in a 5% CO₂ humidified environment. After 5 days and at Day 0, plates were incubated at room temperature for 30 minutes followed by the addition of 30 μl of CellTiter-Glo Reagent. Plates were then mixed for 10 minutes on an orbital shaker followed by incubation at room temperature for 20 minutes. Luminescence was then read using the Envision plate reader. Percent Growth Inhibition was then calculated using the following equation: Percent Growth Inhibition normalized=100−(compound signal−T0)/(max signal−T0)*100%, where Max Signal refers to the DMSO only treated cells and T0 refers to the Day0 viability measurement. Growth Inhibition 50 was then calculated using the following equation: Y=Bottom+(Top−Bottom)/(1+10\hat{ }((Log IC50−X)*hillslope)).

For cell activity, bracket is denoted as: A<1 uM; 1 uM<B<5 uM; C>5 uM.

| | 5 day Kuramochi cell viability |
|---|---|
| Example | IC₅₀ (uM) |
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | B |
| 14 | A |
| 15 | A |
| 16 | B |
| 17 | A |
| 18 | A |
| 19 | A |
| 20 | A |
| 21 | A |
| 22 | A |
| 23 | A |
| 24 | A |
| 25 | — |
| 26 | A |
| 27 | — |
| 28 | C |
| 29 | — |
| 30 | — |
| 31 | B |
| 32 | A |
| 33 | B |
| 34 | A |
| 35 | A |
| 36 | A |
| 37 | A |
| 38 | — |
| 39 | A |
| 40 | A |
| 41 | — |

The Summary an Abstract sections may be set forth one or more but not all exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

With respect to aspects of the invention described as a genus, all individual species are individually considered separate aspects of the invention. If aspects of the invention are described as “comprising” a feature, embodiments also are contemplated “consisting of” or “consisting essentially of” the feature.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments.

All of the various aspects, embodiments, and options described herein can be combined in any and all variations.

All publications, patents, and patent applications mentioned in this specification are herein incorporated by reference to the same extent as if each individual publication, patent, or patent application was specifically and individually indicated to be incorporated by reference. To the extent that any meaning or definition of a term in this document conflicts with any meaning or definition of the same term in a document incorporated by reference, the meaning or definition assigned to that term in this document shall govern.

What is claimed is:

1. A compound of Formula I, or a pharmaceutically acceptable salt thereof: 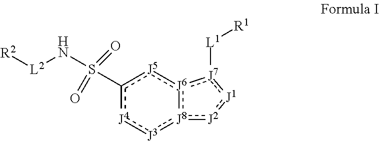
wherein:
J¹ is CR³, NR³, O, S, or N;
J² is CR⁴, NR⁴, O, S, or N;
J³ is CR⁵ or N;
J⁴ is CR⁶ or N;
J⁵ is CR⁷ or N;
J⁶ is C or N;
J⁷ is C or N;
J⁸ is C or N;
provided that the bicyclic ring containing J¹-J⁸ is a heteroaryl ring, preferably, the ring of J¹, J², J⁶, J⁷, and J⁸ has 1, 2, or 3 ring nitrogen atoms;
L¹ is null, an optionally substituted C₁₋₆ alkylene, an optionally substituted C₂₋₆ alkenylene, an optionally substituted C₂₋₆ alkynylene, an optionally substituted 3-8 membered ring, and R¹ is an optionally substituted 3-8 membered ring; or
L¹ is null or an optionally substituted 7-12 membered bicyclic ring structure; and R¹ is hydrogen, deuterium, halogen, CN, OH, NH₂, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-6 membered ring;
L² is an optionally substituted C₁₋₄ alkylene or an optionally substituted 3-5 membered carbocyclic or heterocyclic ring,
R² is hydrogen, deuterium, halogen, CN, OH, an optionally substituted C₁₋₄ alkyl, an optionally substituted C₂₋₄ alkenyl, or an optionally substituted C₂₋₄ alkynyl, or an optionally substituted 3-5 membered carbocyclic or heterocyclic ring
R³ is hydrogen, deuterium, halogen, CN, G¹, OG¹, NHG¹, NG¹G¹, C(O)G¹, C(O)NHG¹, or C(O)NG¹G¹;
R⁴ is hydrogen, deuterium, halogen, CN, an optionally substituted C₁₋₄ alkyl, an optionally substituted C₂₋₄ alkenyl, an optionally substituted C₂₋₄ alkynyl, an optionally substituted C₁₋₄ heteroalkyl, OG¹, NHG¹, NG¹G¹, or an optionally substituted 3-6 membered ring;
wherein G¹ at each occurrence is independently hydrogen, an optionally substituted C₁₋₄ alkyl, an optionally substituted C₂₋₄ alkenyl, an optionally substituted C₂₋₄ alkynyl, an optionally substituted C₁₋₄ heteroalkyl, or an optionally substituted 3-10 membered ring;
or two G¹ together with the nitrogen atom they are both attached to are joined to form an optionally substituted 4-10 membered heterocyclic ring;
R^{S} is hydrogen, halogen, CN, OH, G², or L³-G², wherein L³ is O, NH, CO, C(O)NH, C(O)N(C₁₋₄ alkyl), SO₂, SO₂NH, SO₂N(C₁₋₄ alkyl), an optionally substituted C₁₋₄ alkylene, an optionally substituted C₂₋₄ alkenylene, an optionally substituted C₂₋₄ alkynylene, or an optionally substituted C₁₋₄ heteroalkylene, and G² is an optionally substituted 3-14 membered ring; and
R⁶ and R⁷ are each independently hydrogen, deuterium, halogen, CN, or an optionally substituted C₁₋₄ alkyl.

2. The compound of claim 1, or a pharmaceutically acceptable salt thereof, characterized as having a structure according to Formula I-1:

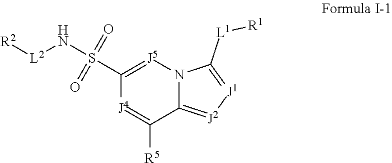

3. The compound of claim 1, or a pharmaceutically acceptable salt thereof, characterized as having a structure according to Formula I-2:

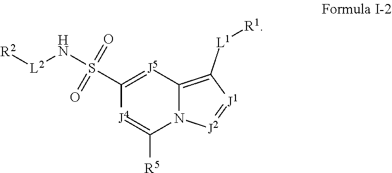

4. The compound of claim 1, or a pharmaceutically acceptable salt thereof, characterized as having a structure according to Formula I-3:

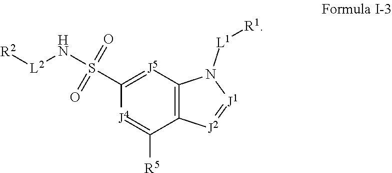

5. The compound of any of claims 1-4, or a pharmaceutically acceptable salt thereof, wherein J¹ is N.

6. The compound of any of claims 1-4, or a pharmaceutically acceptable salt thereof, wherein J¹ is CR³, preferably, CH.

7. The compound of any of claims 1-6, or a pharmaceutically acceptable salt thereof, wherein J² is N.

8. The compound of any of claims 1-6, or a pharmaceutically acceptable salt thereof, wherein J² is CR⁴, preferably, R⁴ is hydrogen or halogen, preferably, R⁴ is hydrogen, F or Cl.

9. The compound of any of claims 1-8, or a pharmaceutically acceptable salt thereof, wherein J⁴ is CH or CF.

10. The compound of any of claims 1-8, or a pharmaceutically acceptable salt thereof, wherein J⁵ is CH.

11. The compound of any of claims 1-8, or a pharmaceutically acceptable salt thereof, wherein J⁴ and J⁵ are both CH.

12. The compound of any of claims 1-11, or a pharmaceutically acceptable salt thereof, wherein L¹ is an optionally substituted 5-membered heteroarylene having 1-3 ring heteroatoms independently selected from S, N, and O, for example, an optionally substituted ring selected from thiazole, oxazole, imidazole, oxadiazole, or thiadiazole, or L¹ is an optionally substituted 6-membered heteroarylene having 1 or 2 ring nitrogens, such as an optionally substituted pyridylene or optionally substituted pyridazylene.

13. The compound of any of claims 1-11, or a pharmaceutically acceptable salt thereof, wherein L¹ is 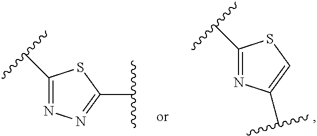 [SMILES: *c1nnc(*)s1 ; *c1csc(*)n1]
wherein the attaching point meta to the S atom is attached to R¹.

14. The compound of any of claims 1-11, or a pharmaceutically acceptable salt thereof, wherein L¹ is

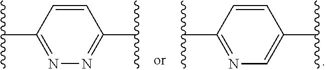 [SMILES: *c1ccc(*)nn1 ; *c1ccc(*)nc1]

15. The compound of any of claims 1-14, or a pharmaceutically acceptable salt thereof, wherein R¹ is a 3-4 membered ring optionally substituted with 1-3 substituents each independently selected from deuterium, F, OH, CN, C₁₋₄ alkyl, C₂₋₄ alkenyl, C₂₋₄ alkynyl, and C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH.

16. The compound of any of claims 1-15, or a pharmaceutically acceptable salt thereof, wherein R¹ is an optionally substituted cyclopropyl, preferably, when substituted, the cyclopropyl is substituted with 1-3 substituents each independently selected from F, OH, CN, or C₁₋₂ alkyl optionally substituted with 1-3 F, for example, R¹ is

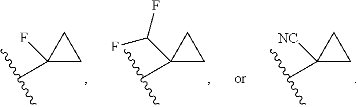

17. The compound of any of claims 1-14, or a pharmaceutically acceptable salt thereof, wherein L¹ is an optionally substituted 8-10 membered fused bicyclic ring structure having a first and second constituent ring, wherein the first constituent ring is a 5-membered heteroaryl ring having 1-3 ring heteroatoms independently selected from N, O, and S, and the second constituent ring is an aryl, heteroaryl, carbocyclic, or heterocyclic ring.

18. The compound of claim 17, or a pharmaceutically acceptable salt thereof, wherein L is an optionally substituted 8 or 9 membered fused bicyclic heteroaryl having a first and second constituent ring, wherein the first constituent ring is a 5-membered heteroaryl ring having 1-3 ring heteroatoms independently selected from N, O, and S, and the second constituent ring is phenyl, a 5-membered heteroaryl, or a 6-membered heteroaryl.

19. The compound of claim 17 or 18, or a pharmaceutically acceptable salt thereof, wherein L¹ attaches to J⁷ through a ring atom of the first constituent ring, and attaches to R¹ through a ring atom of the second constituent ring.

20. The compound of any of claims 17-19, or a pharmaceutically acceptable salt thereof, wherein the first constituent ring is a thiazole ring,

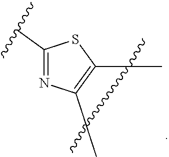

21. The compound of any of claims 17-20, or a pharmaceutically acceptable salt thereof, wherein the second constituent ring is a 5 or 6 membered heteroaryl, for example, L¹ is 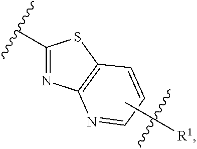
(R¹ is drawn to show direction of attachment).

22. The compound of any of claims 17-21, or a pharmaceutically acceptable salt thereof, wherein R¹ is hydrogen, deuterium, halogen (preferably, F or Cl), OH, CN, C₁₋₄ alkyl, C₂₋₄ alkenyl, C₂₋₄ alkynyl, or C₁₋₄ heteroalkyl, wherein the C₁₋₄ alkyl, C₂₋₄ alkenyl, C₂₋₄ alkynyl, or C₁₋₄ heteroalkyl is optionally substituted with 1-3 substituents independently selected from F and OH.

23. The compound of any of claims 1-14, or a pharmaceutically acceptable salt thereof, wherein L¹-R¹ is selected from the following:

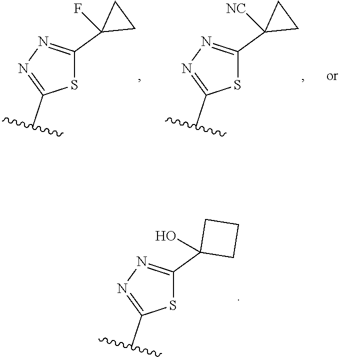 [SMILES: *c1nnc(C2(F)CC2)s1 ; *c1nnc(C2(C#N)CC2)s1 ; *c1nnc(C2(O)CCC2)s1]

24. The compound of any of claims 1-14, or a pharmaceutically acceptable salt thereof, wherein L¹-R¹ is selected from the following:

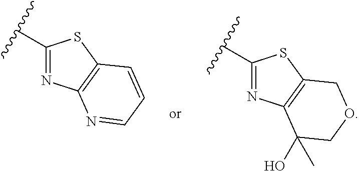 [SMILES: *c1nc2ncccc2s1 ; *c1nc2c(s1)COCC2(C)O]

25. The compound of any of claims 1-24, or a pharmaceutically acceptable salt thereof, wherein R⁵ is an optionally substituted 4-12 membered heterocyclic ring having 1-4 ring heteroatoms independently selected from N, O, and S, wherein the S atom is optionally oxidized.

26. The compound of any of claims 1-25, or a pharmaceutically acceptable salt thereof, wherein R⁵ is an optionally substituted 4-7 membered heterocyclic ring having 1-3 ring heteroatoms independently selected from N, O, and S, wherein the S atom is optionally oxidized.

27. The compound of claim 26, or a pharmaceutically acceptable salt thereof, wherein R⁵ is selected from the following: 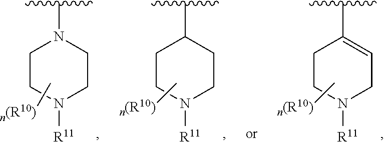
wherein:
n is an integer of 0-4,
(a) R¹⁰ at each occurrence is independently oxo, halogen (e.g., F), OH, CN, G^{A}, OG^{A}, C(O)G^{A}, SO₂G^{A}, P(O)G^{A}G^{A}, C(O)NHG^{A}, C(O)NG^{A}G^{A}, SO₂NHG^{A}, S(═NH)(═O)G^{A}, S(═N-G^{A})(═O)G^{A}, SO₂NG^{A}G^{A}, NHC(O)G^{A}, or N(G^{A})C(O)G^{A}, or
(b) two R¹⁰ are joined to form an optionally substituted 3-6 membered ring (e.g., cyclopropyl, cyclobutyl, or oxetane ring), and any remaining R¹⁰ are as defined in (a); or
(c) one R¹⁰ and R¹¹ are joined to form an optionally substituted 3-6 membered ring, and any remaining R¹⁰ are as defined in (a);
R¹¹ is hydrogen, G^{A}, C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}, or defined in (c) above;
wherein G^{A} at each occurrence is an optionally substituted group independently selected from (i) C₁₋₄ alkyl; (ii) C₁₋₄ heteroalkyl; and (iii) a 3-10 membered ring, or two G^{A} together with the intervening atom(s) are joined to form an optionally substituted 4-10 membered heterocyclic ring;
wherein when substituted, the C₁₋₄ alkyl or Cia heteroalkyl is preferably substituted with 1-3 substituents each independently (1) halogen (preferably F), CN, OH, or NH₂, (2) C₁₋₄ heteroalkyl optionally substituted with F; or (3) an optionally substituted 3-10 membered ring; and
when substituted, the 3-10 membered ring or the 4-10 membered heterocyclic ring is preferably substituted with 1-3 substituents independently (1) oxo (as applicable), halogen (e.g., F, Cl), CN, OH, or NH₂; (2) C₁₋₄ alkyl optionally substituted with F; (3) C₁₋₄ heteroalkyl optionally substituted with F; or (4) a 3-6 membered ring optionally substituted 1-3 substituents independently selected from oxo, F, Cl, CN, OH, C₁₋₄ alkyl optionally substituted with F, and C₁₋₄ heteroalkyl optionally substituted with F.

28. The compound of claim 27, or a pharmaceutically acceptable salt thereof, wherein n is 0.

29. The compound of claim 27 or 28, or a pharmaceutically acceptable salt thereof, wherein G^{A} at each occurrence is independently:
(1) a C₁₋₄ alkyl optionally substituted with 1-3 substituents each independently F, OH, C₁₋₄ alkoxy optionally substituted with 1-3 F, NH(C₁₋₄ alkyl), or N(C₁₋₃ alkyl)(C₁₋₃ alkyl); or
(2) a 3-10 membered ring, (C₁₋₄ alkylene)-(3-10 membered ring), or (C₁₋₄ heteroalkylene)-(3-10 membered ring), preferably, the 3-10 membered ring is a 3-6 membered ring selected from C₃₋₆ cycloalkyl, 4-6 membered heterocyclyl, 5 or 6-membered heteroaryl, or phenyl, wherein the 3-10 membered ring is optionally substituted with 1-3 substituents independently selected from oxo, F, Cl, OH, CN, C₁₋₄ alkyl optionally substituted with F, C₁₋₄ alkoxy optionally substituted with F, and optionally substituted 3-5 membered ring (e.g., cyclopropyl, cyclobutyl, azetidinyl, oxetanyl, etc.).

30. The compound of any of claims 27-29, or a pharmaceutically acceptable salt thereof, wherein R¹¹ is C(O)G^{A}, SO₂G^{A}, C(O)OG^{A}, C(O)NHG^{A}, C(O)NG^{A}G^{A}, SO₂NHG^{A}, S(═NH)(═O)G^{A}, S(═N-G^{A})(═O)G^{A}, or SO₂NG^{A}G^{A}.

31. The compound of claim 30, or a pharmaceutically acceptable salt thereof, where R¹¹ is selected from:

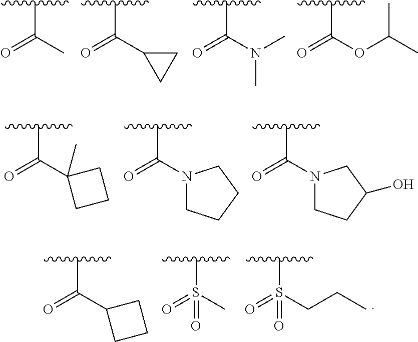

32. The compound of any of claims 27-29, or a pharmaceutically acceptable salt thereof, wherein R¹ is G^{A}, preferably, G^{A} is an optionally substituted 5 or 6-membered heteroaryl.

33. The compound of claim 32, or a pharmaceutically acceptable salt thereof, where R¹¹ is

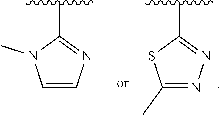

34. The compound of any of claims 1-25, or a pharmaceutically acceptable salt thereof, wherein R⁵ is an optionally substituted 7-12 membered heterocyclic ring having 1-3 ring heteroatoms independently selected from N, O, and S, wherein the S atom is optionally oxidized.

35. The compound of claim 34, or a pharmaceutically acceptable salt thereof, wherein R⁵ includes a spiro oxetane ring, for example, R⁵ can be represented by the structure of 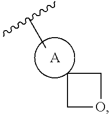
wherein ring A is a 4-8 membered carbocyclic or heterocyclic ring, which shares a single ring carbon atom with the oxetane ring.

36. The compound of claim 35, or a pharmaceutically acceptable salt thereof, wherein R⁵ is selected from:

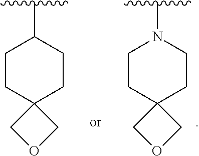 [SMILES: *C1CCC2(CC1)COC2 ; *N1CCC2(CC1)COC2]

37. The compound of any of claims 1-24, or a pharmaceutically acceptable salt thereof, wherein R⁵ is an optionally substituted 5 or 6 membered heteroaryl, e.g., pyrazole, when substituted, the 5 or 6 membered heteroaryl is preferably substituted with 1-3 substituents independently (1) halogen (e.g., F, Cl), CN, OH, or NH₂; (2) C₁₋₄ alkyl optionally substituted with F; (3) C₁₋₄ heteroalkyl optionally substituted with F; or (4) a 3-6 membered ring optionally substituted 1-3 substituents independently selected from oxo, F, Cl, CN, OH, C₁₋₄ alkyl optionally substituted with F, and C₁₋₄ heteroalkyl optionally substituted with F.

38. The compound of any of claims 1-37, or a pharmaceutically acceptable salt thereof, wherein L² is 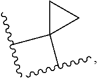 [SMILES: *C1(*)CC1]
optionally substituted with F, e.g.,

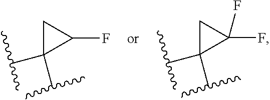 [SMILES: *C1(*)CC1F ; *C1(*)CC1(F)F]

39. The compound of any of claims 1-38, or a pharmaceutically acceptable salt thereof, wherein R² is hydrogen, F, C₁₋₂ alkyl optionally substituted with F (e.g., CH₃, CH₂F, CHF₂, or CF₃), CN, cyclopropyl, or C₂₋₃ alkynyl optionally substituted with F, such as

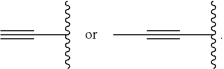

40. The compound of any of claims 1-38, or a pharmaceutically acceptable salt thereof, as applicable, characterized as having a structure according to Formula I-A: 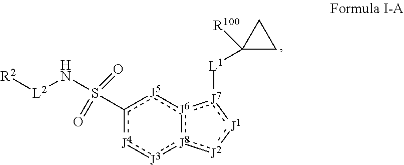
wherein:
R¹⁰⁰ is hydrogen, F, OH, CN, an optionally substituted alkyl, such as a C₁₋₄alkyl optionally substituted with deuterium or F (e.g., CH₃, CD₃, CH₂F, CHF₂, etc.), or an optionally substituted heteroalkyl, such as a C₁₋₄ heteroalkyl optionally substituted with deuterium or F.

41. The compound of claim 40, or a pharmaceutically acceptable salt thereof, as defined in any of Embodiments A2-29 herein.

42. The compound of any of claims 1-38, or a pharmaceutically acceptable salt thereof, as applicable, characterized as having a structure according to Formula I-D: 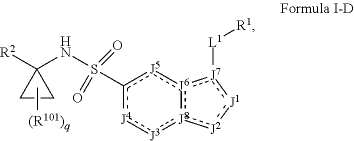
wherein:
q is 1 or 2;
R¹⁰¹ is F or methyl optionally substituted with F.

43. The compound of claim 42, or a pharmaceutically acceptable salt thereof, as defined in any of Embodiments B2-43 herein.

44. The compound of any of claims 1-38, or a pharmaceutically acceptable salt thereof, as applicable, characterized as having a structure according to Formula I-D: 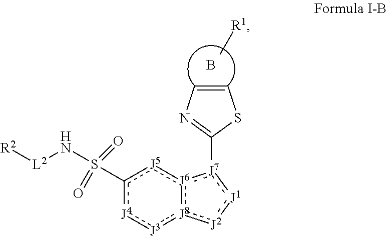
wherein:
Ring B is an optionally substituted 5 or 6 membered heterocyclyl or heteroaryl ring having 1-3 ring heteroatoms independently N, O, or S;
R¹ is hydrogen, deuterium, halogen, CN, OH, NH₂, an optionally substituted C₁₋₄ alkyl, an optionally substituted C₁₋₄ heteroalkyl, or an optionally substituted 3-6 membered ring.

45. The compound of claim 44, or a pharmaceutically acceptable salt thereof, as defined in any of Embodiments C2-26 herein.

46. A compound selected from Table 1, or Example Nos. 1-41, or a pharmaceutically acceptable salt thereof.

47. A pharmaceutical composition comprising the compound of any of claims 1-46, or a pharmaceutically acceptable salt thereof.

48. A method of treating a disease or disorder in which PARG activity is implicated, the method comprising administering an effective amount of the compound of any of claims 1-46, or a pharmaceutically acceptable salt thereof, or the pharmaceutical composition of claim 47.

49. The method of claim 48, wherein the disease or disorder is cancer.
